(19) United States
(12) Patent Application Publication
(10) Pub. No.: US 2009/0048194 A1
(43) Pub. Date: Feb. 19, 2009

(54) Vagal Afferent Neurons as Targets for Treatment

(75) Inventors: Jeroen Marcel Maria Roger Aerssens, Nieuwrode (BE); Pieter Johau Peeters, Dessel (BE); Ann Louise Gabrielle Meulemans, Mol (BE); Bernard Coulie, Herent (BE); Kirk Hillsley, Millbrook (CA); David Grundy, Sheffield (GB); Ronald Stead, Bowmanville (CA)

Correspondence Address: WOODCOCK WASHBURN LLP, CIRA CENTRE, 12TH FLOOR, 2929 ARCH STREET, PHILADELPHIA, PA, 19104-2891, US

(73) Assignee: Janssen Pharmaceutica N.V., Beerse (BE)

(21) Appl. No.: 11/815,688

(22) PCT Filed: Feb. 8, 2006
(86) PCT No.: PCT/GB06/00435

§ 371 Date: Mar. 19, 2008

Related U.S. Application Data
(60) Provisional application No. 60/650,868, filed on Feb. 8, 2005.

(30) Foreign Application Priority Data
Feb. 8, 2005 (GB) 0502588.7

(51) Int. Cl. A61K 31/70; C12Q 1/68; C40B 30/04; A61P 1/00; C40B 40/08

(52) U.S. Cl. 514/44; 435/6; 506/9; 506/17

(57) ABSTRACT

A method of identifying a compound capable of reducing or preventing prolonged sensory neuron hyper-excitability comprising the steps of: (a) administering the compound to an experimental non-human animal having prolonged sensory neuron hyper-excitability; (b) generating an expression profile of the genes modulated in the Nodose Ganglia (NG) of the animal of step (a); (c) comparing the expression profile obtained in (b) with the expression profile of a corresponding panel of genes expressed in the NG of an experimental non-human animal having no prolonged sensory neuron hyper-excitability; wherein a positive correlation of the expression profiles is indicative that the compound is capable of reducing or preventing prolonged sensory neuron hyper-excitability in NG.

### FIELD OF INVENTION

The present invention relates to the treatment of sensory neuron hyper-excitability in Nodose Ganglia (NG), methods for the identification of compounds suitable for this application and pharmaceutical compositions comprising these compounds, as well as their uses in the treatment of G.I tract disorders, depression and other stress related disorders.

### BACKGROUND

### Vagal Versus Spinal Visceral Afferents

The gastrointestinal (GI) tract receives dual extrinsic sensory innervation. Vagal afferents have their cell bodies in the nodose ganglia (NG) and project centrally to make synaptic connections in the brainstem, mainly at the level of the nucleus tractus solitarius; while spinal afferents arise from the dorsal root ganglia (DRG) and project into the dorsal horn of the spinal cord (Grundy D., Gut 2002; 51 Suppl 1:i2-i5). These two types of neurons have different embryonic origins (epibranchial placode versus neural crest), different dependencies upon neurotrophic factors for development and survival (BDNF/NT3 versus NGF/GDNF) and, in the adult form, phenotypically distinct subpopulations that can be recognized by the presence or absence of certain peptides (particularly CGRP and substance P) (Zhuo H. Ichikawa H, Helke C J., 1997; 52:79-107).

Vagal and spinal afferents supplying the GI tract also differ in the pattern of their terminal innervation which, in part determines the stimulus-response properties of the peripheral endings (Berthoud H R, Blackshaw L A, Brookes S J, Grundy D., 2004; 16 Suppl 1:28-33). Vagal afferents terminate close to the mucosal epithelium, where they are exposed to chemicals (e.g. nutrients) absorbed from the lumen or mediators released from enteroendocrine cells or immune cells in the lamina propria. These vagal afferents are termed chemosensitive and can respond to a variety of different chemical stimuli. Vagal afferents also form intramuscular arrays and intraganglionic laminar endings that are thought to detect mechanical activity. Spinal afferents also innervate the mucosa, submucosa and myenteric plexus. Additionally, projections of DRG neurons terminate in the serosa and mesenteric attachments, often in association with blood vessels. These endings are mechanosensitive but the basis of this mechanosensitivity at the molecular level is unknown. Both vagal and spinal afferents respond to distension and contraction but while vagal afferent endings respond to levels of distension that occur during the normal course of digestion, many spinal afferents have thresholds for activation that when applied in humans give rise to discomfort or pain (Gebhart G F., Gut 2000; 47 Suppl 4:iv54-iv55).

These observations are the basis for the common view that vagal and spinal afferents have different functional roles: spinal afferents play a major role in nociception, while vagal afferents mediate physiological responses and behavioural regulation, particularly in a chemosensitive role, in relation to food intake, satiety, anorexia and emesis. However, there is some overlap, and vagal and spinal afferents share a number of features in common. Both have a large proportion of unmyelinated axons that can be ablated by the sensory neurotoxin, capsaicin; and both express the capsaicin receptor (TRPV1) that is often considered a hallmark of nociceptive neurons (Ward S M, Bayguinov J, Won K J, Grundy D, Berthoud H R., J Comp Neurol 2003; 465:121-135). In addition, chemosensitive vagal afferent neurons can also play a nociceptive role in acid signalling (Holzer, P., J Physiol Pharmacol 2003; 54(4), 43-53). Recently, both NG and DRG neurons have been shown to become sensitized following inflammatory insult, demonstrating plasticity in the mechanisms that regulate neuronal excitability which has implications for pain processing (Dang K, Bielefeldt K, Gebhart G F., Am J Physiol Gastrointest Liver Physiol 2004; 286:G573-G579). As both NG and DRG neurons are altered following an inflammatory insult, it is possible that there is both altered chemosensitivity and altered mechanosensitivity in the post-inflammatory gut. Furthermore, there may be an interaction between changes in chemosensitive afferent pathways and changes in mechanosensitive afferent pathways.

Therefore, extrinsic afferent neurons supplying the gut are prime targets for new treatments of chronic visceral pain disorders such as IBS. The pathogenesis of IBS is heterogeneous but there are at least subpopulations of patients where emotional stress and/or enteric infection have been implicated.

Nippostrongylus brasiliensis Infection as a Model for IBS

Brain-gut interactions play a prominent role in the modulation of gut function in health and disease (Mayer E A, Naliboff B D, Chang L, Coutinho S V. V., Am J Physiol Gastrointest Liver Physiol 2001; 280:G519-G524; Tache Y, Martinez V, Million M, Wang L., Am J Physiol Gastrointest Liver Physiol 2001; 280:G173-G177). Therefore, every conceptual model of irritable bowel syndrome (IBS) should take into account that the central nervous system (CNS) and the GI-tract interact with each other under normal conditions and certainly during perturbations of homeostasis. Afferent signals from the gut to the brain (through splanchnic and vagal routes) are primarily involved in reflex regulation of gut function, but may also play an important role in such diverse functions as regulation of emotion, pain sensitivity and immune responses. Conversely, signals from the brain to the gut assure that digestive function is optimal for the overall state of the organism (e.g. stress vs relaxation, sleep vs awake). The fact that the presence of major life events around the time of gastroenteric infection is a risk factor for the development of PI-IBS symptoms underlines the importance of psycho-neuro-immune interactions.

By including a stress paradigm into an animal model we take into account this important aspect of IBS. As a trigger for the development of IBS a mild gastroenteric infection was induced using the nematode Nippostrongylus brasillensis. The neural and cellular changes that occur following intestinal infection have been reasonably well documented. However, the physiological consequences of these changes are not well understood particularly in terms of the post-inflammatory changes which accompany intestinal recovery. Post-inflammatory jejunal hypersensitivity has been reported in the capsaicin-induced depressor response in rats previously infected (day 40-50) with Nippostrongylus brasiliensis (Mathison R, Davison J S., Naunyn Schmiedebergs Arch Pharmacol 1993; 348:638-642). The afore mentioned study is pivotal as it shows that increased sensitivity can be observed in the absence of acute inflammation and this is relevant to and predictive of the pathophysiology of IBS. Indeed, the post-inflammatory changes which occur in the rat intestine post-infection with N. brasiliensis putatively parallel the pathophysiology of IBS (Camilleri M., Drug News Perspect 2001; 14:268-278) and have been shown to include a variety of neuroimmune changes (Stead R H., Ann N Y Acad Sci 1992; 664:443-455). In rats it has been shown that infection with N. brasiliensis leads to changes in intestinal mast cell number and peptidergic neurotransmission eventually leading to visceral hyperalgesia (McLean P G, Picard C, Garcia-Villar R, Ducos dL, More J, Fioramonti J, Bueno L., Neurogastroenterol Motil 1998; 10:499-508). Moreover these neuroimmune alterations lead to an increased intestinal motility response to CCK that involves a vagal pathway probably through CCK_{A} and CCK_{B} receptors (Gay J, Fioramonti J, Garcia-Villar R, Bueno L., Neurogastroenterol Motil 2001; 13:155-162; Gay J, More J, Bueno L, Fioramonti J., Brain Res 2002; 942:124-127).

The present invention is based on the unexpected discovery by the inventors that after transient inflammation of the intestine induced by the nematode Nippostrongylus brasiliensis in mice combined with exposure to stress, gene expression profiles and electrophysiological properties of NO neurons projecting in to the gastrointestinal tract are altered

The discovery is surprising because it has been previously shown that the activity of voltage sensitive sodium channels in DRG neurons in mice is increased after transient inflammation of the intestine with Nb, implicating DRG neurons in a variety of conditions resulting in chronic inflammatory and neuropathic pain.

### SUMMARY OF THE INVENTION

According to a first aspect of the present invention there is provided a method of identifying a compound capable of reducing or preventing prolonged sensory neuron hyper-excitability comprising the steps of:

(a) administering the compound to an experimental non-human animal having prolonged sensory neuron hyper-excitability;

(b) generating an expression profile of the genes modulated in the Nodose Ganglia (NG) of the animal of step (a);

(c) comparing the expression profile obtained in (b)) with the expression profile of a corresponding panel of genes expressed in the NG of an experimental non-human animal having no prolonged sensory neuron hyper-excitability;

wherein a positive correlation of the expression profiles is indicative that the compound is capable of reducing or preventing prolonged sensory neuron hyper-excitability in NG.

It will be apparent that modulation of the expression of NG genes may be either up-regulation or down-regulation of expression. As used herein the term “expression profile” relates to methods that are able to outline the expression levels of various genes either at the transcript level or the protein level. Expression profiles can be obtained for example by Northern blot analysis, Western blot analysis, immunohistochemistry, in situ hybridization or other methods known in the art such as for example described in Sambrook et al. (Molecular Cloning; A laboratory Manual, Second Edition, Cold Spring Harbour Laboratory Press, Cold Spring Harbour NY (1989)) or in Schena (Science 270 (1995) 467-470). Most preferably “expression profile” herein relates to methods using microarrays as e.g. described in the examples hereinafter.

Preferably, the modulation of genes expressed in the NG is compared at the nucleic acid level, in particular at the mRNA level.

It will be apparent to the skilled person that in order to obtain the NG for expression profiling the non human experimental animal is sacrificed.

It will be understood that the genes that are compared are genes whose expression is altered by at least 10%, more preferably the expression is altered by at least 25%, most preferably, the expression is altered by at least 50% in animals having prolonged sensory neuron hyper-excitability. As aforesaid, the expression may be up-regulated or down-regulated.

Preferably, the panel of prolonged sensory neuron hyper-excitability modulated genes are selected from the group consisting of those genes disclosed in Table 1 as shown at the end of the description.

Preferably, the prolonged sensory neuron hyper-excitability modulated signal compared comprises the expression level of at least one nucleic acid sequence encoding a receptor selected from the group consisting of the vanilloid receptor VR1 (Trpv1), cholecystokinin receptor A (Cckar), serotonin receptor 3A (Htr3a) and somatostatin 2 receptor (Sstr2). More preferably, the panel of prolonged sensory neuron hyper-excitability modulated signals compared comprises the expression level of at least nucleic acid molecules encoding the vanilloid receptor VR1 (Trpv1), cholecystokinin receptor A (Cckar), serotonin receptor 3A (Htr3a) and somatostatin 2 receptor (Sstr2). Most preferably, the method comprises comparing the expression of a panel of at least 40 nucleic acid sequences encoding genes having modulated expression in NG associated with having prolonged sensory neuron hyper-excitability. In a more particular embodiment, the method comprises comparing an expression panel of prolonged sensory neuron hyper-excitability modulated genes selected from the group consisting of those genes disclosed in Table 1. A particularly preferred panel of 51 genes whose expression is to be compared is shown in Table 2 below.

In a preferred embodiment of the invention the expression profile of prolonged sensory neuron hyper-excitability modulated genes is assessed at the mRNA level. It will be understood that the presence of the at least 1 nucleic acid molecule may be detected on the basis of a probe capable of hybridizing thereto which may be affixed to a solid support. A panel of probes capable of hybridizing to a panel of nucleic acids can be affixed to a solid support in an arrayed form as described hereinafter.

In a preferred embodiment of the invention, labelled mRNA is hybridized against a panel of different nucleic acids representing or comprising genes expressed in the NG. The term “labelled mRNA” herein refers to methods of labelling mRNA which for example can be performed by fluorescence-labelling using fluorescent dyes or by autoradiographic labeling using e.g. ³²P or ³³P. Labelling methods are well known by those skilled in the art and are described (Sambrook et al., supra; Ausubel et al., supra, Eisen and Brown, Methods Enzymology 303 (1999), 179-205).

The mRNA labelled as indicated above is hybridized against a panel of different “nucleic acids representing or comprising genes” expressed in the NG. The term “nucleic acids representing or comprising genes” denotes for example oligonucleotides, cDNAs, PCR fragments amplified from ORFs, or any other polymeric form of nucleotides of any length, either ribonucleotides or deoxyribonucleotides.

In a preferred embodiment of the invention said panel of different nucleic acids is affixed to a solid support. The solid support herein can be for example represented by polylysine-treated glass slides or activated slides that allow single strand covalent amino-mediated binding of cDNA, however, is not limited to those (Blohm and Guiseppi-Elie, Current Opinion Biotechnology 12 (2001), 41-47).

In a preferred embodiment of the invention said panel of different nucleic acids is affixed to said solid support in arrayed form. The construction of microarrays is described e.g. in the examples hereinafter or in Marton (Nature Medicine 4 (1998), 1293-13).

Any non-human animal model of prolonged sensory neuron hyper-excitability is suitable for use in the screening methods of the present invention. Exemplified herein is a method in which a rodent is infected with Nippostrongylus brasiliensis and subjected to stress. Intestinal inflammation is induced by the infection but once the inflammation has subsided prolonged sensory neuron hyper-excitability remains. These post-inflammatory changes parallel the pathology of human irritable bowel syndrome (IBS). There are however a number of other methods of modelling G. I. tract disorders in which intestinal inflammation can be induced with attendant prolonged sensory neuron hyper-excitability using a variety of infectious or non-infectious agents all of which would be suitable for use in the screening method of the invention and which may or may not be combined with the attendant application of stress.

For example, the relevant inflammatory response can be induced by other parasites, particularly Helminths such as Heligmosomoides polygyrus, Trichuris muris or Leishmania major. Other suitable parasitic Helminths are identified in the Table 3 below.

The prolonged sensory neuron hyper-excitability may begin and end at different times after the initial infection, depending upon the nature and life cycle of the infectious agent and may be further enhanced by repeated or subsequent infections or other factors (physical and chemical stressors—see below).

TABLE 2

| | | | Log2Ratio. | | |
|---|---|---|---|---|---|
| | Gene | | Median.IS. | | |
| Probe Set ID | Symbol | Title | over.SNS | GenBank ID | SwissProt ID |
| 1421195_at | Cckar | cholecystokinin A receptor | 0.80 | BC020534 | O08786 |
| 1437029_at | Tacr3 | tachykinin receptor 3 | 1.65 | AV328460 | AAH66845 /// P47937 |
| 1443392_at | Trpv1 | vanilloid receptor 1 | 1.10 | BB346256 | — |
| 1418268_at | Htr3a | serotonin receptor 3A | −0.38 | NM_013561 | P23979 /// Q8K1F4 |
| 1459850_x_at | Glrb | glycine receptor, beta subunit | 0.66 | BB345174 | BAC38831 /// P48168 |
| 1417489_at | Npy2r | neuropeptide Y receptor Y2 | 1.23 | NM_008731 | P97295 /// Q8BWV1 |
| 1420799_at | Ntsr | neurotensin receptor | −0.71 | NM_018766 | O88319 |
| 1426204_a_at | Oprl | opioid receptor-like | 0.63 | AF043276 | BAC30067 /// BAC37672 /// P35377 |
| | | | | | /// Q80WU7 |
| 1427331_at | Adoral | adenosine A1 receptor | 0.85 | BB518868 | CAD88592 /// Q60612 /// Q8BGU7 /// |
| | | | | | Q8CAH1 /// Q8R0M5 |
| 1434172_at | Cnr1 | cannabinoid receptor 1 (brain) | 1.44 | BQ177934 | — |
| 1433602_at | Gabra5 | gamma-aminobutyric acid (GABA-A) receptor, | 0.71 | BQ175863 | AAH62112 /// O88964 /// Q8BHJ7 |
| | | subunit alpha 5 | | | |
| 1435021_at | Gabrb3 | gamma-aminobutyric acid (GABA-A) receptor, | 0.74 | BQ175666 | BAC30230 /// P15433 /// Q8C446 |
| | | subunit beta 3 | | | |
| 1437968_at | Grin1 | glutamate receptor, ionotropic, NMDA1 (zeta 1) | −1.17 | AI385669 | P35438 /// Q8BZ96 /// Q8CFS4 |
| 1421530_a_at | Grm8 | glutamate receptor, metabotropic 8 | 0.60 | NM_008174 | P47743 |
| 1438613_at | Kcna4 | potassium voltage-gated channel, shaker-related | 0.72 | BB131475 | Q8CBF8 |
| | | subfamily, member 4 | | | |
| 1439204_at | Scn3a | sodium channel, voltage-gated, type III, alpha | 0.85 | BB096886 | Q62204 |
| | | polypeptide | | | |
| 1454768_at | Kcnf1 | potassium voltage-gated channel, subfamily F, | 1.15 | AV337635 | Q7TSH7 |
| | | member 1 | | | |
| 1438093_x_at | Dbi | diazepam binding inhibitor | 0.75 | BB115327 | BAB25730 /// BAB25755 /// BAB32175 |
| | | | | | /// BAC25658 /// P31786 |
| 1420596_at | Cacng2 | calcium channel, voltage-dependent, gamma | −1.18 | NM_007583 | O88602 /// Q8C8F5 |
| | | subunit 2 | | | |
| 1427418_a_at | Hifla | hypoxia inducible factor 1, alpha subunit | 1.37 | X95580 | Q61221 |
| 1449544_a_at | Kcnh2 | potassium voltage-gated channel, subfamily H | −0.77 | NM_013569 | AAQ82708 /// O35219 /// Q80WG1 |
| | | (eag-related), member 2 | | | /// Q80XE8 |
| 1439618_at | Pde10a | phosphodiesterase 10A | 1.51 | AI448308 | Q8C8M0 /// Q8CA95 /// Q9WVI1 |
| 1451707_s_at | Slc41a3 | solute carrier family 41, member 3 | −0.98 | BC011108 | Q921R8 /// Q9DC67 |
| 1437864_at | Adipor2 | adiponectin receptor 2 | −0.70 | BE632137 | AAR08379 /// Q8BQS5 |
| 1437259_at | Slc9a2 | solute carrier family 9 (sodium/hydrogen | 0.80 | AV274006 | Q9WUJ4 |
| | | exchanger), member 2 | | | |
| 1433536_at | Lrp11 | low density lipoprotein receptor-related protein | 1.65 | BB435348 | AAH59874 /// Q8CB67 |
| | | 11 | | | |
| 1457164_at | Anktm1 | ANKTM1 | 1.10 | BB309395 | Q8BLA8 |
| 1420609_at | Axot | axotrophin | −0.38 | NM_020575 | Q9WV66 |
| 1451840_at | Calp | calsenilin-like protein | 0.66 | BG261945 | AAH51130 /// Q8CAD0 /// Q8R4I2 |
| | | | | | /// Q9EQ01 |
| 1422659_at | Camk2d | calcium/calmodulin-dependent protein kinase II, | 1.23 | NM_023813 | AAH52894 /// O70459 /// Q8C3F8 /// |
| | | delta | | | Q8C4I3 /// Q8C8X9 /// Q8CAC5 /// |
| | | | | | Q8CCM0 /// Q9CZE2 |
| 1434034_at | Cerk | ceramide kinase | −0.71 | BI905090 | BAC98226 /// Q8K4Q7 |
| 1449403_at | Pde9a | phosphodiesterase 9A | 0.63 | NM_008804 | AAH61163 /// O70628 /// Q8BSU4 /// |
| | | | | | Q8CB29 |
| 1416013_at | Pld3 | phospholipase D3 | 0.85 | NM_011116 | O35405 |
| 1437861_s_at | Prkce | protein kinase C, epsilon | 1.44 | BB335101 | P16054 |
| 1416339_a_at | Prkcsh | protein kinase C substrate 80K-H | 0.71 | NM_008925 | O08795 /// Q92IX2 |
| 1416294_at | Scamp3 | secretory carrier membrane protein 3 | 0.74 | NM_011886 | O35609 |
| 1418738_at | Scn1b | sodium channel, voltage-gated, type I, beta | −1.17 | BC009652 | P97952 |
| | | polypeptide | | | |
| 1420822_s_at | Sgpp1 | sphingosine-1-phosphate phosphatase 1 | 0.60 | NM_030750 | Q9JI99 |
| 1417622_at | Slc12a2 | solute carrier family 12, member 2 | 0.72 | BG069505 | P55012 |
| 1417600_at | Slc15a2 | solute carrier family 15 (H+/peptide transporter), | 0.85 | NM_021301 | Q80XC0 /// Q8VEK9 /// Q9CXC0 /// |
| | | member 2 | | | Q9JM03 |
| 1448502_at | Slc16a7 | solute carrier family 16 (monocarboxylic acid | 1.15 | NM_011391 | BAC36415 /// O70451 |
| | | transporters), member 7 | | | |
| 1418843_at | Slc30a4 | solute carrier family 30 (zinc transporter), | 0.75 | NM_011774 | O35149 |
| | | member 4 | | | |
| 1419971_s_at | Slc35a5 | solute carrier family 35, member A5 | −1.18 | C86506 | Q921R7 /// Q9DC72 |
| 1453915_a_at | Slc37a3 | solute carrier family 37 (glycerol-3-phosphate | 1.37 | AK012071 | Q8BVX2 /// Q99JR0 |
| | | transporter), member 3 | | | |
| 1454764_s_at | Slc38a1 | solute carrier family 38, member 1 | −0.77 | BF165681 | AAH66815 /// Q8BHI3 /// Q8BXE2 /// |
| | | | | | Q8K2P7 /// Q99PR1 |
| 1426432_a_at | Slc4a4 | solute carrier family 4 (anion exchanger), | 1.51 | BE655147 | O88343 /// Q8QZR9 /// Q9R1C4 |
| | | member 4 | | | |
| 1438673_at | Slc4a7 | solute carrier family 4, sodium bicarbonate | −0.98 | AW555750 | Q8BTY2 /// Q8BWZ4 /// Q9JL09 |
| | | cotransporter, member 7 | | | |
| 1428954_at | Slc9a3r2 | solute carrier family 9 (sodium/hydrogen | −0.70 | AK004710 | AAH65778 /// Q9JHL1 |
| | | exchanger), isoform 3 regulator 2 | | | |
| 1428460_at | Syn2 | synapsin II | 0.76 | AK013810 | AAH66004 /// Q8CE19 /// Q9QWV7 |
| 1415844_at | Syt4 | synaptotagmin 4 | 0.78 | AV336547 | P40749 |
| 1440882_at | Lrp8 | low density lipoprotein receptor-related protein | 1.11 | BB750940 | Q924X6 |
| | | 8, apolipoprotein e receptor | | | |

TABLE 3

| Phylum | Class and order | Family | Genus and Species | Disease |
|---|---|---|---|---|
| Nematoda | Adenophorea; Enoplida | Trichinellidae | Trichinella spiralis | Trichinosis |
| | | (muscleworms) | | |
| | | Trichuridae (whipworms) | Trichuris trichiura | Trichuriasis |
| | Secernentea; Rhabditida | Strongyloididae | Strongyloides stercoralis | Strongyloidiasis |
| | | (threadworms) | | |
| | Secernentea; Strongylida | Ancylostomatoidea | Ancylostoma duodenale | Hookworm |
| | | (hookworms) | | disease |
| | | | Necator americanus | |
| | Secernentea; Ascaridida | Ascarididae | Ascaris lumbricoides | Ascariasis |
| | | (roundworms) | | |
| | | Onchocercidae (filarids) | Wuchereria bancrofti | Filariasis |
| | | | Brugia malayi | |
| Platyhelminthes | Trematoda; Strigeatoida | Schistosomatidae | Schistosoma mansoni | Schistosomiasis |
| | (flukeworms) | | | |
| | | | Schistosoma japonicum | |
| | | | Schistosoma | |
| | | | haematobium | |
| | | Fasciolidea | Fasciola hepatica | Fascioliasis |
| | Cestoidea; Cyclophyllidea | Taeniidae | Taenia solium | Cysticercosis |
| | (tapeworms) | | | |
| | | | Enchinococcus granulosus | Hydatid cyst |

The immunopathology of various of these parasites is described in Gause et al, Trends in Immunology, Vol. 24, No. 5, May 2003.

Other infective agents suitable for inducing inflammatory conditions in the intestinal mucosa of a non-human animal include bacteria such as Campylobacter species, Helicobacter species and E. coli. Since the inflammation may be generated by antigenic determinants or toxins carried by the bacteria, the model may involve the administration of bacteria either dead or alive or the administration of individual inflammatory antigens, such as known bacterial toxins.

Other non-human animal models of prolonged sensory neuron hyper-excitability for use in the invention include those where an irritant material is administered to the intestine at some time prior to assessment of sensory neuron hyper-excitability. Suitable materials include a material selected from the group including: dinitrochlorobenzene, trinitrobenzene sulphonic acid, dinitrobenzene suphonic acid, acetic acid, mustard oil, dextran sodium sulphate, croton oil, carageenan, amylopectin sulphate, oxazalone and indomethacin.

The experimental non-human animal having prolonged sensory neuron hyper-excitability as used herein relates to other known non-human animal models of mucosal inflammation, such as those used to study the pathogenesis of inflammatory bowel disease, such as for example described in Strober et al. (Annu. Rev. Immunol. 2002 20:495-549) and the post-inflammatory states arising therefrom.

Further non-human animal models may also be used in the screening method of the invention where the non-human animal has a particular genetic background or carries a genetic defect or has been otherwise engineered (e.g. a transgenic animal) to exhibit intestinal inflammation and prolonged sensory neuron hyper-excitability.

Examples of genetic background differences in non-human animals include the different responses to various somatic and visceral painful stimuli exhibited by different strains of mice (Mogil et al., Pain 1999; 80:67-82; Kamp et al., Am. J. Physiol., 2003; 284:G434-G444); the heightened sensitivity to wrap restraint and water avoidance exhibited by Fischer rats when compared to Sprague Dawley and Lewis rats, respectively; and the well described depressive phenotype of Flinders rats (Yadid et al., Prog. Neurobiol. 2000; 62:353-378) that results in enhanced viscero-motor responses to colorectal distension (Eisenbruch et al., Neurogastroenterol. Mot. 2004; 16:801-809).

Examples of genetic defect or engineered models are:

Tgₑ26 mice
TCR-α chain deficiency
TNF^{ΔΔRE} mice (TNF-α overproduction)
WASP deficiency
C₃H/HeJBir mice
N-cadhaerin dominant-negative mice
Gi2α-deficient mice
IL-2 deficient mice
Samp1/Yit mice
T-bet Tg mice
STAT4 Tg mice
TGF-β RII dominant-negative Tg mice
HLA-B27 Tg rats
Mdr1α-deficient mice
IL-7 Tg mice

The non-human animal may be a mouse, rat or other rodent, guinea pig, cat, dog, or non-human primate. The aforementioned models of mucosal inflammation may be operated with or without the concurrent application of stress to the animal. Alternatively, stress to the animal may in itself be sufficient to cause prolonged sensory neuron hyper-excitability and accordingly useful in the methods of the invention. Stress may be applied in a number of ways, for example, over-crowded housing, poor handling, absence of tubes or gauze in a cage. Other stressors that may be employed are known in the art as described by Mayer et al.(supra) and Tache et al. (supra) and include: neonatal colonic irritation, maternal separation, foot shock, open field, loud noise, water avoidance, tail shock, wrap restraint, cold water swim, exposure to cold or heat and other environmental stimuli. Such stressors may be employed alone, in combination with each other and/or in combination with inflammation.

In an alternative embodiment this invention provides the comparison of the expression profiles of the prolonged sensory neuron hyper-excitability modulated genes in cell populations capable of expressing one or more of said genes disclosed in Table 1, preferably capable of expressing one or more of said genes disclosed in Table 2, more preferably in cell populations expressing at least one nucleic acid sequence encoding a receptor selected from the group consisting of the vanilloid receptor VR1 (Trpv1), cholecystokinin receptor A (Cckar), serotonin receptor 3A (Htr3a) and somatostatin 2 receptor (Sstr2). More preferably the invention involves comparing the expression profiles of at least nucleic acid molecules encoding the vanilloid receptor VR1 (Trpv1), cholecystokinin receptor A (Cckar), serotonin receptor 3A (Htr3a) and somatostatin 2 receptor (Sstr2). Most preferably the invention involves comparing the expression of a panel of at least 40 nucleic acid sequences encoding genes having modulated expression in NG associated with having prolonged sensory neuron hyper-excitability. A particularly preferred panel of genes whose expression is to be compared is shown in Table 2 supra.

In this alternative embodiment the expression profiles are compared between a test cell, i.e. a cell population known to have an expression profile as observed in the NG of the non-human animal having prolonged sensory neuron hyper-excitability with a reference cell population, i.e. a cell population known to have an expression profile as observed in the NG of the non-human animal not having prolonged sensory neuron hyper-excitability.

Accordingly in a second aspect the invention provides a method for identifying a compound capable of reducing or preventing prolonged sensory neuron hyper-excitability comprising the steps of:

(a) administering the compound to a test cell population;

(b) generating an expression profile of the prolonged sensory neuron hyper-excitability modulated genes in the cell population of step (a);

(c) comparing the expression profile obtained in (b) with the expression profile of the prolonged sensory neuron hyper-excitability modulated genes in a reference cell population;

wherein a positive correlation of the expression profiles is indicative that the compound is capable of reducing or preventing prolonged sensory neuron hyper-excitability in NG.

Preferably the test cell population is derived from the NG of an experimental non-human animal having prolonged sensory neuron hyper-excitability, and the reference cell population is derived from the NG of an experimental non-human animal not having prolonged sensory neuron hyper-excitability. More preferably the cell populations are derived from the NG of a rodent, in particular mice.

It is also an object of the present invention to provide the use of NG sensory neuron activity assays in a method to identify compounds capable of reducing or preventing prolonged sensory neuron hyper-excitability. Such assays are known in the art and typically involve measurement of ionic currents using either

i) electrophysiological techniques such as for example using two-electrode voltage clamp recordings rascal N. (1987) Crit. Rev. Biochem 22, 341-356), patch-clamp recordings (Zhou Z. et al., (1998) Biophysical Journal 74, 230-241), or measurement of action potentials using microelectrodes (Dall'Asta V. et al. (1997) Exp. Cell Research 231, 260-268) or

ii) fluorometric techniques wherein the ion currents, in particular calcium currents, are assessed using several ion-sensitive fluorescent dyes, including fura-2, fluo-3, fluo-4, fluo-5N, fura red, Sodium Green, SBFI and other similar probes from suppliers including Molecular Probes. The ionic currents, in particular calcium, can thus be determined in real-time using fluorometric and fluorescence imaging techniques, including fluorescence microscopy with or without laser confocal methods combined with image analysis algorithms.

In a particular embodiment the NG sensory neuron activity assay consist of the patch clamp recordings as described in the examples hereinafter.

Accordingly in a third aspect the present invention provides a method for identifying a compound capable of reducing or preventing prolonged sensory neuron hyper-excitability comprising the steps of:

(a) administering the compound to NG having prolonged sensory neuron hyper-excitability; and

(b) determining the effect of said compound on the NG sensory neuron activity of said cells.

In a further embodiment of the aforementioned method the NG are derived from mouse previously infected with Nippostrongylus brasiliensis. In the aforementioned method the activity of the NG is assessed using any one of the assays described hereinbefore, in particular the patch clamp recordings as described in the examples hereinafter. Alternatively, the capability of a compound to prevent or reduce prolonged sensory neuron hyper-excitability is assessed using whole animal nociceptive assays. In these assays quantifiable behaviour or physiological responses are used to compare pain perception in the non-human animal.

As described in Example 6 hereinafter, a particular assay to study prolonged sensory neuron hyper-excitability consists of the pressor-depressor model in which changes in arterial blood pressure, recorded during phasic distention of both the jejunum and the colon, is used to measure visceral hypersensitivity.

Further assays to study sensory neuron hyper-excitability are known in the art and include;

i) the abdominal constriction, a.k.a. writhing test, wherein a noxious substance is injected into the peritoneal cavity to score the number of writes—lengthwise stretches of the torso with a concomitant concave arching of the back—as a readout for hyper-excitability (Mogil J. S. et al., Pain 80 (1999) 67-82); or
ii) the colorectal distention test (CRD), wherein electromyographic (EMG) recording is used to determine the contraction of the abdominal musculature in response to phasic colorectal distention. This response is also known as the visceromotor response (Kamp E. et al., Am. J. Physiol. Gastrointest. Liver Physiol. 284 (2003) G434-G444.

It is accordingly a further object of this invention to provide the use of a nociceptive assay in a method to identify the capability of a compound to reduce or prevent prolonged sensory neuron hyper-excitability. It thus provides a method for identifying a compound capable of reducing or preventing prolonged sensory neuron hyper-excitability comprising the steps of:

(a) administering the compound to an experimental non-human animal having prolonged sensory neuron hyper-excitability; and

(b) determining the effect of said compound in a nociceptive assay.

In this method any non-human animal model of prolonged sensory neuron hyper-excitability as described hereinbefore can be used. In particular the experimental non-human animal having prolonged sensory neuron hyper-excitability is a rodent previously infected with Nippostrongylus brasiliensis and subjected to stress, even more particular a mouse previously infected with Nippostrongylus brasiliensis and subjected to stress. The nociceptive assay will typically consist of the pressor-depressor model as provided in example 6 hereinafter.

Further visceral and somatic nociceptive assays, reviewed for example in Mogil J. S et al (supra), which may be used in the current invention include, but are not limited to:—the autotomy following hindlimb denervation (AUT) test; the carrageenan hypersensitivity (CAR_{HT}) test; the formalin test (F_{early}/Fₗₐₜₑ); the hot-plate test (HP); the Hargreaves test of thermal nociception (HT); the Cheung peripheral nerve injury model(PNI_{HT}, PNI_{VF}); the tail withdrawal test (TW); and the Von Frey filament test of mechanical sensitivity (VF).

According to a fourth aspect of the current invention there is provided a method of treating a subject with a disease condition related to prolonged sensory neuron hyper-excitability, comprising administering to a subject an effective amount of an agent that modulates NG sensory neuron activity.

Preferably the agent is one which reduces or prevents prolonged sensory neuron hyper-excitability.

Preferably, the disease condition associated with prolonged sensory neuron hyper-excitability is a gastrointestinal (GI) tract disorder, particularly a bowel disorder, such as but not limited to, ulcerative colitis, Crohn's disease, ileitis, proctitis, celiac disease, enteropathy associated with arthropathies, microscopic or collagenous colitis, eosinophilic gastroenteritis, pouchitis resulting after proctocolectomylpost ileoanal anastomosis, functional dyspepsia, functional vomiting, oesophagitis, gastric ulcer, duodenal ulcer or irritable bowel syndrome. In addition the disease or condition associated with prolonged sensory neuron hyper-sensitivity may be depression or other stress-related disorder.

The agent may be one which modulates the expression or activity of one or more of the genes listed in Table 1 or modulates the activity of any protein or polypeptide expressed from one or more of said genes. Preferably, the agents may be those which modulate the expression or activity of one or more receptors selected from the group consisting of Table 2

Further, suitable agents are any compound identified as capable of reducing or preventing prolonged sensory neuron hyper-excitability which are identified using any one of the compound screening methods described above.

According to a fifth aspect of the present invention, there is provided a pharmaceutical composition for the treatment of a disease or disorder related to prolonged sensory neuron hyper-excitability comprising any one or more of the compounds identified below, any other compound capable of modulating the expression or activity of one or more of the genes listed in Table 1 or any compound identified by the method of first aspect of the invention and at least one pharmaceutically acceptable diluent or excipient.

It will be understood that the pharmaceutical composition may be administered by any suitable means, such as, but not limited to oral or nasal administration, suppository, subcutaneous or intraperitoneal injection or intravenous administration.

In the pharmaceutical composition of the invention, preferred compositions include pharmaceutically acceptable carriers including, for example, non-toxic salts, sterile water or the like. A suitable buffer may also be present allowing the compositions to be lyophilized and stored in sterile conditions prior to reconstitution by the addition of sterile water for subsequent administration. The carrier can also contain other pharmaceutically acceptable excipients for modifying other conditions such as pH, osmolarity, viscosity, sterility, lipophilicity, osmobility or the like. Pharmaceutical compositions which permit sustained or delayed release following administration may also be used.

Compounds which are identified are suitable for use in the methods of the current invention along with derivatives that retain substantially the same activity as the starting material, or more preferably exhibit improved activity, which may be produced according to standard principles of medicinal chemistry, which are well known in the art. Such derivatives may exhibit a lesser degree of activity than the starting material, so long as they retain sufficient activity to be therapeutically effective. Derivatives may exhibit improvements in other properties that are desirable in pharmaceutical active agents such as, for example, improved solubility, reduced toxicity, enhanced uptake, etc.

According to a sixth aspect of the present invention there is provided a method of making a pharmaceutical composition for the treatment of a disease or disorder related to prolonged sensory neuron hyper-excitability, comprising combining a compound identified according to the method of the first aspect of the invention or any of the compounds identified as suitable disclosed above together with a pharmaceutically acceptable diluent or excipient.

According to a seventh aspect of the current invention there is provided the use or one or more of the compounds recited below in the manufacture of a medicament for the treatment of a disease or disorder related to prolonged sensory neuron hyper-excitability.

Preferably, the prolonged sensory neuron hyper-excitability is NG sensory neuron hyper-excitability

Preferably, the disease or disorder related to prolonged sensory neuron hyper-excitability is a GI tract disorder. More preferably the GI tract disorder comprises a bowel disorder, such as but not limited to, ulcerative colitis, Croha's disease, ileitis, proctitis, celiacdisease, enteropathy associated with arthropathies, microscopic or collagenous colitis, eosinophilic gastroenteritis, pouchitis resulting after proctocolectomy and post ileoanal anastomosis, functional dyspepsia, functional vomiting, oesophagitis, gastric ulcer, duodenal ulcer or irritable bowel syndrome. In addition the disease or condition associated with prolonged sensory neuron hyper-sensitivity is depression or other stress-related disorder.

In a preferred embodiment the invention relates to uses of a modulator of scrotonin receptor 3A (Htr3a) such as, for example, Ondansetron, Granisetron, Alosetron, Cilinsetron, or dolasetron in the manufacture of a medicament for the treatment of any one of the above GI tract disorders and in particular the treatment of irritable bowel syndrome.

All of the genes listed in Table 1 are potential pharmaceutical targets whose activity might be modulated to reduce or prevent prolonged sensory neuron hyper-excitability. Modulation of one or more of those genes is likely to be useful in the treatment of G.I.tract disorders or stress-related disorders such as ulcerative colitis, Crohn's disease, ileitis, proctitis, celiac disease, enteropathy associated with arthropathies, microscopic or collagenous colitis, eosinophilic gastroenteritis, pouchitis resulting after proctocolectomy and post ileoanal anastomosis, functional dyspepsia, functional vomiting, oesophagitis, gastric ulcer, duodenal ulcer, irritable bowel syndrome or depression.

Techniques which may be used to validate one or more of the genes of Table 1 as a pharmaceutical target in one or more of the above diseases are antisense technology or gene silencing using, for example, methylation of DNA or RNA interference (RNAi). “RNAi” is a process of sequence-specific down-regulation of gene expression RNAi may be performed using, for example, small interfering RNA (siRNA). This is a specific type of the well-known RNAi. technique. (also referred to as “RNA-mediated gene silencing”) initiated by double-stranded RNA (dsRNA) that is complementary in sequence to a region of the target gene to be down-regulated (Fire, A. Trends Genet. Vol. 15, 358-363, 1999; Sharp, Pa. Genes Dev. Vol. 15, 485-490, 2001).

Over the last few years, down-regulation of target genes in multicellular organisms by means of RNA interference (RNAi) has become a well established technique. In general, RNAi comprises contacting the organism or cell with a double-stranded RNA fragment (generally either as two annealed complementary single-strands of RNA or as a hairpin construct) having a sequence that corresponds to at least part of a gene to be down-regulated (the “target gene”). Reference may be made to International application WO 99/32619 (Carnegie Institute of Washington), International application WO 99/53050 (Benitec), and to Fire et al., Nature, Vol. 391, pp. 806-811, February 1998 for general description of the RNAi technique.

Elbashir et al. (Nature, 411, 494-498, 2001) demonstrated effective RNAi-mediated gene silencing in mammalian cells using dsRNA fragments of 21 nucleotides in length (also termed small interfering RNAs or siRNAs). These short siRNAs demonstrate effective and specific gene silencing, whilst avoiding the interferon-mediated non-specific reduction in gene expression which has been observed with the use of dsRNAs greater than 30 bp in length in mammalian cells (Stark G. R. et al., Ann Rev Biochem. 1998, 67:227-264; Manche, L et al., Mol Cell Diol., 1992, 12:5238-5248). In practice these siRNAs may be between about 19 and about 23 nucleotides in length and can be introduced into the cell by standard transfection techniques or more appropriately be produced in situ using an expression vector for the production of siRNAs within cells. A particularly advantageous embodiment of the technique produces 50mer fragments in such a way that they form hairpin-like structures know as shRNAs. These are more stable than siRNA fragments. Commercial siRNA and shRNA kits are available such as one produced by Invivogen. (San Diego, USA)

In an eighth aspect the invention relates to the use of small interfering RNA (siRNA) to validate as pharmaceutical targets in the treatment of a G.I. tract disorder or stress-related disorder such as any of those already listed above, any one or more of the genes shown in Table 1. It will be appreciated that the silencing of any one of the genes will elucidate its role in the listed disorders thus, being an effective target validation mechanism.

### BRIEF DESCRIPTION OF THE DRAWINGS

The invention will be further understood with reference to the following experimental Examples and the accompanying figures in which:—

FIG. 1A shows the numbers of labelled DRG neurons after injection of CTB488 label into the intestinal musculature (IM).

FIG. 1B shows the numbers of labelled DRG neurons after injection of CTB549 label intraperitonealy (IP).

FIGS. 1C and D are panels showing that all neurons fluorescently labelled following IM injection of CTB488 were co-labelled by IP injection of CTB594.

FIG. 2A shows the serum corticosterone stress enzyme levels in the groups of Nb infected and non infected mice after 5 weeks in a stressed or non stressed environment.

FIG. 2B shows the average serum corticosterone levels in the stressed and non stressed mice after 5 weeks.

FIG. 3A shows mean serum IgE levels in μg/ml in infected and non infected stressed and non stressed mice.

FIG. 3B shows the variation in IgE levels in Nb infected and non infected mice over time.

FIG. 4 shows the variation in mast cell counts in Nb infected and non infected mice over time.

FIG. 5 shows the histology of Nb infection in mouse, the panels showing the gut prior to infection, during acute inflammation and after acute inflammation has subsided.

FIG. 6 shows the conductance of the DRG neurons from infected and non infected animals.

FIG. 7 shows that in DRG neurons the Rheobase was lower in Nb infected mice compared to non infected mice.

FIG. 8 shows that action potential number in DRO neurons following 500 ms at 2× Rheobase was increased in Nb infected mice.

FIG. 9 shows a comparison of the slow afterhyperpolarization (sAHP) in DRG neurons following action potentials in sham and Nb infected mice.

FIG. 10 shows the resting conductance of NG neurons from infected and non infected animals, expressed as raw data and normalized to cell size (capacitance)

FIG. 11 shows that action potential number in NG neurons following 500 ms at 2× Rheobase was increased in Nb infected mice.

FIG. 12 shows the change in antipeak amplitude, action potential half width and maximum decay slope in NG after Nb infection.

FIG. 13 shows that in NG neurons the Rheobase was lower in Nb infected mice compared to non infected mice.

FIG. 14 shows spectral map analysis and principal component plot of gene expression in DRG neurons isolated by laser capture from non infected/non stressed, infected/non stressed, non infected/stressed, and infected/stressed groups of mice.

FIG. 15 shows spectral map analysis of gene expression in NG neurons isolated by laser capture from non infected/non stressed, infected/non stressed, non infected/stressed, and infected/stressed groups of mice.

FIG. 16 shows a Venn diagrammatic representation of the number of genes identified by spectral map analysis (SPM), significance analysis (SAM) and fold difference filtering (FD). The selection of 1996 genes was based on the fulfilment of at least two of these three criteria.

FIG. 17A shows the effect on expression of vanilloid receptor VR1 mRNA of Nb infection in DRG and NG neurons measured on an Affymatrix microarray.

FIG. 17B show expression level of Trpv1 mRNA as assessed by quantitative PCR.

FIG. 18A shows the effect on expression of 5-HT3 receptor of Nb infection in NG and DRG neurons.

FIG. 18B shows the effect on expression of cholecystokinin receptor A of Nb infection in NG neurons.

FIG. 19A shows the effect on expression of somatostatin 2 receptor of Nb infection in NG neurons.

FIG. 19B shows expression level of Sstr2 mRNA as assessed by quantitative PCR in DRG an NG neurons.

FIG. 20A shows immunohistochemical staining of VR1 protein level in sham and Nb infected NG and DRG neuron sections.

FIG. 20B shows a graphical representation of the level of VR1 protein staining seen in FIG. 20B, showing that there is a significant increase in VR1 expression in Nb infected NG neurons.

FIG. 21 shows the effect of jejunal phasic distension on pressor responses responses in Sham vs. Day 21 Post Nb infection animals.

FIG. 22 shows the effect of colonic phasic distension on pressor responses in Sham vs. Day 21 Post Nb infection animals.

FIG. 23 shows the effect of 1 μM of the somatostatin antagonist octreotide on evoked action potential discharge in sham and infected NG-neurons.

FIG. 24 shows the mean effects of 1 μM of the somatostatin antagonist octreotide on evoked action potential discharge in sham and infected NG neurons.

FIG. 25 shows in panel A the mean afferent nerve activity and in Panel B the IP response to intraluminal acid infusion in sham and Nb infected mice.

FIG. 26 shows the acute and prolonged increase over baseline of nerve firing (Panel A) and IP (Panel B) in response to intraluminal acid infusion.

### MATERIALS AND METHODS

### Retrograde Labelling of Sensory Neurons

Female balb-c mice (20±2 g, n=4) were anaesthetized with ketamine/xylazine/acepromazine (80/50/1 mg/kg, IP, respectively). Anaesthesia was subsequently maintained by top-up doses of 20 mg/kg ketamine (IP) as required. Following a midline laparotomy, a 5 cm section of the jejunum was exposed to enable intramuscular (1M) injections of fluorescently labelled cholera toxin subunit B (CTB488, Molecular Probes, Eugene, Oreg.). A Hamilton syringe was used to inject 2-4 μl of CTB488 into the intestinal musculature at ten distinct sites along both sides of the jejunal segment. Following suturing of the abdominal incision and recovery from the surgery, mice were injected IP with a contrasting fluorophore (CTB594, 100 μl, Molecular Probes). After a 4 day recovery period, animals were euthanized. NG and DRG from T1-L4 were removed. Each ganglion was placed on a slide and a coverslip was used to cover and squash the ganglia to enable counts of CTB488- and CTB594-labelled neurons in the same ganglia, using a Leica fluorescence microscope equipped with TX2 (for CTB594) and L5 (for CTB488) filter blocks (Leica, Toronto, Canada). All procedures were approved by the institutional Animal Care Committee.

### Assessment of Numbers of Neurons in Sensory Ganglia

CTB488 was administered IP (100 μl) to mice and the animals were euthanized four days later. One of each pair of DRG from T10 to T13 were harvested and frozen, prior to sectioning on the cryostat at 10 μm. The paired ganglia from the contra-lateral side were squashed on slides beneath cover slips, as described above. Photomicrographs of at least 10 cryostat sections per ganglion and the squash preparations were prepared using a Leica fluorescence microscope and filter block L5. The numbers of fluorescent cells were counted from the resultant photomicrographs. The cryostat sections were then stained with methylene blue and the total numbers of neurons (ganglion cells containing a recognizable nucleus) were counted. From these measurements it was possible to determine the percentage of fluorescent neurons in the cryostat sections (number of fluorescent cells×100/total of number of neurons). This factor could then be applied to the squash preparation counts to determine the total numbers of neurons per ganglion (number of fluorescent neurons in squash preparations×100/percentage fluorescent neurons).

### Laser Capture Microdissection (LCM)

All ganglia harvested for microarray studies were removed 3-4 days after a single IP injection of CTB488 (100 μl). Nodose and T10 to T13 dorsal root ganglia were procured from balb-c mice. Each labelled sensory ganglion was placed in tissue freezing medium (TFM™, Triangle Biomedical Sciences, Durham, N.C.), frozen and stored at −80° C. until the sample was used for RNA extraction or laser capture microdissection (LCM). Cryostat sections (12 μm) were attached to RNAse-free PEN membrane-covered glass slides (P.A.L.M. Microlaser Technologies AG, Bernried, Germany), fixed with 100% ethanol and air dried prior to LCM. Microdissection was performed on a P.A.L.M. microbeam-equipped microscope (Axiovert 135, Zeiss, Gottingen, Germany). Fluorescent neuronal cells were detected and subsequently marked by cutting the contours of the cell with low laser energy. Marked cells were excised after Nissl staining (0.5% Cresyl violet Acetate [Sigma-Aldrich, St. Louis, Mo.]/0.1M SodiumAcetate [Fluka, Buchs, Switzerland]). Cells were catapulted in 75 μl Rneasy lysis buffer (RLT, Qiagen GmbH, Hilden, Germany) containing 0.14M β-mercaptoethanol and 200 ng polyinosinic acid (Sigma) and stored at −80° C.

### RNA Isolation

Laser captured samples were incubated at 42° C. for 20 min and then chilled on ice. An equal volume of 70% ethanol was added to each sample and then transferred to RNeasy MinElute Spin Columns (Qiagen). RNA was cleaned up according to manufacturer's instructions, eluted in 14 μl of RNAse free water and adjusted to 4 μl by vacuum drying.

### RNA Amplification

As “spike-in” controls, the GeneChip Poly-A RNA control kit (Affymetrix, Santa Clara, Calif.) was used. Serial dilutions were made of the prokaryotic Poly-A control using the following dilution steps; 1:20, 1:50, 1:50, 1:20 and 1:10. This dilutions series was based on a estimated starting amount of 0.5 ng total RNA in the laser captured material. First strand cDNA was prepared as described by the Affymetrix two cycle cDNA synthesis protocol except for the use of Superscript III (Invitrogen, Carlsbad, Calif.) and incubation at 50° C. for 30 minutes. Second strand master mix consisted of 1 μl 10× Bst polymerase buffer (Epicentre, Madison, Wis.), 1 μl of 10 mM dNTP (Invitrogen), 0.5 μl (1U) thermostable RnaseH (Invitrogen), 1 μl (5U) Bst DNA polymerase Epicentre) and water to 10 μl. This master mix was added to the first strand cDNA reaction and incubated at 65° C. for 10 min before heat inactivation at 80° C. for 3 min. Subsequently 2 μl of exonuclease mix was added containing ExoI and ExoVII and incubated at 37° C. for 10 min followed by heat inactivation at 80° C. for 3 min. Double-stranded cDNA was transcribed at 42° C. for 3 hours using the AmpliScribe T7 High Yield Transcription Kit (Epicentre) in a total volume of 100 μl (final concentration of all reagents 0.2× less than described in manufacturer's instructions). The resulting amplified RNA was incubated with DNAse I (4 Units/μl) at 37° C. for 15 minutes. Amplified RNA was purified after adding 100 ng polyinosinic acid using RNeasy MinElute Cleanup Kit (Qiagen). RNA was eluted in 14 μl of RNAse-free water and adjusted to 4 μl by vacuum drying. The second round of amplification was performed as described above except that 50 ng of random hexamer primers was used to prime the reverse-transcription reaction and that the second strand cDNA reaction was primed with 0.25 ng T7 oligo.

### RNA Labelling and Microarray Hybridisation

The third round amplification, including biotin labelling, was performed on 500 ng of second round amplified RNA. First strand cDNA synthesis was performed as described above except that Superscript II was used and incubated at 37° C. for 1 hour. Subsequently RNAse H (1U) (Invitrogen) was added and incubated at 37° C. for 20 min followed by denaturation at 95° C. for 2 min. Second strand cDNA synthesis was performed using 1 μl T7 oligo dT24 (Affymetrix 100 μmol/μl) annealed for 5 min at 70° C., and the reaction was then incubated at 42° C. for 10 min. A master mix was prepared consisting of 10× second strand buffer, dNTPs (200 mM final), E. coli RNAse H (2U) and 10U E. coli DNA polymerase (Invitrogen) and added to the first strand reaction to obtain a 50 μl reaction volume. Following incubation at 37° C. for 10 min, denaturation was done at 80° C. for 3 min. Cleanup of second strand cDNA synthesis was performed using Qiagen PCR purification kit according to manufacturer's instructions. For synthesis of biotin-labelled RNA the BioArray HighYield RNA transcript labelling Kit (Enzo Life Sciences, Farmingdale, N.Y.) was used according to manufacturer's instructions. Clean-up of biotin labelled RNA was performed using the RNeasy Mini Kit (Qiagen). Labelled RNA was hybridized to either mouse genome MG-U74v2 (12.000 transcripts) or MG-430_{—}2.0 (39.000 transcripts) GeneChip arrays (Affymetrix). Hybridisation of microarrays was performed using 12.5 μg biotin labelled RNA at 45° C. for 16 h under continuous rotation. Arrays were stained in Affymetrix Fluidics stations using Streptavidin/Phycoerythrin (SAPE) followed by staining with anti-streptavidin antibody and a second SAPE staining. Subsequently arrays were scanned with a Agilent Laserscanner (Affymetrix) and data were analysed with the Microarray Suite Software 5.0 (Affymetrix). No scaling or normalization was performed at this stage.

### Data Analysis and Selection of Genes

Normalization: Genes that were called absent in all samples according to Affymetrix′ MAS 5.0 software (p-value of >0.06) were removed from further analysis. Raw intensities from each chip were log₂ transformed and all data from the samples were quantile normalized per type of ganglion using the method described by Amaratunga and Cabrera (Amaratunga D, Cabrera J., J Am Stat Assoc 2001; 96:1161-1170). Following the group-wise quantile normalization, a second quantile normalization was carried out across the data of all DRG and NG derived samples. This alignment sets the range of intensities of one array to the range measured across all arrays, compensating for array to array variations in hybridisation, washing and staining, ultimately allowing a reasonable comparison between arrays.

Spectral map analysis: Spectral map analysis is a recently introduced special type of multivariate projection method that helps to reduce the complexity (dimensions) of highly dimensional data (n genes versus p samples) (Wouters L, Göhlmann H W, Bijnens L, Kass S U, Molenberghs G, Lewi P J., Biometrics 2003; 59:1133-1141). This unsupervised method allows the reduction of the complexity of large microarray datasets and provides a means to visually inspect and thereby identify clusters of genes and/or subjects in the data without any bias from the observer. The aim of the technique is to retrieve the most predominant differences in the dataset, disregarding genes that do not contribute to the difference.

Signficance analysis: Individual genes with different expression levels between groups (Sh/NS vs I/S) were identified using Significance Analysis of Microarray data (SAM) (Tusher V G, Tibshirani R, Chu G., Proc Natl Acad Sci USA 2001; 98:5116-5121). SAM assigns a score to each gene based on the difference in gene expression level relative to the standard deviation of repeated measurements. SAM uses permutations of the repeated measurements to estimate the percentage of genes identified by chance; i.e. the false discovery rate (FDR). An extension of this FDR is the so-called q-value introduced by Storey (Storey J D, Tibshirani R., Proc Natl Acad Sci USA 2003; 100:9440-9445). Whereas the p-value is commonly used for performing a single significance test, the q-value is useful for assigning a measure of significance to each of many tests performed simultaneously, as in microarray experiments. We applied a 10% threshold (q=0.1) for our analysis (http://faculty.washington.edu/˜jstorey/qvalue/manual.pdf; http://faculty.washington.edu/˜jstorey/qvalue/manual.pdf 2004).

Fold-difference filtering: A fold-difference filter was applied excluding all genes that exhibited a difference in expression below 50% (1.5 fold difference filter).

### Effect of Amplification and CTB488 Injection on Gene Expression

Effect of CTB488: The effect of CTB488 labelling on gene expression profiles in sensory ganglia was assessed by comparing expression profiles of ganglia isolated from three vehicle treated animals to those of three combined intradermal and IP injected mice (resulting in labelling of almost all neurons). Although a clear difference in expression profile was observed between NG and DRG, no significant effect of the dye injection was noted.

Effect of amplification: In order to obtain sufficient material for microarray experiments, RNA isolated from laser captured neurons was amplified using a three round amplification protocol. Efficiency and sensitivity of amplification were assessed by adding to the amplification reaction “spike-in” controls, consisting of four exogenous, pre-mixed, polyadenylated prokaryotic sequences. The resultant array signal intensities of the “spike-in” controls served as sensitive indicators of the amplification and labelling efficiency, independent of starting sample quality. In agreement with previous reports, “spike-in” controls revealed a detection limit of 1 copy in 1,000,000 and a direct correlation between signal intensity and copy number.

### Quantitative RT-PCR

Microarray data were confirmed using real time PCR analysis. First strand cDNA synthesis was performed on 50 ng second round amplified RNA using random hexamer primers and Superscript II RT (Invitrogen). Quantitative PCR was performed on a ABIPrism 7900 cycler (Applied Biosystems, Foster City, Calif.) using a Taqman PCR kit (Applied Biosystems). Serial dilutions of cDNA were used to generate standard curves of threshold cycles versus the logarithms of concentration for ATPSase and the genes of interest (see Table 4 for sequences of primers (Eurogentec, Seraing, Belgium)).

TABLE 4

| | | |
|---|---|---|
| CCKA | Forward | 5′-CTGGGCAAGGGTGGTAACAT-3′ |
| CCKA | probe | 5′-Fam-CCCAAGGAAAACTAGCATGTGGGACTC |
| | | A-Tamra-3′ |
| CCKA | Reverse | 5′-AGTTTTGGCATTCAAAGCTACTTATTAA-3′ |
| HTR3a | Forward | 5′-TGTGCTCGCTTACAGCATCAC-3′ |
| HTR3a | probe | 5′-Fam-CTGGTCACTCTCTGGTCCATTTGGCA- |
| | | Tamra-3′ |
| HTR3a | Reverse | 5′-GGCTGTGCCCACTCAAGAAT-3′ |
| Trpv1 | Forward | 5′-GCTCCAGGCCCAGAACTTG-3′ |
| Trpv1 | Probe | 5′-fam-TTGGGACGCTCCTTCCTAGCT- |
| | | Tamra-3′ |
| Trpv1 | Reverse | 5′-GGCAGTCTCTCCACCTCTCAGT-3′ |
| Sstr2 | Forward | 5′-TCCGGAGCGGAAGACATC-3′ |
| Sstr2 | Probe | 5′-fam-ACCAGGTCACACCCAGGCAA- |
| | | Tamra-3′ |
| Sstr2 | Reverse | 5′-GCCGGGCAGCTGTTTTC-3′ |
| ATPSase | Forward | 5′-GCACTGCAACTGATCTCTCCAT-3′ |
| ATPSase | Probe | 5′-Fam-CAAGCGAGAGCTCAGGTTTCCTTC- |
| | | Tamra-3′ |
| ATPSase | Reverse | 5′-GCTCTTGTGTGGCCTGCAT-3′ |

### Murine Environmental Stressor

Balb/c mice were housed under different environmental conditions to produce ‘stressed’ and ‘non-stressed’ animals (Table5). Non-stressed animals were housed 3 mice to a cage and cages were supplied with gauze to make bedding and tubing for environmental enrichment. These animals were assimilated to human handling. Stressed animals were housed 5 animals to a cage and were not supplied with gauze or tubing in their cages, and were not assimilated to human handling.

TABLE 5

| Stressed | Non-stressed |
|---|---|
| 5 per cage | 3 per cage |
| No Tubes | Tubes |
| No Gauze | Gauze |
| Handle by tail only | Handle with support |
| Open access | Restricted access |
| Irregular handling | Habituation by repeated handling |

### Blood Pressure-Distension Experiments

Balb/c mice were anaesthetized with isofluorane. The carotid artery was cannulated for monitoring blood pressure and heart rate. Following a mid line laparotomy, a 5 cm section of the mid jejunum was intubated to allow infusion of saline in order to distend the jejunum. A 5 cm section of the proximal colon was also intubated to allow colonic distensions. The exposed and cannulated segments of gut were covered in gauze moistensed with saline to prevent dehydration. Blood pressure was allowed to stabilize for at least 20 minutes prior to starting experimental stimuli. Phasic distensions were performed manually by attaching a syringe to the end of the intraluminal cannulae and injecting saline into the gut until the desired pressure is reached. This pressure was maintained manually for 30 secs before release and the intraluminal pressure returned to baseline (˜0 mmHg). The pressures attained were 12.5, 25, 50, 75, 100 mmHg, and there was a 10 minutes interval left between each stimulus. The volume injected during each distension was recorded. This series of phasic distensions from 12.5-100 mmHg were performed in the jejunum first, then after a 10 minute interval, in the proximal colon. The resultant deviations in the arterial blood pressure were recorded in response to each individual stimulus. With balb/c mice under isofluorane aneasthesia there was typically an increase in blood pressure (pressor response) followed by a decrease in blood pressure (depressor response). Each of these parameters was measured separately and dose response curves of the changes in blood pressures at increasing intraluminal pressures were plotted for both the jejunum and the colon.

### Patch Clamp Experiments

Balb/c mice were injected intraperitoneally with the retrograde labelling agent cholera toxin B 488 3-7 days prior to experiments. Mice were then anaesthetized with ketamine/xylazine, the spinal cord removed and DRG neurons isolated (T10-T13) for electrophysiological recordings 18-24 hours after their dissociation and incubation, and mounted on the stage of an inverted microscope (Leica DMIRE2)) for both bright-field and fluorescence observation. Cholera toxin labelled neurons were identified by their green fluorescence under the N3 filter system (Leica). Whole cell currents and voltage clamp experiments were performed by using MultiClamp 7A amplifier and digitized with a DigiData 1322A converter (Axon Instruments). Stimulation and data acquisition were obtained by the pClamp 9 program (Axon Instruments). Signals were sampled at 10 kHz or 20 kHz, and low-pass filtered at 4 KHz. The series resistance was compensated. Neurons were excluded from analysis if the seal resistance or access resistance was unstable, or if they fired spontaneous action potentials.

Borosilicate glass (Harvard) was pulled with a P97 micropipette puller (Sutter, Calif.), and fire polished by a M 200 microforge (World Precision Instrument) to a tip resistance of 5-10 MΩ. A silver-silver chloride pellet (world Precision Instrument) was placed in the recording dish as the reference electrode. The normal extracellular Kreb's solution contained (in mM): NaCl 118.0, KCl 4.7, NaH₂PO₄ 1.0, NaHCO₃ 25.0, MgSO₄ 1.2, CaCl₂ 2.5, D-Glucose 11.1, with pH adjusted to 7.3 by using NaOH. The normal intracellular solution contained (in mM): HEPES 10.0, KCl 130.0, MgCl₂ 1.0, CaCl₂ 1.0, EGTA 2.0, K₂ATP 2, Na₃GTP 0.2, titrated with KOH to pH 7.25. The extracellular solution for isolating TTX-resistance Na currents composed of (in mM): NaCl 145.0, KCl 4.8, HEPES 10.0, MgCl₂ 1.0, CaCl₂ 2.5, D-glucose 11.1, TTX 0.0003, CdCl 0.5, 4-AP 1.0, TEA-Cl 5.0, CsCl 2.0, pH adjusted to 7.3 by using NaOH, and the corresponding intracellular solution was (in mM): EPES 10.0, CsCl 130.0, MgCl₂ 1.0, CaCl₂ 1.0, EGTA 2.0, K₂ATP 2.0, Na₃GTP 0.2, pH adjusted to 7.25 by using CsOH. All experiments were performed at temperature of 30° C.-33° C.

Data were analyzed by using pClamp 9 software (Axon Instruments). Neurons were recorded in both current-clamp and voltage-clamp configurations. Voltage clamp recordings were used to generate current-voltage relationships for cells. Current clamp recordings were used to determine the rheobase for action potential firing of neurons. The number of action potentials elicited at 2× rheobase was subsequently assessed in current clamp mode.

### Corticosterone Assay

Balb/c mice were anesthetized by ketamine/xylazine solution and blood was collected by a cardiac puncture to 3 ml vacutainer tubes containing EDTA (BD Scientific). Tubes were placed at 4° C. for 2 hours and then plasma was separated by centrifugation at 15,000 RPMI for 15 minutes, transferred to an Eppendorff tubes and frozen at −20° C. for up to 1 month prior to ELISA assay.

Corticosterone levels in mouse plasma were determined by OCTEIA EIA assay (ALPCO Diagnostics, Windham N.H., USA). Briely, plasma was diluted 1:10 with sample dilutent in a glass tube (10×75 mm) and mixed on vortex. One hundred μl of such diluted samples were loaded on pre-coated 96-well plates and 100 μl of enzyme conjugated solution was added to each well. Plates were incubated overnight at 4° C. Samples were run simultaneously with provided corticosterone calibrators. After incubation the contents of the plates were dumped and the plates were washed 3 times with 250 μl of the washing buffer. TMB substrate (200 μl) was added to each well and incubation continued for additional 30 minutes at room temperature. Reaction was stopped by adding 100 μl of stop solution HCl and the plates were read at 450 nm in an automated ELISA reader ELx808. Data were analyzed using KCjunior software (Bio-Tek Instruments, Winooski V E, USA) and expressed in ng/ml.

### Non-Recovery Surgical Procedures

General anaesthesia in mice was induced with 3% isoflurane and maintained with 2% isoflurane. The right external jugular vein was cannulated to allow maintenance anaesthesia and the left external jugular vein was cannulated for systemic administration of drugs. Body temperature was monitored with a rectal thermometer and maintained at around 37° C. by means of a heating blanket. A midline laparotomy was performed and the caecum was excised. A 5 cm loop of proximal jejunum was isolated and cannulated at the proximal end with a cannula connected to a syringe pump to allow infusion of intraluminal solutions. This inlet cannula was also connected to a pressure transducer to allow monitoring of intraluminal pressure. The jejunal loop was cannulated at the distal end to allow drainage of intraluminal solutions to waste. The abdominal incision was sutured to a 20 nm diameter steel ring to form a well that was subsequently filled with pre-warmed (37° C.) light liquid paraffin.

### Nerve Preparation and Afferent Recording

A mesenteric arcade was placed on a black Perspex platform and a single nerve bundle was dissected from the surrounding tissue. This was severed distal from the wall of the jejunum (approximately 5-10 mm) to eliminate efferent nerve activity. It was then attached to one of a pair of platinum electrodes, with a strand of connective tissue wrapped around the other to act as a differential. The electrodes were connected to a 1902 amplifier (Cambridge Electronic Design (CED), Cambridge, UK), filtered and differentially amplified with the resulting signal digitized via a 1401 plus interface (CED) and captured on a PC using Spike2 software (CED).

### Quantitative Immunohistochemistry for VR1

To localize VR1-immunoreactivity, dorsal root ganglia and nodose ganglia were harvested from mice infected or sham-infected with Nb 21 days previously and sacrificed by an overdose of ketamine/xylazine (n=4 per group). Immediately after removal, the ganglia were immersed in 10% neutral buffered formalin (NBF) for 48 hours, before processing to paraffin. After embedding, sections were cut at 2 μm and collected on aminopropyltriethoxysilane-coated slides. Sections were dewaxed and endogenous peroxidase was blocked in 0.5% hydrogen peroxide in methanol. After rinsing in Tris buffered saline (TBS), sections were pre-treated with citrate buffer, pH6.0, for 30 minutes at 98° C. and then incubated in 20% normal goat serum in TBS for 20 minutes, followed by anti-VR1 (PC420, Oncogene, now Calbiochem, San Diego, Calif., USA) overnight at room temperature. Sites of primary antibody binding were detected using double-cycled, goat anti-rabbit Igs and streptavidin-peroxidase (Zymed Laboratories, South San Francisco, Calif.). Colour was developed in aminoethylcarbazole and the nuclei were counterstained in haematoxilyn. Sections were coverslipped in glycerine jelly. Quantitation was performed using Quantimet Image Analysis software (Version 2.7, Leica, Toronto, Canada). Integrated optical densities were determined at 20× objective magnification. The total integrated optical densities of the specific staining were used for comparison between animals and groups.

### Chemosensitivity Experiments

Balb/c mice were injected subcutaneously with 500 L3 Nb larvae in PBS, or with PBS only (shams). Experiments were performed 3-4 weeks post-infection. Mesenteric afferent recordings were obtained from isoflurane anaesthetized mice using conventional extracellular recording techniques. A 5 cm section of the jejunum was intubated to allow continuous intraluminal perfusion (0.15 ml/min) of either 0.9% saline or 50 mM hydrochloric acid (HCl). Jejunal afferent nerve activity and intraluminal pressure (IP) was recorded in response to a 2.5 min HCl application (at time 0s). Baseline activity (−100 to 0s), acute acid response (50 to 110 s) and prolonged acid response (410 to 560 s) were measured and compared between sham and Nb infected mice.

### EXAMPLES

### Example 1

### Labelling of Visceral Sensory Neurons

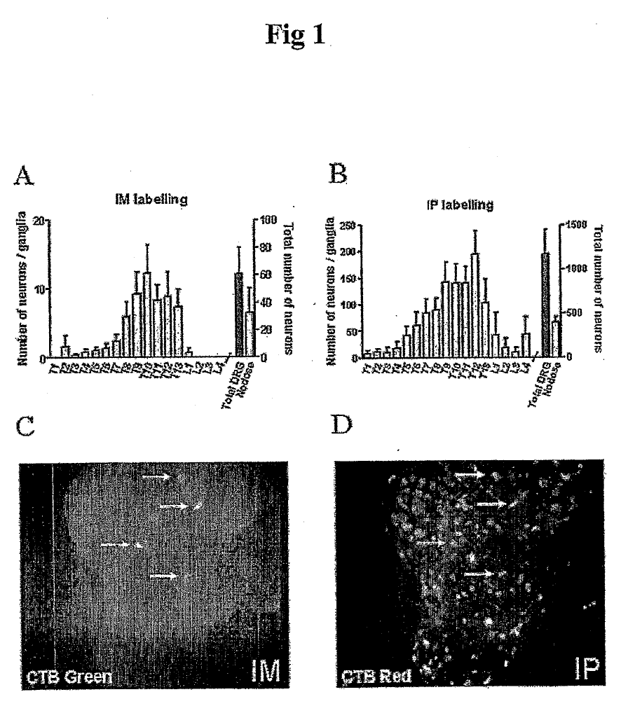

Intramuscular injection of abdominal tissues necessitates invasive surgery that is likely to alter the expression of a variety of genes. Initial experiments were thus performed to evaluate intraperitoneal (IP) injection of label as an alternative to injection into the intestinal musculature (IM), by comparing the retrograde labelling characteristics of DRG and NG after IM and IP injections of CTB488 and CTB594. Injection of CTB488 IM labelled DRG neurons from T2-L1, with 61% of neurons labelled between T10-T13 (FIG. 1A). In comparison, IP injection of CTB594 labelled DRG neurons over a slightly larger range, from T1-L4, but with 50% of neurons labelled still located between T10-T13 (FIG. 1B). FIGS. 1C and 1D show that every neuron labelled following IM injection of CTB488 was co-labelled by IP injection of CTB594. FIGS. 1A and 1B also show that the total number of T10-T13 DRG neurons labelled following IM injection was 37±12 ganglion cells, which was only 6.4% of the neurons labelled following IP injection (580±132 ganglion cells). There was also a similar percentage (8.2%) of nodose neurons labelled with 1M injection compared to IP injection (32±18 vs. 398±62 neurons respectively). There was no significant difference between the number of NG and DRO neurons labelled by IM injection (p=0.55). Similarly there was no significant difference between the number of NG and DRO neurons labelled by IP injection (p=0.15). Table 6 shows the numbers of fluorescent T10-T13 neurons counted in squash preparations labelled after IP injection, along with the percentage of fluorescent neurons as determined in cryostat sections. All four levels of dorsal root ganglia produced similar results, with ≦3% of the neurons being labelled following IP injection. By extrapolation, the total numbers of neurons per ganglion were estimated to be in the region of 7,000 to 9,000. In conclusion, since IM injections only cover a limited section of the GI tract and IP label injection may avoid any alterations in neuronal expression and/or function that may occur following the surgery necessary for IM label injection, IP injection of CTB was used to label DRG and NG for subsequent microarray studies.

### Full Legend for FIG. 1

FIG. 1: CTXB labelling of sensory neurons. A—Bar graph shows the mean number of neurons (n=4-6 experiments) labelled by IM injection in DRGs, and nodose neurons. B—Bar graph showing the same data as A except following an IP injection. C&D—All neurons that are labelled by IP injection are co-labelled by IM injection. An example of a squash preparations of the same DRG illuminated through a FITC filter (C—IM injection with CTB Green 488) and a Cy3 filter (D—IP injection with CTB Red 594). More neurons are labelled by IP injection than by IM injection. However, all of the fluorescent neurons that are labelled by IM injection (arrows) are also labelled by IP injection.

TABLE 6

| Parameter | T10 | T11 | T12 | T13 |
|---|---|---|---|---|
| % Fluo. Cells | 2.7 ± 0.7 | 2.5 ± 0.5 | 2.9 ± 0.8 | 3.0 ± 0.4 |
| Mean # | 194 ± 34.4 | 225 ± 30.9 | 233 ± 27.4 | 252 ± 15.9 |
| Fluo. Cells | | | | |
| # Neurons | 7266 | 9066 | 8084 | 8508 |
| per Ganglion | | | | |

### Example 2

### Mouse Model of Irritable Bowel Syndrome (IBS)

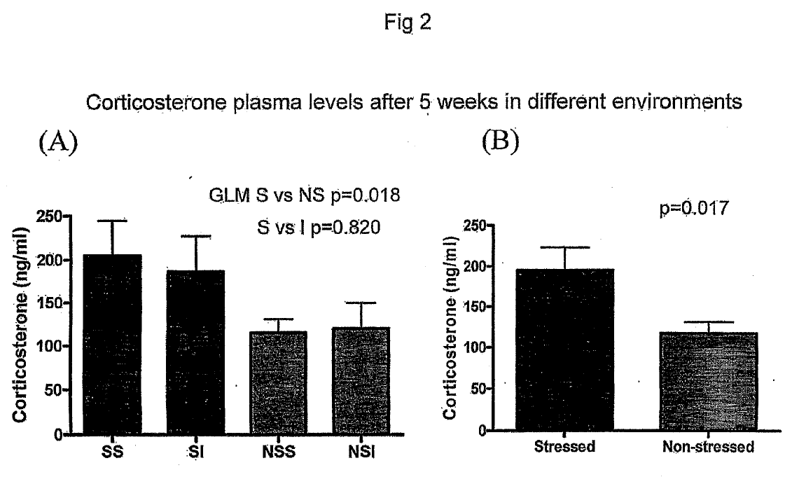

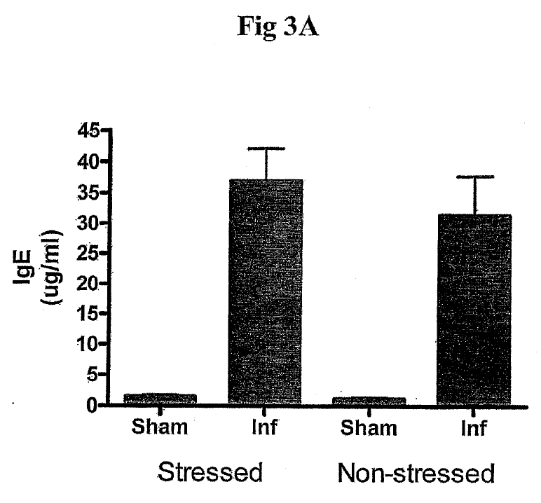

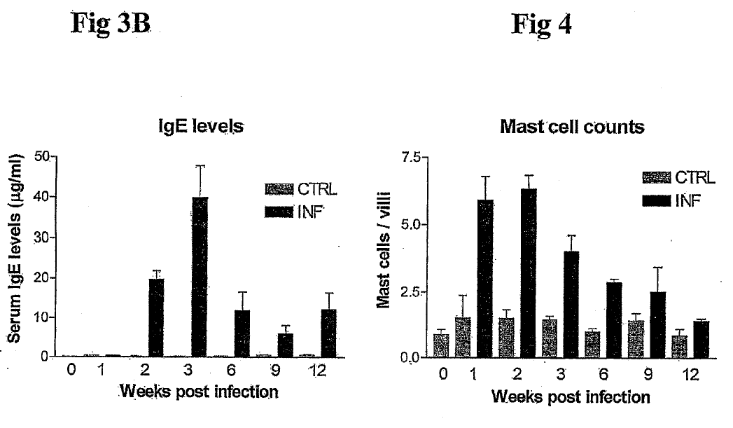

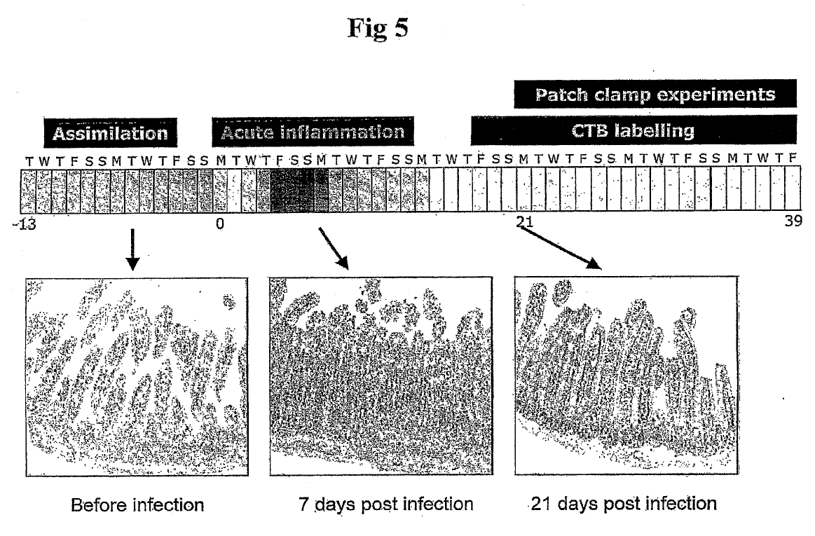

A conceptual mouse model of IBS was set up by combining infection and exposure to stress. Transient jejunitis was induced in Balb/c mice by infection with Nippostrongylus brasiliensis (Nb) larvae in PBS. Sham animals were injected with PBS only. Different levels of stress were obtained by combination of all of the following factors concerning housing of the animals; number of animals per cage, presence/absence of tubes and gauze, method of handling (Table 5). Combination of stress and infection resulted in four groups of animals including sham/non-stressed (Sh/NS), infected/non-stressed (I/NS), sham/stressed (Sh/S) and infected/stressed (I/S) animals. Although considered as a mild stressor, FIGS. 2A and 2B shows that the differences in housing conditions resulted in pronounced differences in stress hormone levels after five weeks in different environments as indicated by plasma corticosterone levels. In agreement with observations in the rat, FIGS. 3B and 4 show that both serum IgE levels and mast cell counts were elevated in mice after Nb infection when compared to non infected mice. FIG. 5 shows that three to six weeks after the infection all signs of acute inflammation disappeared: the epithelium is no longer regenerative; the lamina propria is no longer hypercellular nor oedematous; neutrophils are not evident; and the muscularis propria has returned to normal thickness. All further experiments were performed after day 21.

### Full Figure Legends for FIGS. 2 to 5

FIGS. 2A and 2B: Mice were housed under stressed or non stressed conditions for 5 weeks. After two weeks animals were infected with Nippostrongylus brasiliensis or sham infected with vehicle. Plasma corticosterone levels were measured by ELISA. Data are expressed in ng/ml±SEM. (A). Mean plasma corticosterone levels for each of the four experimental groups: SS—stressed sham; SI—stressed infected; NSS—non-stressed sham; NSI—non-stressed infected. Using a general linear model (GLM), there was no significant difference between sham vs. infected, but there was an elevation on corticosterone levels in stressed vs. non-stressed animals. (B) Averaged data pooling the two stressed populations vs. the two non-stressed population. There was an increase in stressed vs. non-stressed animals.

FIG. 3A Serum IgE levels in μg/ml (mean ±SEM) in four different experimental groups as indicated. All animals were kept in the appropriate housing conditions for 5 weeks prior to measurements being taken. IgE levels were measured using ELISA 21 days after s.c. infection with either sham or 500 L3 Nb larvae. IgE levels were only increased in Nb infected animals.

FIGS. 3B and 4: Serum IgE levels in μg/ml (3) and mast cell counts (4) at different times post-infection. Mice were infected with Nippostrongylus brasiliensis (INF) or sham infected (CTRL). IgE levels were detectable 2 weeks after infection, peaked at week 3-4 and remained elevated 12 weeks post-infection. Mast cell numbers increased at week 1; peaked at week 2 and returned to near normal levels at week 12 post-infection.

FIG. 5: Histological time course of mouse jejunum with Nb infection. Mice were infected sub-cutaneously on day 0 with 500 stage L3 larvae of Nippostrongylus brasiliensis after a two week assimilation period. Jejunum was collected on day 0, 7 and 21 days post infection. Tissue was fixed in formalin and stained with hematoxylinleosin. Severity of inflammation was determined and expressed on different color intensity scale. Inflammation peaked at day 7 and returned to normal on day 21. Histological photographs of the representative time points are presented below the time scale.

### Example 3

### Persistent Alterations in Neuron Excitability in Mice Infected with Nb

In order to study changes in electrophysiological properties, patch clamp recordings were performed on isolated NG and DRG neurons after stress exposure and Nb infection. NG were harvested on day 20-24 post infection, i.e., after histological and biochemical signs of acute gut inflammation are gone. Dispersed ganglion cells were plated on coverslips and incubated for 4-24 hr before mounting for patch clamp recording, using physiological extracellular saline and a K⁺-rich intracellular saline. Visceral DRG and NG neurons were identified by retrograde transport of a labelled cholera toxin subunit (Alexa Fluor-488-CTB), which had been injected IP, 3 to 8 days prior to sacrifice.

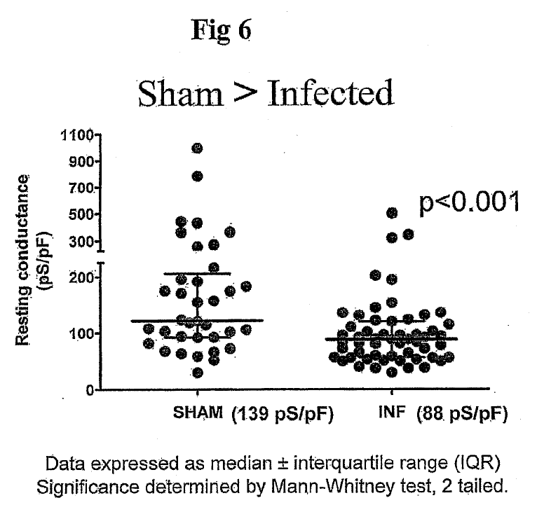

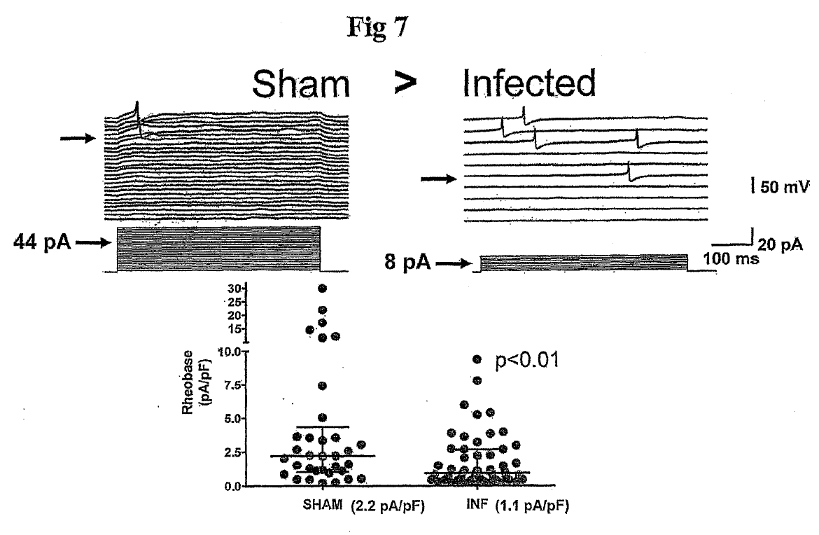

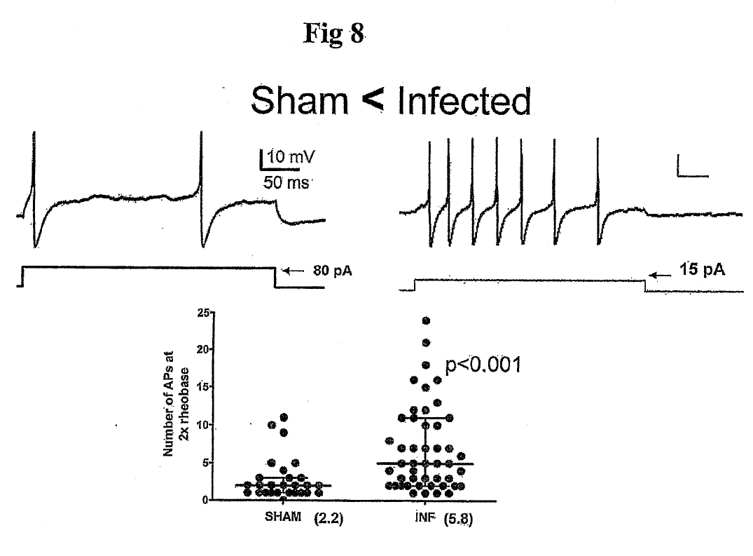

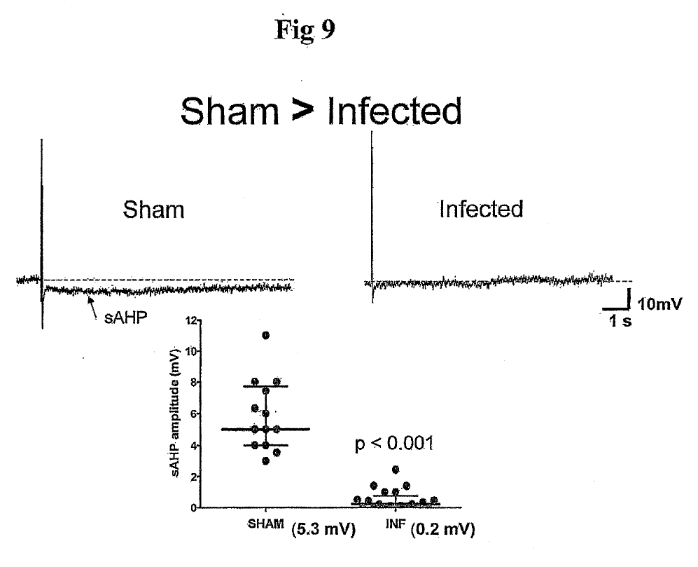

For DRG neurons recordings were made from small neurons (whole cell C<40 pF, 91 neurons in total), which consistently showed a hump during spike repolarization. Spike shape and amplitude was not altered by Nb infection. FIG. 6 shows that overall DRG neurons (n=55) derived from Nb infected animals had a lower resting conductance (88, 64 cf. 139, 132 pS/pF; median, IQR, P<0.001) than those (n=36) derived from sham infected animals, but Vᵣₑₛₜ did not differ (−50 cf. −51 mV). FIG. 7 shows that Rheobase was lower (1.1, 2.1 cf. 2.2, 4.5 pA/pF, P<0.001) in Nb mice. FIG. 8 shows that action potential number evoked during 500 ms at 2× rheobase was increased from 2, 2 to 5, 8, P<0.0001) in Nb infected. Action potentials recorded from sham neurons were followed by a slow (0.2-1 s duration) afterhyperpolarization (sAHP) with maximal amplitude of 5, 3 mV. The sAHP amplitude was greatly reduced in neurons taken from Nb mice (0.2, 0.4 mV, P<0.001) (FIG. 9).

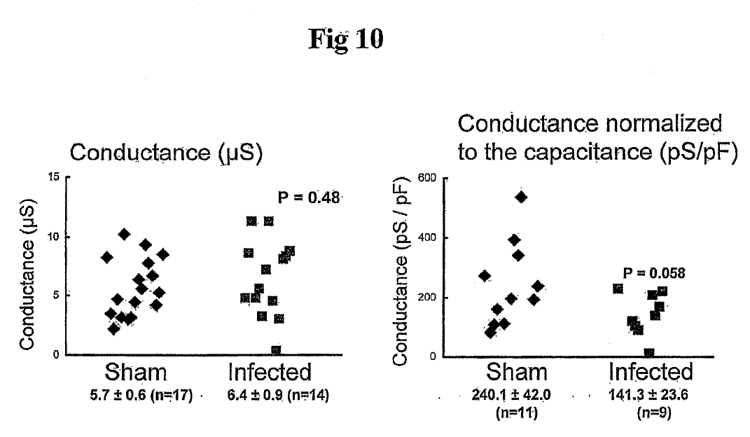

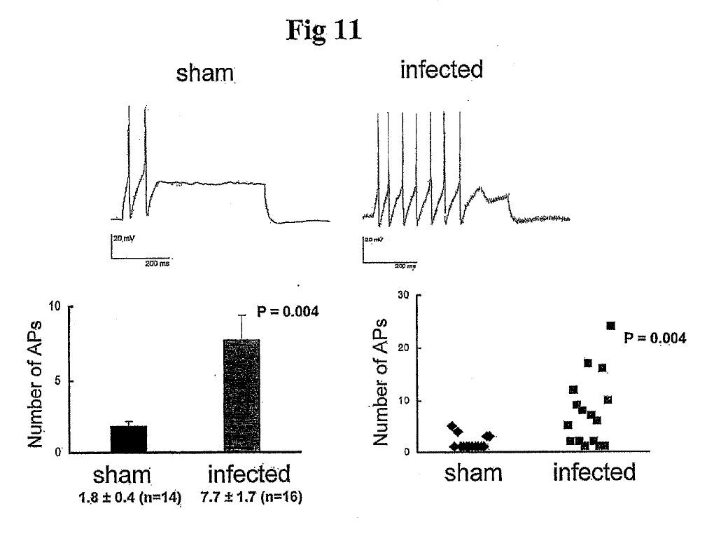

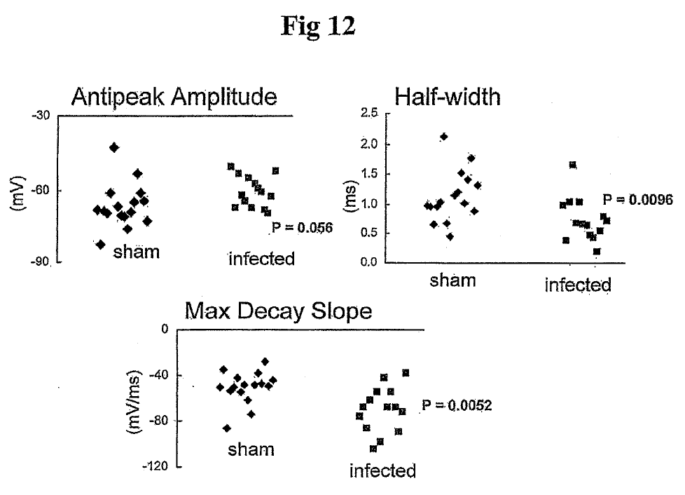

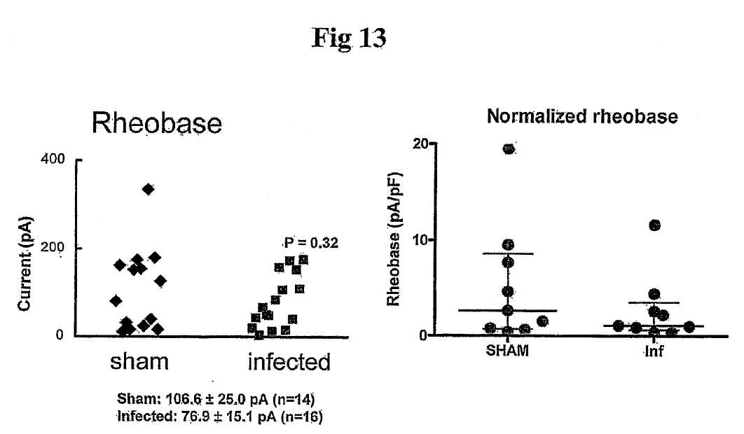

With respect to NG neurons, electrophysiological recordings were made from 31 neurons (17 sham vs. 14 infected) with a mean capacitance of 33.2±3.8 pF. Resting conductance was also decreased with Nb infection as shown in FIG. 10, (sham 240.1±42, infected 141.3±23.6 pS/pF, p=0.058) but there was no change in the resting membrane potential. FIG. 11 shows that the number of action potentials evoked during a 500 ms pulse at 2× rheobase was increased from 1.8±0.4 to 7.7±1.7 (p=0.004) with Nb infection. FIG. 12 shows that action potential half-width was decreased from 1.1±0.1 to 0.7±0.1 ms (p=0.01) in Nb infected neurons. FIG. 13 shows that Rheobase was decreased in Nb neurons but was not significantly different (sham 5.2±2.1, infected 2.7±1.2 pA/pF, p=0.31). Taken together these data clearly demonstrate that a mild, transient, intestinal inflammatory episode can lead to long term excitability (LTE) in both DRG and NG neurons, persisting for weeks after resolution of the gut inflammation.

### Full Figure Legends for FIGS. 6 to 13

FIG. 6: A scatterplot of the normalized resting conductance levels of sham and Nb infected DRG neuron populations. The conductance of each neuron under resting conditions at the beginning of each experiment is measured and divided by the capacitance of the cell in order to normalize the conductance level to cell size. Using a Mann-Whitney test, there is a significant reduction in the resting conductance of Nb infected neurons. Mean data is expressed as median ± interquartile range.

FIG. 7: DRG neuron rheobase is decreased in Nb infected cells. The top half of this figure shows example traces of rheobase measurements in individual sham and Nb infected DRG neurons. The blue bars indicate increasing amounts of current injected into the cells, with the amount of current necessary to elicit an action potential (AP) highlighted. The green and red traces show the resulting membrane potential trace of sham and infected neurons respectively. In these particular examples, an AP was elicited at 44 pA in the sham neuron and at 8 pA in the infected neuron. The scatterplot below shows the entire population data normalized to cell capacitance. There is a significant decrease in the rheobase of Nb infected neurons.

FIG. 8: DRG excitability is increased in Nb infected neurons. The top half of this figure shows example traces of sham and Nb infected DRG neurons in response to a current injection equivalent to 2× rheobase. The blue bars indicate the amount of current injected into each cells, whilst the green and red traces show the resulting number of APs fired in sham and infected neurons respectively. In these particular examples, 2 APs were elicited in the sham neuron and 7 APs evoked in the infected neuron. The scatterplot below shows the entire population data. There is a significant increase in the number of APs evoked at 2× rheobase of Nb infected neurons.

FIG. 9: sAHP amplitude is decreased in Nb infected neurons. The top half of this figure shows example traces of the sAHP elicited after a burst of APs in sham and Nb infected DRG neurons. The scatterplot below shows the entire population data. There is a significant decrease in the SAHP amplitude in Nb infected neurons.

FIG. 10: Scatterplots of the resting conductance levels of sham and Nb infected nodose neurons. The conductance of each neuron under resting conditions at the beginning of each experiment is measured and plotted on the left. This data is then normalized by dividing by the capacitance of the cell as plotted on the right. Once normalized, the resting conductance of Nb infected neurons is shown to be decreased compared to sham, but this fall just outside of statistical significance.

FIG. 11: Nodose neuron excitability is increased in Nb infected neurons. The top half of this figure shows example traces of sham and Nb infected DRG neurons in response to a current injection equivalent to 2× rheobase. In these particular examples, 2 APs were elicited in the sham neuron and 7 APs evoked in the infected neuron. The scatterplot below shows the entire population data. There is a significant increase in the number of APs evoked at 2× rheobase of Nb infected neurons.

FIG. 12: Action potential shape parameters are altered in nodose neurons by Nb infection. These scatterplots demonstrate an increase (not statistically significant) in the antipeak amplitude of the AP (equivalent to the fast afterhyperpolarization), with a decrease in both the AP half-width and the AP maximum decay slope following Nb infection. The decreases in half width and decay slope are indicative of faster APs lacking a hump on the downward slope of the AP.

FIG. 13: Nodose neuron rheobase is not significantly altered by Nb infection. The rheobase of each neuron is measured and plotted on the left. This data is then normalized by dividing by the capacitance of the cell as plotted on the right. Once normalized, although there is a slight decrease, there is no significant difference in the rheobase of Nb infected neurons.

### Example 4

### Gene Expression Profiling of Nodose and Dorsal Root Ganglia

Taking into account that only 3% of the neurons in DRG and NG project to the abdominal viscera, laser capture microdissection was applied to isolate these specific neurons out of the entire ganglion. In this way visceral afferent specific gene expression profiles in DRG and NG were identified in Sh/NS, I/NS, Sh/S and I/S mice.

### (1) Gene Expression Profiles of Visceral Sensory Neurons in Dorsal Root Ganglia:

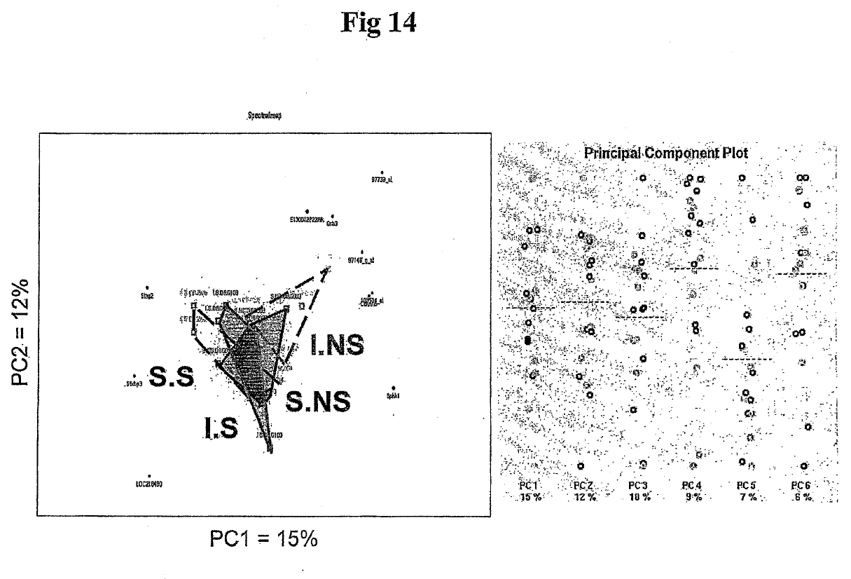

RNA extracted from laser captured DRG neurons was amplified and hybridised to MG-430V2.0 whole genome arrays interrogating expression levels of 39,000 gene transcripts simultaneously. FIG. 14 shows a graphical exploration of microarray data using spectral map analysis (SPM). As can be seen from the overlapping nature of the quadrants this revealed no differences in gene expression between the four studies groups. In order to identify individual genes that could be differentially expressed, Significance Analysis of Microarray data (SAM, q-value<0.1) and fold-difference filtering were applied (>1,5 fold difference). However, in agreement with SPM results no significantly differentially expressed genes were identified.

### (2) Gene Expression Profiles of Visceral Sensory Neurons in Nodose Ganglia:

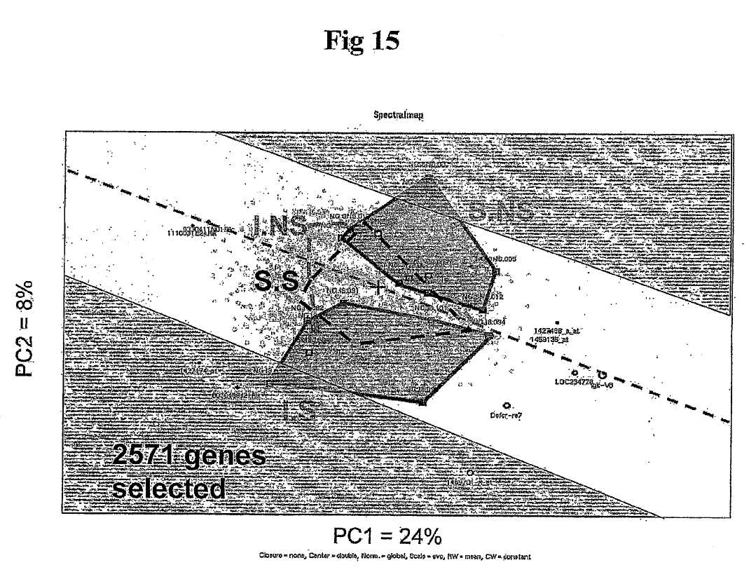

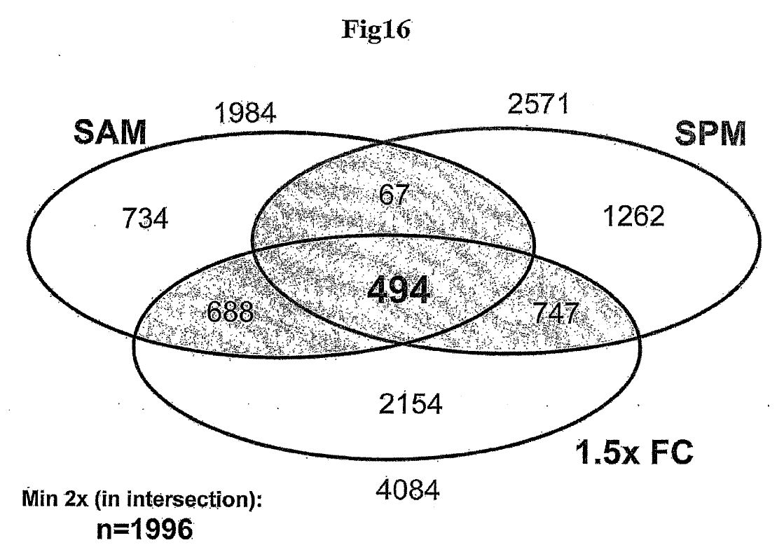

Laser captured material from NG was hybridised to MG-430V2.0 arrays. Spectral map analysis on the expression of 28,920 reliably detected genes as can be seen in FIG. 15 showed a clear difference between Sh/NS and I/S, whereas overall expression profile of the Sh/S and the I/NS are in the transition zone between the two outer groups. Spectral map analysis revealed 2571 genes of which the expression profile contributes to the difference between Sh/NS vs I/S. Combining those with genes that are identified by SAM (q<0.1) and fold-difference filtering (>1.5 fold difference) lead to the identification of 1994 genes, as represented in FIG. 16 that are significantly differently expressed after Nb infection, 1377 of which were increased and 617 were decreased. Altered NG genes included 19 G-protein coupled receptors, 23 ion channel genes, 80 kinases, and 118 other receptor-related genes.

Unexpectedly these data indicate that changes in gene expression are observed in NG rather than DRG neurons in an animal model for IBS. This strongly suggests that molecular changes at the level of the vagus could underlie symptoms observed in IBS.

### Full Figure Legends for FIGS. 14, 15 and 16

### FIG. 14—DRG SPM

Panel A: First two principal components (PC) of the weighted Spectral map analysis (SPM) applied on normalized microarray data for gene expression profiles of DRG neurons in all four animal groups (Sh/NS, I/NS, SH/S and IS). On the spectral map squares depict different samples whereas circles depict genes (size of circle correspond to intensity). Distances between squares are a measure for similarity between samples. A positive association of a gene with a given sample (i.e. an upregulation of that gene in that particular sample) results in the positioning of the gene and sample on a common line through the centroid (depicted by a cross). Genes contributing significantly (measured by their distance form the centroid) to difference between samples are annotated with their Affymetrix identifier (www.affymetrix.com/analysis/netaffx). Only the first two principle components are plotted against each other, together explaining 27% of the variance in the data. As indicated by the coloured lines, no separation between the groups is observed indicating no differences in overall gene expression pattern is presented at the level of visceral DRG neurons.

Panel B: Distribution of the samples over the different principal components in the spectral map analysis showing that none of the principal components differentiates the groups. The percentages of variance explained by each component are indicated at the bottom of the graph.

FIG. 15—NG SPM: Spectral map biplot of gene expression profiles of DRG neurons in all four animal groups (Sh/NS, I/NS, SH/S and IS). Only the first two principle components are plotted against each other, together explaining 32% of the variance in the data. As indicated by the coloured lines and the dotted line, a clear separation between the Sh/NS and the I/S groups is observed indicating a clear differences in overall gene expression pattern is presented at the level of visceral NG neurons. Indicated by the shaded area are the 2571 genes contributing the most to this overall difference in expression profile.

FIG. 16—NG SPM-SAM-FC: Venn diagrams summarizing the number of genes identified by spectral map analysis (SPM), significance analyis (SAM) and fold difference filtering (FD). The selection of 1996 genes was based on the fulfilment of at least two of the three criteria mentioned above.

### Example 5

### Changes in VR1, CCK_{A}, SST₂ and 5-HT3_{A}

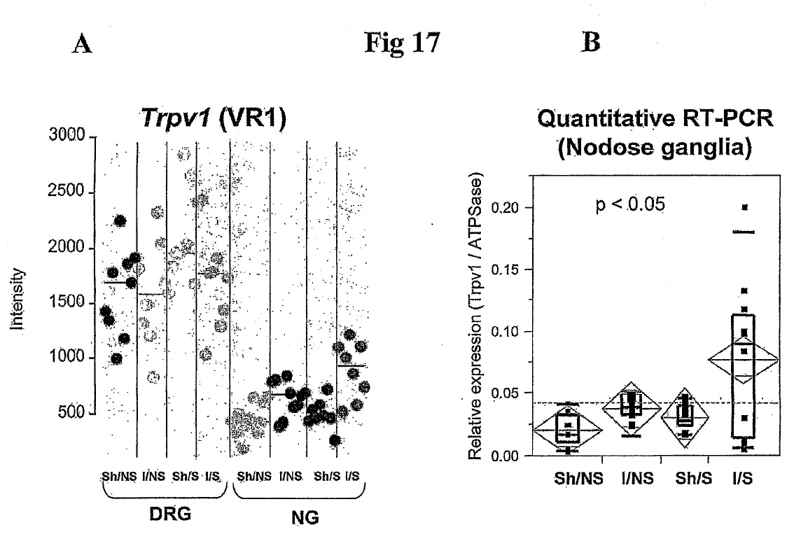

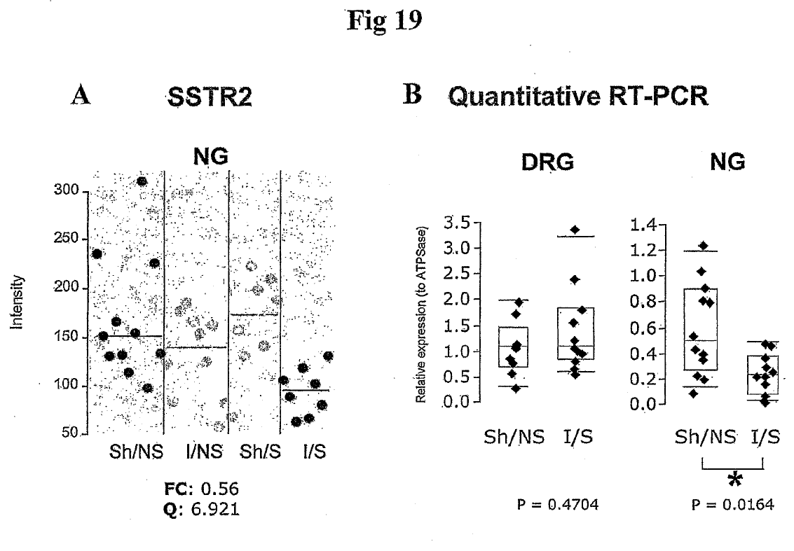

FIGS. 17 to 20 show that both the vanilloid receptor VR1 (Trpv1) and cholecystokinin receptor A (Cckar) were upregulated in Nb infected NG neurons, whilst serotonin receptor 3A (Htr3a) and somatostatin 2 receptor (Sstr2) were downregulated. It is also noted that the effect of Nb infection alone on expression level of these genes was enhanced in infected stress-exposed animals. Changes in mRNA levels measured on the arrays were confirmed using quantitative PCR. FIG. 17B shows that expression of Trpv1 mRNA was significantly increased in infected/stressed animals when compared to sham/non stressed. FIG. 19B shows expression levels for SST₂ receptor in infected and non infected DRG and NG neurons from the same animal as assessed by quantitative PCR. It can be seen that there is no significant change in expression between infected and non infected neurons in DRG neurons, whereas, a significant decrease in expression is seen in NG neurons of infected/stressed animals when compared to non infected/non stressed animals.

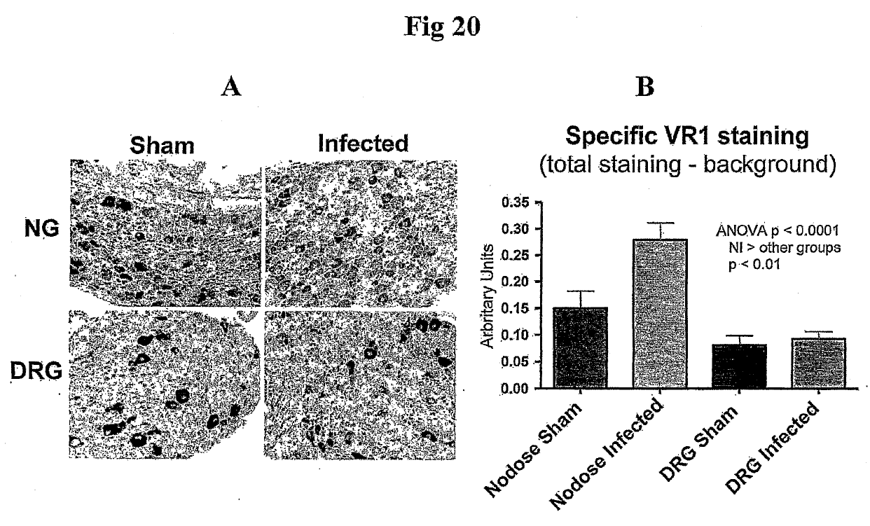

In respect to the vanilloid receptor VR1 (encoded by Trpv1) FIGS. 20A and B show that increased mRNA levels were confirmed at the protein level using immunohistochemical staining of NG sections. In addition the lack of differences at the level of DRG neurons was confirmed with no difference in immunoreactivity in infected versus sham neurons.

### Full Figure Legends for FIGS. 17 to 20

### FIG. 17—NG TRPV1

Panel A: Signal intensities of Vanilloid Receptor 1 (Trpv1) mRNA levels as measured on the arrays. As indicated levels in DRG neurons did not differ whereas there was an obvious increase in expression level observed in NG neurons in infected stressed (I/S) animals compared to sham non stressed animals (Sh/NS).

Panel B: Expression levels for Trpv1 as assessed by quantitative PCR. A significant increase in Trpv1 mRNA levels was confirmed in infected stressed (I/S) animals compared to sham non stressed animals (Sh/NS).

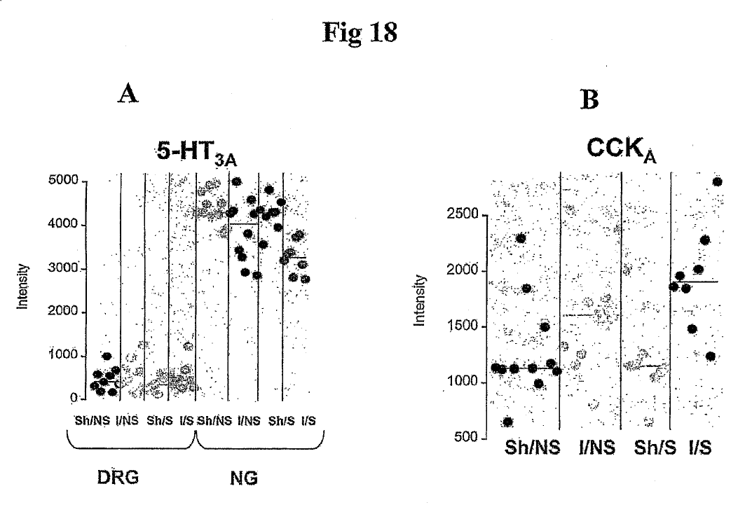

### FIG. 18—NG 5HT CCK_{A}

Panel A: Signal intensities of the 5HT_{3A} receptor mRNA levels as measured on the arrays. Each dot represents expression level in a single animal. As indicated levels in DRG neurons did not differ whereas there was an obvious decrease in expression level observed in NG neurons in infected stressed (I/S) animals compared to sham non stressed animals (Sh/NS).

Panel B: Expression levels for CCK_{A} receptor. An increase in CCK_{A} receptor mRNA levels was observed in infected stressed (I/S) animals compared to sham non stressed animals (Sh/NS).

### FIG. 19—NG SST2

Panel A: Signal intensities of SST₂ receptor (Sst2r) mRNA levels as measured on the arrays. As indicated there was an obvious decrease in expression level observed in NG neurons in infected stressed (I/S) animals compared to sham non stressed animals (Sh/NS).

Panel B: Expression levels for SST₂ receptor (Sst2r) mRNA as assessed by quantitative PCR. A significant decrease in SST₂ mRNA levels was confirmed in infected stressed (I/S) animals compared to sham non stressed animals (Sh/NS) whereas no difference could be detected in DRG neurons of the same animals.

### FIG. 20-NG TRPV1 Quantitative Immunohistochemistry

Panel A: Representative images of Vanilloid Receptor 1 (VR1, Trpv1) immunoreactivity observed in sections of DRG an NG ganglia of infected and sham animals.

Panel B: Quantitation of VR1 immunoreactivity. A significant increase in immunoreactivity was observed in NG after in infection, confirming array and quantitative PCR data.

### Example 6

### Changes in Pressor-Depressor Response in Nb Infected Mice

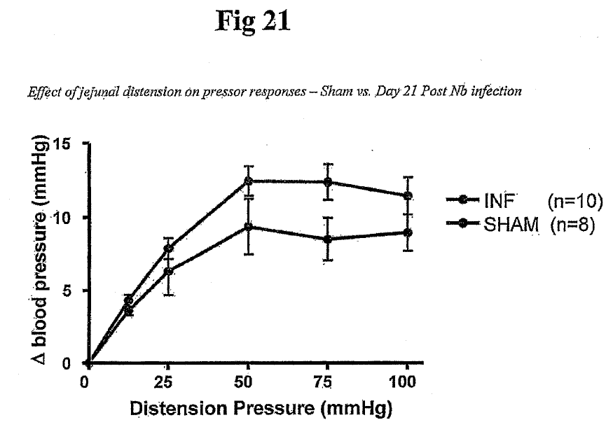

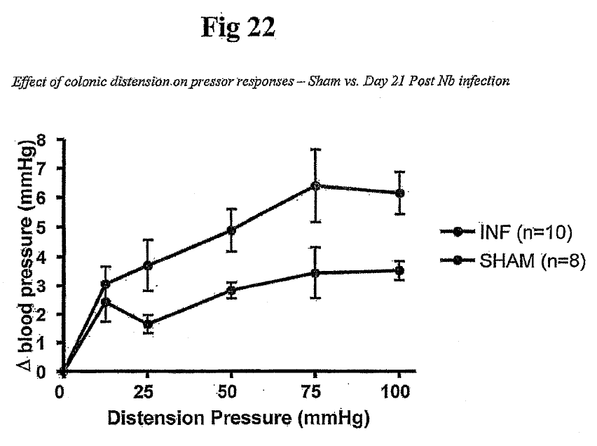

In order to measure visceral hypersensitivity in Nb infected mice changes in arterial blood pressure were recorded during phasic distention of both the jejunum and the colon. FIG. 21 illustrates the increase in blood pressure (pressor response) to jejunal distension of sham non-stressed vs. infected stressed mice at 21 days post Nb infection. The pressor response is increased in infected animals when compared to sham: a 2-way ANOVA demonstrates that there is a significant increase in the overall response profile with infection (p=0.0019). FIG. 22 illustrates the pressor response to colonic distension of sham non-stressed vs. infected stressed mice at 21 days post Nb infection. The pressor response is increased in infected animals when compared to sham: a 2-way ANOVA demonstrates that there is a significant increase in the overall response profile with infection (p<0.0001).

It has been shown that a mild, transient, intestinal inflammatory episode inflicted by Nb can lead to long term excitability (LTE) in both DRG and NG neurons, persisting for weeks after resolution of the gut inflammation. However, at the molecular level, changes in mRNA and protein level were only observed in NG sensory neurons. Blood pressure recordings confirmed that LTE resulted in visceral hypersensitivity in mice post Nb infection. It is to be expected that these changes can be reversed by treating with modulators of molecules shown to be altered in vagal afferents. This data demonstrates a new and powerful model of sensory neuron plasticity that may be applied to the study of visceral pain. Moreover strong evidence is provided that vagal afferents are the major targets mediating visceral hypersensitivity and thus constitute an important target for the treatment of IBS.

Further work undertaken by the inventors on jejunal mechanosensitivity using balloon ramp distension to 60 mmHg has suggested that although there was a difference in initial studies, in repeated studies there was no difference. Therefore, any jejunal mechanosensitivity is inconsistent and a reason for this variability has yet to be elucidated

### Full Figure Legends for FIGS. 21 and 22

FIG. 21—PR in jejunum: Effect of jejunal phasic distension on pressor responses in Sham vs. Day 21 Post Nb infection animals. Number of animals in each group is indicated between brackets.

FIG. 22-PR in colon: Effect of colonic phasic distension on pressor responses in Sham vs. Day 21 Post Nb infection animals. Number of animals in each group is indicated between brackets.

### Example 7

### Compound Testing

The compound octreotide was tested in the non-human animal screen of the invention as follows:

Nodose neurons were dissociated and cultured in preparation for patch clamp experiments as has been described elsewhere. These nodose neurons were either obtained from Balb/c mice 21 days after infection with Nb or from sham mice, thus enabling a comparison of the effects of octreotide on both sham and Nb-infected nodose neurons.

Octreotide (1 μM) was applied to individual neurons via a fast perfusion system. Octreotide's effects were recorded on the cell's resting membrane potential (RMP) and the number of action potentials fired at 2× rheobase of each neuron.

Electrophysiological recordings were obtained in total from 30 sham neurons and 37 infected neurons. Of these, recordings were sustained during octreotide application in 27 sham neurons and 25 infected neurons. Octreotide had no significant effect on the RMP of either sham neurons (control: −57.6±1.8 mV; octreotide: −54.7±2.1 mV) or infected neurons (control: −51.6±1.7 mV; octreotide: −51.7±2.1 mV).

The number of action potentials evoked by a 2× rheobase current stimulus was significantly increased in infected neurons when compared to sham neurons. Octreotide reduced the number of action potentials at 2× rheobase in both sham and infected nodose neurons. Hence octreotide reduced neuronal excitability in both sham and infected neurons. These results suggest that the hyperexcitability observed in infected nodose neurons can be normalized by octreotide treatment.

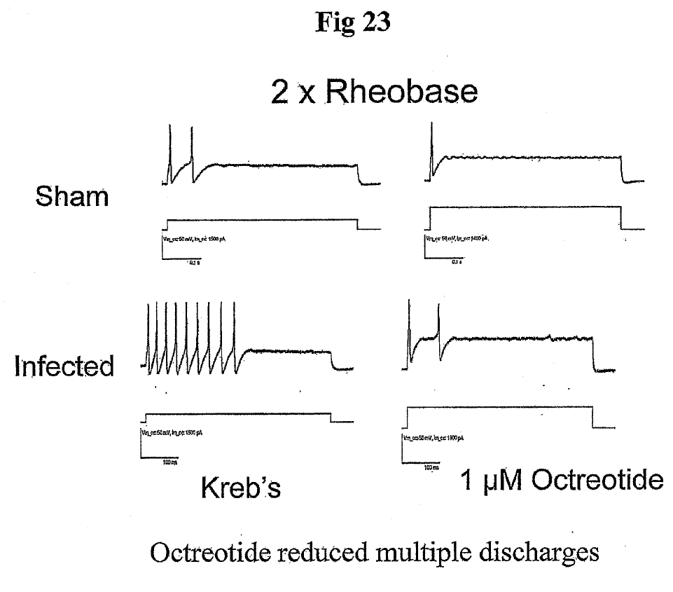

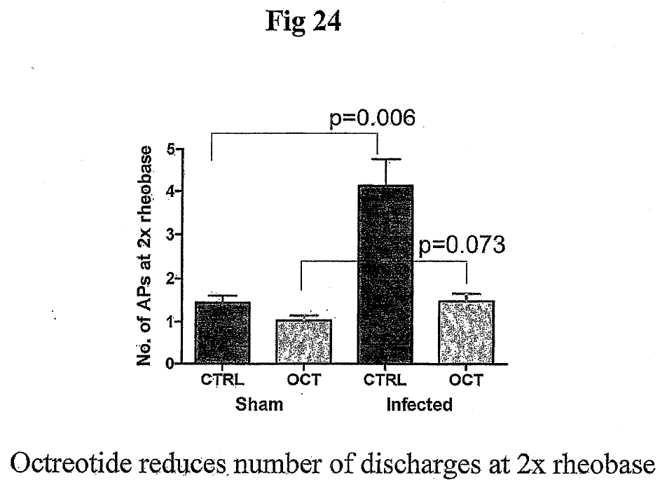

The data confirming these results is shown in FIGS. 23 and 24.

### Full Figure Legends for FIGS. 23 and 24

FIG. 23 shows the effects of 1 μM octreotide on evoked action potential discharge in sham and infected neurons. In control conditions in the presence of Krebs, a current that is 2× the rheobase of the neuron evokes 2 action potentials in a sham nodose neuron and 9 action potentials in an infected nodose neuron. After addition of 1 μM octreotide, the number of action potentials evoked is reduced in both sham neurons (1 action potential) and infected (2 action potentials) neurons.

FIG. 24 shows the mean effects of 1 μM octreotide on evoked action potential discharge in sham and infected neurons. Infection significantly increases the number of action potentials evoked at 2× rheobase in nodose neurons. Addition of octreotide reduces the number of action potentials in both sham and infected neurons. There is no significant difference between the effect of octreotide on sham and infected neurons.

### Example 8

### Investigation of Chemical Hypersensitrivity

In light of the conclusion that there is no consistent change in the mechanosensitivity of the jejunum following Nb infection, further work was undertaken to investigate if there is any change in the chemical sensitivity.

Balb/c mice were injected subcutaneously with 500 L3 Nb larvae in PBS, or with PBS only (shams). Experiments were performed 3-4 weeks post-infection. Mesenteric afferent recordings were obtained from isoflurane anaesthetized mice using conventional extracellular recording techniques. A 5 cm section of the jejunum was intubated to allow continuous intraluminal perfusion (0.15 ml/min) of either 0.9% saline or 50 mM hydrochloric acid (HCl). Jejunal afferent nerve activity and intraluminal pressure (IP) was recorded in response to a 2.5 min HCl application (at time 0s). Baseline activity (−100 to 0s), acute acid response (50 to 110 s) and prolonged acid response (410 to 560 s) were measured and compared between sham and Nb infected mice.

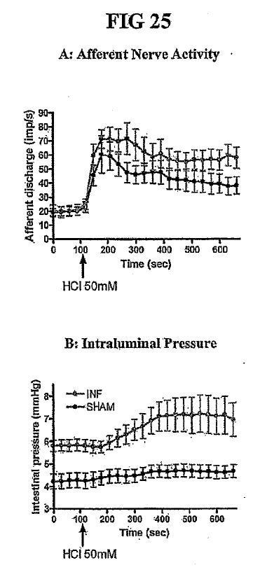

As shown in FIGS. 25 & 26, The experiments showed that in response to HCl perfusion there was an acute nerve response that peaked after 120±14.9 s after the response onset, with no significant change in IP. As this response gradually decreased over ˜10 mins, there was a concomitant increase in IP. Afferent nerve activity and IP never returned to pre-HCl exposure levels. There was no significant difference between baseline nerve activity in sham and Nb infected animals, but there was a significantly higher baseline IP in infected mice. The acute nerve response following HCl infusion was not significantly different between sham and infected mice. However, in the prolonged response period there was a significant increase in the nerve activity in infected animals. In addition there was a significantly greater prolonged increase in (IP) in Nb infected animals. Although it is possible that the increased IP may contribute to the increased prolonged nerve response in Nb infected mice, there was no significant direct correlation between the two measures.

The results indicate that Nb infection leads to an increased intestinal chemical sensitivity. Jejunal acidification elicits an acute nerve response which was similar in sham and infected groups and had no associated IP changes. This is followed by a prolonged nerve response that was significantly greater in infected groups than sham groups, and an uncorrelated prolonged IP response that was only clearly present in infected groups.

It is to be expected that these changes can be reversed by treating with modulators of molecules shown to be altered in vagal afferents. This data demonstrates a new and powerful model of sensory neuron plasticity that may be applied to the study of visceral pain. Moreover strong evidence is provided that vagal afferents are the major targets mediating visceral hypersensitivity and thus constitute an important target for the treatment of IBS.

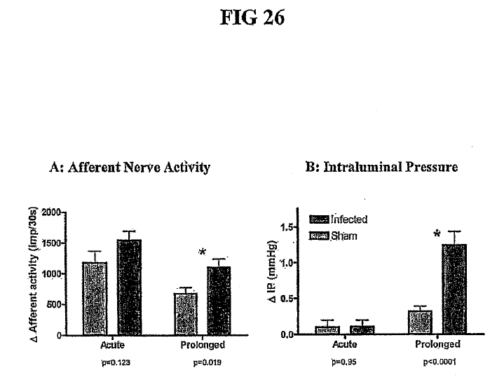

### Full Figure Legends for FIGS. 25 and 26

FIG. 25—Timecourse response to intraluminal administration of 50 mM HCl. A—Mesenteric afferent response to 50 mM HCl. Upon exposure of the nerves to acid (marked by ↑) there is a rapid increase in afferent activity that peaks after 120±14.9 s and gradually decrease after this point, but never returns to spontaneous nerve activity levels. The afferent response in infected animals (n=28) is larger (2-way ANOVA, p<0.001) than that recorded in sham animals (n=28). B—Intraluminal pressure response to 50 mM HCl. Both the resting IP and the response to acid were greater (2-way ANOVA, p<0.001) in infected animals (n=28) than in sham animals (n=28).

FIG. 26—Response to intraluminal administration of 50 mM HCl. A—Increase over baseline in the acute (1-2 min post-acid) and prolonged (7-10 min post-acid) phases of the afferent response to acid. There was a significant increase in the prolonged afferent response to acid. B—Increase over baseline in the acute (1-2 min post-acid) and prolonged (7-10 min post-acid) phases of the IP response to acid. There was a significant increase in the prolonged IP response to acid.

| | | | Log2Ratio.- | | |
|---|---|---|---|---|---|
| | | | Median.IS.- | | |
| Probe Set ID | Gene Symbol | Description | over.SNS | GenBank ID | SwissProt ID |
| 1456319_at | — | — | −2.84 | BG065719 | — |
| 1460241_a_at | Siat9 | sialyltransferase 9 (CMP-NeuAc:lactosylceramide alpha | −2.41 | BB829192 | O88829 /// Q9CZ65 /// Q9QWF8 /// Q9QWF9 |
| | | 2,3-sialyltransferase) | | | |
| 1421508_at | Odz1 | odd Oz/ten-m homolog 1 (Drosophila) | −2.34 | NM_011855 | Q8CAT1 /// Q9WTS4 |
| 1430203_at | Usp16 | ubiquitin specific protease 16 | −2.12 | BG067256 | Q99KM0 /// Q99LG0 |
| 1437757_at | Mizf-pending | MBD2 (methyl-CpG-binding protein)-interacting zinc | −2.08 | BB402190 | Q8BWY0 /// Q8K1K9 |
| | | finger protein | | | |
| 1450252_at | Onecut1 | one cut domain, family member 1 | −2.07 | NM_008262 | O08755 /// Q8K1C8 |
| 1447359_at | — | Mus musculus transcribed sequence with weak | −2.05 | AI326876 | — |
| | | similarity to protein sp: Q14587 (H. sapiens) | | | |
| | | Z268_HUMAN ZINC FINGER PROTEIN 268 (ZINC | | | |
| | | FINGER PROTEIN HZF3) | | | |
| 1449311_at | Bach1 | BTB and CNC homology 1 | −2.00 | NM_007520 | P97302 |
| 1417548_at | Sart3 | squamous cell carcinoma antigen recognized by T-cells 3 | −1.94 | BB546730 | AAH57156 /// BAC97877 /// Q8BPK9 /// Q8C3B7 /// Q8CFU9 /// Q9JLI8 |
| 1419173_at | Acy1 | aminoacylase 1 | −1.94 | NM_025371 | Q99JW2 /// Q9CR15 |
| 1423557_at | Ifngr2 | Interferon gamma receptor 2 | −1.92 | BF537076 | Q63953 /// Q8C352 |
| 1447382_at | Pigt | phosphatidylinositol glycan, class T | −1.88 | BB780056 | Q8BXQ2 |
| 1453247_at | 2810040O04Rik | RIKEN cDNA 2810040O04 gene | −1.87 | BE949501 | Q9CZ99 |
| 1453886_a_at | Slc25a26 | solute carrier family 25 (mitochondrial carrier, | −1.76 | AK017037 | Q8JZT2 |
| | | phosphate carrier), member 26 | | | |
| 1450332_s_at | Fmo5 | flavin containing monooxygenase 5 | −1.75 | NM_010232 | Q8R1W6 |
| 1427456_at | Wdfy3 | WD repeat and FYVE domain containing 3 | −1.73 | BF150771 | AAH58274 /// Q8C8H7 /// Q8CHB9 |
| 1425556_at | Crk7 | CDC2-related kinase 7 | −1.71 | BG070845 | BAC98047 /// Q8R457 /// Q9CVL4 |
| 1451676_at | Drap1 | Dr1 associated protein 1 (negative cofactor 2 alpha) | −1.71 | BC002090 | Q9D6N5 |
| 1422733_at | Fjx1 | four jointed box 1 (Drosophila) | −1.71 | AV230815 | Q8BQB4 |
| 1425050_at | 2610034N03Rik | RIKEN cDNA 2610034N03 gene | −1.66 | AK010892 | Q91V64 /// Q9D096 |
| 1453612_at | Nek1 | NIMA (never in mitosis gene a)-related expressed | −1.62 | AV254337 | — |
| | | kinase 1 | | | |
| 1455646_at | 2010004M13Rik | RIKEN cDNA 2010004M13 gene | −1.62 | BI904583 | — |
| 1448472_at | Vars2 | valyl-tRNA synthetase 2 | −1.59 | AF087680 | Q7TPT7 /// Q9Z1Q9 |
| 1439365_at | Myt1 | myelin transcription factor 1 | −1.59 | BB800584 | AAH63252 /// O08995 /// Q8CFC2 /// Q8CFH1 |
| 1422455_s_at | Nsf | N-ethylmaleimide sensitive fusion protein | −1.59 | BB400581 | P46460 /// Q8C3R2 /// Q8CCT9 /// Q8CEF0 /// Q923C6 |
| 1449578_at | Supt16h | suppressor of Ty 16 homolog (S. cerevisiae) | −1.58 | AW536705 | Q920B9 /// Q921H4 |
| 1427210_at | Baz2a | bromodomain adjacent to zinc finger domain, 2A | −1.57 | AW910654 | AAH58241 /// Q80U42 /// Q80VL8 /// Q8BRP6 /// Q8CGH2 /// Q91YE5 |
| 1456281_at | Fbxl18 | F-box and leucine-rich repeat protein 18 | −1.57 | BB401012 | — |
| 1416227_at | Arpc1b | actin related protein 2/3 complex, subunit 1B | −1.56 | BE979985 | Q91Z25 /// Q9CRC4 /// Q9WV32 |
| 1447920_at | — | Mus musculus transcribed sequences | −1.55 | BB420276 | — |
| 1437648_at | Pcyt1b | phosphate cytidylyltransferase 1, choline, beta isoform | −1.53 | BB541022 | Q80Y63 /// Q811Q8 /// Q811Q9 /// Q8BKD2 /// Q8C085 |
| 1422948_s_at | Hist1h3a | histone 1, H3a | −1.51 | NM_013550 | AAH58529 /// Q811M0 |
| 1455040_s_at | 1110062M06Rik | RIKEN cDNA 1110062M06 gene | −1.49 | BI965045 | — |
| 1444412_at | — | Mus musculus transcribed sequences | −1.49 | BM246867 | — |
| 1430173_x_at | Cyp4f16 | cytochrome P450, family 4, subfamily f, polypeptide 16 | −1.48 | AK009445 | Q99N17 |
| 1439615_at | Gan | giant axonal neuropathy | −1.48 | BB187898 | Q8CA72 |
| 1432871_at | 4932429P19Rik | RIKEN cDNA 4932429P19 gene | −1.47 | AK016536 | — |
| 1434588_x_at | Tbca | tubulin cofactor a | −1.46 | AI181686 | BAB27228 /// P48428 |
| 1421136_at | Edn3 | endothelin 3 | −1.46 | NM_007903 | BAC33211 /// BAC33915 /// BAC37561 /// P48299 |
| 1422696_at | Ttyh1 | tweety homolog 1 (Drosophila) | −1.45 | NM_021324 | AAH65694 /// Q8BQD6 /// Q8BRL4 /// Q8C7M4 /// Q9D3A9 /// Q9D5D1 /// Q9EQN7 /// |
| | | | | | Q9ESC3 |
| 1442106_at | C730036B14Rik | RIKEN cDNA C730036B14 gene | −1.44 | BB667730 | Q8BGE5 /// Q8BKB7 /// Q8BUA8 /// Q8BYF4 |
| 1453865_a_at | DXImx46e | DNA segment, Chr X, Immunex 46, expressed | −1.44 | AK010750 | Q91YL5 /// Q9CV50 /// Q9JIG6 |
| 1447250_a_at | 2610301F02Rik | RIKEN cDNA 2610301F02 gene | −1.44 | BB830098 | Q8BLD3 /// Q8BLW1 /// Q8BUK9 /// Q9D003 |
| 1417699_at | Gtf2f1 | general transcription factor IIF, polypeptide 1 | −1.43 | AV325174 | Q8BVJ2 /// Q8R5B7 /// Q9CSF1 |
| 1439252_at | Incenp | inner centromere protein | −1.42 | AV301185 | Q7TN28 /// Q9WU62 |
| 1428148_s_at | 0610011B16Rik | RIKEN cDNA 0610011B16 gene | −1.42 | BB203098 | AAH61006 /// Q8C9V7 /// Q8CA54 /// Q9D2V7 |
| 1443241_at | — | Mus musculus 13 days embryo stomach cDNA, RIKEN | −1.42 | AW544264 | — |
| | | full-length enriched library, clone: D530023N15 | | | |
| | | product: unclassifiable, full insert sequence | | | |
| 1452333_at | Smarca2 | SWI/SNF related, matrix associated, actin dependent | −1.41 | BM230202 | O35846 /// Q7TND4 /// Q8R1W7 /// Q99KH6 /// Q9CTU8 /// Q9D007 |
| | | regulator of chromatin, subfamily a, member 2 | | | |
| 1426361_at | 5730454B08Rik | RIKEN cDNA 5730454B08 gene | −1.41 | AV328883 | AAH58552 /// AAH66163 /// AAH66848 /// Q80TU7 /// Q8C1U4 /// Q8C5L5 /// Q8CBM9 /// |
| | | | | | Q99JN6 |
| 1448531_at | Lmnb2 | lamin B2 | −1.40 | NM_010722 | P21619 /// P48680 /// Q8CGB1 |
| 1422805_a_at | Ing3 | inhibitor of growth family, member 3 | −1.40 | BB020556 | Q8VEK6 /// Q99JS6 /// Q9ERB2 |
| 1429070_at | 4933440H19Rik | RIKEN cDNA 4933440H19 gene | −1.39 | AK009216 | AAH57613 /// Q8CEZ2 |
| 1439021_at | Centb5 | centaurin, beta 5 | −1.39 | BI412223 | AAH67016 /// Q8C8T5 |
| 1424054_at | Btbd2 | BTB (POZ) domain containing 2 | −1.38 | BC016566 | Q7TNF6 /// Q91YK4 |
| 1416871_at | Adam8 | a disintegrin and metalloprotease domain 8 | −1.36 | NM_007403 | Q05910 /// Q8C269 /// Q8R3D3 |
| 1436813_x_at | Khsrp | KH-type splicing regulatory protein | −1.36 | BB332580 | AAH64454 /// Q8CEN4 |
| 1450450_at | Dscr1l2 | Down syndrome critical region gene 1-like 2 | −1.35 | AF237888 | Q9JKK0 |
| 1435699_at | Ppm1l | protein phosphatase 1 (formerly 2C)-like | −1.35 | BG074188 | Q810H0 /// Q8BHN0 /// Q8C021 /// Q8C1D5 /// Q9Z0T1 |
| 1432402_at | 4930402F11Rik | RIKEN cDNA 4930402F11 gene | −1.35 | AK015048 | — |
| 1424142_at | Ikbkap | inhibitor of kappa light polypeptide enhancer in B-cells, | −1.34 | AF367244 | Q7TQH1 /// Q7T737 /// Q8C6B3 /// Q8CBI3 /// Q8CH82 /// Q8VHU5 /// Q8VHV9 /// Q9CT81 |
| | | kinase complex-associated protein | | | |
| 1424486_a_at | Txnrd1 | thioredoxin reductase 1 | −1.33 | BB284199 | Q8CF34 /// Q8CI31 /// Q99P49 /// Q9CSV5 /// Q9CVN8 /// Q9JMH6 |
| 1426786_s_at | Dhx38 | DEAH (Asp-Glu-Ala-His) box polypeptide 38 | −1.32 | BM195397 | O89064 /// Q80X98 /// Q8R1J6 |
| 1421013_at | Pltpnb | phosphotidylinositol transfer protein, beta | −1.32 | NM_019640 | BAC25830 /// P53811 /// Q8JZZ5 |
| 1456800_a_at | D130029J02Rik | RIKEN cDNA D130029J02 gene | −1.32 | BE685813 | — |
| 1417379_at | Iqgap1 | IQ motif containing GTPase activating protein 1 | −1.32 | NM_016721 | BAC97854 /// Q07230 /// Q80UW7 /// Q8BPA6 /// Q8CC64 /// Q8CDT3 /// Q8CGH5 /// |
| | | | | | Q9D408 /// Q9JKF1 |
| 1460616_at | Slco4c1 | solute carrier organic anion transporter family, member | −1.31 | BB400146 | Q8BGD4 |
| | | 4C1 | | | |
| 1437107_at | D9Bwg0185e | DNA segment, Chr 9, Brigham & Women's Genetics | −1.30 | AV220161 | AAH60618 /// BAC29230 /// P61294 |
| | | 0185 expressed | | | |
| 1445813_at | 0610012K18Rik | RIKEN cDNA 0610012K18 gene | −1.28 | BB205459 | Q8C5V5 /// Q8CA00 |
| 1418521_a_at | Mtx1 | metaxin 1 | −1.28 | NM_013604 | P47802 /// Q8R5C0 |
| 1417820_at | Tortb | torsin family 1, member B | −1.27 | BB004887 | Q8CBP2 /// Q8VEI4 /// Q9ER41 |
| 1416601_a_at | Dscr1 | Down syndrome critical region homolog 1 (human) | −1.26 | AF282255 | BAC36729 /// Q7TNY3 /// Q9JHG6 |
| 1447278_at | — | Mus musculus transcribed sequence with moderate | −1.25 | BB822306 | — |
| | | similarity to protein ref: NP_055771.1 (H. sapiens) | | | |
| | | KIAA1052 protein [Homo sapiens] | | | |
| 1449281_at | Nrtn | neurturin | −1.25 | NM_008738 | P97463 |
| 1453261_at | 2610035D17Rik | RIKEN cDNA 2610035D17 gene | −1.24 | BB760848 | — |
| 1433653_at | BC029169 | cDNA sequence BC029169 | −1.24 | BG173681 | Q8CID3 |
| 1426326_at | Zfp91 | zinc finger protein 91 | −1.23 | U05343 | AAH57323 /// AAH64766 /// BAC30862 /// P51642 /// Q62509 /// Q8BPY3 /// Q8C2B4 /// |
| | | | | | Q8CDZ3 |
| 1442775_at | — | Mus musculus transcribed sequences | −1.23 | AI481700 | — |
| 1455622_at | Podxl2 | podocalyxin-like 2 | −1.23 | BB461988 | Q8CAE9 /// Q8CFW3 |
| 1426432_a_at | Slc4a4 | solute carrier family 4 (anion exchanger), member 4 | −1.23 | BE655147 | O88343 /// Q8QZR9 /// Q9R1C4 |
| 1447055_at | Dnajc11 | DnaJ (Hsp40) homolog, subfamily C, member 11 | −1.22 | BB769600 | Q8BP83 /// Q8C1Z4 /// Q8C6U5 |
| 1445854_at | C230004F18 | hypothetical protein C230004F18 | −1.22 | BB380166 | Q8C4l6 |
| 1433479_at | 5730410I19Rik | RIKEN cDNA 5730410I19 gene | −1.22 | AV030071 | AAH58535 /// Q8BU04 |
| 1429034_at | Eme2 | essential meiotic endonuclease 1 homolog 2 (S. pombe) | −1.21 | AK012738 | — |
| 1421955_a_at | Nedd4 | neural precursor cell expressed, developmentally down- | −1.21 | NM_010890 | P46935 /// Q8BNU7 |
| | | regulted gene 4 | | | |
| 1432291_at | 0610033M10Rik | RIKEN cDNA 0610033M10 gene | −1.21 | AK002748 | — |
| 1431191_a_at | Syt1 | synaptotagmin 1 | −1.19 | AK018163 | P46096 /// Q8BRM4 |
| 1424151_at | Jtv1-pending | JTV1 gene | −1.19 | BC026972 | Q8R010 /// Q8R2Y6 /// Q8R3V2 |
| 1447065_at | 9630041C05 | hypothetical protein 9630041C05 | −1.19 | BB129691 | — |
| 1420596_at | Cacng2 | calcium channel, voltage-dependent gamma subunit 2 | −1.18 | NM_007583 | O88602 /// Q8C8F5 |
| 1454309_at | Bag5 | BCL2-associated athanogene 5 | −1.18 | BB646622 | Q8CDX7 /// Q9CQW7 /// Q9CVQ6 |
| 1445307_at | — | Mus musculus 12 days embryo male wolffian duct | −1.18 | BB051515 | — |
| | | includes surrounding region cDNA, RIKEN full-length | | | |
| | | enriched library, clone: 6720430F13 | | | |
| | | productunclassifiable, full insert sequence | | | |
| 1437617_x_at | 1110034G24Rik | RIKEN cDNA 1110034G24 gene | −1.17 | BB387677 | Q9D112 |
| 1437968_at | Grin1 | glutamate receptor, ionotropic, NMDA1 (zeta 1) | −1.17 | AI385669 | P35438 /// Q8BZ96 /// Q8CFS4 |
| 1460704_at | Ring | radical fringe gene homolog (Drosophila) | −1.17 | AK004573 | AAH66023 /// O09009 |
| 1418414_at | Kcnh1 | potassium voltage-gated channel, subfamily H (eag- | −1.16 | NM_010600 | Q60603 |
| | | related), member 1 | | | |
| 1419502_at | D11Lgp1e | DNA segment, Chr 11, Lothar Hennighausen 1, | −1.16 | NM_031871 | Q99J23 /// Q99J92 |
| | | expressed | | | |
| 1425163_at | LOC224833 | hypothetical protein BC006605 | −1.16 | BC006605 | Q91Z58 |
| 1459881_at | — | Similar to fibrillarin (LOC237730), mRNA | −1.16 | AI595406 | Q80WS3 |
| 1432952_at | 4930448E22Rik | RIKEN cDNA 4930448E22 gene | −1.14 | AK015416 | — |
| 1447612_x_at | — | Mus musculus transcribed sequences | −1.14 | BB494168 | — |
| 1445718_at | — | Mus musculus transcribed sequences | −1.14 | BM237480 | — |
| 1439434_x_at | BC036961 | cDNA sequence BC036961 | −1.13 | BB317673 | — |
| 1457280_at | — | Mus musculus transcribed sequences | −1.13 | BB249354 | Q80V87 /// Q9CZ86 |
| 1435481_at | E430039K05Rik | RIKEN cDNA E430039K05 gene | −1.13 | BM194940 | AAL66764 /// Q8BHR7 /// Q8BHT8 /// Q8CI02 |
| 1424112_at | Igf2r | Insulin-like growth factor 2 receptor | −1.12 | BG092290 | AAA16037 /// Q07113 /// Q7TMR1 /// Q80VF2 /// Q8C2F9 /// Q8C6V9 /// Q8K0J1 |
| 1424359_at | Oplah | 5-oxoprolinase (ATP-hydrolysing) | −1.12 | BC025120 | Q8K010 /// Q8R3K2 |
| 1459536_at | Calcrl | calcitonin receptor-like | −1.12 | BB223961 | Q9R1W5 |
| 1442277_at | Chka | choline kinase alpha | −1.12 | BB546429 | O54804 /// Q99KD4 |
| 1436167_at | — | MRNA similar to SHB (Src homology 2 domain | −1.11 | BB798279 | Q8CG80 |
| | | containing) adaptor protein B (cDNA clone MGC: 30399 | | | |
| | | IMAGE: 4488005), complete cds | | | |
| 1455474_at | D6Wsu116e | DNA segment, Chr 6, Wayne State University 116, | −1.10 | BM197316 | AAH56942 /// Q80TW8 /// Q80UQ4 /// Q8BRP9 /// Q8CAP0 /// Q9CT54 |
| | | expressed | | | |
| 1427079_at | Mapre3 | microtubule-associated protein, RP/EB family, member 3 | −1.10 | U51204 | AAH57918 /// Q61167 |
| 1433635_at | Wdr18 | WD repeat domain 18 | −1.10 | BG073188 | Q8BHQ0 /// Q8K265 |
| 1434794_at | Arhf | ras homolog gene family, member f (in filopodia) | −1.10 | BM241811 | Q8BYP3 |
| 1418284_at | Tcfl1 | transcription factor-like 1 | −1.10 | NM_009336 | Q62481 /// Q810A9 /// Q99K81 |
| 1457597_at | — | Mus musculus transcribed sequences | −1.09 | AW121529 | — |
| 1441962_at | — | Mus musculus cDNA clone MGC: 58861 | −1.09 | BB079625 | Q810M3 |
| | | IMAGE: 6774557, complete cds | | | |
| 1425383_a_at | Fbx1 | pre B-cell leukemia transcription factor 1 | −1.08 | L27453 | AAB71192 /// P41778 /// Q8BFR8 /// Q99LS8 /// Q9D621 |
| 1424277_at | 1110020L19Rik | RIKEN cDNA 1110020L19 gene | −1.08 | AY029337 | Q8BKT8 /// Q924Z7 |
| 1460304_a_at | Ubtf | upstream binding transcription factor, RNA polymerase I | −1.08 | BB832806 | P25976 /// Q9DBH1 |
| 1449150_at | A930040G15Rik | RIKEN cDNA A930040G15 gene | −1.08 | NM_133922 | AAH66816 /// Q9JJG3 |
| 1417619_at | Gadd45gip1 | growth arrest and DNA-damage-inducible, gamma | −1.08 | BE368753 | AAH61069 /// Q8BT05 /// Q9CR59 |
| | | interacting protein 1 | | | |
| 1427510_at | Gnai1 | guanine nucleotide binding protein, alpha inhibiting 1 | −1.08 | U38501 | Q61018 |
| 1428516_a_at | 2310045B01Rik | RIKEN cDNA 2310045B01 gene | −1.07 | BI903628 | Q8K1H3 /// Q9CY41 /// Q9D6Z0 /// Q9D942 |
| 1443816_s_at | — | Mus musculus adult male bone cDNA, RIKEN full- | −1.07 | BB240086 | Q8K323 |
| | | length enriched library, clone: 9830142N16 | | | |
| | | product: unclassifiable, full insert sequence | | | |
| 1431885_a_at | Mus81 | MUS81 endonuclease homolog (yeast) | −1.07 | AK004647 | Q91ZJ0 |
| 1426514_at | 4631426J05Rik | RIKEN cDNA 4631426J05 gene | −1.07 | AK019474 | Q80TW4 /// Q8BLQ5 /// Q91XQ5 /// Q9D2N6 |
| 1427635_at | Kif5a | kinesin family member 5A | −1.07 | AU067277 | AAH67051 /// P28738 /// Q8CHF1 |
| 1451278_a_at | 2610205E22Rik | RIKEN cDNA 2610205E22 gene | −1.06 | BC027220 | Q8R2U4 |
| 1459865_x_at | — | — | −1.06 | AV278384 | — |
| 1429416_at | 2900074C18Rik | RIKEN cDNA 2900074C18 gene | −1.05 | AK013779 | Q9D6E4 |
| 1426249_at | Adrbk1 | adrenergic receptor kinase, beta 1 | −1.05 | AF333028 | Q7TS64 /// Q99MK8 |
| 1425558_at | Klc3 | kinesin light chain 3 | −1.05 | BC017147 | Q91W40 |
| 1441456_at | Mmp24 | matrix metalloproteinase 24 | −1.04 | BB335489 | — |
| 1456571_at | 1700001E16Rik | RIKEN cDNA 1700001E16 gene | −1.04 | AV101812 | Q8C5I7 /// Q9DAR7 |
| 1446947_at | — | — | −1.04 | BG072149 | — |
| 1442100_at | Inpp5f | inositol polyphosphate-5-phosphatase F | −1.04 | BB619843 | AAH67200 /// BAC98059 /// Q8C8G7 /// Q8CBW2 /// Q8CDA1 |
| 1451621_at | 5830417C01Rik | RIKEN cDNA 5830417C01 gene | −1.04 | BC002200 | Q8BIB9 /// Q9D291 |
| 1438410_at | A230098A12Rik | RIKEN cDNA A230098A12 gene | −1.04 | BB295128 | Q8BJK6 /// Q8BYL5 |
| 1459430_at | Gpr158 | G protein-coupled receptor 158 | −1.04 | BB429778 | Q8BSU1 /// Q8C3D0 /// Q8C419 /// Q8CHB0 |
| 1444702_at | — | Adult male epididymis cDNA, RIKEN full-length | −1.04 | AV381472 | — |
| | | enriched library, clone: 9230116A06 product: unknown | | | |
| | | EST, full insert sequence | | | |
| 1458193_at | Fabp9 | fatty acid binding protein 9, testis | −1.03 | AV278565 | — |
| 1421846_at | Wsb2-pending | WD-40-repeat-containing protein with a SOCS box 2 | −1.03 | BM730566 | AAH55100 /// O54929 |
| 1440817_x_at | G630024C07Rik | RIKEN cDNA G630024C07 gene | −1.03 | BB242445 | AAH62882 /// Q8BJ90 |
| 1451286_s_at | Fus | fusion, derived from t(12; 16) malignant liposarcoma | −1.02 | AF224264 | AAH58247 /// P56959 /// Q8CFQ9 /// Q91VQ2 |
| | | (human) | | | |
| 1425875_a_at | Lepr | leptin receptor | −1.01 | U58862 | P48356 |
| 1433496_at | 2810024B22Rik | RIKEN cDNA 2810024B22 gene | −1.01 | AV122321 | AAH56951 /// Q8K297 |
| 1441263_a_at | A930005H10Rik | RIKEN cDNA A930005H10 gene | −1.00 | AV009179 | Q8CEK0 |
| 1423416_at | Smarcc1 | SWI/SNF related, matrix associated, actin dependent | −1.00 | BI558117 | P97496 /// Q7TS80 /// Q7TT29 |
| | | regulator of chromatin, subfamily c, member 1 | | | |
| 1435135_at | B230106I24Rik | RIKEN cDNA B230106I24 gene | −1.00 | AV369935 | Q8BLF1 /// Q8BYQ0 /// Q8BZK3 |
| 1428954_at | Slc9a3r2 | solute carrier family 9 (sodium/hydrogen exchanger), | −1.00 | AK004710 | AAH65778 /// Q9JHL1 |
| | | isoform 3 regulator 2 | | | |
| 1437545_at | 5730409O11 | hypothetical protein 5730409O11 | −0.99 | BM194994 | Q8BK28 /// Q8CFE3 /// Q8CHI2 |
| 1459705_at | — | Mus musculus transcribed sequences | −0.99 | BE980857 | — |
| 1437524_x_at | 0610011B16Rik | RIKEN cDNA 0610011B16 gene | −0.99 | BB534801 | AAH61006 /// Q8C9V7 /// Q8CA54 /// Q9D2V7 |
| 1425311_at | 4930432F04Rik | RIKEN cDNA 4930432F04 gene | −0.99 | BC016220 | Q9D2l6 |
| 1456725_x_at | Vil2 | villin 2 | −0.99 | BB114808 | P26040 /// Q8CBU4 |
| 1434946_at | C330021A05Rik | RIKEN cDNA C330021A05 gene | −0.98 | BB303415 | Q8BX00 /// Q8CFP6 /// Q923I0 |
| 1451707_s_at | Slc41a3 | solute carrier family 41, member 3 | −0.98 | BC011108 | Q921R8 /// Q9DC67 |
| 1416845_at | Hspa5bp1 | heat shock 70 kDa protein 5 binding protein 1 | −0.98 | NM_133804 | Q8BX93 /// Q922P8 |
| 1447766_x_at | 0610025L06Rik | RIKEN cDNA 0610025L06 gene | −0.98 | AV003249 | AAH68130 /// Q8BGB5 |
| 1459230_at | Plod2 | procollagen lysine, 2-oxoglutarate 5-dioxygenase 2 | −0.98 | BB525112 | Q8BIK8 /// Q9R0B9 |
| 1438489_at | Smn | survival motor neuron | −0.98 | BM068889 | P97801 |
| 1426790_at | Ssrp1 | structure specific recognition protein 1 | −0.98 | BC024835 | Q8CGA6 |
| 1422320_x_at | — | — | −0.98 | NM_008836 | — |
| 1422977_at | Gp1bb | glycoprotein lb, beta polypeptide | −0.98 | NM_010327 | — |
| 1451778_at | BC011210 | cDNA sequence BC011210 | −0.97 | BC011210 | Q91X84 |
| 1435083_at | Ctxn | cortexin | −0.97 | BI155559 | Q8K129 |
| 1427334_s_at | 2810474O19Rik | RIKEN cDNA 2810474O19 gene | −0.97 | BE196832 | Q8CCW3 /// Q8CCZ9 /// Q8CFR7 /// Q9CSA5 /// Q9CU82 |
| 1438576_x_at | 2810454L23Rik | RIKEN cDNA 2810454L23 gene | −0.96 | BG143413 | — |
| 1440255_at | Mizf-pending | MBD2 (methyl-CpG-binding protein)-interacting zinc | −0.96 | BB826899 | Q8BWY0 /// Q8K1K9 |
| | | finger protein | | | |
| 1416122_at | Ccnd2 | cyclin D2 | −0.96 | NM_009829 | P30280 /// Q9D8L9 |
| 1453349_at | 2410019P08Rik | RIKEN cDNA 2410019P08 gene | −0.96 | AK010559 | Q9CWL2 |
| 1424657_at | MGC29021 | hypothetical protein MGC29021 | −0.96 | BB151477 | Q8JZX2 /// Q8VE26 /// Q91VG7 /// Q9D3K9 |
| 1458623_at | — | Mus musculus transcribed sequences | −0.95 | AI413154 | — |
| 1431035_at | Daam1 | dishevelled associated activator of morphogenesis 1 | −0.95 | AW988556 | AAR05118 /// BAC97995 /// Q8BPM0 /// Q8BTF1 |
| 1447902_at | 1810013A23Rik | RIKEN cDNA 1810013A23 gene | −0.95 | AV050195 | — |
| 1415784_at | Vps35 | vacuolar protein sorting 35 | −0.95 | BI654068 | Q9EQH3 |
| 1451433_at | 2310010G13Rik | RIKEN cDNA 2310010G13 gene | −0.94 | BC019171 | Q8VED1 /// Q9D7F8 |
| 1424077_at | 2610020H15Rik | RIKEN cDNA 2610020H15 gene | −0.94 | AK016023 | Q9CRY7 /// Q9CT14 /// Q9D4X7 |
| 1429686_at | Polr3f | polymerase (RNA) III (DNA directed) polypeptide F | −0.94 | BG070811 | BAC29327 /// BAC36385 /// Q8C108 /// Q921X6 |
| 1449082_at | Mfap5 | microfibrillar associated protein 5 | −0.94 | NM_015776 | Q9QZJ6 |
| 1436909_at | B430110G05Rik | RIKEN cDNA B430110G05 gene | −0.94 | AW542746 | Q7TSU9 /// Q8BGF9 |
| 1424675_at | Slc39a6 | solute carrier family 39 (metal ion transporter), member 6 | −0.94 | BB825002 | Q7TPP9 /// Q7TQE0 /// Q8C145 /// Q8R518 |
| 1436468_at | Zdhhc8 | zinc finger, DHHC domain containing 8 | −0.93 | BB553914 | Q7TNF7 /// Q8CCU8 /// Q99KF7 |
| 1456054_a_at | Pum1 | pumillo 1 (Drosophila) | −0.93 | BB314559 | Q80U78 |
| 1439214_a_at | Api5 | apoptosis inhibitor 5 | −0.93 | AV118744 | O35841 /// Q922L2 |
| 1456663_x_at | 2410018G23Rik | RIKEN cDNA 2410018G23 gene | −0.93 | BB718785 | Q8BJJ1 /// Q8R0I4 /// Q9CWL9 |
| 1437607_at | Gcnt2 | glucosaminyltransferase, I-branching enzyme | −0.93 | BB357165 | AAR95649 /// AAR95650 /// AAR95651 /// P97402 /// Q7TPQ8 /// Q8BK09 /// Q8BW63 /// |
| | | | | | Q9D2A8 |
| 1429023_at | 2900042E01Rik | RIKEN cDNA 2900042E01 gene | −0.92 | AK013537 | BAC38147 |
| 1436343_at | Chd4 | chromodomain helicase DNA binding protein 4 | −0.92 | BM502696 | AAH58578 /// Q8BM83 /// Q99JM0 /// Q9CTT2 |
| 1453172_at | Stch | stress 70 protein chaperone, microsome-associated, | −0.92 | BE533039 | Q8BM72 /// Q9D1X5 |
| | | human homolog | | | |
| 1460255_at | Tnfsf13b | tumor necrosis factor (ligand) superfamily, member 13b | −0.92 | NM_033622 | Q7TQ58 /// Q8BVA3 /// Q8BWP2 /// Q8BZM8 /// Q9WU72 |
| 1434351_at | MGC37347 | hypothetical protein MGC37347 | −0.92 | BF021398 | Q80U93 /// Q8CHS9 |
| 1438422_at | Lrrc20 | leucine rich repeat containing 20 | −0.92 | BB143476 | — |
| 1427421_at | Tcp10 | t-complex protein-10 complex | −0.91 | AV257292 | AAH61173 /// Q62184 /// Q80YU2 /// Q8C5S9 /// Q8C641 |
| 1439828_x_at | Rab38 | Rab38, member of RAS oncogene family | −0.91 | AV364767 | Q8QZZ8 |
| 1446910_at | — | Mus musculus transcribed sequences | −0.91 | BG073901 | — |
| 1421499_a_at | Ptpn14 | protein tyrosine phosphatase, non-receptor type 14 | −0.91 | NM_008976 | Q62130 /// Q8C3A0 /// Q8CAV9 /// Q8CE88 /// Q9JLJ6 /// Q9JLJ7 /// Q9JLJ8 /// Q9JLJ9 |
| 1449423_at | Mast1 | microtubule associated serine/threonine kinase 1 | −0.91 | NM_019945 | Q7TQ97 /// Q7TQG9 /// Q80TN0 /// Q9R1L5 |
| 1427143_at | Jarid1b | jumonji, AT rich interactive domain 1B (Rbp2 like) | −0.91 | BC019446 | AAH57318 /// Q80Y84 /// Q8BLU1 /// Q8C1P6 /// Q8JZL8 /// Q8VCQ4 |
| 1456343_at | Slc35f1 | solute carrier family 35, member F1 | −0.91 | BB540579 | AAH59075 /// Q8BGK5 /// Q8BKD4 /// Q8BX52 |
| 1421056_at | Dnase1l3 | deoxyribonuclease 1-like 3 | −0.91 | BC012671 | O55070 |
| 1451386_at | Blvrb | biliverdin reductase B (flavin reductase (NADPH)) | −0.90 | BC027279 | Q923D2 |
| 1438416_at | Thrap5 | thyroid hormone receptor associated protein 5 | −0.90 | BM238407 | AAH57056 |
| 1418738_at | Scn1b | sodium channel, voltage-gated, type I, beta polypeptide | −0.90 | BC009652 | P97952 |
| 1451285_at | Fus | fusion, derived from t(12;16) malignant liposarcoma | −0.90 | AF224264 | AAH58247 /// P56959 /// Q8CFQ9 /// Q91VQ2 |
| | | (human) | | | |
| 1448907_at | Thop1 | thimet oligopeptidase 1 | −0.90 | NM_022653 | Q8C1A5 /// Q8K0J9 /// Q8K2D4 /// Q99LK5 /// Q9EPX1 |
| 1453111_a_at | 3010027G13Rik | RIKEN cDNA 3010027G13 gene | −0.89 | AK019396 | Q9D8K8 |
| 1455084_x_at | Shmt2 | serine hydroxymethyl transferase 2 (mitochondrial) | −0.89 | BB758291 | Q99K87 /// Q9CZN7 |
| 1437034_x_at | Marcks | myristoylated alanine rich protein kinase C substrate | −0.89 | BB332426 | P26645 |
| 1430221_at | 9130008F23Rik | RIKEN cDNA 9130008F23 gene | −0.89 | BB763680 | Q9D2Z6 |
| 1449258_at | D11Wsu99e | DNA segment, Chr 11, Wayne State University 99, | −0.89 | AV225714 | AAH60985 /// Q8BKU4 /// Q9CQP1 |
| | | expressed | | | |
| 1456911_at | Clasp2 | CLIP associating protein 2 | −0.89 | BB831639 | Q8BRT1 /// Q8BSE7 /// Q8CHE3 /// Q8R337 /// Q99JI3 /// Q9DB80 |
| 1423117_at | Pum1 | pumillo 1 (Drosophila) | −0.89 | BB837171 | Q80U78 |
| 1420397_a_at | Mint-pending | Msx2 interacting nuclear target protein | −0.89 | NM_019763 | Q62504 |
| 1438112_at | — | Mus musculus transcribed sequence | −0.89 | AA546727 | Q8BT43 |
| 1449685_s_at | 4933425A18Rik | RIKEN cDNA 4933425A18 gene | −0.89 | C80494 | Q9D404 |
| 1451134_a_at | 2410018G23Rik | RIKEN cDNA 2410018G23 gene | −0.88 | BC026789 | Q8BJJ1 /// Q8R0I4 /// Q9CWL9 |
| 1427718_a_at | Mdm2 | transformed mouse 3T3 cell double minute 2 | −0.88 | X58876 | P23804 /// Q91XK7 |
| 1417144_a_at | Tubg1 | tubulin, gamma 1 | −0.88 | NM_134024 | P83887 |
| 1418003_at | 1190002H23Rik | RIKEN cDNA 1190002H23 gene | −0.88 | NM_025427 | Q9D0U0 /// Q9DBX1 |
| 1429440_at | 1810041L15Rik | RIKEN cDNA 1810041L15 gene | −0.88 | BI734299 | AAH62953 /// BAC98225 |
| 1450145_at | Dbpht1 | DNA binding protein with his-thr domain | −0.87 | NM_019416 | Q64150 |
| 1439630_x_at | Sbsn | suprabasin | −0.87 | AI844734 | AAR20795 /// Q80WB4 /// Q8C7L5 /// Q8CIT9 /// Q8K2V9 |
| 1417236_at | Ehd3 | EH-domain containing 3 | −0.87 | BM234719 | Q8K590 /// Q8R0V6 /// Q9QXY6 |
| 1419379_x_at | Fxyd2 | FXYD domain-containing ion transport regulator 2 | −0.87 | NM_052823 | BAC24982 /// Q04646 |
| 1436341_at | F830020C16Rik | RIKEN cDNA F830020C16 gene | −0.87 | BM125569 | Q80WC2 /// Q8BJA3 /// Q8BWE7 /// Q99LV1 |
| 1433713_at | Gcn1l1 | GCN1 general control of amino-acid synthesis 1-like 1 | −0.87 | BB794873 | AAH56933 /// AAH68244 /// Q8BIX2 /// Q8BJ26 /// Q8BTM7 /// Q8CHH7 |
| | | (yeast) | | | |
| 1454775_at | Hdac10 | histone deacetylase 10 | −0.87 | AW548891 | AAH64018 |
| 1451484_a_at | Syn1 | synapsin 1 | −0.87 | BC022954 | O88935 /// Q8QZT8 |
| 1435560_at | Itgal | integrin alpha L | −0.87 | BI554446 | P24063 /// Q9R200 /// Q9WTV4 |
| 1449615_s_at | Hdlbp | high density lipoprotein (HDL) binding protein | −0.87 | C77256 | Q8VDJ3 |
| 1419747_at | Asgr2 | asialoglycoprotein receptor 2 | −0.87 | NM_007493 | P24721 |
| 1460639_a_at | Atox1 | ATX1 (antioxidant protein 1) homolog 1 (yeast) | −0.86 | NM_009720 | O08997 |
| 1423250_a_at | Tgfb2 | transforming growth factor, beta 2 | −0.86 | BF144658 | P27090 /// Q8CDZ9 /// Q91VP5 /// Q921T1 |
| 1436014_a_at | Rusc1 | RUN and SH3 domain containing 1 | −0.86 | BB806780 | AAH56360 /// AAH57034 /// Q8BG26 /// Q9CVB4 |
| 1421016_at | Ighmbp2 | immunoglobulin mu binding protein 2 | −0.86 | AW259474 | P40694 |
| 1430058_at | — | Mus musculus transcribed sequence with strong | −0.86 | AK016826 | — |
| | | similarity to protein pir: S12207 (M. musculus) S12207 | | | |
| | | hypothetical protein (B2 element) - mouse | | | |
| 1436320_at | — | Mus musculus, clone IMAGE: 4206343, mRNA | −0.86 | W45978 | — |
| 1444022_at | — | Mus musculus transcribed sequences | −0.86 | BF782342 | — |
| 1447938_at | — | Mus musculus cDNA clone MGC: 69869 | −0.86 | BB379724 | Q8BQ57 /// Q8C3Q3 /// Q8C5T7 /// Q8C8H0 |
| | | IMAGE: 6822098, complete cds | | | |
| 1436022_at | Endogl1 | endonuclease G-like 1 | −0.86 | BB089035 | Q8C163 |
| 1442590_at | Tnfrsf22 | tumor necrosis factor receptor superfamily, member 22 | −0.86 | BB366863 | Q8BFY5 /// Q9ER62 |
| 1424052_at | Thap4 | THAP domain containing 4 | −0.86 | BC013538 | AAH57963 /// AAH63758 /// AAH66042 /// Q91WR2 /// Q9CVE5 /// Q9CVG3 |
| 1451421_a_at | Lzf | leucine zipper domain protein | −0.86 | BC006914 | Q8BL37 /// Q922N4 /// Q923H8 |
| 1435888_at | 9030024J15Rik | RIKEN cDNA 9030024J15 gene | −0.86 | AV369812 | — |
| 1424749_at | Wdfy1 | WD40 and FYVE domain containing 1 | −0.86 | BC025226 | Q8R3I5 /// Q9DAD3 |
| 1445218_at | — | Mus musculus transcribed sequences | −0.86 | BE955408 | — |
| 1440801_s_at | — | Mus musculus transcribed sequences | −0.85 | BB391602 | Q8BVT9 /// Q8BX71 |
| 1436868_at | Rtn4rl1 | reticulon 4 receptor-like 1 | −0.85 | BM508396 | AAP82835 /// Q8K0S5 |
| 1419299_at | 2010012O05Rik | RIKEN cDNA 2010012O05 gene | −0.85 | NM_025563 | Q9CRC6 |
| 1447739_x_at | Klhdc4 | kelch domain containing 4 | −0.85 | AV294746 | AAH58359 /// Q8CIK0 /// Q921I2 |
| 1417475_at | Atp13a | ATPase type 13A | −0.85 | NM_133224 | Q810K8 /// Q9EPE9 |
| 1446374_at | Cln8 | ceroid-lipofuscinosis, neuronal 8 | −0.85 | BB460605 | AAH59212 /// AAH66074 /// AAO89218 /// BAC33570 /// BAC40269 /// BAC40944 /// |
| | | | | | Q80VH8 /// Q8BJ42 /// Q8BNW2 /// Q8BW76 /// Q8C033 /// Q922S7 /// Q9QUK3 |
| 1460378_a_at | Tes | testis derived transcript | −0.85 | BC010465 | P47226 /// Q921B1 /// Q921W7 /// Q99L61 |
| 1438858_x_at | H2-Aa | histocompatibility 2, class II antigen A, alpha | −0.85 | AV018723 | AAC17908 /// AAC17909 /// AAR19089 /// P01910 /// P04227 /// P04228 /// P14434 /// |
| | | | | | P14435 /// P14436 /// P14437 /// P14438 /// P23150 /// Q860C1 /// Q8K2X0 /// Q9TQ71 /// |
| | | | | | Q9TQ72 |
| 1435549_at | Trpm4 | transient receptor potential cation channel, subfamily M, | −0.85 | BI685685 | AAH58632 /// BAC81769 /// BAC81770 /// Q7TN37 /// Q80Y94 /// Q80YB3 /// Q811E2 /// |
| | | member 4 | | | Q8BLM7 |
| 1415732_at | Bat5 | HLA-B associated transcript 5 | −0.85 | BG071718 | Q9Z1Q2 |
| 1422369_at | V1ra6 | vomeronasal 1 receptor, A6 | −0.85 | NM_053221 | — |
| 1434594_at | B230373P09Rik | RIKEN cDNA B230373P09 gene | −0.85 | BB497449 | Q8BWG2 |
| 1442103_at | C79399 | expressed sequence C79399 | −0.85 | AW554925 | — |
| 1422972_s_at | Gcn5l2 | general control of amino acid synthesis-like 2 (yeast) | −0.84 | NM_020004 | AAH63752 /// Q99KW4 /// Q9JHD2 |
| 1436618_at | Sfxn5 | sideroflexin 5 | −0.84 | BB379739 | Q8BRQ9 /// Q925N0 |
| 1423875_at | AI450540 | expressed sequence AI450540 | −0.84 | BB321867 | AAH62949 /// Q80TB5 /// Q80VI3 /// Q8BKS4 /// Q8C8M2 /// Q8CDM8 /// Q8R1V3 |
| 1446973_at | — | Mus musculus transcribed sequences | −0.84 | BG076107 | — |
| 1424755_at | Hip1 | huntingtin interacting protein 1 | −0.84 | BB320674 | Q8C303 /// Q8VD75 /// Q9D1Z6 |
| 1459863_x_at | Gga1 | golgi associated, gamma adaptin ear containing, ARF | −0.84 | BB006096 | Q8R0H9 |
| | | binding protein 1 | | | |
| 1429631_at | Sirt6 | sirtuin 6 (silent mating type information regulation 2, | −0.84 | AK013316 | P59941 |
| | | homolog) 6 (S. cerevisiae) | | | |
| 1426095_a_at | Tnfrsf22 | tumor necrosis factor receptor superfamily, member 22 | −0.84 | AY046551 | Q8BFY5 /// Q9ER62 |
| 1445460_at | Bach2 | BTB and CNC homology 2 | −0.84 | BE457827 | — |
| 1447074_at | — | Mus musculus transcribed sequences | −0.84 | BG068627 | — |
| 1426920_x_at | Itgb1 | integrin beta 1 (fibronectin receptor beta) | −0.83 | BM120341 | P09055 /// Q60993 /// Q8BTU0 /// Q8BUD1 /// Q8BVU1 /// Q8BY44 |
| 1423981_x_at | Slc25a29 | solute carrier family 25 (mitochondrial carrier, | −0.83 | BC006711 | Q8BL03 |
| | | palmitoylcamitine transporter), member 29 | | | |
| 1445564_at | — | — | −0.83 | BE688513 | — |
| 1456271_at | — | Mus musculus transcribed sequences | −0.83 | BB039066 | Q80V96 |
| 1459897_a_at | Sbsn-pending | suprabasin | −0.83 | AI507307 | AAR20795 /// Q80WB4 /// Q8C7L5 /// Q8CIT9 /// Q8K2V9 |
| 1457564_at | Dffa | DNA fragmentation factor, alpha subunit | −0.83 | BB194910 | AAH58213 /// O54786 /// Q8BQC7 /// Q8C535 /// Q8CA98 |
| 1434610_at | Plec1 | plectin 1 | −0.83 | BM210485 | Q923J2 /// Q9QXS1 |
| 1417289_at | Plekha2 | pleckstrin homology domain-containing, family A | −0.83 | NM_031257 | Q9ERS5 |
| | | (phosphoinositide binding specific) member 2 | | | |
| 1428147_at | 0610011B16Rik | RIKEN cDNA 0610011B16 gene | −0.82 | BB203098 | AAH61006 /// Q8C9V7 /// Q8CA54 /// Q9D2V7 |
| 1427128_at | Ptpn23 | protein tyrosine phosphatase, non-receptor type 23 | −0.82 | BM195862 | AAH59902 /// Q8R1Z5 /// Q923E6 |
| 1451452_a_at | Rgs16 | regulator of G-protein signaling 16 | −0.82 | U72881 | BAC37678 /// P97428 /// Q7TNU9 /// Q80V16 |
| 1417959_at | Pdlim7 | PDZ and LIM domain 7 | −0.82 | NM_026131 | Q80ZY6 /// Q810S3 /// Q8BVJ7 /// Q8C1S4 /// Q9CRA1 |
| 1423068_at | ift172 | intraflagellar transport 172 | −0.82 | AK006007 | AAH60948 /// AAH66096 /// AAR05390 /// Q80TI4 /// Q80W19 /// Q9DAB0 |
| 1419038_a_at | Csnk2a1 | casein kinase II, alpha 1 polypeptide | −0.82 | BB283759 | AAH60742 /// Q60737 /// Q61177 /// Q8CD20 /// Q8R0X4 /// Q9D0E8 |
| 1416360_at | Snag1 | sorting nexin associated golgi protein 1 | −0.82 | AV344473 | Q8C788 /// Q91ZR2 |
| 1460743_at | Tigd5 | tigger transposable element derived 5 | −0.82 | BB553398 | Q8BQA1 /// Q8C381 /// Q8CBD5 |
| 1452835_a_at | Polrmt | polymerase (RNA) mitochondrial (DNA directed) | −0.82 | AK003792 | Q8BJE0 /// Q8BKF1 /// Q9D196 |
| 1439216_at | — | Mus musculus adult male aorta and vein cDNA, RIKEN | −0.82 | BB211804 | Q8BRV0 |
| | | full-length enriched library, clorne: A530095A18 | | | |
| | | product: hypothetical protein, full insert sequence | | | |
| 1435680_a_at | Dpp7 | dipeptidylpeptidase 7 | −0.82 | BG067113 | Q8R082 /// Q9ET22 |
| 1426777_a_at | Wasl | Wiskott-Aldrich syndrome-like (human) | −0.81 | BF466143 | AAH58642 /// Q7TPN5 /// Q80VV6 /// Q91YD9 /// Q9CXQ9 |
| 1435681_s_at | Homer3 | homer homolog 3 (Drosophila) | −0.81 | AI647511 | — |
| 1459658_at | — | Mus musculus transcribed sequence with weak | −0.81 | BB785334 | P49718 /// Q8BQ03 /// Q8C2I9 |
| | | similarity to protein pir: I58401 (M. musculus) I58401 | | | |
| | | protein-tyrosine kinase (EC 2.7.1.112) JAK3 - mouse | | | |
| 1429345_at | D2Ertd435e | DNA segment, Chr2, ERATO Doi 435, expressed | −0.81 | AK016563 | BAC32303 /// Q8BKL6 /// Q8BYN2 /// Q9D4F8 |
| 1435105_at | 110061N23Rik | RIKEN cDNA 1110D61N23 gene | −0.81 | BG066986 | Q8BTC4 /// Q8K0W3 |
| 1453427_at | Csnk2a1 | casein kinesa II, alpha 1 polypeptide | −0.81 | AK011501 | AAH60742 /// Q60737 /// Q61177 /// Q8CD20 /// Q8R0X4 /// Q9D0E8 |
| 1424460_s_at | BC005662 | cDNA sequence BC005662 | −0.81 | BG068664 | AAH66809 /// Q8BG23 /// Q8BJT4 /// Q8BUX7 /// Q99JU6 |
| 1451083_s_at | Aars | alanyl-1RNA synthetase | −0.81 | BC026611 | AAH58620 /// AAP57355 /// Q8BGQ7 /// Q8R346 |
| 1415750_at | Tbl3 | transducin (beta)-like 3 | −0.81 | BC019504 | Q8C4J7 /// Q8CE86 /// Q8VE90 |
| 1455394_at | Piasg-pending | protein inhibitor of activated STAT gamma | −0.81 | BI412631 | Q9JM05 |
| 1438188_x_at | Slc25a29 | solute carrier family 25 (mitochondrial carrier, | −0.81 | BB832209 | Q8BL03 |
| | | palmitoylcarnitine transporter), member 29 | | | |
| 1436106_x_at | 2310015A05Rik | RIKEN cDNA 2310015A05 gone | −0.81 | BI689456 | — |
| 1447112_s_at | Cryl1 | crystallin, lamda 1 | −0.81 | C85932 | BAC31583 /// BAC37964 /// Q8R4W7 /// Q99KP3 |
| 1426502_s_at | Gpt1 | glutamic pyruvic transaminase 1, soluble | −0.80 | AK008086 | Q8QZR5 |
| 1418627_at | Gclm | glutamate-cysteine ligase, modifier subunit | −0.80 | NM_008129 | BAC25831 /// O09172 |
| 1452170_at | 2010209O12Rik | RIKEN cDNA 2010209O12 gene | −0.80 | BC019714 | Q80TE1 /// Q80VD4 /// Q8C228 /// Q8VCJ5 |
| 1444775_at | 9930033D15Rik | RIKEN cDNA 9930033D15 gene | −0.80 | BB660772 | — |
| 1419907_s_at | BB219290 | expressed sequence BB219290 | −0.80 | BB219290 | AAH64708 /// Q8K3U9 /// Q8VHP5 /// Q920A9 |
| 1429772_at | Plxna2 | plexin A2 | −0.80 | BB085537 | AAH56475 /// AAH68155 /// P70207 /// Q80TZ7 /// Q80XE5 /// Q8R1I4 |
| 1425964_x_at | Hspb1 | heat shock protein 1 | −0.80 | U03561 | BAB22579 /// BAB27099 /// P14602 /// Q9Z2L2 /// Q9Z2L3 |
| 1434263_at | — | Mus musculus, clone IMAGE: 1246018, mRNA | −0.80 | AV307274 | — |
| 1459578_at | — | Mus musculus transcribed sequence with weak | −0.80 | BG063140 | — |
| | | similarity to protein pir: I58401 (M. musculus) I58401 | | | |
| | | protein-tyrosine kinase (EC 2.7.1.112) JAK3 - mouse | | | |
| 1425429_s_at | Hif3a | hypoxia indicible factor 3 alpha subunit | −0.80 | AF416641 | Q8VHR1 /// Q9Z215 |
| 1448844_at | 1810044O22Rik | RIKEN cDNA 1810044O22 gene | −0.80 | NM_025558 | AAH58812 /// AAH62980 /// Q9CQX2 /// Q9D1M6 /// Q9D8R3 |
| 1449738_s_at | P38ip-pending | transcription factor (p38 Interacting protein) | −0.80 | C80158 | AAR87814 /// Q7TT00 /// Q8BG53 /// Q9JLS9 |
| 1439833_at | 3110018K01Rik | RIKEN cDNA 3110018K01 gene | −0.80 | BQ176645 | — |
| 1451769_s_at | Pcdha11 | protocadherin alpha 11 | −0.80 | BB265776 | AAH60211 /// BAC97930 /// O88190 /// O88191 /// O88192 /// O88193 /// O88194 /// O88195 |
| | | | | | /// O88689 /// O88690 /// Q8BRP3 /// Q8BRR0 /// Q8K490 /// Q8K491 /// Q8K492 /// Q8K493 |
| | | | | | /// Q8K495 /// Q8K496 /// Q8K4A3 /// Q8K4A7 /// Q91Y09 /// Q91Y10 /// Q91Y11 /// Q91Y12 |
| | | | | | /// Q91Y13 /// Q91Y14 /// Q91Y15 /// Q91Y16 /// Q91Y17 /// Q91Y18 /// Q91Y19 /// |
| | | | | | Q91Y20 /// Q91Y21 |
| 1439812_at | 4930402H24Rik | RIKEN cDNA 4930402H24 gene | −0.79 | BQ173880 | AAH30418 /// Q8BIK2 /// Q8BIT3 /// Q9D5P8 |
| 1425525_a_at | P2rx4 | purinergic receptor P2X, ligand-gated ion channel 4 | −0.79 | AF089751 | Q9JJX3 /// Q9JJX4 /// Q9JJX5 /// Q9JJX6 /// Q9WUN9 /// Q9Z256 /// Q9Z257 |
| 1424922_a_at | Brd4 | bromodomain containing 4 | −0.79 | BC008532 | O35692 /// Q8BS78 /// Q8VHF7 /// Q8VHF8 /// Q9ESU6 |
| 1445303_at | — | Mus musculus transcribed sequences | −0.79 | BG066334 | — |
| 1441750_x_at | — | Mus musculus transcribed sequences | −0.79 | BB796499 | — |
| 1421859_at | Adam17 | a disintegrin and metalloproteinase domain 17 | −0.78 | C76813 | Q9Z0F8 |
| 1428180_at | 2810422J05Rik | RIKEN cDNA 2810422J05 gene | −0.78 | AK013135 | Q80XH1 |
| 1423944_at | Hpxn | hemopexin | −0.78 | BC011246 | Q91X72 |
| 1449362_a_at | Map4k6-pending | mitogen-activated protein kinase kinase kinase kinase 6 | −0.78 | NM_016713 | Q61165 /// Q7TT13 /// Q9JM52 |
| 1441190_at | — | Mus musculus transcribed sequence with moderate | −0.78 | AV381444 | Q9D898 |
| | | similarity to protein pir: S12207 (M. musculus) S12207 | | | |
| | | hypothetical protein (B2 element) - mouse | | | |
| 1427825_at | Slco1b2 | solute carrier organic anion transporter family, member | −0.78 | AB037192 | Q9JJL3 |
| | | 1b2 | | | |
| 1426866_at | D4st1 | dermatan 4 sulfotransferase 1 | −0.78 | AK011230 | Q80V53 /// Q8R304 /// Q9D0P2 |
| 1418089_at | Stx8 | syntaxin 8 | −0.78 | NM_018768 | AAH61118 /// O88983 /// Q8BS59 |
| 1437146_x_at | 0610011B16Rik | RIKEN cDNA 0610011B16 gene | −0.78 | AV025980 | AAH61006 /// Q8C9V7 /// Q8CA54 /// Q9D2V7 |
| 1426699_at | AU040320 | expressed sequence AU040320 | −0.77 | BG071197 | Q8BHR5 /// Q8BHU7 /// Q8BHZ3 /// Q8K135 /// Q8VBZ9 |
| 1449544_a_at | Kcnh2 | potassium voltage-gated channel subfamily H (eag- | −0.77 | NM_013569 | AAQ82708 /// O35219 /// Q80WG1 /// Q80XE8 |
| | | related), member 2 | | | |
| 1420221_at | — | Mus musculus, Similar to putative regulation protein | −0.77 | BB192718 | — |
| | | GS3, clone IMAGE: 5388383, mRNA | | | |
| 1429552_at | 1700019F09Rik | RIKEN cDNA 1700019F09 gene | −0.77 | AK006118 | Q9D432 /// Q9DA68 |
| 1427193_at | Brd8 | bromodomain containing 8 | −0.77 | BM219644 | Q8C049 /// Q8R3B7 /// Q8R583 /// Q8VDP0 /// Q9CXF6 |
| 1440312_at | Elovl7 | ELOVL family member 7, elongation of long chain fatty | −0.77 | BQ174957 | Q8BX38 /// Q8BYY8 /// Q9D2Y9 |
| | | acids (yeast) | | | |
| 1447476_at | — | Mus musculus transcribed sequences | −0.77 | BB079952 | — |
| 1439429_x_at | Dtx2 | deltex 2 homolog (Drosophila) | −0.77 | BB518874 | Q8R3P2 |
| 1427286_at | — | Mus musculus cDNA clone MGC: 62856 | −0.77 | BB130195 | Q7TQI4 /// Q8VI61 |
| | | IMAGE: 6494361, complete cds | | | |
| 1422853_at | Shc1 | src homology 2 domain-containing transforming protein | −0.77 | BB753533 | P98083 |
| | | C1 | | | |
| 1427699_a_at | Ptpn11 | protein tyrosine phosphatase, non-receptor type 11 | −0.77 | L08663 | AAH57398 /// AAH59278 /// P35235 /// Q63848 /// Q64509 /// Q99KW7 /// Q9CT18 |
| 1450214_at | Adora2b | adenosine A2b receptor | −0.77 | NM_007413 | Q60614 /// Q8BK41 /// Q8BXI2 |
| 1444433_at | — | Mus musculus transcribed sequences | −0.77 | BM246582 | — |
| 1417209_at | Sertad2 | SERTA domain containing 2 | −0.77 | NM_021372 | BAC97869 /// Q91VV6 /// Q9JJG5 |
| 1454370_at | 4930557B21Rik | RIKEN cDNA 4930557B21 gene | −0.77 | BB015975 | — |
| 1424429_s_at | Doc2a | double C2, alpha | −0.77 | BG065288 | Q7TMJ7 /// Q8R359 |
| 1425958_at | Il1f9 | interleukin 1 family, member 9 | −0.76 | AY071843 | Q8R460 |
| 1432515_at | 2410124H12Rik | RIKEN cDNA 2410124H12 gene | −0.76 | AK010774 | Q9CWF8 |
| 1428708_x_at | 2610009E16Rik | RIKEN cDNA 2610009E16 gene | −0.76 | AK011360 | Q80WS9 /// Q8VDQ2 /// Q9D0J8 |
| 1418423_s_at | Serpinb9f | serine (or cysteine) proteinase inhibitor, clade B, | −0.76 | AF425083 | AAH64758 /// AAH64759 /// Q8VHQ1 /// Q9DAZ7 |
| | | member 9f | | | |
| 1451634_at | 2810051F02Rik | RIKEN cDNA 2810051F02 gene | −0.76 | BC009123 | Q8BGE0 /// Q91VT2 /// Q9CZ68 |
| 1437219_at | — | Mus musculus transcribed sequences | −0.76 | AW553541 | — |
| 1431251_at | 1300011L04Rik | RIKEN cDNA 1300011L04 gene | −0.76 | AI451838 | — |
| 1436622_at | — | Similar to KIAA0522 protein (LOC245666), mRNA | −0.76 | AW492241 | — |
| 1451761_at | Hoxb4 | homeo box B4 | −0.76 | AV307188 | P10284 |
| 1427480_at | Leap2 | liver-expressed antimicrobial peptide 2 | −0.76 | AA571276 | Q91V13 |
| 1436695_x_at | Rbed1 | RNA binding motif and ELMO domain 1 | −0.76 | BB557382 | Q91YP6 |
| 1433464_at | Ipo13 | importin 13 | −0.76 | BB475675 | BAC98010 /// Q8K0C1 |
| 1430875_a_at | Pak1ip1 | PAK1 interacting protein 1 | −0.76 | AK017959 | Q80UT4 /// Q8C5N6 /// Q923K2 /// Q9DCE5 |
| 1418298_s_at | Dpysl4 | dihydropyrimidinase-like 4 | −0.76 | NM_011993 | O35098 |
| 1455992_at | Vgll4 | vestigial like 4 (Drosophila) | −0.75 | BG967636 | AAH60305 /// Q80V24 /// Q8BGS8 |
| 1439799_at | — | Mus musculus transcribed sequences | −0.75 | BE953350 | — |
| 1421210_at | C2ta | class II transactivator | −0.75 | AF042158 | P79621 /// Q8HW99 |
| 1452745_at | 1810044A24Rik | RIKEN cDNA 1810044A24 gene | −0.75 | AK007766 | Q8CD01 /// Q8CFV8 /// Q9D6K1 /// Q9D8R6 |
| 1430188_at | 1700037C18Rik | RIKEN cDNA 1700037C18 gene | −0.75 | AK012792 | Q8BT88 /// Q9D9P5 |
| 1422521_at | Dctn1 | dynactin 1 | −0.75 | NM_007835 | AAH66061 /// O08788 |
| 1437554_at | Plec1 | plectin 1 | −0.75 | BM232239 | Q923J2 /// Q9QXS1 |
| 1445517_at | — | Mus musculus transcribed sequence with week | −0.75 | BB144876 | — |
| | | similarity to protein ref: NP_081764.1 (M. musculus) | | | |
| | | RIKEN cDNA 5730493B19 [Mus musculus] | | | |
| 1446448_at | Pias1 | protein inhibitor of activated STAT 1 | −0.75 | AW547576 | — |
| 1449403_at | Pde9a | phosphodiesterase 9A | −0.75 | NM_008804 | AAH61163 /// O70628 /// Q8BSU4 /// Q8CB29 |
| 1437861_s_at | Prkce | protein kinase C, epsilon | −0.75 | BB335101 | P16054 |
| 1417967_at | Mms19l | MMS19 (MET18 S. cerevisiae)-like | −0.75 | NM_028152 | Q925N8 /// Q9D071 |
| 1452110_at | Mtrr | 5-methyltetrahydrofolate-homocysteine | −0.75 | BB757908 | Q8C1A3 /// Q8R0Y3 |
| | | methyltransferase reductase | | | |
| 1424428_at | AI854876 | expressed sequence AI854876 | −0.75 | BG065288 | Q7TMJ7 /// Q8R359 |
| 1422687_at | Nras | neuroblastoma ras oncogene | −0.74 | BB018528 | AAH58755 /// P08556 /// Q9D091 |
| 1428977_at | Chst8 | carbohydrate (N-acetylgalactosamine 4-0) | −0.74 | AK005217 | BAC87753 /// Q80XD4 /// Q8BQ86 |
| | | sulfotransferase 8 | | | |
| 1422944_a_at | Diap3 | diaphanous homolog 3 (Drosophila) | −0.74 | NM_019670 | Q8K331 /// Q9Z207 |
| 1416900_s_at | Lass1 | longevity assurance homolog 1 (S. cerevisiae) | −0.74 | NM_138647 | P20863 /// P27545 |
| 1430081_at | Phf15 | PHD finger protein 15 | −0.74 | AK004823 | BAC97907 /// Q8C7J4 |
| 1416294_at | Scamp3 | secretory carrier membrane protein 3 | −0.74 | NM_011886 | O35609 |
| 1417248_at | Ralbp1 | ralA binding protein 1 | −0.74 | NM_009067 | AAH67073 /// Q62172 |
| 1451703_s_at | Aprt | adenine phosphoribosyl transferase | −0.74 | M11310 | AAH05667 /// P08030 /// Q9DCY3 |
| 1444120_at | Bin1 | bridging integrator 1 | −0.74 | BG293813 | AAH65160 /// O08539 /// Q8C5M9 /// Q8C9N3 |
| 1416991_at | Mto1 | mitochondrial translation optimization 1 homolog | −0.74 | NM_026658 | AAH63256 /// Q8C6J8 /// Q923Z3 /// Q9CYK7 /// Q9D2Q5 |
| | | (S. cerevisiae) | | | |
| 1416965_at | Pcsk1n | proprotein convertase subtilisin/kexin type 1 inhibitor | −0.74 | AE181560 | Q91W26 /// Q9ESU4 /// Q9QXV0 |
| 1420419_a_at | Otof | otoferlin | -0.73 | NM_031875 | Q8CCE7 /// Q9ESF1 |
| 1424255_at | Supt5h | suppressor of Ty 5 homolog (S. cerevisiae) | −0.73 | BC007132 | AAH57449 /// AAH58598 /// AAH59849 /// O55201 |
| 1439234_a_at | 2410018G23Rik | RIKEN cDNA 2410018G23 gene | −0.73 | BE200117 | Q8BJJ1 /// Q8R0I4 /// Q9CWL9 |
| 1417628_at | Supt6h | suppressor of Ty 6 homolog (S. cerevisiae) | −0.73 | NM_009297 | BAC97879 /// Q62383 /// Q8BQY6 |
| 1423396_at | Agt | angiotensinogen | −0.73 | AK018763 | Q8VCN0 |
| 1427762_x_at | Hist1h2bp | histone 1, H2bp | −0.73 | M25487 | AAH61044 /// Q64477 /// Q8C622 |
| 1440253_at | Psmd11 | proteasome (prosome, macropain) 26S subunit, non- | −0.73 | AV136581 | Q7TMI0 /// Q8BG32 /// Q8BK73 /// Q8C0Z6 /// Q8K2N7 |
| | | ATPase, 11 | | | |
| 1444974_at | — | Mus musculus transcribed sequences | −0.73 | BG068713 | — |
| 1448330_at | Gstm1 | glutathione S-transferase, mu 1 | −0.73 | NM_010358 | P10649 |
| 1455801_x_at | Tbcd | tubulin-spacific chaperone d | −0.73 | BB392080 | AAH59843 /// Q8BYA0 /// Q8CHC0 /// Q8R199 |
| 1448810_at | Gne | glucosamine | −0.73 | BC015277 | Q91WG8 |
| 1449853_at | Sfxn2 | sideroflexin 2 | −0.73 | NM_053196 | Q925N2 |
| 1447877_x_at | Dnmt1 | DNA methyltransferase (cytosine-5) 1 | −0.73 | BB116018 | P13864 /// Q7TSJ0 |
| 1451395_at | D5Bwg0834e | DNA segment, Chr 5, Brigham & Woman's Genetics | −0.73 | BC021492 | Q8VDN4 |
| | | 0834 expressed | | | |
| 1453146_at | 4432404J10Rik | RIKEN cDNA 4432404J10 gene | −0.73 | BM123170 | AAH57164 /// AAH60123 /// BAC98191 /// Q80V37 /// Q80ZK4 /// Q8BTS5 /// Q9CRS2 /// |
| | | | | | Q9CTI1 |
| 1455888_at | B230217C06Rik | RIKEN cDNA B230217C06 gene | −0.73 | BB125202 | Q8BLC0 /// Q8BZD4 |
| 1424728_at | BC011248 | cDNA sequence BC011248 | −0.73 | BC011248 | Q91X71 |
| 1438154_x_at | 2610002J02Rik | RIKEN cDNA 2610002J02 gene | −0.72 | AV218922 | — |
| 1450505_a_at | 1810015C04Rik | RIKEN cDNA 1810015C04 gene | −0.72 | NM_025459 | Q7TMY5 /// Q8VE91 /// Q9CUJ4 /// Q9D8Z5 |
| 1430561_at | 5730496F10Rik | RIKEN cDNA 5730496F10 gene | −0.72 | BE952491 | — |
| 1452650_at | 6330414G21Rik | RIKEN cDNA 6330414G21 gene | −0.72 | AK018173 | Q80V85 |
| 1434006_at | Fksg24 | hypothetical protein Fksg24 | −0.72 | BQ030992 | Q80UR1 /// Q8VIK2 |
| 1424073_at | 5430437P03Rik | RIKEN cDNA 5430437P03 gene | −0.72 | BC005692 | Q8C5Q8 /// Q99JU2 /// Q9CTJ4 |
| 1417725_a_at | Sssca1 | Sjogren's syndrome/scleroderma autoantigen 1 | −0.72 | BC021593 | BAA87050 /// BAB23917 /// BAB28340 /// P56873 /// Q9CZE1 /// Q9D002 |
| | | homolog (human) | | | |
| 1448054_at | — | Mus musculus transcribed sequences | −0.72 | BE854760 | Q8C0D7 /// Q8C1S7 /// Q8K3Q5 /// Q8K3Q6 /// Q8K3Q7 /// Q9D7F9 |
| 1460018_at | — | Mus musculus adult male testis cDNA, RIKEN full- | −0.72 | AV278039 | Q8BVK6 /// Q8CI06 |
| | | length enriched library, clone: 4932704A10 | | | |
| | | product: unclassifiable, full insert sequence | | | |
| 1440161_at | — | Mus musculus transcribed sequences | −0.71 | BB378819 | — |
| 1415886_at | Sh2d3c | SH2 domain containing 3C | −0.71 | AB043953 | Q9JME1 /// Q9QZS8 |
| 1442280_at | D2Ertd750e | DNA segment, Chr 2, ERATO Doi 750, expressed | −0.71 | BM251033 | Q8K2D9 /// Q9CYZ4 /// Q9D9Z1 |
| 1416602_a_at | Rad52 | RAD52 homolog (S. cerevisiae) | −0.71 | NM_011236 | P43352 /// Q8VEE2 |
| 1446683_at | Eps15-rs | epidermal growth factor receptor pathway substrate 15, | −0.71 | BB098038 | Q60902 /// Q8CB60 /// Q8CB70 /// Q91WH8 |
| | | related sequence | | | |
| 1417467_a_at | Tada3l | transcriptional adaptor 3 (NGG1 homolog, yeast)-like | −0.71 | AK003405 | Q8R0L9 |
| 1451689_a_at | Sox10 | SRY-box containing gene 10 | −0.71 | BC018551 | AAH23356 /// O88418 /// Q04888 /// Q80V12 /// Q8C916 |
| 1423925_at | Dhx16 | DEAH (Asp-Glu-Ala-His) box polypeptide 16 | −0.71 | BC009147 | Q80TX4 /// Q921Y1 /// Q9CRI3 |
| 1451847_s_at | Arid4b | AT rich interactive domain 4B (Rbp1 like) | −0.71 | BC024724 | Q8BMI8 /// Q8BV50 /// Q8BXV6 /// Q8BYA5 /// Q8BYB0 /// Q8R1E4 |
| 1448567_at | Pl6-pending | PL6 protein | −0.71 | NM_019704 | BAC31672 /// Q9WUH1 |
| 1423685_at | Aars | aianyl-tRNA synthetase | −0.71 | BC026611 | AAH58620 /// AAP57355 /// Q8BGQ7 /// Q8R346 |
| 1423690_s_at | Gpsm1 | G-protein signalling modulator 1 (AGS3-like, | −0.71 | BC026486 | Q61366 /// Q8BUK4 /// Q8BX78 /// Q8R0E6 /// Q8R0R9 |
| | | C. elegans) | | | |
| 1416975_at | Stam2 | signal transducing adaptor molecule (SH3 domain and | −0.71 | BB125321 | O88811 /// Q8C8Y4 |
| | | ITAM motif) 2 | | | |
| 1420799_at | Nsr | neurotensin receptor | −0.71 | NM_018766 | O88319 |
| 1459223_at | B930095G15Rik | RIKEN cDNA B930095G15 gene | −0.71 | BB376007 | Q8C3S9 |
| 1418261_at | Syk | spleen tyrosine kinase | −0.70 | AW907526 | AAH65121 /// P48025 |
| 1434652_at | Cdc42bpb | Cdc42 binding protein kinase beta | −0.70 | BI154551 | Q7TT50 /// Q80W33 |
| 1419456_at | Dcxr | dicarbonyl L-xylulose reductase | −0.70 | BC012247 | Q91X52 /// Q9D129 /// Q9D8W1 |
| 1434154_at | Kctd13 | potassium channel tetramerisation domain containing | −0.70 | BQ177107 | Q8BGV7 |
| | | 13 | | | |
| 1434611_at | Rnf123 | ring finger protein 123 | −0.70 | BB765679 | AAH57082 |
| 1453874_at | 4933401B06Rik | RIKEN cDNA 4933401B06 gene | −0.70 | AV278276 | — |
| 1443452_at | — | Mus musculus transcribed sequences | −0.70 | BM212484 | — |
| 1437864_at | Adipor2 | adiponectin receptor 2 | −0.70 | BE632137 | AAR08379 /// Q8BQS5 |
| 1430986_at | Farsl | phenylalanine-tRNA synthetase-like | −0.70 | AK012154 | Q8C644 /// Q9CWZ8 /// Q9CZU5 /// Q9WUA2 |
| 1435183_at | 3110043L15Rik | RIKEN cDNA 3110043L15 gene | −0.70 | AW050349 | AAH57590 |
| 1458826_at | — | Mus musculus transcribed sequences | −0.70 | BG066316 | — |
| 1422943_a_at | Hspb1 | heat shock protein 1 | −0.70 | NM_013560 | BAB22579 /// BAB27099 /// P14602 /// Q9Z2L2 /// Q9Z2L3 |
| 1439546_at | 4933417O08Rik | RIKEN cDNA 4933417O08 gene | −0.70 | BB807546 | Q9D428 |
| 1452258_at | 6820402O20Rik | RIKEN cDNA 6820402O20 gene | −0.70 | BB308157 | AAH60121 /// Q8BLG0 /// Q8BZI4 |
| 1447607_at | — | — | −0.69 | AV045102 | — |
| 1452637_a_at | 1810037G04Rik | RIKEN cDNA 1810037G04 gene | −0.69 | BC027558 | Q9DB89 |
| 1436923_at | Rab2b | RAB2B, member RAS oncogene family | −0.69 | BF466486 | AAH61513 /// P59279 /// Q7TQF6 /// Q9DB48 |
| 1458594_at | Shprh | SNF2 histone linker PHD RING helicase | −0.69 | BB539406 | Q7TPQ3 /// Q7TQ27 /// Q7TQ28 /// Q7TQ29 /// Q8BKE2 /// Q8BUW0 /// Q8BXM1 /// Q922Q3 |
| 1426136_x_at | Klra8 | killer cell lectin-like receptor, subfamily A, member 8 | −0.69 | AF288380 | Q64329 /// Q9JHN9 |
| 1451284_at | D17Wsu94e | DNA segment, Chr 17, Wayne State University 94, | −0.69 | BC019384 | Q8C998 /// Q8K5D1 /// Q8VCS2 |
| | | expressed | | | |
| 1417811_at | Slc24a6 | solute carrier family 24 (sodium/potassium/calcium | −0.69 | NM_133221 | Q80XM7 /// Q925Q3 |
| | | exchanger), member 6 | | | |
| 1424432_at | Ubtd1 | ubiquitin domain containing 1 | −0.69 | BC016129 | Q91WB7 |
| 1436053_at | BC045600 | cDNA sequence BC045600 | −0.69 | BB272520 | AAH60066 /// Q80VE5 |
| 1435809_at | — | CDNA clone MGC: 56962 IMAGE: 6391322, complete | −0.69 | BE947974 | Q7TST1 |
| | | cds | | | |
| 1443569_at | 4930430E16Rik | RIKEN cDNA 4930430E16 gene | −0.69 | BB214806 | Q8QZV6 /// Q9CUP0 |
| 1441398_at | — | Mus musculus transcribed sequence with moderate | −0.69 | BG068080 | — |
| | | similarity to protein ref: NP_003263.1 (H. sapiens) | | | |
| | | transmembrane 7 superfamily member 1 (upregulated | | | |
| | | in kidney); transmembrane 7 superfamily member 1 | | | |
| | | (upregulated in [Homo sapiens] | | | |
| 1426805_at | Smarca4 | SWI/SNF related, matrix associated, actin dependent | −0.69 | AW701251 | AAH60229 /// AAH61214 /// O35845 /// Q7TQL1 /// Q8BQ54 /// Q8CGJ5 /// Q8R0K1 /// |
| | | regulator of chromatin, subfamily a, member 4 | | | Q8R569 |
| 1438105_at | — | Mus musculus transcribed sequences | −0.69 | BB667172 | — |
| 1445027_at | D030068L24 | hypothetical protein D030068L24 | −0.69 | BG073163 | — |
| 1428707_at | 2610009E16Rik | RIKEN cDNA 2610009E16 gene | −0.69 | AK011360 | Q80WS9 /// Q8VDQ2 /// Q9D0J8 |
| 1434128_a_at | Zfp574 | zinc finger protein 574 | −0.69 | BB131266 | AAH59044 /// Q8BKB5 /// Q8BY46 |
| 1430777_a_at | Golph3 | golgi phosphoprotein 3 | −0.69 | AK014644 | Q99KY1 /// Q9CRA5 |
| 1422715_s_at | Acp1 | acid phosphatase 1, soluble | −0.69 | AW554438 | Q9D358 |
| 1454018_at | Tlk2 | tousled-like kinase 2 (Arabidopsis) | −0.69 | AK014829 | AAH66198 /// O55047 /// P70320 /// Q9D9L6 |
| 1442600_at | — | Mus musculus 12 days embryo spinal ganglion cDNA, | −0.69 | BB456595 | — |
| | | RIKEN full-length enriched library, clone: D130047J24 | | | |
| | | product: inferred: ORF2 consensus sequence encoding | | | |
| | | endonuclease and reverse transcriptase minus | | | |
| | | RNaseH {Rattus norvegi, full insert sequence | | | |
| 1457313_at | 9530014D17Rik | RIKEN cDNA 9530014D17 gene | −0.68 | BG074373 | AAH68146 /// Q7TPR1 /// Q80XM6 /// Q8BXC9 /// Q8BXT3 |
| 1423769_at | Ptcd2 | pentatricopeptide repeat domain 2 | −0.68 | BC025110 | Q8R3K3 /// Q91VG3 /// Q9D0S7 |
| 1456313_x_at | Mrpl28 | mitochondrial ribosomal protein L28 | −0.68 | BB257397 | Q9D1B9 |
| 1428445_at | 9430029K10Rik | RIKEN cDNA 9430029K10 gene | −0.68 | AK020444 | Q9CX30 |
| 1449381_a_at | Pacsin1 | protein kinase C and casein kinase substrate in | −0.68 | BI731319 | BAC31717 /// Q61644 |
| | | neurons 1 | | | |
| 1454732_at | 6430517J16Rik | RIKEN cDNA 6430517J16 gene | −0.68 | AV340862 | Q7TMW8 /// Q8C052 |
| 1421309_at | Mgmt | O-6-methylguanine-DNA methyltransferase | −0.68 | NM_008598 | BAC16763 /// BAC16764 /// P26187 |
| 1438760_x_at | Adam15 | a disintegrin and metalloproteinase domain 15 | −0.68 | BB392633 | AAH57909 /// O88639 /// Q8CA82 |
| | | (metargidin) | | | |
| 1426783_at | Gcn5l2 | GCN5 general control of amino acid synthesis-like 2 | −0.68 | AW212720 | AAH63752 /// Q99KW4 /// Q9JHD2 |
| | | (yeast) | | | |
| 1454599_at | 4930425F17Rik | RIKEN cDNA 4930425F17 gene | −0.68 | AK019583 | Q9CTX4 |
| 1440860_at | Mab21l1 | mab-21-like 1 (C. elegans) | −0.68 | BB126987 | O70299 |
| 1417001_a_at | D4Wsu53e | DNA segment, Chr 4, Wayne State University 53, | −0.68 | BE447520 | AAH56986 /// Q80Y97 /// Q9CSN6 /// Q9D194 /// Q9JJF1 |
| | | expressed | | | |
| 1448803_at | Golga4 | golgl autoantigen, golgin subfamily a, 4 | −0.68 | NM_018748 | Q8C0A4 /// Q91VW5 |
| 1415987_at | Hdlbp | high density lipoprotein (HDL) binding protein | −0.68 | BG065877 | Q8VDJ3 |
| 1426297_at | Tcfe2a | transcription factor E2a | −0.68 | AF352579 | P15806 /// Q8CAH9 /// Q8VCY4 /// Q922S2 /// Q99MB8 /// Q9CRT1 /// Q9CYJ4 |
| 1426943_at | 1110015K06Rik | RIKEN cDNA 1110015K06 gene | −0.68 | AK003728 | Q80UQ7 /// Q91Z01 /// Q9CTF7 |
| 1455271_at | 1500011J06Rik | RIKEN cDNA 1500011J06 gene | −0.68 | BB560177 | Q8K1E6 |
| 1429051_s_at | 6230403H02Rik | RIKEN cDNA 6230403H02 gene | −0.68 | BE825056 | — |
| 1417978_at | 1300018P11Rik | RIKEN cDNA 1300018P11 gene | −0.67 | BC027014 | Q9D983 /// Q9DBB5 |
| 1460118_at | — | Mus musculus transcribed sequences | −0.67 | BF455409 | — |
| 1436132_at | D430036N24Rik | RIKEN cDNA D430036N24 gene | −0.67 | BB486539 | — |
| 1432622_a_at | 4930507D05Rik | RIKEN cDNA 4930507D05 gene | −0.67 | BB646733 | — |
| 1456078_x_at | 4930542G03Rik | RIKEN cDNA 4930542G03 gene | −0.67 | BB012080 | AAH61039 /// Q9D4K5 /// Q9DCR1 |
| 1440740_at | — | — | −0.67 | AV006603 | — |
| 1444355_at | Atp8a1 | ATPase, aminophosphollpid transporter (APLT), class I, | −0.67 | AW125445 | P70704 /// Q8BR88 /// Q8CA15 |
| | | type 8A, member 1 | | | |
| 1453476_at | 1700060J05Rik | RIKEN cDNA 1700060J05 gene | −0.67 | AK006643 | — |
| 1452372_at | 1110063F24Rik | RIKEN cDNA 1110063F24 gene | −0.67 | BF729638 | Q80Y55 /// Q8BI04 /// Q8VDP1 |
| 1455049_at | Igsf2 | immunoglobulin superfamily, member 2 | −0.67 | BB484576 | BAC32470 /// BAC35000 /// BAC97961 /// Q7TPV3 |
| 1443877_a_at | C030018K18Rik | RIKEN cDNA C030018K18 gene | −0.67 | BB306788 | Q8BLC8 /// Q8BX14 |
| 1456530_x_at | Elovl1 | elongation of very long chain fatty acids (FEN1/Elo2, | −0.67 | BB748075 | Q9JLJ5 /// Q9WU14 |
| | | SUR4/Elo3, yeast)-like 1 | | | |
| 1448880_at | Ube2l3 | ubiquitin-conjugating enzyme E2L 3 | −0.67 | BG066549 | P51966 |
| 1422404_x_at | — | — | −0.67 | NM_008334 | Q80SS5 |
| 1433802_at | AW125688 | expressed sequence AW125688 | −0.67 | BM114677 | — |
| 1423689_a_at | Gpsm1 | G-protein signalling modulator 1 (AGS3-like, | −0.67 | BC026486 | Q61366 /// Q8BUK4 /// Q8BX78 /// Q8R0E6 /// Q8R0R9 |
| | | C. elegans) | | | |
| 1459808_at | Fkbp4 | FK506 binding protein 4 | −0.66 | BB087569 | BAC39057 /// P30416 /// Q8CBS1 |
| 1428898_at | 2810468K17Rik | RIKEN cDNA 2810468K17 gene | −0.66 | AK013387 | AAH58717 /// Q80UP6 /// Q9CYS2 |
| 1417348_at | 2310039H08Rik | RIKEN cDNA 2810039H08 gene | −0.66 | NM_025966 | — |
| 1418865_at | Zfp385 | zinc finger protein 385 | −0.66 | NM_013866 | Q8VD12 /// Q9QY68 |
| 1429328_at | Nsfl1c | NSFL1 (p97) cofactor (p47) | −0.66 | BG922397 | Q9CZ44 |
| 1426257_a_at | Sars1 | seryl-aminoacyl-tRNA synthetase 1 | −0.66 | BC008612 | BAC35990 /// P26638 /// Q8C483 /// Q8CEH3 |
| 1452338_s_at | Itsn | intersectin (SH3 domain protein 1A) | −0.66 | AA172344 | AAH66105 /// Q80WF1 /// Q8C4B5 /// Q8CGU2 /// Q8CGU5 /// Q8CJ43 /// Q8CJ54 /// |
| | | | | | Q8CJ55 /// Q8CJ62 /// Q8R358 /// Q9Z0R4 |
| 1435685_x_at | Abcc5 | ATP-binding cassette, sub-family C (CFTR/MRP), | −0.66 | AV150520 | AAH61132 /// Q8CFP9 /// Q9JL43 /// Q9R1X5 |
| | | member 5 | | | |
| 1421843_at | Ilrap | interleukin 1 receptor accessory protein | −0.66 | BE285634 | Q61730 |
| 1430004_s_at | Wdr20 | WD repeat domain 20 | −0.66 | AK015014 | Q80X67 /// Q9D5R2 |
| 1437311_at | A930034L06Rik | RIKEN cDNA A930034L06 gene | −0.65 | BB281971 | Q8BV32 |
| 1447247_at | — | Mus musculus transcribed sequences | −0.65 | BE957311 | — |
| 1448187_at | Pold1 | polymerase (DNA directed), delta 1, catalytic subunit | −0.65 | BC009128 | P52431 /// Q8C2N0 /// Q91VT0 |
| 1436674_at | Rap2ip | Rap2 interacting protein | −0.65 | AW489945 | O08576 /// Q80Y95 |
| 1417079_s_at | Lgals2 | lectin, galactose-binding, soluble 2 | −0.65 | NM_025622 | Q8K1I1 /// Q9CQW5 |
| 1444299_at | A430093F15Rik | RIKEN cDNA A430093F15 gene | −0.65 | BB209605 | Q8C505 |
| 1447107_at | Ddx55 | DEAD (Asp-Glu-Ala-Asp) box polypeptide 55 | −0.65 | BB756348 | BAC98212 /// Q810A4 /// Q8BK20 /// Q8BZR1 /// Q9CS87 /// Q9CSI0 |
| 1453662_at | B230205O20Rik | RIKEN cDNA B230205O20 gene | −0.65 | AK020987 | — |
| 1440497_at | 1110021J02Rik | RIKEN cDNA 1110021J02 gene | −0.65 | BE956898 | Q8C7V0 /// Q9DBC3 |
| 1454857_at | Rnf122 | ring finger protein 122 | −0.65 | AW551457 | Q80VA7 /// Q8BGD3 /// Q8BP31 |
| 1426301_at | Alcam | activated leukocyte cell adhesion molecule | −0.65 | U95030 | Q61490 |
| 1453014_a_at | Sec31I1 | SEC31-like 1 (S. cerevisiae) | −0.65 | BM222383 | Q7TQJ7 /// Q811J4 /// Q9CVL3 |
| 1428443_a_at | Rap1ga1 | Rap1, GTPase-activating protein 1 | −0.65 | AK005063 | Q80VZ8 /// Q8K2L6 |
| 1433757_a_at | Nisch | nischarin | −0.64 | BB025231 | Q80TM9 /// Q8C354 /// Q8C4X9 /// Q8CBH0 /// Q8CF63 /// Q91XW6 /// Q99LG6 /// Q9EPW8 |
| | | | | | /// Q9WUM6 |
| 1444228_s_at | Herc2 | hect (homologous to the E6-AP (UBE3A) carboxyl | −0.64 | BB333568 | O88473 /// Q7TPR5 /// Q80VV7 |
| | | terminus) domain and RCC1 (CHC1)-like domain | | | |
| | | (RLD) 2 | | | |
| 1448378_at | Fscn1 | fascin homolog 1, actin bundling protein | −0.64 | NM_007984 | Q61553 /// Q7TN32 /// Q80V75 |
| | | (Strongylocentrotus) purpuratus) | | | |
| 1420524_a_at | Masp2 | mannan-binding lectin serine protease 2 | −0.64 | NM_010767 | Q91WP0 /// Q9QXA4 /// Q9QXD2 /// Q9QXD5 /// Q9Z338 |
| 1428955_x_at | Slc9a3r2 | solute carrier family 9 (sodium/hydrogen exchanger), | −0.64 | AK004710 | AAH65778 /// Q9JHL1 |
| | | isoform 3 regulator 2 | | | |
| 1451680_at | Npn3 | neoplastic progression 3 | −0.64 | BC011325 | Q62368 /// QGD975 |
| 1426665_at | Katnb1 | katanin p80 (WD40-containing) subunit B 1 | −0.64 | AK010364 | Q8B40 /// Q8CD18 /// Q8R1J0 /// Q9CWV2 |
| 1439219_at | A730082K24Rik | RIKEN cDNA A730082K24 gene | −0.64 | BB258061 | — |
| 1441371_at | 9330117B14 | hypothetical protein 9330117B14 | −0.64 | BQ174019 | — |
| 1423834_s_at | Gga1 | golgi associated, gamma adaptin ear containing, ARF | −0.64 | BC026802 | Q8R0H9 |
| | | binding protein 1 | | | |
| 1453101_at | 2810402K13Rik | RIKEN cDNA 2810402K13 gene | −0.64 | AK012967 | Q8K1D5 /// Q9CZ64 |
| 1429135_at | 1110059M19Rik | RIKEN cDNA 1110059M19 gene | −0.64 | AV015858 | Q9D0W7 |
| 1422028_a_at | Ets1 | E26 avian leukemia oncogene 1, 5′ domain | −0.64 | BC010588 | AAR00342 /// AAR87824 /// P27577 /// Q8BVW8 /// Q8K3Q9 /// Q921D8 |
| 1417316_at | Them2 | thioesterase superfamily membar 2 | −0.64 | NM_025790 | Q9CQR4 |
| 1457816_at | Casp9 | caspase 9 | −0.64 | BB781510 | AAH56372 /// AAH56447 /// Q8C3Q0 /// Q8C3Q9 /// Q9R0S9 /// Q9R0T0 |
| 1451019_at | Ctsf | cathepsin F | −0.64 | AK017474 | BAC36013 /// Q99KQ9 /// Q9ES93 /// Q9R013 |
| 1457272_at | — | Mus musculus transcribed sequences | −0.64 | BB284000 | — |
| 1424061_at | Manbal | mannosidase, beta A, lysosomal-like | −0.64 | BC013803 | Q9D8X0 |
| 1448262_at | Psmb2 | proteasome (prosome, macropain) subunit, beta type 2 | −0.64 | NM_011970 | Q8BJX0 /// Q9R1P3 |
| 1427928_s_at | BC028278 | cDNA sequence BC028278 | −0.64 | AW538039 | Q8K358 |
| 1460109_at | D8Ertd325e | DNA segment, Chr 8, ERATO Doi 325, expressed | −0.63 | AV253069 | Q80TL2 /// Q80ZX3 /// Q8BR80 /// Q8C200 /// Q8C2M3 /// Q8C6B4 /// Q8R3L2 /// Q9CUW0 |
| | | | | | /// Q9ER19 |
| 1454237_at | 1700030K01Rik | RIKEN cDNA 1700030K01 gene | −0.63 | AK016416 | Q9D4M8 /// Q9D9R3 |
| 1427617_at | Fut10 | fucosyltransferase 10 | −0.63 | BC022579 | AAH62113 /// Q8C457 /// Q8K0S3 /// Q8R247 |
| 1426465_at | Dlgap4 | discs, large homolog-associated protein 4 (Drosophila) | −0.63 | BG066219 | AAH58946 /// AAO89220 /// Q80TN3 /// Q8R3U9 |
| 1437004_at | 1700096K11Rik | RIKEN cDNA 1700096K11 gene | −0.63 | BG069841 | — |
| 1421018_at | 1110018J18Rik | RIKEN cDNA 111001BJ18 gene | −0.63 | NM_025370 | Q9D1A0 |
| 1423146_at | Hes5 | hairy and enhancer of split5 (Drosophila) | −0.63 | AV337579 | BAC39904 /// P70120 |
| 1457232_at | Fbxi21 | F-box and leucine-rich repeat protein 21 | −0.62 | BE946365 | Q8BFZ4 |
| 1428520_at | 1110032A13Rik | RIKEN cDNA 1110032A13 gene | −0.62 | AK004019 | AAH64066 |
| 1459638_at | — | Mus musculus transcribed sequences | −0.62 | BE852843 | — |
| 1457032_at | Ak5 | adenylate kinase 5 | −0.62 | BB546359 | Q920P5 |
| 1442652_at | — | Mus musculus transcribed sequences | −0.62 | BM935317 | — |
| 1434322_at | A930021H16Rik | RIKEN cDNA A930021H16 gene | −0.61 | BB513585 | Q80UK4 |
| 1423937_at | Kctd5 | potassium channel tetramerisation domain containing 5 | −0.61 | BF577853 | BAC97887 /// Q8VC57 /// Q9CSZ1 |
| 1416013_at | Pld3 | phospholipase D3 | −0.61 | NM_011116 | O35405 |
| 1416766_at | 2810484M10Rik | RIKEN cDNA 2810484M10 gene | −0.61 | NM_133684 | Q8C6F6 /// Q922Q1 |
| 1418786_at | Mapk8ip2 | mitogen-activated protein kinase 8 interacting protein 2 | −0.61 | AF220195 | AAH29704 /// AAL50331 /// O35287 /// Q924X2 /// Q9CUY3 /// Q9ERE9 /// Q9QYP4 |
| 1430978_at | Rps25 | ribosomal protein S25 | −0.61 | BM729504 | AAH27208 /// BAC3S806 /// P25111 |
| 1447663_at | — | — | −0.61 | BB044824 | — |
| 1443104_at | — | Mus musculus 0 day neonate eyeball cDNA, RIKEN full- | −0.61 | BB541236 | — |
| | | length enriched library, clone: E130112O04 | | | |
| | | product: unknown EST, full insert sequence | | | |
| 1455157_a_at | 2310061F22Rik | RIKEN cDNA 2310061F22 gene | −0.61 | AV173117 | BAC97905 /// Q7TQL5 /// Q80XT7 /// Q811I7 /// Q8BTG9 /// Q8CI68 |
| 1417460_at | Ifitm3l | interferon induced transmembrane protein 3-like | −0.61 | NM_030694 | Q99J93 |
| 1457819_at | — | Mus musculus transcribed sequence with strong | −0.61 | AI549833 | — |
| | | similarity to protein pir: S54771 (H. sapiens) S54771 | | | |
| | | sodium channel alpha subunit-human | | | |
| 1417104_at | Emp3 | epithelial membrane protein 3 | −0.61 | BC001999 | O35912 |
| 1415991_a_at | Klhdc3 | kelch domain containing 3 | −0.61 | NM_027910 | Q8VEM9 /// Q91XU6 /// Q99JH9 /// Q9DBG8 |
| 1416335_at | Mif | macrophage migration inhibitory factor | −0.61 | NM_010798 | BAB26980 /// BAB27123 /// BAB28792 /// P34884 |
| 1448479_at | Psmd3 | proteasome (prosome, macropain) 26S subunit, non- | −0.61 | NM_009439 | P14585 /// Q8BK46 |
| | | ATPase, 3 | | | |
| 1449028_at | Rhou | ras homolog gene family, member U | −0.61 | AF378088 | Q9D778 /// Q9EQT3 |
| 1423889_at | Rbm7 | RNA binding motif protein 7 | −0.61 | BC011344 | Q7TQE3 /// Q91VN2 /// Q9CQT2 |
| 1442944_at | — | — | −0.61 | BG065699 | — |
| 1420844_at | Ubqin2 | ubiquilin 2 | −0.60 | AV171029 | Q9QZM0 |
| 1418454_at | Mfap5 | microfibrillar associated protein 5 | −0.60 | NM_015776 | Q9QZJ6 |
| 1421918_at | Anp32a | acidic (leucine-rich) nuclear phosphoprotein 32 family, | −0.60 | AF022957 | AAH62899 /// O35381 |
| | | member A | | | |
| 1437322_at | Rbm14 | RNA binding motif protein 14 | −0.60 | BM218282 | O08752 /// Q8BN66 /// Q8C2Q3 /// Q8C7Q4 /// Q91Z21 /// Q9DBI6 |
| 1424187_at | 2610001E17Rik | RIKEN cDNA 2610001E17 gene | −0.60 | BG074158 | AAH58751 /// Q8C043 /// Q8C8E1 /// Q8R2G6 /// Q9CRM1 /// Q9C739 /// Q9D6Z4 |
| | | | | | AAH57302 /// P70678 /// Q8BHA5 /// Q8BIA4 /// Q8BTH9 /// Q8BXI9 /// Q8C658 /// Q8CHC6 |
| 1436732_s_at | Fbxw8 | F-box and WD-40 domain protein 8 | −0.60 | BB750997 | /// Q8VDH1 /// Q9D5H7 |
| 1437118_at | Usp7 | ubiquitin specific protease 7 | −0.60 | C77542 | Q8BW01 |
| 1458464_at | Nedl2 | NEDD4-related E3 ubiquitin ligase NEDL2 | −0.60 | BB445169 | Q8BQD5 |
| 1422155_at | Hist2h3c2 | histone 2, H3c2 | −0.60 | BC015270 | — |
| 1418136_at | Tgfb1i1 | transforming growth factor beta 1 induced transcript 1 | −0.60 | NM_009365 | AAH56362 /// Q62219 |
| 1434575_at | Epb4.1l1 | erythrocyte protein band 4.1-like 1 | −0.60 | BB794965 | Q80U34 /// Q8C8P2 /// Q8K204 /// Q9Z2H5 |
| 1418570_at | Ncstn | nicastrin | −0.59 | BC019998 | BAC97912 /// P57716 |
| 1428383_a_at | 2310021P13Rik | RIKEN cDNA 2310021P13 gene | −0.59 | BC026504 | AAH58666 /// AAH59058 /// Q80Y41 /// Q8CE12 /// Q8CHC3 /// Q9D789 |
| 1452221_a_at | Cxxc1 | CXXC finger 1 (PHD domain) | −0.59 | BB447351 | AAM28246 /// BAC38986 /// Q9CWW7 |
| 1425511_at | Mark1 | MAP/microtubule affinity-regulating kinase 1 | −0.59 | BM213279 | Q8VHJ5 |
| 1451745_a_at | Znhit1 | zinc finger, HIT domain containing 1 | −0.59 | BC026751 | Q8R331 |
| 1425054_a_at | 2510006D16Rik | RIKEN cDNA 2510006D16 gene | −0.59 | BC024696 | Q8CEZ5 /// Q8R1E7 /// Q9D484 |
| 1460405_at | 2810441C07Rik | RIKEN cDNA 2810441C07 gene | −0.59 | AV238183 | AAH59220 /// BAC98218 /// Q8C1A1 /// Q8VDH5 /// Q922V5 |
| 1425304_s_at | Prima1 | proline rich membrane anchor 1 | −0.59 | AY043275 | Q810F0 /// Q9D1X7 |
| 1417038_at | — | septin 9 | −0.59 | NM_017380 | Q80UG5 /// Q9QYX9 |
| 1427142_s_at | Jarid1b | jumonji, AT rich interactive domain 1B (Rbp2 like) | −0.59 | BC019446 | AAH57318 /// Q80Y84 /// Q8BLU1 /// Q8C1P6 /// Q8JZL8 /// Q8VCQ4 |
| 1436377_at | — | Mus musculus mRNA similar to chromosome 11 | −0.59 | BI410102 | Q80UB6 /// Q80ZU9 |
| | | hypothetical protein ORF4 (cDNA clone MGC: 56861 | | | |
| | | IMAGE: 6308873), complete cds | | | |
| 1426393_a_at | Sdf4 | stromal cell derived factor 4 | −0.59 | BM198177 | AAH68152 /// Q61112 |
| 1450432_s_at | Mus81 | MUS81 endonuclease homolog (yeast) | −0.59 | AF425647 | Q91ZJ0 |
| 1451409_at | 2210021J22Rik | RIKEN cDNA 2210021J22 gene | −0.59 | BC025858 | Q8CEZ9 /// Q8R3A2 |
| 1423940_at | YIf1 | Yip1 interacting factor homolog (S. cerevisiae) | −0.59 | BC011117 | Q91XB7 /// Q9CWB2 |
| 1431412_at | 2810455B08Rik | RIKEN cDNA 2810455B08 gene | −0.59 | BI692111 | — |
| 1419628_at | Chx10 | C. elegans ceh-10 homeo domain containing homolog | −0.59 | NM_007701 | AAH58806 /// Q61412 /// Q80WF9 |
| 1420585_a_at | Nxf2 | nuclear RNA export factor 2 | −0.59 | NM_031259 | Q99JG4 /// Q99MW6 /// Q99NI0 |
| 1426686_s_at | Map3k3 | mitogen activated protein kinase kinase kinase 3 | −0.59 | BG175594 | Q61084 |
| 1445333_at | — | — | −0.59 | BG066013 | — |
| 1441878_s_at | 1810049H13Rik | RIKEN cDNA 1810049H13 gene | −0.59 | BB401085 | Q9CR10 |
| 1455764_at | Lrpap1 | low density lipoprotein receptor-related protein | −0.59 | AV309553 | AAH46641 /// AAH57979 /// AAH59887 /// BAA00500 /// CAG25840 /// Q8C252 /// Q8K295 |
| | | associated protein 1 | | | |
| 1452257_at | Bdh | 3-hydroxybutyrate dehydrogenase (heart, mitochondrial) | −0.59 | BF322712 | Q80XN0 /// Q8BK53 /// Q8R0C8 |
| 1440792_x_at | Oprs1 | oploid receptor, sigma 1 | −0.59 | BB405850 | O55242 /// Q9JKU9 |
| 1446938_at | — | Mus musculus transcribed sequences | −0.59 | BG063210 | — |
| 1452370_s_at | B230208H17Rik | RIKEN cDNA B230208H17 gene | −0.59 | BB449608 | AAH58585 /// Q8BFS4 /// Q8CGJ9 |
| 1425784_a_at | Olfm1 | olfactomedin 1 | −0.59 | D78264 | O88998 /// Q8R357 |
| 1456040_at | Sf3b2 | splicing factor 3b, subunit 2 | −0.59 | BB473131 | Q80W39 /// Q8BL33 /// Q9CS24 |
| 1432648_at | 4930468F19Rik | RIKEN cDNA 4930466F19 gene | −0.59 | AV044111 | Q9D5C1 |
| 1443707_at | 2900046B09Rik | RIKEN cDNA 2900046B09 gene | −0.59 | BB816172 | — |
| 1425081_at | Zfp286 | zinc finger protein 286 | −0.58 | BE651907 | Q8C0E6 /// Q8R0E0 |
| 1437957_at | 7030407O06Rik | RIKEN cDNA 7030407O06 gene | −0.58 | AW539719 | — |
| 1422876_at | Capn9 | calpain 9 (nCL-4) | −0.58 | NM_023709 | AAH58748 /// Q9D805 |
| 1428824_at | 2310003C23Rik | RIKEN cDNA 2310003C23 gene | −0.58 | AK009106 | Q9D7M1 |
| 1437300_at | 2210408E11Rik | RIKEN cDNA 2210408E11 gene | −0.58 | BG067616 | — |
| 1416339_a_at | Prkcsh | protein kinase C substrate 80K-H | −0.58 | NM_008925 | O08795 ///Q921X2 |
| 1460334_at | Dbnl | drebrin-like | −0.58 | AV328035 | Q62418 /// Q80WP1 /// Q8BH56 |
| 1434417_at | Solh | small optic lobes homolog (Drosophila) | −0.58 | BB022975 | AAH58094 /// Q8R200 |
| 1418268_at | Htr3a | 5-hydroxytryptamine (serotonin) receptor 3A | −0.38 | NM_013561 | P23979 /// Q8K1F4 |
| 1422256_at | Sstr2 | somatostatin receptor 2 | −0.34 | NM_009217 | P30875 |
| 1438921_at | — | Mus musculus transcribed sequence with moderate | 0.00 | BM197239 | — |
| | | similarity to protein prf.2211433A (H. sapiens) | | | |
| | | 2211433A FRP1 protein [Homo sapiens] | | | |
| 1434423_at | Gulp1 | GULP, engulfment adaptor PTB domain containing 1 | 0.01 | BB138485 | Q8K2A1 /// Q9CRV4 /// Q9CYD2 |
| 1418849_x_at | Aqp7 | aquaporin 7 | 0.10 | AB056091 | BAB68537 /// O54794 |
| 1459606_at | — | Mus musculus transcribed sequences | 0.17 | BB752953 | — |
| 1417704_a_at | Arhgap6 | Rho GTPase activating protein 6 | 0.17 | NM_009707 | O54834 /// Q8BG83 /// Q8C842 /// Q8C8B2 |
| 1459856_at | — | Mus musculus transcribed sequences | 0.19 | BB444619 | — |
| 1419171_at | 2310044D20Rik | RIKEN cDNA 2310044D20 gene | 0.20 | BB667295 | Q8CC46 /// Q8VDR1 /// Q9D238 /// Q9D3L0 /// Q9D6W5 /// Q906Z3 |
| 1453807_at | 6330563C09Rik | RIKEN cDNA 6330563C09 gene | 0.21 | BI730484 | — |
| 1417753_at | Pkd2 | polycystic kidney disease 2 | 0.32 | AF014010 | AAH62969 /// O35245 /// Q7TSI7 /// Q8BPR6 |
| 1447173_at | — | — | 0.34 | BB704012 | — |
| 1421882_a_at | Elavl2 | ELAV (embryonic lethal, abnormal vision, Drosophila)- | 0.34 | BB105998 | AAH58393 /// AAK74154 /// Q60899 /// Q80UJ0 /// Q80Y51 /// Q91XI8 /// Q91XI9 |
| | | like 2 (Hu antigen B) | | | |
| 1437250_at | — | Mus musculus transcribed sequence with weak | 0.34 | AV298358 | AAH68125 |
| | | similarity to protein ref: NP_060470.1 (H. sapiens) | | | |
| | | hypothetical protein FLJ10116 [Homo sapiens] | | | |
| 1418318_at | Rnf128 | ring finger protein 128 | 0.36 | AK004847 | Q9CVG1 /// Q9D304 /// Q9DBN3 /// Q9JJF8 |
| 1449007_at | Btg3 | B-cell translocation gene 3 | 0.37 | NM_009770 | P50615 |
| 1456504_at | 6330583I20Rik | RIKEN cDNA 6330583I20 gene | 0.38 | BM248637 | AAH63066 /// Q8CCU4 |
| 1442019_at | B230343A10Rik | RIKEN cDNA B230343A10 gene | 0.38 | BB627097 | — |
| 1419207_at | Zfp37 | zinc finger protein 37 | 0.38 | NM_009554 | AAH63757 /// P17141 /// Q8CCM5 /// Q8R1B1 |
| 1460707_at | Ptp4a2 | protein tyrosine phosphatase 4a2 | 0.40 | AV049645 | O70274 |
| 1454826_at | — | Mus musculus cDNA clone IMAGE: 6485438, | 0.41 | BM195115 | Q8BXA4 /// Q8BZQ5 |
| | | partial cds | | | |
| 1436841_at | B230380D07Rik | RIKEN cDNA B230380D07 gene | 0.42 | AV229336 | AAH58683 /// Q7TML6 /// Q8BK25 /// Q8BL22 /// Q8BL47 /// Q8BZC1 |
| 1460017_at | 9930105H17Rik | RIKEN cDNA 9930105H17 gene | 0.42 | BB371300 | — |
| 1416612_at | Cyp1b1 | cytochrome P450, family 1, subfamily b, polypeptide 1 | 0.43 | BI251808 | Q64429 /// Q80V82 /// Q8BRY0 /// Q8C685 /// Q9CUA1 |
| 1438133_a_at | Cyr61 | cysteine rich protein 61 | 0.43 | BM202770 | AAH66019 /// P18406 |
| 1428907_at | 2600011C06Rik | RIKEN cDNA 2600011C06 gene | 0.43 | BG228787 | AAH66150 /// AAH67400 /// Q8BU35 /// Q8BVT8 /// Q9CT49 |
| 1427934_at | 2610208E05Rik | RIKEN cDNA 2610208E05 gene | 0.43 | AA250510 | Q8R033 |
| 1436330_x_at | — | hypothetical protein 6720451E15 | 0.44 | BG244780 | Q8BIQ6 |
| 1448141_at | 1110014J01Rik | RIKEN cDNA 1110014J01 gene | 0.45 | NM_029101 | — |
| 1417221_at | Ppm1a | protein phosphatase 1A, magnesium dependent, alpha | 0.45 | BC008595 | P49443 /// Q8R4T7 /// Q9EQE2 /// Q9EQE3 |
| | | isoform | | | |
| 1434034_at | Cerk | ceramide kinase | 0.46 | BI905090 | BAC98226 /// Q8K4Q7 |
| 1450064_at | Fmn2 | formin 2 | 0.46 | BM228488 | Q9JL04 |
| 1433991_x_at | Dbi | diazepam binding inhibitor | 0.46 | AV007315 | BAB25730 /// BAB25755 /// BAB32175 /// BAC25658 /// P31786 |
| 1454741_s_at | — | Mus musculus cDNA clone MGC: 67308 | 0.46 | BG064061 | AAH56470 /// Q8C237 /// Q8C4K1 |
| | | IMAGE: 5706838, complete cds | | | |
| 1428512_at | 2700087I09Rik | RIKEN cDNA 2700087I09 gene | 0.47 | AK012577 | AAH59871 |
| 1443052_at | C330019L16 | hypothetical protein C330019L16 | 0.47 | BB400711 | — |
| 1428468_at | 3110043O21Rik | RIKEN cDNA 3110043O21 gene | 0.48 | AK014175 | Q8K3B2 |
| 1422653_at | C030018L16Rik | RIKEN cDNA C030018L16 gene | 0.48 | NM_023873 | Q9CRL9 /// Q9CTS4 /// Q9JIC1 |
| 1448743_at | Ssx2ip | synovial sarcoma, X breakpoint 2 interacting protein | 0.48 | NM_138744 | Q8BG59 /// Q8C7X0 /// Q8K2F7 /// Q8VC66 |
| 1416422_a_at | Ssb | Sjogren syndrome antigen B | 0.48 | BG796845 | BAC28092 /// BAC40478 /// P32067 /// Q8BTU4 /// Q8BTY4 /// Q9CYB9 |
| 1434307_at | 9630015D15Rik | RIKEN cDNA 9630015D15 gene | 0.49 | AW489972 | AAN05738 /// Q8CBJ4 /// Q8K2Q6 |
| 1436794_at | C330026N02Rik | RIKEN cDNA C330026N02 gene | 0.49 | BG069844 | Q8BWZ1 |
| 1415964_at | Scd1 | stearoyl-Coenzyme A desaturase 1 | 0.49 | NM_009127 | AAM34744 /// AAM34747 /// P13516 |
| 1426270_at | Smc5l1 | SMC5 structural maintenance of chromosomes 5-like 1 | 0.49 | AV257384 | Q80TW7 /// Q8BKX5 /// Q8CG46 /// Q8CHX5 /// Q922K3 |
| | | (yeast) | | | |
| 1416195_at | Pps | putative phosphatase | 0.49 | NM_008916 | AAH66112 /// Q8C5L6 |
| 1428233_at | Cpsf6 | cleavage and polyadenylation specific factor 6 | 0.50 | BB425379 | AAH68133 |
| 1425486_s_at | Mtmr6 | myotubularin related protein 6 | 0.50 | BC020019 | Q8VE11 |
| 1425484_at | Tox | thymocyte selection-associated HMG box gene | 0.51 | BB547854 | Q8BKH9 /// Q8BYQ5 /// Q8R4H0 |
| 1435235_at | Txnl | thioredoxin-like | 0.51 | BI662855 | AAH61123 /// O70379 /// Q8CDN6 |
| 1428075_at | Ndufb4 | NADH dehydrogenase (ubiquinone) 1 beta subcomplex 4 | 0.51 | BG968046 | Q9CQC7 /// Q9DBH2 |
| 1416179_a_at | Rdx | radixin | 0.52 | NM_009041 | AAR87801 /// P26043 /// Q7TSG6 /// Q8C2N4 |
| 1452675_at | Rbm22 | RNA binding motif protein 22 | 0.52 | BB758922 | Q8BHS3 /// Q9CXA0 |
| 1448558_a_at | Pla2g4a | phospholipase A2, group IVA (cytosolic, calcium- | 0.53 | NM_008869 | P47713 /// Q9DBX5 |
| | | dependent) | | | |
| 1416705_at | Rpe | ribulose-5-phosphate-3-epimerase | 0.53 | BG916066 | Q62505 /// Q8VEE0 /// Q91VZ4 |
| 1435123_at | mKIAA0953 | mKIAA0953 protein | 0.54 | BB795377 | BAC98057 |
| 1448269_a_at | Klhl13 | kelch-like 13 (Drosophila) | 0.54 | NM_026167 | Q80TF4 /// Q8BKJ6 /// Q8BLH8 /// Q9CSA7 |
| 1448358_s_at | Snrpg | small nuclear ribonucleoprotein polypeptide G | 0.55 | NM_026506 | AAH51470 /// Q15357 |
| 1427376_a_at | Map4k5 | mitogen-activated protein kinase kinase kinase kinase 5 | 0.55 | BC002309 | AAH57930 /// Q8BPM2 /// Q8BRE4 |
| 1433446_at | Hmgcs1 | 3-hydroxy-3-methylglutaryl-Coenzyme A synthase 1 | 0.55 | BB705380 | Q8C5F4 /// Q8JZK9 /// Q8K0I5 |
| 1452061_s_at | Spnr | spermatid perinuclear RNA binding protein | 0.55 | AK006314 | AAQ88431 /// Q62262 /// Q8BFT4 /// Q8C5B7 /// Q91WM1 /// Q9CVW0 |
| 1460432_a_at | Eif3s6 | eukaryotic translation initiation factor 3, subunit 6 | 0.56 | AK002576 | AAC53346 // P60229 /// Q8BNE6 /// Q9CT23 |
| 1425628_a_at | Gtf2i | general transcription factor II I | 0.57 | AF043220 | Q9ESZ8 |
| 1448195_at | Taf5l | TAF5-like RNA polymerase II, p300/CBP-associated | 0.57 | NM_133966 | Q91WQ5 |
| | | factor (PCAF)-associated factor | | | |
| 1415973_at | Marcks | myristoylated alanine rich protein kinase C substrate | 0.57 | AW546141 | P26645 |
| 1448236_at | Rdx | radixin | 0.58 | NM_009041 | AAR87801 /// P26043 /// Q7TSG6 /// Q8C2N4 |
| 1426083_a_at | Btg1 | B-cell translocation gene 1, anti-proliferative | 0.58 | L16846 | P31607 |
| 1436139_at | — | Mus musculus adult male medulla oblongata cDNA, | 0.58 | AV328974 | — |
| | | RIKEN full-length enriched library, clone: 6330445K22 | | | |
| | | product: unknown EST, full insert sequence | | | |
| 1453180_at | 6530404N21Rik | RIKEN cDNA 6530404N21 gene | 0.58 | AK018322 | Q80W75 |
| 1434842_s_at | Upf3b | UPF3 regulator of nonsense transcripts homolog B | 0.58 | AV294165 | Q80UI8 /// Q9CS15 |
| | | (yeast) | | | |
| 1427129_a_at | Hnrpr | heterogeneous nuclear ribonucleoprotein R | 0.58 | AW701147 | Q8BL32 /// Q8VHM5 /// Q99KG1 /// Q9CT37 |
| 1433825_at | Ntrk3 | neurotrophic tyrosine kinase, receptor, type 3 | 0.58 | BM245880 | AAP94280 /// Q9Z2P9 /// Q9Z2Q0 |
| 1454632_at | 6330442E10Rik | RIKEN cDNA 6330442E10 gene | 0.58 | AV328515 | AAH66067 /// Q8BFQ2 /// Q8CCD3 |
| 1433648_at | Spag9 | sperm associated antigen 9 | 0.58 | BM938614 | AAH60100 /// AAH60506 /// Q8BSD1 /// Q8C7W0 /// Q8CJC2 |
| 1421033_a_at | Tcerg1 | transcription elongation regulator 1 (CA150) | 0.58 | AW046403 | Q8C490 /// Q8CGF7 /// Q8CHT8 /// Q9R0R5 |
| 1415689_s_at | Zfp307 | zinc finger protein 307 | 0.59 | BC007473 | O88252 /// Q8BSQ2 /// Q8CD81 /// Q91VW9 /// Q9CSC5 /// Q9ESY5 |
| 1428207_at | Bcl7a | B-cell CLL/lymphoma 7A | 0.59 | AK014498 | Q8C361 /// Q8C8M8 /// Q8VD15 /// Q9CXE2 |
| 1429519_at | Fpgt | fucose-1-phosphate guanylyltransferase | 0.59 | BB303906 | CAC81971 /// Q8C1A2 |
| 1449557_at | 1600012F09Rik | RIKEN cDNA 1600012F09 gene | 0.59 | NM_025904 | Q8BPG2 /// Q9CS38 /// Q9D033 /// Q9D053 /// Q9D064 /// Q9DAY6 |
| 1418380_at | Terf1 | telomeric repeat binding factor 1 | 0.59 | NM_009352 | P70371 /// Q7TSK8 |
| 1417030_at | 2310028N02Rik | RIKEN cDNA 2310028N02 gene | 0.59 | NM_025864 | Q9CZV9 /// Q9D771 |
| 1451146_at | Zfp386 | zinc finger protein 386 (Kruppel-like) | 0.59 | BC004747 | Q99KB9 /// Q9QZP7 |
| 1448104_at | Aldh6a1 | aldehyde dehydrogenase family 6, subfamily A1 | 0.59 | NM_134042 | Q8CIB4 /// Q8K0L1 /// Q9EQ20 |
| 1429897_a_at | D16Ertd472e | DNA segment, Chr 16, ERATO Doi 472, expressed | 0.59 | AK009258 | Q8VE27 /// Q9D7G4 |
| 1448600_s_at | Vav3 | vav 3 oncogene | 0.59 | BC027242 | Q7TS85 /// Q8BRV2 /// Q8CCF5 /// Q8R076 /// Q9JLS6 /// Q9R0C8 |
| 1425934_a_at | B4galt4 | UDP-Gal:betaGlcNAc beta 1,4-galactosyltransferase, | 0.59 | AF158746 | Q8BR54 /// Q9JJ04 /// Q9QY12 |
| | | polypeptide 4 | | | |
| 1425498_at | Prpf4b | PRP4 pre-mRNA processing factor 4 homolog B (yeast) | 0.59 | U48737 | AAH59713 /// Q61136 /// Q8BND8 /// Q8C5G1 /// Q99L76 |
| 1453221_at | Gopc | golgi associated PDZ and coiled-coil motif containing | 0.59 | AA437924 | Q8BH60 /// Q8BSV4 /// Q8R025 /// Q920R1 /// Q9ET11 |
| 1427108_at | 9530068E07Rik | RIKEN cDNA 9530068E07 gene | 0.59 | BM233467 | Q8K201 /// Q922L7 /// Q9CVN1 |
| 1417981_at | Insig2 | insulin induced gene 2 | 0.59 | AV257512 | Q8BWP1 /// Q91WG1 |
| 1452989_at | 2900009J20Rik | RIKEN cDNA 2900009J20 gene | 0.59 | BB315961 | — |
| 1423572_at | Bcl2l2 | Bcl2-like 2 | 0.59 | BB485989 | BAB23468 /// P70345 /// Q8CFR2 /// Q8CGL4 /// Q9CYW5 /// Q9D1Y5 |
| 1445194_at | Cnk2-pending | connector enhancer of KSR2 | 0.59 | BB355006 | AAH60716 /// Q80TP2 /// Q80YA9 |
| 1437030_at | Plcd4 | phospholipase C, delta 4 | 0.59 | AV257260 | — |
| 1420822_s_at | Sgpp1 | sphingosine-1-phosphate phosphatase 1 | 0.59 | NM_030750 | Q9JI99 |
| 1433457_s_at | Grsf1 | G-rich RNA sequence binding factor 1 | 0.59 | AV090328 | Q8BR05 /// Q8BRG7 /// Q8C298 /// Q8C5Q4 |
| 1438071_at | Pms1 | postmeiotic segregation increased 1 (S. cerevisiae) | 0.59 | BM200777 | Q8BLI9 /// Q8K119 |
| 1455954_x_at | Gpaa1 | GPI anchor attachment protein 1 | 0.59 | BB332286 | Q9WTK3 |
| 1430526_a_at | Smarca2 | SWI/SNF related, matrix associated, actin dependent | 0.59 | AK011935 | O35846 /// Q7TND4 /// Q8R1W7 /// Q99KH6 /// Q9CTU8 /// Q9D007 |
| | | regulator of chromatin, subfamily a, member 2 | | | |
| 1440818_s_at | Sf3b1 | splicing factor 3b, subunit 1 | 0.59 | BB161546 | Q8C2Y9 /// Q99NB9 |
| 1456433_at | Rcbtb1 | regulator of chromosome condensation (RCC1) and | 0.59 | BB000798 | AAH67005 /// Q8BTZ6 /// Q8BZV0 |
| | | BTB (POZ) domain containing protein 1 | | | |
| 1423301_at | Copb1 | coatomer protein complex, subunit beta 1 | 0.59 | BF147382 | AAH30837 /// Q9JIF7 |
| 1428409_at | Mak3p | Mak3p homolog (S. cerevisiae) | 0.60 | AK013287 | AAH57117 /// Q7TML2 /// Q80VE3 /// Q9D0Q8 |
| 1434228_at | — | Mus musculus, Similar to pyruvate dehydrogenase | 0.60 | AV255921 | — |
| | | phosphatase, clone IMAGE: 6492665, mRNA | | | |
| 1441879_x_at | Mkm1 | makorin, ring finger protein, 1 | 0.60 | AV218897 | Q8C5B6 /// Q8C5V4 /// Q99LD7 /// Q9DB86 /// Q9QXP6 |
| 1421812_at | Tapbp | TAP binding protein | 0.60 | AF043943 | Q8C6N4 /// Q91WI5 /// Q9D679 /// Q9R233 |
| 1437980_at | 9130230N09Rik | RIKEN cDNA 9130230N09 gene | 0.60 | BB814947 | — |
| 1434580_at | Enpp4 | ectonucleotide pyrophosphatase/phosphodiesterase 4 | 0.60 | AV280361 | Q8BTJ4 /// Q8K1L3 |
| 1458261_at | — | Mus musculus transcribed sequence with weak | 0.60 | BB701997 | — |
| | | similarity to protein ref: NP_081764.1 (M. musculus) | | | |
| | | RIKEN cDNA 5730493B19 [Mus musculus] | | | |
| 1452661_at | Trfr | transferrin receptor | 0.60 | AK011596 | BAC40674 /// Q62351 /// Q8C872 /// Q8JZS3 |
| 1438349_at | LOC381067 | Similar to zinc finger protein 52 | 0.60 | BG069331 | Q80ZY7 |
| 1415686_at | Rab14 | RAB14, member RAS oncogene family | 0.60 | AV339290 | AAH56648 /// Q91V41 |
| 1434062_at | 8430421H08Rik | RIKEN cDNA 8430421H08 gene | 0.60 | AV226672 | — |
| 1455342_at | 6330414G02Rik | RIKEN cDNA 6330414G02 gene | 0.60 | BM232966 | — |
| 1421530_a_at | Grm8 | glutamate receptor, metabotropic 8 | 0.60 | NM_008174 | P47743 |
| 1426939_at | 2310007F12Rik | RIKEN cDNA 2310007F12 gene | 0.60 | BG070464 | Q8C8T8 /// Q8R3M9 |
| 1436051_at | 9630007J19Rik | RIKEN cDNA 9630007J19 gene | 0.60 | BQ174518 | — |
| 1455261_at | Luc7l | Luc7 homolog (S. cerevisiae)-like | 0.60 | BB400102 | Q9CYI4 |
| 1434839_s_at | 8030499H02Rik | RIKEN cDNA 8030499H02 gene | 0.60 | BG071620 | Q8BHJ5 /// Q8C4A2 |
| 1428235_at | Sdhd | succinate dehydrogenase complex, subunit D, integral | 0.60 | AK013962 | Q9CXV1 /// Q9D6J9 |
| | | membrane protein | | | |
| 1433891_at | Gpr48 | G protein-coupled receptor 48 | 0.60 | BI107632 | AAH56637 /// Q80T31 /// Q8BXS9 /// Q8BZR7 |
| 1417999_at | Itm2b | integral membrane protein 2B | 0.60 | NM_008410 | BAB22220 /// BAB22877 /// BAB23828 /// BAC36212 /// BAC36822 /// O89051 /// Q9CW90 /// |
| | | | | | Q9D1Q3 /// Q9JME4 |
| 1424366_at | Tmem15 | transmembrane protein 15 | 0.60 | BC026973 | Q8R2Y3 |
| 1418591_at | Dnaja4 | DnaJ (Hsp40) homolog, subfamily A, member 4 | 0.60 | NM_021422 | BAC32747 /// BAC36232 /// Q8R1X2 /// Q9JMC3 |
| 1451668_at | C530043G21Rik | RIKEN cDNA C530043G21 gene | 0.60 | BG060641 | BAC97964 /// Q8VCS3 |
| 1460359_at | Armcx3 | armadillo repeat containing, X-linked 3 | 0.60 | AK004598 | Q8BHS6 /// Q91VP8 /// Q9DC32 |
| 1434383_at | Pja2 | praja 2, RING-H2 motif containing | 0.60 | BM114949 | Q80U04 /// Q810E3 /// Q91W46 /// Q99KC0 |
| 1455257_at | Itgb3 | integrin beta 3 | 0.60 | AV352983 | — |
| 1415741_at | Tparl | TPA regulated locus | 0.61 | NM_011626 | P52875 |
| 1454723_at | 1110033M05Rik | RIKEN cDNA 1110033M05 gene | 0.61 | AV141095 | AAH57380 /// Q8BVS2 /// Q8C770 /// Q9DAC9 /// Q9Z106 |
| 1420441_at | Cenpc | centromere autoantigen C | 0.61 | NM_007683 | P49452 /// Q9CRZ7 |
| 1428970_at | Mak3p | Mak3p homolog (S. cerevisiae) | 0.61 | AV113878 | AAH57117 /// Q7TML2 /// Q80VE3 /// Q9D0Q8 |
| 1440423_at | D430004I08Rik | RIKEN cDNA D430004I08 gene | 0.61 | AV363211 | Q8C3U9 /// Q8C5F2 |
| 1440926_at | — | Mus musculus transcribed sequences | 0.61 | BB555042 | — |
| 1437168_at | Srrp-pending | serine-arginine repressor protein | 0.61 | BB335578 | Q8C8K3 |
| 1448123_s_at | Tgfbi | transforming growth factor, beta induced | 0.61 | NM_009369 | P82198 |
| 1451096_at | Ndufs2 | NADH dehydrogenase (ubiquinone) Fe—S protein 2 | 0.61 | BC016097 | Q91WD5 /// Q99L23 |
| 1423350_at | Socs5 | suppressor of cytokine signaling 5 | 0.61 | AA510713 | O54928 /// Q7TSK1 |
| 1423599_a_at | Pdcl | phosducin-like | 0.61 | AK004704 | BAC26056 /// BAC26133 /// Q923E8 |
| 1441139_at | B330003H21 | hypothetical protein B330003H21 | 0.61 | BB321858 | Q8C8M4 |
| 1427475_a_at | Nrap | nebulin-related anchoring protein | 0.61 | BC002020 | O35884 /// Q80V40 /// Q80XB4 |
| 1434052_at | AI593442 | expressed sequence AI593442 | 0.61 | AV327193 | Q8BPR8 /// Q8CC42 |
| 1431873_a_at | Tube1 | epsilon-tubulin 1 | 0.61 | AK010005 | AAH62179 /// Q8BYF9 /// Q9D6T1 |
| 1436044_at | Scn7a | sodium channel, voltage-gated, type VI, alpha | 0.61 | BB452990 | Q62467 |
| | | polypeptide | | | |
| 1425662_at | Cdadc1 | cytidine and dCMP deaminase domain containing 1 | 0.61 | BC006901 | Q8BMD5 /// Q8BYL2 /// Q8BYN1 /// Q8C014 /// Q922P4 /// Q99KL2 /// Q9D7F3 |
| 1422906_at | Abcg2 | ATP-binding cassette, sub-family G (WHITE), member 2 | 0.61 | NM_011920 | Q7TMS5 /// Q9R004 /// Q9Z1T0 |
| 1448702_at | 1110057H19Rik | RIKEN cDNA 1110057H19 gene | 0.61 | BE287896 | Q9CR20 |
| 1422895_at | Vamp4 | vesicle-associated membrane protein 4 | 0.61 | NM_016796 | O70480 /// Q8BSN6 /// Q9D095 |
| 1423812_s_at | AW146242 | expressed sequence AW146242 | 0.61 | BC024822 | Q8C0B7 /// Q8R1C3 |
| 1438169_a_at | Frmd4b | FERM domain containing 4B | 0.62 | BB009122 | AAH58262 /// BAC98072 /// Q8BIH9 /// Q8C0E8 /// Q8K0I1 /// Q920B0 /// Q920B1 /// |
| | | | | | Q9ESP9 |
| 1449494_at | Rab3c | RAB3C, member RAS oncogene family | 0.62 | AY026947 | BAC37689 /// Q63482 /// Q9CXS2 |
| 1438358_x_at | Pfdn5 | prefoldin 5 | 0.62 | AV124256 | BAB24185 /// BAC25814 /// Q9DAJ0 /// Q9WU28 |
| 1434485_a_at | Ugp2 | UDP-glucose pyrophosphorylase 2 | 0.62 | AW146314 | AAH61208 /// Q8R0M2 /// Q8R3D2 /// Q91ZJ5 |
| 1449983_a_at | Nqo2 | NAD(P)H dehydrogenase, quinone 2 | 0.62 | NM_020282 | Q9CVF5 /// Q9CVI1 /// Q9JI75 |
| 1429600_at | 1110019K23Rik | RIKEN cDNA 1110019K23 gene | 0.62 | AK003824 | — |
| 1416200_at | 9230117N10Rik | RIKEN cDNA 9230117N10 gene | 0.62 | NM_133775 | Q8BVZ5 /// Q99L46 |
| 1449227_at | Ch25h | cholesterol 25-hydroxylase | 0.62 | NM_009890 | Q8CHQ2 /// Q9Z0F5 |
| 1453312_at | 1200006M05Rik | RIKEN cDNA 1200006M05 gene | 0.62 | BB264725 | Q8BK50 /// Q9DC22 |
| 1420821_at | Sgpp1 | sphingosine-1-phosphate phosphatase 1 | 0.62 | NM_030750 | Q9JI99 |
| 1449203_at | Slco1a5 | solute carrier organic anion transporter family, member | 0.62 | NM_130861 | Q91YY5 /// Q99K89 |
| | | 1a5 | | | |
| 1460632_at | Rdh10 | retinol dehydrogenase 10 (all-trans) | 0.62 | BG069583 | — |
| 1455976_x_at | Dbi | diazepam binding inhibitor | 0.62 | AV019984 | BAB25730 /// BAB25755 /// BAB32175 /// BAC25658 /// P31786 |
| 1418927_a_at | Habp4 | hyaluronic acid binding protein 4 | 0.62 | NM_019986 | Q9D450 /// Q9JKS5 |
| 1455785_at | — | Mus musculus transcribed sequences | 0.62 | BQ175978 | P16388 /// Q8CA58 |
| 1454805_at | Wtap | Wilms' tumour 1-associating protein | 0.62 | AV141160 | AAH46416 /// BAC36191 /// Q9ER69 |
| 1425197_at | Ptpn2 | protein tyrosine phosphatase, non-receptor type 2 | 0.62 | BG076152 | Q06180 /// Q922E7 |
| 1452598_at | 2810418N01Rik | RIKEN cDNA 2810418N01 gene | 0.62 | AK013116 | BAC97891 /// Q8K1A2 /// Q9CZ15 |
| 1433565_at | 2410002M20Rik | RIKEN cDNA 2410002M20 gene | 0.62 | BM209793 | AAH59875 /// Q8BVY2 |
| 1451415_at | 1810011O10Rik | RIKEN cDNA 1810011O10 gene | 0.62 | BC016562 | Q9D915 |
| 1426899_at | 4930451A13Rik | RIKEN cDNA 4930451A13 gene | 0.62 | AV209678 | Q8K0F1 /// Q8VE48 |
| 1436599_at | — | Mus musculus adult male corpora quadrigemina cDNA, | 0.62 | BB314596 | — |
| | | RIKEN full-length enriched library, clone: B230348D21 | | | |
| | | product: unknown EST, full insert sequence | | | |
| 1437855_at | Mtap4 | microtubule-asaociated protein 4 | 0.62 | BB280360 | P27546 /// Q60638 /// Q7TPC6 /// Q7TPD4 /// Q80YQ5 /// Q8CFP5 |
| 1438295_at | — | Mus musculus 3 days neonate thymus cDNA, RIKEN | 0.62 | BM247146 | — |
| | | full-length enriched library, clone: A630066H14 | | | |
| | | product: unknown EST, full insert sequence | | | |
| 1457139_at | — | Mus musculus transcribed sequences | 0.62 | AV021813 | AAH58110 /// BAC97954 /// Q8BZC7 /// Q8C173 /// Q8VDM3 /// Q9CSL7 |
| 1433490_s_at | Epb4.1l2 | erythrocyte protein band 4.1-like 2 | 0.62 | BE951907 | O70318 /// Q7TPN6 /// Q80UE3 /// Q80UE4 /// Q80UE5 /// Q811B2 /// Q811C0 /// Q8BSR4 /// |
| | | | | | Q8C928 /// Q8CGJ6 /// Q9EPM7 /// Q9EPM8 |
| 1459774_at | — | Mus musculus transcribed sequences | 0.62 | AI662002 | — |
| 1429005_at | Mfhas1 | malignant fibrous histiocytoma amplified sequence 1 | 0.62 | BB107412 | Q8C4N5 |
| 1455014_at | — | Mus musculus adult male hippocampus cDNA, RIKEN | 0.63 | BM213104 | — |
| | | full-length enriched library, clone: C630020C21 | | | |
| | | product: unknown EST, full insert sequence | | | |
| 1419975_at | Scp2 | sterol carrier protein 2, liver | 0.63 | C76618 | P32020 |
| 1434229_a_at | Polb | polymerase (DNA directed), beta | 0.63 | BG094331 | AAH60998 /// Q8K409 |
| 1423489_at | Mmd | monocyte to macrophage differentiation-associated | 0.63 | BC021914 | AAR08388 /// Q9CQY7 |
| 1435556_at | 4933407K12Rik | RIKEN cDNA 4933407K12 gene | 0.63 | AV270881 | — |
| 1425048_a_at | Hmgb1 | high mobility group box 1 | 0.63 | U00431 | AAH64790 /// BAC29902 /// BAC39289 /// P07155 /// Q8BNM0 /// Q8BQ02 /// Q8C7C4 |
| 1417948_s_at | Ilf2 | interleukin enhancer binding factor 2 | 0.63 | NM_026374 | Q99KS3 /// Q9CXY6 |
| 1458528_at | — | Mus musculus transcribed sequences | 0.63 | AW491643 | — |
| 1443665_at | — | Mus musculus transcribed sequences | 0.63 | BE994639 | — |
| 1450484_a_at | Tyki | thymidylate kinase family LPS-inducible member | 0.63 | AK004595 | AAH57565 /// Q9DC34 |
| 1436300_at | C430014H23Rik | RIKEN cDNA C430014H23 gene | 0.63 | BB435342 | — |
| 1423952_a_at | Krt2-7 | keratin complex 2, basic, gene 7 | 0.63 | BC010337 | Q9DCV7 |
| 1455961_at | Mme | membrane metallo endopeptidase | 0.63 | AV174022 | AAH66840 /// Q61391 /// Q8BNU9 /// Q8K251 |
| 1453766_a_at | 4931407K02Rik | RIKEN cDNA 4931407K02 gene | 0.63 | AK016516 | AAH56955 /// Q9D2D8 |
| 1423025_a_at | Schip1 | schwannomin interacting protein 1 | 0.63 | NM_013928 | AAH60529 /// Q9JLR0 |
| 1417365_a_at | Calm1 | calmodulin 1 | 0.63 | AU079514 | AAH54805 /// BAB23462 /// BAC40168 /// P02593 /// Q9D6G4 |
| 1426204_a_at | Oprl | opiold receptor-like | 0.63 | AF043276 | BAC30067 /// BAC37672 /// P35377 /// Q80WU7 |
| 1429036_at | Otop3 | otopetrin 3 | 0.63 | AK009293 | Q810B4 /// Q9D7E9 |
| 1420478_at | Nap1l1 | nucleosome assembly protein 1-like 1 | 0.63 | BG064031 | P28656 /// Q8BSH9 /// Q9CSP8 |
| 1433626_at | Plscr4 | phospholipid scramblase 4 | 0.63 | BB826296 | P58196 /// Q8BH62 /// Q8BV91 /// Q8BW59 /// Q8BZC5 |
| 1452763_at | 1110027G09Rik | RIKEN cDNA 1110027G09 gene | 0.63 | BB770774 | — |
| 1436072_at | — | — | 0.64 | BG070468 | — |
| 1436561_at | Suv39h2 | suppressor of variegation 3-9 homolog 2 (Drosophila) | 0.64 | BB440055 | Q8BNK2 /// Q8K085 /// Q9EQQ0 |
| 1439526_at | — | Mus musculus adult male cecum cDNA, RIKEN full- | 0.64 | AV375160 | — |
| | | length enriched library, clone: 9130022D06 | | | |
| | | product: unknown EST, full insert sequence | | | |
| 1420846_at | Mrps2 | mitochondrial ribosomal protein S2 | 0.64 | AV031454 | Q8BQ99 /// Q924T2 |
| 1451724_at | Ankmy2 | ankyrin repeat and MYND domain containing 2 | 0.64 | BC024959 | Q8BK14 /// Q8BYW5 /// Q8R3N4 /// Q921J1 |
| 1420808_at | Ncoa4 | nuclear receptor coactivator 4 | 0.64 | NM_019744 | Q8BSH1 /// Q8K2F6 /// Q9CUF2 /// Q9CXF3 /// Q9WV42 |
| 1442138_at | 4933402E03Rik | RIKEN cDNA 4933402E03 gene | 0.64 | BE955672 | — |
| 1449915_at | Zfp202 | zinc finger protein 202 | 0.64 | NM_030713 | Q8C879 /// Q99PG8 /// Q99PG9 |
| 1438156_x_at | Cpt1a | carnitine palmitoyltransferase 1, liver | 0.64 | BB119196 | P97742 /// Q7TQD5 /// Q80SW3 /// Q8BP98 /// Q8C7H8 |
| 1423200_at | Ncor1 | nuclear receptor co-repressor 1 | 0.64 | U22016 | Q60974 /// Q8CHB6 /// Q8VDE8 /// Q9CUV3 |
| 1424550_at | Zfyve27 | zinc finger, FYVE domain containing 27 | 0.64 | BB663137 | Q8CFP8 /// Q8R1D3 |
| 1440057_at | Hsd17b7 | hydroxysteroid (17-beta) dehydrogenase 7 | 0.64 | AV322070 | BAC25918 /// BAC34124 /// O88736 /// Q8C5N9 /// Q921L1 |
| 1429476_s_at | Dnaja2 | DnaJ (Hsp40) homolog, subfamily A, member 2 | 0.64 | BG063818 | BAC36946 /// BAC38809 /// Q9QYJ0 |
| 1453174_at | 2310076G13Rik | RIKEN cDNA 2310076G13 gene | 0.64 | AK010199 | — |
| 1454939_at | E130113K22Rik | RIKEN cDNA E130113K22 gene | 0.64 | BB268102 | — |
| 1430226_at | 2900036K24Rik | RIKEN cDNA 2900036K24 gene | 0.64 | AK013623 | — |
| 1419291_x_at | Gas5 | growth arrest specific 5 | 0.64 | NM_013525 | Q99KJ3 |
| 1448537_at | Ttc1 | tetratricopeptide repeat domain 1 | 0.64 | NM_133795 | Q91Z38 /// Q9CTZ9 |
| 1455384_x_at | D030056L22 | hypothetical protein D030056L22 | 0.64 | BB256746 | Q8BJJ5 /// Q8VCE4 |
| 1424616_s_at | Frag1 | FGF receptor activating protein 1 | 0.64 | BG063931 | — |
| 1452899_at | Rian | RNA imprinted and accumulated in nucleus | 0.64 | AK017440 | — |
| 1460602_at | Dlc1 | deleted in liver cancer 1 | 0.64 | BB768194 | Q9R0Z9 |
| 1450897_at | AU014947 | expressed sequence AU014947 | 0.64 | BM248774 | — |
| 1435695_a_at | A030007L17Rik | RIKEN cDNA A030007L17 gene | 0.64 | AA673177 | Q9D7X8 |
| 1428905_at | Rraga | Ras-relaled GTP binding A | 0.64 | AI118026 | Q80X95 /// Q8C1S2 /// Q8CFU3 |
| 1460286_at | — | septin 6 | 0.64 | NM_019942 | BAC40453 /// Q8C2L2 /// Q8C406 /// Q8C848 /// Q9R1T4 |
| 1417307_at | Dmd | dystrophin, muscular dystrophy | 0.64 | NM_007868 | P11531 /// Q8BHM1 |
| 1436740_at | 2610005L07Rik | RIKEN cDNA 2610005L07 gene | 0.64 | AI585679 | — |
| 1438368_a_at | Matr3 | matrin 3 | 0.65 | BB390675 | BAC98009 /// Q7TN66 /// Q8K310 |
| 1441693_at | 1100001H14Rik | RIKEN cDNA 1100001H14 gene | 0.65 | BB193360 | — |
| 1455781_at | BC027231 | cDNA sequence BC027231 | 0.65 | AU067804 | Q8R2U2 |
| 1456582_x_at | 5230400G24Rik | RIKEN cDNA 5230400G24 gene | 0.65 | BB024498 | AAH59229 /// Q91VG0 /// Q9D3P5 |
| 1446594_at | — | Mus musculus transcribed sequence with weak | 0.65 | BB205215 | — |
| | | similarity to protein pir: S60335 (H. sapiens) S60335 | | | |
| | | TGF-beta receptor interacting protein 1-human | | | |
| 1415908_at | Tspyl | testis-specific protein, Y-encoded-like | 0.65 | AF042180 | O88852 |
| 1416669_s_at | Naca | nascent polypeptide-sssociated complex alpha | 0.65 | NM_013608 | Q60817 |
| | | polypeptide | | | |
| 1437455_a_at | Btg1 | B-cell translocation gene 1, anti-proliferative | 0.65 | AW322026 | P31607 |
| 1418651_at | Spata6 | spermatogenesis associated 6 | 0.65 | AK005819 | Q8BW97 /// Q99MU6 /// Q9D9J1 /// Q9DAI3 |
| 1429358_at | 4921533L14Rik | RIKEN cDNA 4921533L14 gene | 0.65 | AK019549 | Q8BXS8 /// Q8BZL9 /// Q8K2K2 /// Q9D2J6 |
| 1435047_at | — | Mus musculus transcribed sequences | 0.65 | AI666801 | — |
| 1456795_at | — | Mus musculus 13 days embryo heart cDNA, RIKEN full- | 0.65 | BB449568 | — |
| | | length enriched library, clone: D330027G24 | | | |
| | | product: unclassifiable, full insert sequence | | | |
| 1434609_at | B930007L02Rik | RIKEN cDNA B930007L02 gene | 0.65 | BQ174167 | — |
| 1426328_a_at | Scn3b | sodium channel, voltage-gated, type III, beta | 0.65 | AY049036 | Q8BHK2 |
| 1457034_at | Rap140-pending | retinoblastoma-associated protein 140 | 0.65 | BM209908 | Q9CUD3 |
| 1447818_x_at | 1810036J22Rik | RIKEN cDNA 1810036J22 gene | 0.65 | AV271831 | Q9D8T3 |
| 1434712_at | AI452372 | expressed sequence AI452372 | 0.65 | W34859 | Q8BGV8 /// Q8C4Y9 |
| 1442281_at | — | Mus musculus transcribed sequences | 0.65 | BG069783 | — |
| 1423157_at | Gnpnat1 | glucosamine-phosphate N-acetyltransferase 1 | 0.65 | AK008566 | Q9JK38 |
| 1450966_at | Crot | carnitine O-octanoyltransferase | 0.65 | BB283187 | Q921I4 /// Q9DC50 |
| 1450983_at | Akap8 | A kinase (PRKA) anchor protein 8 | 0.65 | BG069776 | Q8BP29 /// Q9DBR0 /// Q9R0L8 |
| 1438407_at | 9330132E09Rik | RIKEN cDNA 9330132E09 gene | 0.65 | AV336691 | Q8BZP3 |
| 1428830_at | C030026E19Rik | RIKEN cDNA C030026E19 gene | 0.65 | AK021102 | — |
| 1435389_at | — | Mus musculus transcribed sequences | 0.65 | BM899236 | — |
| 1434343_at | 5730403M16Rik | RIKEN cDNA 5730403M16 gene | 0.66 | AV173406 | Q7TNU5 /// Q8BIA9 |
| 1434931_at | Neo1 | neogenin | 0.66 | BB667778 | P97798 /// Q7TQG5 |
| 1434403_at | Spred2 | sprouty protein with EVH-1 domain 2, related sequence | 0.66 | AV229054 | AAH66013 /// Q8K2N1 /// Q924S7 |
| 1454149_a_at | Ccnl2 | cyclin L2 | 0.66 | AK008585 | Q60995 /// Q8BLP2 /// Q8CIJ8 /// Q99L73 /// Q9CVZ6 /// Q9D814 /// Q9JJA7 /// Q9QXH5 |
| 1423678_at | BC017643 | cDNA sequence BC017643 | 0.66 | BC017643 | Q8VD13 |
| 1433452_at | B630019K06Rik | RIKEN cDNA B630019K06 gene | 0.66 | BB179847 | Q7TNS5 /// Q8C8L2 |
| 1431337_a_at | 1810055E12Rik | RIKEN cDNA 1810055E12 gene | 0.66 | AK004643 | Q91WS5 /// Q9D8N2 |
| 1428682_at | 4631426G04Rik | RIKEN cDNA 4631426G04 gene | 0.66 | AK019473 | Q8BYK8 |
| 1438106_at | Pcdhb22 | protocadherin beta 22 | 0.66 | AV336932 | Q8C8Z3 /// Q91XZ8 /// Q925L0 |
| 1419737_a_at | Ldh1 | lactate dehydrogenase 1, A chain | 0.66 | NM_010699 | AAH66858 /// BAC41000 /// P06151 /// Q99K20 |
| 1455048_at | Igsf2 | immunoglobulin superfamily, member 2 | 0.66 | BB484576 | BAC32470 /// BAC35000 /// BAC97961 /// Q7TPV3 |
| 1416659_at | Eif3s10 | eukaryotic translation initiation factor 3, subunit 10 | 0.66 | AW701127 | P23116 |
| | | (theta) | | | |
| 1428921_at | 2810021B07Rik | RIKEN cDNA 2810021B07 gene | 0.66 | AK021189 | Q9CZC6 /// Q9D011 |
| 1452209_at | Pkp4 | plakophilin 4 | 0.66 | AV286396 | Q8BK47 /// Q8BVH1 /// Q9CRE3 |
| 1420798_s_at | Pcdha1 | protocadherin alpha 1 | 0.66 | NM_054072 | — |
| 1437075_at | Frmd3 | FERM domain containing 3 | 0.66 | BB099015 | Q8BHD4 /// Q8BV94 /// Q8C045 /// Q9D7L5 /// Q9D7M6 |
| 1459850_x_at | Glrb | glycine receptor, beta subunit | 0.66 | BB345174 | BAC38831 /// P48168 |
| 1424310_at | Mocs2 | molybdenum cofactor synthesis 2 | 0.66 | AI447812 | Q8C5E5 /// Q8R1M7 /// Q9Z223 /// Q9Z224 |
| 1417275_at | Mal | myelin and lymphocyte protein, T-cell differentiation | 0.66 | NM_010762 | BAB23430 /// O09198 /// Q9D2R2 |
| | | protein | | | |
| 1441165_s_at | Clstn2 | calsyntenin 2 | 0.66 | AI448973 | AAH63058 /// Q9ER65 |
| 1439899_at | Gaint13 | UDP-N-acetyl-alpha-D-galactosamine:polypeptide N- | 0.66 | BE995677 | Q8BLE4 /// Q8BYT3 /// Q8CF93 |
| | | acetylgalactosaminyltransferase 13 | | | |
| 1448628_at | Scg3 | secretogranin III | 0.66 | NM_009130 | P47867 /// Q8R1D7 |
| 1425580_a_at | Pik3c3 | phosphoinositide-3-kinase, class 3 | 0.66 | BC024675 | AAH57678 /// Q8R3S8 |
| 1434486_x_at | Ugp2 | UDP-glucose pyrophosphorylase 2 | 0.66 | AW146314 | AAH61208 /// Q8R0M2 /// Q8R3D2 /// Q91ZJ5 |
| 1437539_at | C130083N04Rik | RIKEN cDNA C130083N04 gene | 0.66 | BM236715 | Q8BUX6 |
| 1427050_at | 5730420B22Rik | RIKEN cDNA 5730420B22 gene | 0.66 | BC027108 | Q7TN22 /// Q8BL40 /// Q8R2W8 /// Q9CS82 |
| 1450072_at | Ash1l | ash1 (absent, small, or homeotic)-like (Drosophila) | 0.66 | BG694892 | Q80VY5 /// Q8BM69 /// Q8BTX0 /// Q8BZY6 |
| 1438772_at | Zfp367 | zinc finger protein 367 | 0.67 | BB227141 | Q8BH90 /// Q8BI44 /// Q8BI53 /// Q8BI88 |
| 1450939_at | Entpd1 | ectonucleoside triphosphate diphosphohydrolase 1 | 0.67 | BI151440 | BAC27039 /// P55772 /// Q8CDV7 /// Q8CEB1 /// Q921Q6 |
| 1453035_at | Lnp | limb and neural patterns | 0.67 | BM200788 | AAH57961 /// AAH60153 /// Q7TQ95 |
| 1419031_at | Fads2 | fatty acid desaturase 2 | 0.67 | NM_019699 | AAH57189 /// Q9Z0R9 |
| 1456097_a_at | Itgb3bp | integrin beta 3 binding protein (beta3-endonexin) | 0.67 | BB830191 | Q9CQ82 |
| 1460697_s_at | 2610209M04Rik | RIKEN cDNA 2610209M04 gene | 0.67 | BC027564 | Q8K194 |
| 1433583_at | Zfp365 | zinc finger protein 365 | 0.67 | AV327246 | AAQ11828 /// CAD56774 /// Q80TQ4 /// Q8BG89 /// Q8BK39 /// Q8BXM9 /// Q8BXT2 |
| 1434841_at | 7330405I11 | hypothetical protein 7330405I11 | 0.67 | AI117751 | Q8BQK5 |
| 1448763_at | Atad1 | ATPase family, AAA domain containing 1 | 0.67 | NM_026487 | Q9D5T0 /// Q9D7A4 /// Q9D9C1 |
| 1436214_at | C430010P07Rik | RIKEN cDNA C430010P07 gene | 0.67 | AV023018 | Q8BNM1 /// Q8C4R5 |
| 1431055_a_at | Snx10 | sorting nexin 10 | 0.67 | AK010399 | Q8BY15 /// Q8C1E0 /// Q9CWT3 |
| 1424114_s_at | Lamb1-1 | laminin B1 subunit 1 | 0.67 | BG970109 | P02469 /// Q8K271 /// Q9CRX6 |
| 1435051_at | 2610034K17Rik | RIKEN cDNA 2610034K17 gene | 0.67 | AV375936 | AAH68151 /// Q8BIZ7 /// Q8BTS1 /// Q8BZS8 |
| 1453160_at | 1110067M05Rik | RIKEN cDNA 1110067M05 gene | 0.67 | BB244704 | — |
| 1419655_at | Tle3 | transducin-like enhancer of split 3, homolog of | 0.67 | NM_009389 | AAH56465 /// Q08122 /// Q80TC1 |
| | | Drosophila E(spl) | | | |
| 1418839_at | Glmn | glomulin, FKBP associated protein | 0.67 | NM_133248 | Q8BZM1 |
| 1434097_at | — | Mus musculus 0 day neonate thymus cDNA, RIKEN | 0.67 | BM218328 | — |
| | | full-length enriched library, clone: A430088G18 | | | |
| | | product: hypothetical Zinc finger, C2H2 type containing | | | |
| | | protein, full insert sequence | | | |
| 1419081_at | Apg10l | autophagy 10-like (S. cerevisiae) | 0.67 | NM_025770 | Q8BPA9 /// Q8R1P4 /// Q9D3J7 |
| 1418942_at | Ccdc2 | coiled-coil domain containing 2 | 0.67 | NM_026319 | Q80ZI3 /// Q8BKE9 /// Q8CGJ7 /// Q9CSY1 /// Q9CUS0 /// Q9D9T5 |
| 1441063_at | — | Mus musculus adult male corpora quadrigemina cDNA, | 0.67 | BB229155 | Q8CJF9 |
| | | RIKEN full-length enriched library, clone: B230383O11 | | | |
| | | product: unknown EST, full insert sequence | | | |
| 1427450_x_at | Myo1b | myosin 1B | 0.67 | BI080370 | P46735 /// Q7TQD7 /// Q80VD8 /// Q91ZI6 |
| 1434558_at | 1810073M12Rik | RIKEN cDNA 1810073M12 gene | 0.67 | BG075633 | Q80TP4 /// Q8C8E3 /// Q8CEH9 /// Q8CGF6 |
| 1418434_at | Mkm1 | makorin, ring finger protein, 1 | 0.68 | BQ176661 | Q8C5B6 /// Q8C5V4 /// Q99LD7 /// Q9DB86 /// Q9QXP6 |
| 1420608_at | Rbm18 | RNA binding motif protein 18 | 0.68 | AV116216 | Q8CBD4 /// Q9CR83 |
| 1457954_at | — | Mus musculus transcribed sequences | 0.68 | BE980601 | Q8C986 /// Q8K244 |
| 1457161_at | 9530029O12Rik | RIKEN cDNA 9530029O12 gene | 0.68 | BB111383 | — |
| 1455970_at | — | Mus musculus transcribed sequences | 0.68 | BE370618 | — |
| 1457118_at | 6230417E10Rik | RIKEN cDNA 6230417E10 gene | 0.68 | AV353605 | AAR19362 /// Q8CB26 |
| 1435171_at | — | Mus musculus transcribed sequences | 0.68 | BB667085 | Q9CRK3 |
| 1431772_a_at | Sh3d1B | SH3 domain protein 1B | 0.68 | AK015445 | Q80TG5 /// Q8C9C3 /// Q8CD59 /// Q9CQD9 /// Q9Z0R6 |
| 1434729_at | mKIAA1166 | mKIAA1166 protein | 0.68 | BM120178 | BAC98112 |
| 1417520_at | Nfe2l3 | nuclear factor, erythroid derived 2, like 3 | 0.68 | NM_010903 | Q9D246 /// Q9D3M5 /// Q9WTM4 |
| 1423130_a_at | Sfrs5 | splicing factor, arginine/serine-rich 5 (SRp40, HRS) | 0.68 | AW212917 | Q9D8S5 |
| 1418823_at | Arf6 | ADP-ribosylation factor 6 | 0.68 | BI248938 | P26438 |
| 1418397_at | Zfp275 | Zinc finger protein 275 | 0.68 | BC019962 | Q8VE24 /// Q9D3I9 |
| 1425350_a_at | Myef2 | myelin basic protein expression factor 2, repressor | 0.68 | U13262 | AAH60946 /// BAC98146 /// Q60690 /// Q8BS80 /// Q8C854 /// Q8QZZ1 /// Q9JLR3 |
| 1428779_at | 8430415N23Rik | RIKEN cDNA 8430415N23 gene | 0.68 | BB526541 | — |
| 1418545_at | Wasf1 | WASP family 1 | 0.68 | NM_031877 | Q8R5H6 /// Q91W51 /// Q9ERQ9 |
| 1426723_at | 8430408H12Rik | RIKEN cDNA 8430408H12 gene | 0.68 | BE570732 | AAH62967 /// Q80TD4 /// Q80XI0 /// Q8BH57 /// Q8BRM0 /// Q922Z9 /// Q9CRR1 /// Q9CSL0 |
| 1456030_at | Klf13 | Kruppel-like factor 13 | 0.68 | BE949230 | Q9JJZ6 |
| 1428347_at | Cyfip2 | cytoplasmic FMR1 interacting protein 2 | 0.68 | AK005148 | AAH56974 /// Q810V4 /// Q8BSW0 /// Q8CHA9 /// Q8K118 /// Q924D3 /// Q9R181 |
| 1455184_at | B230364F10 | hypothetical protein B230364F10 | 0.68 | BG071991 | — |
| 1426961_at | 6820402O20Rik | RIKEN cDNA 6820402O20 gene | 0.68 | BB308157 | AAH60121 /// Q8BLG0 /// Q8BZI4 |
| 1434843_at | C130034K06 | hypothetical protein C130034K06 | 0.68 | BG070968 | AAH62107 /// Q8CA10 |
| 1418433_at | Cab39 | calcium binding protein 39 | 0.68 | AK005226 | Q8K312 /// Q8VDZ8 |
| 1449152_at | Cdkn2b | cyclin-dependent kinase inhibitor 2B (p15, inhibits | 0.68 | AF059567 | AAC14569 /// P55271 |
| | | CDK4) | | | |
| 1458341_x_at | — | Mus musculus transcribed sequences | 0.68 | BB397841 | — |
| 1460712_s_at | Ap1g1 | adaptor protein complex AP-1, gamma 1 subunit | 0.68 | C86561 | P22892 /// Q8BSZ7 /// Q8CBB7 /// Q8CC03 |
| 1448525_a_at | Bnip3l | BCL2/adenovirus E1B 19 kDa-interacting protein 3-like | 0.69 | AK018668 | BAB23456 /// BAB25351 /// BAB28869 /// Q91Z78 /// Q9Z2F7 |
| 1429146_at | 6620401M08Rik | RIKEN cDNA 6620401M08 gene | 0.69 | BF011349 | — |
| 1436098_at | Bche | butyrylcholinesterase | 0.69 | BB667452 | — |
| 1433791_at | Rab9b | RAB9B, member RAS oncogene family | 0.69 | BB084626 | Q8BHH2 |
| 1448473_at | Bub3 | budding uninhibited by benzimidazoles 3 | 0.69 | BE986800 | AAH25089 /// BAC40409 /// Q8BH42 /// Q9CSI6 /// Q9WVA3 |
| | | homolog (S. cerevisiae) | | | |
| 1444077_at | — | Mus musculus transcribed sequences | 0.69 | BE993694 | — |
| 1455746_at | Kif13a | kinesin family member 13A | 0.69 | BF166390 | Q8CA55 /// Q8CDQ6 /// Q9EQW7 |
| 1429899_at | 5730414N17Rik | RIKEN cDNA 5730414N17 gene | 0.69 | BB039237 | — |
| 1458940_at | 9430076K19Rik | RIKEN cDNA 9430076K19 gene | 0.69 | BF147707 | — |
| 1454607_s_at | Psat1 | phosphoserine aminotransferase 1 | 0.69 | AV216491 | BAC33959 /// Q8BTJ1 /// Q99JU9 /// Q99K85 |
| 1439590_at | — | Mus musculus adult male testis cDNA, RIKEN full- | 0.69 | AV273072 | AAH57035 /// Q8C0T0 |
| | | length enriched library, clone: 4931440N07 | | | |
| | | product: hypothetical Type-1 copper (blue) | | | |
| | | domain/Leucine-rich region containing protein, full | | | |
| | | insert sequence | | | |
| 1452677_at | Pnpt1 | polyribonucleotide nucleotidyltransferase 1 | 0.69 | BB777815 | Q810U7 /// Q812B3 /// Q8K1R3 /// Q8R2U3 |
| 1452130_at | 2310042M24Rik | RIKEN cDNA 2310042M24 gene | 0.69 | BI790903 | Q8BW13 /// Q9D710 |
| 1434285_at | Frmd4a | FERM domain containing 4A | 0.69 | BB701578 | — |
| 1417847_at | Ulk2 | Unc-51 like kinase 2 (C. elegans) | 0.69 | NM_013881 | Q80TV7 /// Q9QY01 /// Q9WTP4 |
| 1425537_at | Ppm1a | protein phosphatase 1A, magnesium dependent, alpha | 0.69 | AF259672 | P49443 /// Q8R4T7 /// Q9EQE2 /// Q9EQE3 |
| | | isoform | | | |
| 1418983_at | Cipp | channel-interacting PDZ domain protein | 0.69 | AV287690 | AAH57124 /// AAH62194 /// O70471 /// Q80YR8 /// Q8BPB9 /// Q8VE63 |
| 1434292_at | E130013N09Rik | RIKEN cDNA E130013N09 gene | 0.69 | BI731047 | — |
| 1415788_at | BC002236 | cDNA sequence BC002236 | 0.69 | BF158817 | AAH56652 /// Q8BGR9 /// Q99LT3 |
| 1455337_at | 9030023J02Rik | RIKEN cDNA 9030023J02 gene | 0.69 | BQ175875 | — |
| 1418603_at | Avpr1a | arginine vasopressin receptor 1A | 0.69 | D49729 | Q62463 |
| 1436034_at | Rab1 | RAB1, member RAS oncogane family | 0.69 | AW550283 | BAC28697 /// BAC98287 /// P11476 /// Q811M4 |
| 1451413_at | Cast | calpastatin | 0.69 | AB026997 | P51125 /// Q8BS37 /// Q8C281 /// Q8CB83 /// Q8CE04 /// Q8CE80 /// Q921U7 |
| 1450729_at | Hs2st1 | heparan sulfate 2-O-sulfotransferase 1 | 0.69 | AV346600 | AAH59008 /// O88464 /// Q8R3H7 /// Q9JLK2 |
| 1416458_at | Arf2 | ADP-ribosylation factor 2 | 0.69 | NM_007477 | BAC31426 /// BAC35273 /// BAC36882 /// P16500 /// Q8BSL7 /// Q91VR9 |
| 1419693_at | Colec12 | collectin sub-family member 12 | 0.69 | NM_130449 | AAH57936 /// Q8K4Q8 /// Q8VIF6 |
| 1449229_a_at | Cdkl2 | cyclin-dependent kinase-like 2 (CDC2-related kinase) | 0.69 | NM_016912 | Q9QUK0 /// Q9QYI1 /// Q9QYI2 |
| 1418071_s_at | Cdyl | chromodomain protein, Y chromosome-like | 0.69 | AF081260 | AAH62123 /// Q9WTK2 |
| 1437543_at | D3Ertd330e | DNA segment, Chr 3, ERATO Doi 330, expressed | 0.69 | BB488001 | Q91WJ8 |
| 1428543_at | Ppat | phosphoribosyl pyrophosphate amidotransferase | 0.69 | AV305746 | Q8CIH9 |
| 1419736_a_at | Elf1ay | eukaryotic translation initiation factor 1A, Y-linked | 0.69 | NM_025437 | AAH27284 /// BAC41066 /// Q60872 /// Q8BJZ2 /// Q8BMH8 /// Q8BMJ3 /// Q9CSL9 |
| 1448527_at | Pdcd10 | programmed cell death 10 | 0.69 | AV094856 | Q8VE70 /// Q9DAR3 /// Q9WV43 |
| 1455102_at | D330037H05Rik | RIKEN cDNA D330037H05 gene | 0.70 | BB213860 | Q8BWW4 /// Q9D6P9 |
| 1438306_at | 3110001E11Rik | RIKEN cDNA 3110001E11 gene | 0.70 | AV340072 | Q80ZX1 /// Q8CCR1 /// Q9CXV6 |
| 1435701_at | — | Mus musculus 10 days neonate medulla oblongata | 0.70 | DM118858 | — |
| | | cDNA, RIKEN full-length enriched library, | | | |
| | | clone: B830010l11 product: unclassifiable, full Insert | | | |
| | | sequence | | | |
| 1448761_a_at | Copg2 | coatomer protein complex, subunit gamma 2 | 0.70 | NM_017478 | — |
| 1435446_a_at | Chpt1 | choline phosphotransferase 1 | 0.70 | BF180212 | AAR16089 /// Q8C025 /// Q8K0H2 /// Q91W91 |
| 1427105_at | 2610510J17Rik | RIKEN cDNA 2610610J17 gene | 0.70 | BM230253 | AAH58243 /// Q8R2W7 /// Q9CZW2 |
| 1443119_at | 6330570A01Rik | RIKEN cDNA 6330670A01 gene | 0.70 | AV335221 | — |
| 1423911_at | Ppp2r5a | protein phosphatase 2, regulatory subunit B (B56), | 0.70 | BC023062 | AAH59026 /// Q8R1U7 |
| | | alpha isoform | | | |
| 1416161_at | Rad21 | RAD21 homolog (S. pombe) | 0.70 | AF332085 | BAC97860 /// Q61550 /// Q810A8 |
| 1427160_at | 2500001H09Rik | RIKEN cDNA 2600001H09 gene | 0.70 | AV374246 | Q80V88 /// Q8K0S1 /// Q8R2P5 /// Q99KH9 |
| 1431328_at | Ppp1cb | protein phosphatase 1, catalytic subunit, beta isoform | 0.70 | AK017392 | AAH46832 /// BAC40636 /// P37140 /// Q8C285 /// Q9DBY2 |
| 1428460_at | Syn2 | synapsin II | 0.70 | AK013810 | AAH66004 /// Q8CE19 /// Q9QWV7 |
| 1425262_at | Cebpg | CCAAT/enhancer binding protein (C/EBP), gamma | 0.70 | AB012273 | BAA25311 /// BAC34355 /// P53568 |
| 1428317_at | 4833415N24Rik | RIKEN cDNA 4833415N24 gene | 0.70 | AI510221 | AAH61464 /// Q80VT2 /// Q80YD9 // Q8BSM8 /// Q9D617 /// Q9D6K8 |
| 1435603_at | SST3 | secreted protein SST3 | 0.70 | BB487754 | CAE48492 /// Q810H2 /// Q8BMD9 |
| 1444097_at | BC019776 | cDNA sequence BC019776 | 0.70 | BB544962 | Q8R1J2 /// Q8VE43 |
| 1443282_at | 2410002M20Rik | RIKEN cDNA 2410002M20 gene | 0.71 | BB565693 | AAH59875 /// Q8BVY2 |
| 1456665_at | B130023L16Rik | RIKEN cDNA B130023L16 gene | 0.71 | BB476944 | — |
| 1459332_at | — | Mus musculus transcribed sequence with moderate | 0.71 | BM197626 | — |
| | | similarity to protein pir: S12207 (M. musculus) S12207 | | | |
| | | hypothetical protein (B2 element) - mouse | | | |
| 1460163_at | — | Mus musculus transcribed sequences | 0.71 | BB039211 | — |
| 1417166_at | Psip2 | PC4 snd SFRS1 interacting protein 2 | 0.71 | NM_133948 | Q80WQ7 /// Q99JF7 /// Q99JFB /// Q99LR4 /// Q9CT03 |
| 1428237_at | 2700059D21Rik | RIKEN cDNA 2700059D21 gene | 0.71 | BI689227 | — |
| 1424357_at | BC018222 | cDNA sequence BC018222 | 0.71 | BC026654 | Q8VCZ2 |
| 1417962_s_at | Ghr | growth hormone receptor | 0.71 | NM_010284 | P16882 /// Q8BPQ3 |
| 1428203_at | C030002O17Rik | RIKEN cDNA C030002O17 gene | 0.71 | AI844535 | Q8BT41 |
| 1445173_at | mKIAA1377 | mKIAA1377 protein | 0.71 | BF472675 | BAC98151 |
| 1451493_at | Ndfip1 | Nedd4 family interacting protein 1 | 0.71 | BC026372 | Q8R0W6 /// Q9EQH8 |
| 1454754_a_at | Aamp | anglo-associated migratory protein | 0.71 | BF468097 | Q8K2C1 |
| 1433602_at | Gabra5 | gamma-aminobutyric acid (GABA-A) receptor subunit | 0.71 | BQ175863 | AAH62112 /// O88964 /// Q8BHJ7 |
| | | alpha 5 | | | |
| 1423295_at | Tm9sf2 | transmembrane 9 superfamily member 2 | 0.71 | BB747462 | BAC33215 /// BAC40645 /// P58021 /// Q8C6H4 /// Q8C7F9 |
| 1423251_at | Luc7l2 | LUC7-like2 (S. cerevisiae) | 0.71 | BG075618 | AAH56383 /// AAH56970 /// Q7TNC4 |
| 1435946_at | D5Ertd135e | DNA segment, Chr 5, ERATO Dol 135, expressed | 0.72 | BB401993 | AAH62133 /// Q8BZS6 |
| 1446959_at | — | Mus musculus transcribed sequences | 0.72 | BG068073 | — |
| | | potassium voltage-gated channel shaker related | | | |
| 1438613_at | Kcna4 | subfamily, member 4 | 0.72 | BB131475 | Q8CBF8 |
| 1460003_at | — | Mus musculus transcribed sequences | 0.72 | AV234963 | — |
| 1426369_at | 3732409C05Rik | RIKEN cDNA 3732409C05 gene | 0.72 | BG094874 | Q8BZS2 /// Q922J9 /// Q9CXE8 /// Q9D0Q1 /// Q9DAU2/ |
| 1438886_at | Heyl | hairy/enhancer-of-split related with YRPW motif-like | 0.72 | BB310549 | Q9DBX7 /// Q9JJV6 /// Q9QXW8 |
| 1429430_at | 8430411F12Rik | RIKEN cDNA 8430411F12 gene | 0.72 | AK018402 | AAH64801 /// P59913 |
| 1425202_a_at | Ank3 | ankyrin 3, epithelial | 0.72 | BC021657 | Q61307 /// Q8CBN3 /// Q8CCD5 /// Q8VC68 /// Q9QXH1 |
| 1433669_at | Akap8 | A kinsee (PRKA) anchor protein 8 | 0.72 | BB037566 | Q8BP29 /// Q9DBR0 /// Q9R0L8 |
| 1450863_a_at | Dcamkl1 | double cortin and calcium/calmodulin-dependent | | | AAH64783 /// Q80VB6 /// Q8BQN2 /// Q8CCN4 /// Q8CHG1 /// Q8VDT3 /// Q9JLM6 /// |
| | | protein kinase-like 1 | 0.72 | BQ174703 | Q9JLM7 /// Q9JLM8 |
| 1454720_at | Apba3 | amyloid beta (A4) precursor protein-binding, family A, | | | |
| | | member 3 | 0.72 | AV328620 | O88888 /// Q8BR09 |
| 1426216_at | Cog6 | component of oligomeric golgi complex 6 | 0.72 | BC025427 | Q8BRB0 /// Q8BSN7 /// Q8C7Y2 /// Q8CHB1 /// Q8R313 |
| 1429159_at | 4631408O11Rik | RIKEN cDNA 4631408O11 gene | 0.72 | AK018605 | — |
| 1424693_at | 4933407N01Rik | RIKEN cDNA 4933407N01 gene | 0.72 | BC018468 | Q8BVN6 /// Q8VEH8 /// Q9CWA6 |
| 1452395_at | 2410018M14Rik | RIKEN cDNA 2410018M14 gene | 0.72 | AA111022 | — |
| 1435436_at | — | Mus musculus transcribed sequences | 0.72 | BI647951 | — |
| 1451971_at | Cul4a | cullin 4A | 0.72 | BC024113 | Q8BJG5 /// Q8R1T2 /// Q91VY0 /// Q91Z44 /// Q9CTG1 /// Q9CW15 |
| 1450479_x_at | Ptpn12 | protein tyrosine phosphatase, non-receptor type 12 | 0.72 | X63440 | P35831 /// Q80UM4 |
| 1418310_a_at | Ribp1 | retinaldehyde binding protein 1 | 0.72 | NM_020599 | BAB29216 /// Q9Z275 |
| 1423646_at | Zdhhc3 | zinc finger, DHHC domain containing 3 | 0.72 | BB815190 | AAO27359 /// Q8R173 |
| 1454794_at | Spg4 | spastic paraplegia 4 homolog (human) | 0.72 | AV298495 | BAC98092 /// Q80VE0 /// Q9CVK0 /// Q9QYY8 |
| 1436889_at | Gabra1 | gamma-aminobutyric acid (GABA-A) receptor subunit | 0.72 | BQ268470 | BAC28585 /// BAC30368 /// P18504 |
| | | alpha 1 | | | |
| 1451679_at | 6530401D17Rik | RIKEN cDNA 6530401D17 gene | 0.72 | BC016270 | Q8BK31 /// Q9D395 |
| 1448430_a_at | Naca | nascent polypeptide-associated complex alpha | 0.72 | NM_013608 | Q60817 |
| | | polypeptide | | | |
| 1452371_at | 2610019N13Rik | RIKEN cDNA 2610019N13 gene | 0.72 | AW261583 | Q8B8V04 /// Q8BYD3 /// Q8C0Z3 /// Q8CFF2 /// Q8CGI3 /// G9CT17 |
| 1450723_at | isl1 | ISL1 transcription factor, LIM/homeodomain (islet 1) | 0.72 | BQ176915 | P61372 /// Q8BTH7 |
| 1434503_a_at | Lamp2 | lysosomal membrane glycoprotein 2 | 0.73 | BB490768 | P17047 /// Q8BSG8 /// Q8C876 /// Q9CZU7 |
| 1423684_at | Hnrpk | heterogeneous nuclear ribonucleoprotein K | 0.73 | BC006694 | Q07244 /// Q8BGQ8 |
| 1460449_at | C030032C09Rik | RIKEN cDNA C030032C09 gene | 0.73 | BQ174247 | Q8BIZ1 /// Q8BJ47 /// Q8BJ49 /// Q8BZM2 |
| 1450038_s_at | Usp9x | ubiquitin specific protease 9, X chromosome | 0.73 | AW107303 | P70398 /// Q8BR77 /// Q8BS89 |
| 1454651_x_at | Mbp | myelin basic protein | 0.73 | BE994609 | AAH51094 /// BAC30679 /// BAC37705 /// P04370 |
| 1422457_s_at | Sumo3 | SMT3 suppressor of mif two 3 homolog 3 (yeast) | 0.73 | NM_019929 | Q9Z172 |
| 1418723_at | Edg7 | endothelial differentiation, lysophosphatidic acid G- | 0.73 | NM_022983 | BAC31159 /// Q9EQ31 |
| | | protein-coupled receptor 7 | | | |
| 1422520_at | Nef3 | neurofilament 3, medium | 0.73 | NM_008691 | P08553 /// Q8BQ20 |
| 1439341_at | — | Mus musculus transcribed sequence with weak | 0.73 | BB203252 | — |
| | | similarity to protein pir: T00340 (H. sapiens) T00340 | | | |
| | | hypothetical protein KIAA057 - human | | | |
| 1423078_a_at | Sc4mol | sterol-C4-methyl oxidase-like | 0.73 | AK005441 | BAC32201 /// Q9CRA4 |
| 1448261_at | Cdh1 | cadherin 1 | 0.73 | NM_009864 | P09803 |
| 1437132_x_at | Nedd9 | neural precursor cell expressed, developmentally down- | 0.73 | BB535494 | O35177 |
| | | regulated gene 9 | | | |
| 1436511_at | BC031781 | cDNA sequence BC031781 | 0.73 | BM935102 | Q8BJX1 /// Q8K1J5 |
| 1452804_at | 1700010A17Rik | RIKEN cDNA 1700010A17 gene | 0.73 | BG298252 | Q9DAI9 |
| 1431296_at | 4933439K08Rik | RIKEN cDNA 4933439K08 gene | 0.73 | AA555873 | — |
| 1455907_x_at | — | Mus musculus transcribed sequences | 0.73 | BB059017 | — |
| 1456904_at | Gas5 | growth arrest specific 5 | 0.73 | BI650268 | Q99KJ3 |
| 1426607_at | 3110070M22Rik | RIKEN cDNA 3110070M22 gene | 0.73 | BG068672 | Q8VDR5 /// Q9CXM1 /// Q9DAQ7 |
| 1417663_a_at | Ndr3 | N-myc downstream regulated 3 | 0.74 | BE631549 | BAB23475 /// BAC29818 /// Q8CDY0 /// Q8VCV2 // Q9QYF9 |
| 1436302_at | 2410193C02Rik | RIKEN cDNA 2410193C02 gene | 0.74 | BB770006 | Q8BV58 /// Q8BYD0 // Q9CWD6 |
| 1422744_at | Phka1 | phosphorylasa kinase alpha 1 | 0.74 | NM_008832 | P18826 /// Q8BSV5 /// Q8CBA7 |
| 1423176_at | Tob1 | transducer of ErbB-2.1 | 0.74 | BQ266486 | — |
| 1452062_at | Prpsap2 | phosphoribosyl pyrophosphate synthetase-associated | 0.74 | BB246540 | BAC30096 /// BAC36491 /// Q8BK37 /// Q8R574 |
| | | protein 2 | | | |
| 1430500_s_at | Mtx2 | metaxin 2 | 0.74 | AK005233 | BAC40046 /// O88441 /// Q8C454 |
| 1435058_x_at | Stxbp3 | syntaxin binding protein 3 | 0.74 | AI528529 | AAH62901 /// Q60770 /// Q8C7H4 |
| 1415844_at | Syt4 | synaptotagmin 4 | 0.74 | AV336547 | P40749 |
| 1435061_at | Nudt11 | nudix (nucleoside diphosphate linked moiety X)-type | 0.74 | AI853080 | Q8BKF4 /// Q9JJD3 |
| | | motif 11 | | | |
| 1445519_at | Kcns3 | potassium voltage-gated channel, delayed-rectifier | 0.74 | C77819 | Q8BQZ8 |
| | | subfamily S, member 3 | | | |
| 1455446_x_at | Acadsb | acyl-Coenzyme A dehydrogenese, short/branched | 0.74 | BF228057 | Q7TMY2 /// Q9DBL1 |
| | | chain | | | |
| 1453365_at | 8430421H08Rik | RIKEN cDNA 8430421H08 gene | 0.74 | AK018430 | — |
| 1460116_s_at | Spred1 | sprouty protein with EVH-1 domain 1, related sequence | 0.74 | AI450584 | AAH57874 /// Q924S8 |
| 1438024_at | 6230416A05Rik | RIKEN cDNA 6230416A05 gene | 0.74 | AW554392 | — |
| 1435021_at | Gabrb3 | gamma-aminobutyric acid (GABA-A) receptor subunit | 0.74 | BQ175666 | BAC30230 /// P15433 /// Q8C446 |
| | | beta3 | | | |
| 1443992_at | 4921518A06Rik | RIKEN cDNA 4921518A06 gene | 0.74 | BB203972 | Q7TNS4 /// Q8BKV4 /// Q8CES9 /// Q9CUC6 |
| 1452872_at | 2900054D09Rik | RIKEN cDNA 2900054D09 gene | 0.74 | BM217754 | — |
| 1453328_at | 2700008G24Rik | RIKEN cDNA 2700008G24 gene | 0.74 | AW495672 | — |
| 1428086_at | Dnm1l | dynamin 1-like | 0.74 | BM249101 | Q8BNQ5 /// Q8BQ64 /// Q8CGD0 /// Q8K1A1 /// Q8K1M6 |
| 1416253_at | Cdkn2d | cyclin-dependent kinase inhibitor 2D (p19, inhibits | 0.74 | BC013898 | Q60773 /// Q91YV3 |
| | | CDK4) | | | |
| 1428453_at | 5730533P17Rik | RIKEN cDNA 5730533P17 gene | 0.74 | AK017805 | Q7TN07 /// Q8CES0 |
| 1451620_at | Pter | phosphotriesterase related | 0.75 | BB768838 | — |
| 1438413_at | 2810413I22Rik | RIKEN cDNA 2810413I22 gene | 0.75 | AV231698 | AAH58593 /// AAH64127 /// Q80TA3 /// Q8BUH8 /// Q9CQN9 /// Q9CRF0 /// Q9CX65 |
| 1426948_at | Tpr | translocatad promoter region | 0.75 | BM214109 | Q8BIX0 /// Q8BK71 /// Q8BU18 /// Q8K2T9 /// Q8R4A0 /// Q91ZA5 |
| 1438093_x_at | Dbi | diazepam binding inhibitor | 0.75 | BB115327 | BAB25730 /// BAB25755 /// BAB32175 /// BAC25658 /// P31786 |
| 1453565_at | Ndufab1 | NADH dehydrogenase (ubiquinone) 1, alpha/beta | 0.75 | AV221509 | AAH60951 /// BAC40751 /// Q9CR21 |
| | | subcomplex, 1 | | | |
| 1452230_at | Dnajc10 | DnaJ (Hsp40) homolog, subfamily C, member 10 | 0.75 | AV114239 | AAQ14555 /// Q8CH78 /// Q8CIB0 /// Q99LV4 /// Q9CRX9 /// Q9CUG0 /// Q9DC23 |
| 1419362_at | Mrpl35 | mitochondrial ribosomal protein L35 | 0.75 | BF787384 | Q9CQL6 |
| 1453212_at | 1110003H10Rik | RIKEN cDNA 1110003H10 gene | 0.75 | BB705379 | — |
| 1448670_at | Ube2e3 | ubiquitin-conjugating enzyme E2E 3, UBC4/5 homolog | 0.75 | AW120830 | P52483 /// Q8BXB0 |
| | | (yeast) | | | |
| 1419650_at | Zfr | zinc finger RNA binding protein | 0.75 | NM_011767 | AAH58570 /// O88532 /// Q8BS85 /// Q8CGG5 /// Q91VZ0 /// Q9CT34 |
| 1417702_a_at | Hnmt | histamine N-methyltransferase | 0.75 | NM_080462 | Q91VF2 |
| 1452214_at | 9130011J04Rik | RIKEN cDNA 9130011J04 gene | 0.75 | AK018608 | — |
| 1439817_at | AI451465 | expressed sequence AI451465 | 0.75 | AI451465 | — |
| 1440879_at | Abca9 | ATP-binding cassette transporter sub-family A member 9 | 0.75 | AW046072 | Q8BIS4 /// Q8C114 /// Q8K449 |
| 1452592_at | Mgst2 | microsomal glutathione S-transferase 2 | 0.75 | AV066880 | Q8R032 |
| 1428124_at | Gtf2e1 | general transcription factor II E, polypeptide 1 (alpha | 0.75 | AK011543 | BAC27436 /// Q8BV40 /// Q9D0D5 |
| | | subunit) | | | |
| 1424872_at | 2310001H12Rik | RIKEN cDNA 2310001H12 gene | 0.75 | BC012405 | Q8BJ78 |
| 1425332_at | Zfp106 | zinc finger protein 106 | 0.75 | BI452653 | O88465 /// O88466 /// Q8C235 /// Q8CDZ8 /// Q8R3I4 |
| 1419170_at | 2310044D20Rik | RIKEN cDNA 2310044D20 gene | 0.75 | BB667295 | Q8CC46 /// Q8VDR1 /// Q9D238 /// Q9D3L0 /// Q9D6W5 /// Q9D6Z3 |
| 1425542_a_at | Ppp2r5c | protein phosphatase 2, regulatory subunit B (B56), | 0.75 | BC003979 | AAR26474 /// BAC97852 /// Q60996 /// Q8C8H4 /// Q99KW8 /// Q99N67 /// Q99N68 |
| | | gamma isoform | | | |
| 1450779_at | Fabp7 | fatty acid binding protein 7, brain | 0.75 | NM_021272 | P51880 |
| 1434284_at | G630013P12Rik | RIKEN cDNA G630013P12 gene | 0.75 | BQ031214 | Q8BTI4 /// Q8K0U7 |
| 1433540_x_at | Ppp1cb | protein phosphatase 1, catalytic subunit, beta isoform | 0.75 | AW823525 | AAH46832 /// BAC40636 /// P37140 /// Q8C285 /// Q9DBY2 |
| 1426978_at | Klhl2 | kelch-like 2, Mayven (Drosophila) | 0.75 | AW682368 | Q8CCU0 /// Q8JZP3 /// Q8R3U4 |
| 1416412_at | Nsmaf | neutral sphingomyelinase (N-SMase) activation | 0.76 | NM_010945 | O35242 |
| | | associated factor | | | |
| 1438725_at | Thrap1 | thyroid hormone receptor associated protein 1 | 0.76 | BB212816 | BAC97978 |
| 1438324_at | 9330182L06Rik | RIKEN cDNA 9330182L06 gene | 0.76 | AW550882 | Q8BJM9 /// Q8BJN9 /// Q8BJT7 /// Q8BKX9 /// Q8BL89 /// Q8BLT1 /// Q8BM91 /// Q8K107 |
| 1437259_at | Slc9a2 | solute carrier family 9 (sodium/hydrogen exchanger), | 0.76 | AV274006 | Q9WUJ4 |
| | | member 2 | | | |
| 1420748_a_at | Adat1 | adenosine deaminase, tRNA-specific 1 | 0.76 | NM_013925 | Q8VE23 /// Q9JHI2 |
| 1417481_at | Ramp1 | receptor (calcitonin) activity modifying protein 1 | 0.76 | NM_016894 | Q8VII7 /// Q9CT60 /// Q9WTJ5 |
| 1425468_at | Plp | proteolipid protein (myelin) | 0.76 | BB768495 | P60202 /// Q62079 |
| 1422510_at | Ctdspl | CTD (carboxy-terminal domain, RNA polymerase II, | 0.76 | NM_133710 | CAC69078 /// P58465 |
| | | polypeptide A) small phosphatase-like | | | |
| 1441946_at | itih5 | inter-alpha (globulin) inhibitor H5 | 0.76 | AV239969 | AAH62196 /// Q80VG0 /// Q8BJD1 /// Q8BK33 /// Q8BMS5 |
| 1460292_a_at | Smarca1 | SWI/SNF related, matrix associated, actin dependent | 0.76 | NM_053123 | AAH57115 /// Q8BS67 /// Q8BSS1 /// Q91Y58 |
| | | regulator of chromatin, subfamily a, member 1 | | | |
| 1435303_at | 4932409F03Rik | RIKEN cDNA 4932409F03 gene | 0.76 | AV373814 | Q8C0S6 |
| 1455003_at | — | Mus musculus mRNA similar to hypothetical protein | 0.76 | BQ032496 | — |
| | | FLJ10477 (cDNA clone MGC: 47985 IMAGE: | | | |
| | | 5118383), complete cds | | | |
| 1452015_at | 6330416G13Rik | RIKEN cDNA 6330416G13 gene | 0.76 | AV326978 | Q8BMN2 /// Q8C0I7 /// Q8CIJ7 /// Q8R239 |
| 1429383_at | Csnk1g3 | casein kinase 1, gamma 3 | 0.76 | BM195380 | Q8BM57 /// Q8C001 /// Q8K079 |
| 1435234_at | Ncoa2 | nuclear receptor coactivator 2 | 0.76 | BM234716 | Q61026 /// Q7TPU7 /// Q8C961 /// Q8CBM5 /// Q8CE59 |
| 1456137_at | Nrxn3 | neurexin III | 0.76 | BB132137 | AAH60719 /// BAC98015 /// O88724 /// Q8C985 /// Q8CBZ2 /// Q8CCT8 |
| 1429685_at | C030002O17Rik | RIKEN cDNA C030002O17 gene | 0.76 | BB313857 | — |
| 1423246_at | Txndc4 | thioredoxin domain containing 4 (endoplasmic | 0.76 | BI100077 | Q9D1Q6 |
| | | reticulum) | | | |
| 1451531_at | BC018472 | cDNA sequence BC018472 | 0.77 | BC018472 | Q7TSA8 /// Q8K399 /// Q8VEH6 |
| 1445438_at | Ddhd1 | DDHD domain containing 1 | 0.77 | BB132393 | BAC98235 /// Q80YA3 |
| 1452168_x_at | Gspt1 | G1 to phase transition 1 | 0.77 | AB003502 | Q8BPH0 /// Q8CAS6 /// Q8CCV1 /// Q8K2E1 /// Q8R050 |
| 1436298_x_at | Paics | phosphoribosylaminoimidazole carboxylase, | 0.77 | BB066556 | Q9CQ38 /// Q9DCL9 |
| | | phosphoribosylaminoribosylaminoimidazole, | | | |
| | | succinocarboxamide synthetase | | | |
| 1423856_at | Rpl17 | ribosomal protein L17 | 0.77 | BC003896 | Q9ES81 |
| 1433872_at | 2410042D21Rik | RIKEN cDNA 2410042D21 gene | 0.77 | BB143137 | Q8C7R6 /// Q9CWJ3 |
| 1417214_at | Rab27b | RAB27b, member RAS oncogene family | 0.77 | BB121269 | BAB86914 /// BAC87832 /// Q99P58 |
| 1446331_at | Ptgfr | prostaglandin F receptor | 0.77 | AV244075 | — |
| 1426260_a_at | Ugt1a1 | UDP-glucuronosyltransferase 1 family, member 1 | 0.77 | D87867 | — |
| 1440209_at | 2900024D24Rik | RIKEN cDNA 2900024D24 gene | 0.77 | AI449126 | AAH66008 /// Q8C294 /// Q8CBA1 |
| 1430535_at | 1810043J12Rik | RIKEN cDNA 1810043J12 gene | 0.77 | BB045401 | AAH58221 /// Q9CVK3 |
| 1416872_at | Tm4sf6 | transmembrane 4 superfamily mamber 6 | 0.77 | NM_019656 | O70401 /// Q99L96 |
| 1429158_at | Fbxo28 | F-box protein 28 | 0.77 | AV302798 | Q7TMH9 /// Q8BIF6 /// Q8BIG4 |
| 1433465_a_at | AI467606 | expressed sequence AI467606 | 0.77 | BB234337 | AAH38694 /// AAH64101 /// Q8C708 |
| 1451355_at | CRG-L1 | cancer related gene-liver 1 | 0.77 | AF282864 | AAH59819 /// Q8BUG3 /// Q8VD53 |
| 1429284_at | 8430436F23Rik | RIKEN cDNA 8430436F23 gene | 0.77 | BB248684 | — |
| 1437408_at | Gpr126 | G protein-coupled receptor 126 | 0.77 | BB812574 | Q811E4 |
| 1428642_at | Slc35d3 | solute carrier family 35, member D3 | 0.77 | AK018094 | Q8BGF8 /// Q9CXD4 |
| 1433527_at | Ireb2 | iron responsive element binding protein 2 | 0.77 | BB080732 | O70235 /// Q811J3 /// Q8BWZ6 /// Q8BZL2 |
| 1426819_at | Fosb | FBJ osteosarcoma oncogene B | 0.77 | BG076079 | P13346 |
| 1426578_s_at | Snap25bp | synaptosomal-associated protein 25 binding protein | 0.77 | BB667523 | Q9Z266 |
| 1438816_at | Elys | embryonic large molecule derived from yolk sac | 0.78 | BM247201 | Q8BVJ5 /// Q8R1T9 /// Q8VD55 |
| 1452249_at | Prickle1 | prickle like 1 (Drosophila) | 0.78 | BC022643 | Q8CGJ0 |
| 1416923_a_at | Bnip3l | BCL2/adenovirus E1B 19 kDa-interacting protein 3-like | 0.78 | AK018668 | BAB23456 /// BAB25351 /// BAB28869 /// Q91Z78 /// Q9Z2F7 |
| 1422094_a_at | 2810439M05Rik | RIKEN cDNA 2810439M05 gene | 0.78 | NM_026046 | Q8BKL5 /// Q9CYV4 /// Q9D459 |
| 1442757_at | Chdc1 | calponin homology (CH) domain containing 1 | 0.78 | AI552548 | BAC98074 |
| 1433536_at | Lrp11 | low density lipoprotein receptor-related protein 11 | 0.78 | BB435348 | AAH59874 /// Q8CB67 |
| 1451744_a_at | Zadh1 | zinc binding alcohol dehydrogenase, domain containing 1 | 0.78 | BC021466 | Q8BZA2 /// Q8VDQ1 /// Q9D1W8 |
| 1450135_at | Fzd3 | frizzied homolog 3 (Drosophila) | 0.78 | AU043193 | Q61086 |
| 1458379_at | 9330159F19 | hypothetical protein 9330159F19 | 0.78 | BB079733 | — |
| 1434866_x_at | Cpt1a | carniline palmitoyltransferase 1, liver | 0.78 | BB021753 | P27742 /// Q7TQD5 /// Q80SW3 /// Q8BP98 /// Q8C7H8 |
| 1460360_at | Asrgl1 | asparaginase like 1 | 0.78 | AU040643 | Q8C0M9 /// Q91WC8 /// Q9CVX3 |
| 1417565_at | Abhd5 | abhydrolase domain containing 5 | 0.78 | AK007421 | Q922Z5 /// Q9CTY3 /// Q9DBL9 |
| 1427084_a_at | Map4k5 | mitogen-activated protein kinase kinase kinase kinase 5 | 0.78 | BG067961 | AAH57930 /// Q8BPM2 /// Q8BRE4 |
| 1452074_at | 2810439K08Rik | RIKEN cDNA 2810439K08 gene | 0.78 | AV225967 | Q8BSY5 /// Q8C8G3 /// Q8CCZ6 /// Q8CE78 /// Q9CYV5 |
| 1423046_s_at | Ncbp2 | nuclear cap binding protein subunit 2 | 0.78 | BE285362 | Q9CQ49 |
| 1418382_at | AB023957 | cDNA sequence AB023957 | 0.78 | BB770932 | Q9R0R2 |
| 1434376_at | Cd44 | CD44 antigen | 0.78 | AW146109 | P15379 /// Q80X37 |
| 1454074_a_at | 1500011J06Rik | RIKEN cDNA 1500011J06 gene | 0.78 | AK005213 | — |
| 1419918_at | 3930401E15Rik | RIKEN cDNA 3930401E15 gene | 0.79 | AW545765 | — |
| 1460119_at | — | — | 0.79 | BB245904 | — |
| 1426325_s_at | Eif3s1 | eukaryotic translation initiation factor 3, subunit 1 alpha | 0.79 | BB379268 | Q8BUW6 /// Q99JK5 |
| 1423606_at | Postn | periostin, osteoblast specific factor | 0.79 | BI110565 | Q62009 |
| 1449677_s_at | D4Ertd89e | DNA segment, Chr 4, ERATO Doi 89, expressed | 0.79 | C77858 | Q9DAV9 |
| 1417372_a_at | Peli1 | pellino 1 | 0.79 | BC016515 | Q8C669 |
| 1421137_a_at | Pkib | protein kinese inhibitor beta, cAMP dependent, testis | 0.79 | AV047342 | AAH61162 /// Q04758 /// Q8BNE5 |
| | | specific | | | |
| 1455406_at | — | Mus musculus 0 day neonate head cDNA, RIKEN full- | 0.79 | AV251542 | — |
| | | length enriched library, clone: 4833431M13 | | | |
| | | product: unknown EST, full insert sequence | | | |
| 1437243_at | AI328454 | expressed sequence AI325464 | 0.79 | AV349520 | Q8BU06 /// Q8CIM3 |
| 1459274_at | Gpr135 | G protein-coupled receptor 135 | 0.79 | AV221890 | Q7TQP2 |
| 1426015_s_at | Asph | aspartate-beta-hydroxylase | 0.79 | AF302653 | AAH61098 /// Q8BQK0 /// Q8BSY0 /// Q8CBM2 /// Q8CH79 /// Q91WG6 /// Q920F7 /// |
| | | | | | Q920F8 /// Q920F9 /// Q9CR06 /// Q9D7J8 /// Q9EPA5 /// Q9EQ62 /// Q9EQ63 /// Q9EQ64 /// |
| | | | | | Q9EQ65 |
| 1428785_at | Amotl1 | angiomotin-like 1 | 0.79 | BG917015 | Q9D4H4 |
| 1423220_at | Eif4e | eukaryotic translation initiation factor 4E | 0.79 | BB406487 | P20415 /// Q8C470 |
| 1422484_at | Cycs | cytochrome c, somatic | 0.79 | NM_007808 | BAB22313 /// BAB22617 /// BAB23959 /// BAB27091 /// P00009 |
| 1448503_at | Mcl1 | myeloid cell leukemia sequence 1 | 0.79 | BC003839 | P97287 /// Q9CRI4 |
| 1448830_at | Dusp1 | dual spacificity phosphatase 1 | 0.79 | NM_013642 | P28563 |
| 1434765_at | Ep300 | E1A binding protein P300 | 0.79 | AI844868 | Q8BJ14 |
| 1445531_at | Csmd1 | CUB and Sushi multiple domains 1 | 0.79 | BB179947 | Q923L3 |
| 1454722_at | 2310035O07Rik | RIKEN cDNA 2310035O07 gene | 0.79 | BG792618 | — |
| 1444501_at | G6pdx | glucose-6-phosphate dehydrogenase X-linked | 0.80 | AIB53202 | — |
| 1429468_at | 1110018F16Rik | RIKEN cDNA 1110018F16 gene | 0.80 | AK003775 | — |
| 1422631_at | Ahr | aryl-hydrocarbon receptor | 0.80 | BE989096 | P30561 /// Q8R4S5 /// Q8R4S6 |
| 1426987_at | 5430417L22Rik | RIKEN cDNA 5430417L22 gene | 0.80 | BB028755 | — |
| 1421195_at | Cckar | cholecystokinin A receptor | 0.80 | BC020534 | O08786 |
| 1450954_at | Yme1I1 | YME1-like 1 (S. cerevisiae) | 0.80 | BB826168 | O88967 /// Q8C597 |
| 1437382_at | Acvr2 | activin receptor IIA | 0.80 | BG066107 | P27038 /// Q8BRV4 |
| 1433835_at | Ppp3cb | protein phosphatese 3, catalytic subunit, beta isoform | 0.80 | BE825122 | AAH66000 /// P48453 |
| 1436405_at | 6330411N01Rik | RIKEN cDNA 6330411N01 gene | 0.80 | BG068753 | P59764 /// Q8BMP2 |
| 1448471_a_at | Ctla2a | cytotoxic T lymphocyte-associated protein 2 alpha | 0.80 | NM_007796 | — |
| 1427675_a_at | Grik1 | glutamate receptor ionotropic kainate 1 | 0.80 | X66118 | Q60934 /// QBBQZ0 /// Q8BRQ3 /// Q8BRT2 /// Q8C825 /// Q8C9A0 /// Q8K0C2 |
| 1453187_at | 1810027I20Rik | RIKEN cDNA 1810027I20 gene | 0.80 | AV062896 | Q9D8W7 |
| 1429144_at | 2310032D16Rik | RIKEN cDNA 2310032D16 gene | 0.80 | AV291259 | Q80TD5 /// Q8BKJ7 /// Q8BKW7 /// Q8C0L9 /// Q8CFW2 /// Q9D759 |
| 1418780_at | Cyp39a1 | cytochrome P450, family 39, subfamily a, polypeptide 1 | 0.80 | NM_018887 | BAC27530 /// Q8CFY8 /// Q9JKJ9 |
| 1423177_a_at | Abl1 | abl-interactor 1 | 0.80 | AW912678 | Q60747 /// Q8CBW3 /// Q91ZM5 /// Q923I9 /// Q99KH4 |
| 1441229_at | D230019N24Rik | RIKEN cDNA D230019N24 gene | 0.80 | BB468551 | — |
| 1418968_at | Rb1cc1 | RB1-inducible coiled-coil 1 | 0.80 | BE570980 | AAH66152 /// Q61384 /// Q8BRY9 /// Q8BT47 /// Q8CHH8 /// Q9ESK9 /// Q9JK14 |
| 1421851_at | Ddx26 | DEAD/H (Asp-Glu-Ala-Asp/His) box polypeptide 26 | 0.80 | BB731480 | AAH58637 /// AAH59263 /// Q61204 /// Q8BQZ7 /// Q8C9M9 |
| 1459205_at | — | Mus musculus transcribed sequences | 0.80 | BI076746 | — |
| 1429106_at | 4921509J17Rik | RIKEN cDNA 4921509J17 gene | 0.80 | AK014853 | — |
| 1450684_at | Etv1 | ets variant gene 1 | 0.80 | NM_007960 | P41164 |
| 1423298_at | Add3 | adducin 3 (gamma) | 0.80 | BM239842 | Q8BJH2 /// Q8BM29 /// Q8JZT6 /// Q9JLE2 /// Q9QYB5 |
| 1416060_at | Tbc1d15 | TBC1 domain family, member 15 | 0.81 | BF577643 | Q7TPU5 /// Q8BHS5 /// Q9CRG4 /// Q9CXF4 |
| 1416539_at | Ysg2 | yolk sac gene 2 | 0.81 | NM_011734 | BAC26049 /// P70665 /// Q8CBM6 /// Q8CC41 /// Q8CEB7 |
| 1451726_at | Mtmr6 | myotubularin related protein 6 | 0.81 | BC020019 | Q8VE11 |
| 1423591_at | Fgfr1op2 | FGFR1 oncogene partner 2 | 0.81 | AK004662 | Q9CRA9 /// Q9D7R0 |
| 1423672_at | 2510042P03Rik | RIKEN cDNA 2510042P03 gene | 0.81 | BC026507 | Q8R0Q9 /// Q9CY00 |
| 1450840_a_at | Rpl39 | ribosomal protein L39 | 0.81 | AV107150 | P02404 |
| 1447875_x_at | — | Mus musculus transcribed sequences | 0.81 | BB332055 | — |
| 1443260_at | Meis1 | myeloid ecotropic viral integration site 1 | 0.81 | BB055155 | Q60954 /// Q8CIL0 |
| 1453106_a_at | Rnmt | RNA (guanine-7-) mathyltransferase | 0.81 | AK015403 | BAC97040./// Q9D0L8 /// Q9D5F1 |
| 1424925_at | Sec63 | SEC63-like (S. cerevisiae) | 0.81 | C76103 | Q80YG4 /// Q8K2U5 /// Q8VHE0 |
| 1420021_s_at | D11Ertd530e | DNA segment, Chr 11, ERATO Doi 530, expressed | 0.81 | AU022339 | Q80U70 |
| 1429573_at | 4921520P21Rik | RIKEN cDNA 4921520P21 gene | 0.81 | AK014934 | Q9D5U3 /// Q9D9R7 |
| 1428438_s_at | 2700023B17Rik | RIKEN cDNA 2700023B17 gene | 0.81 | BI662680 | Q8K2F8 /// Q9CTG8 |
| 1454885_at | 2610021A01Rik | RIKEN cDNA 2610021A01 gene | 0.81 | BM211194 | — |
| 1427371_at | Abca8a | ATP-binding cassette, sub-family A (ABC1), member 8a | 0.81 | BC026496 | AAH60032 /// Q8C0A9 /// Q8K442 /// Q8R0R4 |
| 1425956_a_at | Cdadc1 | cytidine and dCMP deaminase domain containing 1 | 0.81 | BC004588 | Q8BMD5 /// Q8BYL2 /// Q8BYN1 /// Q8C014 /// Q922P4 /// Q99KL2 /// Q9D7F3 |
| 1437461_s_at | 2810441O16Rik | RIKEN cDNA 2810441O16 gene | 0.81 | BB557441 | Q80VS9 /// Q91YJ1 /// Q9CSC1 |
| 1446735_at | Sh3d1B | SH3 domain protein 1B | 0.81 | BB559054 | Q80TG5 /// Q8C9C3 /// Q8CD59 /// Q9CQD9 /// Q9Z0R6 |
| 1435496_at | 5730462M10Rik | RIKEN cDNA 5730460M10 gene | 0.81 | AI429609 | AAH56635 /// Q9CYH2 |
| 1440370_at | Abca13 | ATP-binding cassette, sub-family A (ABC1), member 13 | 0.81 | BB277120 | Q80T20 /// Q8BHZ2 |
| 1427183_at | Efemp1 | epidermal growth factor-containing fibulin-like | 0.81 | BC023060 | AAO37642 /// AAP79577 /// Q8BPB5 /// Q8K0J4 /// Q8R1U8 |
| | | extracellular matrix protein 1 | | | |
| 1429084_at | Vezf1 | vascular endothelial zinc finger 1 | 0.81 | AV308858 | Q8K1B7 /// Q9Z162 |
| 1417411_at | Nap1l5-pending | nucleosome assembly protein 1-like 5 | 0.81 | NM_021432 | Q80U90 /// Q8CFQ0 /// Q9CTE1 /// Q9CYM1 /// Q9JJF0 |
| 1434424_at | 9630055N22Rik | RIKEN cDNA 9630055N22 gene | 0.81 | BB276950 | Q8CAM2 /// Q91XE2 |
| 1449310_at | Ptger2 | prostaglandin E receptor 2 (subtype EP2) | 0.81 | BC005440 | AC35664 /// Q62053 |
| 1429451_at | 2610301B20Rik | RIKEN cDNA 2610301B20 gene | 0.82 | AK011950 | AAH58777 /// Q9D005 |
| 1434136_at | 6332401O19Rik | RIKEN cDNA 6332401O19 gene | 0.82 | BE571820 | Q8BN70 |
| 1425210_s_at | Zfp84 | zinc finger protein 84 | 0.82 | AI455811 | Q60911 /// Q8BL69 /// Q922D0 /// Q9D654 |
| 1424769_s_at | Cald1 | caldesmon 1 | 0.82 | BI248947 | Q7TMN5 /// Q8VCQ8 /// Q8VD79 |
| 1439829_at | Adcy5 | adenylate cyclase 5 | 0.82 | BE946363 | — |
| 1452762_at | 8430436O14Rik | RIKEN cDNA 8430436O14 gene | 0.82 | AK018466 | — |
| 1420971_at | Ubr1 | ubiquitin protein ligase E3 component n-recognin 1 | 0.82 | BQ173927 | O70481 /// Q8BN40 /// Q8C5K3 |
| 1439268_x_at | Eif3s6 | eukaryotic translation initiation factor 3, subunit 6 | 0.82 | BB032885 | AAC53346 /// P60229 /// Q8BNE6 /// Q9CT23 |
| 1448955_s_at | Cadps | Ca<2+>dependent activator protein for secretion | 0.82 | NM_012061 | AAH57065 /// Q61374 /// Q80TJ1 |
| 1419549_at | Arg1 | arginase 1, liver | 0.82 | NM_007482 | Q61176 /// Q80VI4 |
| 1455914_at | AI987944 | expressed sequence AI987944 | 0.82 | AW554430 | — |
| 1426856_at | 2610207I16Rik | RIKEN cDNA 2610207I16 gene | 0.82 | BM200015 | Q8C3H3 /// Q99JH2 /// Q99LV2 |
| 1455590_at | 4930482L21Rik | RIKEN cDNA 4930482L21 gene | 0.82 | AV380561 | Q60854 /// Q8BZR2 |
| 1437671_x_at | 2310046G15Rik | RIKEN cDNA 2310046G15 gene | 0.82 | BB378796 | BAC28708 /// BAC37319 /// Q8BZS4 /// Q8CF39 /// Q9D6X6 |
| 1434732_x_at | 1110020J08Rik | RIKEN cDNA 1110020J08 gene | 0.82 | AV044898 | Q9D173 |
| 1424232_a_at | BC025546 | cDNA sequence BC025546 | 0.82 | BC025546 | Q8BWQ4 /// Q8R3E7 |
| 1429175_at | 2810417M05Rik | RIKEN cDNA 2810417M05 gene | 0.82 | AK014196 | Q8CEU4 /// Q9CZ16 |
| 1438201_at | — | Mus musculus, Similar to pyruvate dehydrogenase | 0.82 | AV290622 | — |
| | | phosphatase, clone IMAGE: 6492665, mRNA | | | |
| 1460544_at | Mak10 | MAK10 homolog, amino-acid N-acetyltransfarase | 0.82 | BG083730 | AAH56435 /// Q8BYJ5 /// Q8BYJ9 /// Q8K3H2 |
| | | subunit, (S. cerevisiae) | | | |
| 1422936_at | Mas1 | MAS1 oncogene | 0.82 | NM_008552 | P30554 /// Q8BHI8 |
| 1439185_x_at | — | Mus musculus transcribed sequences | 0.82 | BB433489 | Q8VCF0 |
| 1430219_at | Fts | fused toes | 0.82 | AK017861 | Q64362 |
| 1436761_s_at | 4921522K17Rik | RIKEN cDNA 4921522K17 gene | 0.82 | BB461323 | AAH66835 /// Q8BLV7 /// Q99LJ4 /// Q9DBR2 |
| 1434761_at | Ids | iduronate 2-sulfatase | 0.82 | BB493523 | Q08890 /// Q8CJ15 |
| 1460320_at | Becn1 | beclin 1 (coiled-coil, myosin-like BCL2-interacting | 0.82 | NM_019584 | O88597 /// Q99J03 |
| | | protein) | | | |
| 1428156_at | Gng2 | guanine nucleotide binding protein (G protein), gamma | 0.82 | AV021455 | P16874 |
| | | 2 subunit | | | |
| 1419286_s_at | Cdv1 | camitine deficiency-associated gene expressed in | 0.82 | NM_009879 | O35594 |
| | | ventricle 1 | | | |
| 1426736_at | Gspt1 | G1 to phase transition 1 | 0.83 | AB003502 | Q8BPH0 /// Q8CAS6 /// Q8CCV1 /// Q8K2E1 /// Q8R050 |
| 1451177_at | Dnajb4 | DnaJ (Hsp40) homolog, subfamily B, member 4 | 0.83 | BC017161 | BAC25720 /// Q8BP73 /// Q9D832 |
| 1449407_at | Cdv1 | carnitine deficiency-associated gene expressed in | 0.83 | NM_009879 | O35594 |
| | | ventricle 1 | | | |
| 1449530_at | Trps1 | trichorhinophalangeal syndrome I (human) | 0.83 | NM_032000 | Q80V18 /// Q8BZ62 /// Q8K1J0 /// Q925H1 |
| 1420367_at | Denr | density-regulated protein | 0.83 | AK010394 | Q9CQJ6 |
| 1428791_at | Ube2h | ubiquitin-conjugating enzyme E2H | 0.83 | BB183512 | AAH08517 /// P37286 |
| 1452411_at | Lrrc1 | leucine rich repeat containing 1 | 0.83 | BG966295 | Q80VQ1 /// Q8BKR1 /// Q8BUS9 /// Q8QZU1 |
| 1447861_x_at | Mrg1 | myeloid ecotropic viral integration site-related gene 1 | 0.83 | AV329643 | AAH17375 /// P97367 |
| 1449643_s_at | Btf3 | basic transcription factor 3 | 0.83 | AA220626 | AAH08233 /// AAH64010 /// Q64152 /// Q9D9L3 |
| 1437067_at | Phtt2 | putative homeodomain transcription factor 2 | 0.83 | BM228625 | Q7TPX6 /// Q8C975 /// Q8C9D2 /// Q8CB19 /// Q8CBQ3 |
| 1428603_at | Glcci1 | glucocorticoid induced tranacript 1 | 0.83 | AK009885 | Q80YT1 /// Q8CEA5 /// Q8K3I9 /// Q925C1 /// Q9D6W9 |
| 1427058_at | Eif4a1 | eukaryotic translation initiation factor 4A1 | 0.83 | AK010644 | AAH49915 /// BAB27678 /// BAC36796 /// P60843 /// Q64341 /// Q99LR0 |
| 1433853_at | Mib1 | mindbomb homolog 1 (Drosophila) | 0.83 | BG063791 | AAN18022 /// BAC98141 /// Q80SY4 /// Q8BNR1 /// Q8C6W2 /// Q921Q1 |
| 1426717_at | 3830408P04Rik | RIKEN cDNA 3830408P04 gene | 0.83 | BF787442 | Q8K0M0 /// Q921M1 /// Q9CSH1 /// Q9D0N5 /// Q9JJC8 |
| 1443269_at | — | Mus musculus 12 days embryo spinal ganglion cDNA, | 0.83 | BB451348 | — |
| | | RIKEN full-length enriched library, clone: D130009B15 | | | |
| | | product: unknown EST, full insert sequence | | | |
| 1447927_at | AI595338 | expressed sequence AI595338 | 0.83 | BG092512 | AAH57969 /// BAC87667 /// Q61594 /// Q7TMV8 /// Q8K0G1 /// Q9D3E4 |
| 1433575_at | Sox4 | SRY-box containing gene 4 | 0.84 | BG083485 | Q06831 /// Q8BPK5 /// Q8BQ53 /// Q8CE56 |
| 1435239_at | Gria1 | glutamate receptor ionotropic AMPA1 (alpha 1) | 0.84 | BQ175316 | AAH60702 /// P23818 /// Q7TNB5 |
| 1459325_at | — | Mus musculus 7 days neonate cerebellum cDNA, | 0.84 | AV329840 | — |
| | | RIKEN full-length enriched library, clone: A730031J22 | | | |
| | | product: unknown EST, full insert sequence | | | |
| 1425149_s_at | Pdcl | phosducin-like | 0.84 | BC006578 | BAC26056 /// BAC26133 /// Q923E8 |
| 1415834_at | Dusp6 | dual specificity phosphatase 6 | 0.84 | NM_026268 | BAC40372 /// BAC40489 /// Q9DBB1 |
| | | proteasome (prosome, macropain) 26S subunit, | | | |
| 1417769_at | Psmc6 | ATPase, 6 | 0.84 | AW208944 | AAH57997 /// Q810A6 /// Q8QZS9 /// Q92524 /// Q9CXH9 |
| 1441565_at | — | Mus musculus adult male testis cDNA, RIKEN full- | 0.84 | BB016866 | — |
| | | length enriched library, clone: 4930564M06 | | | |
| | | product: inferred: cadherin-11 {Mus musculus}, | | | |
| | | full insert sequence | | | |
| 1429596_at | 2400002F02Rik | RIKEN cDNA 2400002F02 gene | 0.84 | AK010254 | — |
| 1459793_s_at | 4930469P12Rik | RIKEN cDNA 4930469P12 gene | 0.84 | AV301944 | — |
| 1438073_at | — | Mus musculus 10 days neonate cerebellum CDNA, | 0.84 | AW047633 | — |
| | | RIKEN full-length enriched library, clone: 6530427L06 | | | |
| | | product: unknown EST, full insert sequence | | | |
| 1437076_at | A930017M01 | hypothetical protein A930017M01 | 0.84 | BB279424 | Q8C4W1 |
| 1452366_at | 4732435N03Rik | RIKEN cDNA 4732435N03 gene | 0.84 | AV371987 | Q8BJQ9 /// Q8BWV9 /// Q8BZU7 /// Q8C195 /// Q8CBT0 /// Q8R0M6 |
| 1420091_s_at | Zcwcc3 | zinc finger, CW-type with coiled-coil domain 3 | 0.84 | AI452146 | Q8R0R0 |
| 1433929_at | Nhlrc2 | NHL repeat containing 2 | 0.84 | BB795641 | Q80XU0 /// Q8BZW8 /// Q8C1S8 /// Q9CW64 |
| 1422414_a_at | Calm2 | calmodulin 2 | 0.84 | NM_007589 | AAH21347 /// AAH51444 /// BAB28116 /// BAB28319 /// BAB28631 /// P02593 /// Q91VQ9 |
| 1455372_at | Cpeb3 | cytoplasmic polyadenylation element binding protein 3 | 0.84 | BB770826 | Q7TN99 /// Q8CHC2 |
| 1426747_at | Abcf3 | ATP-binding cassette, sub-family F (GCN20), member 3 | 0.84 | AI552141 | Q8K268 /// Q9JL49 |
| 1456700_x_at | Marcks | myristoylated alanine rich protein kinase C substrate | 0.84 | BB100920 | P26645 |
| 1428973_s_at | 0610012D17Rik | RIKEN cDNA 0610012D17 gene | 0.84 | AK007178 | AAH59716 /// Q9CQ66 /// Q9CQJ9 |
| 1420402_at | Atp2b2 | ATPase, Ca++ transporting, plasma membrane 2 | 0.84 | NM_009723 | Q9R0K7 |
| 1422561_at | Adamts5 | a disintegrin-like and metalloprotease (reprolysin type) | 0.84 | BB658835 | Q8BGP4 /// Q9R001 |
| | | with thrombospondin type 1 motif, 5 (aggrecanase-2) | | | |
| 1418066_at | Cfl2 | cofilin 2, muscle | 0.84 | AI323758 | P45591 |
| 1439204_at | — | Mus musculus 16 days embryo head CDNA, RIKEN | 0.85 | BB096886 | Q62204 |
| | | full-length enriched library, clone: C13005811 | | | |
| | | product: unknown EST full insert sequence | | | |
| 1427331_at | — | Mus musculus transcribed sequences | 0.85 | BB518868 | CAD88592 /// Q60612 /// Q8BGU7 /// Q8CAH1 /// Q8R0M5 |
| 1419245_at | Rab14 | RAB14, member RAS oncogene family | 0.85 | AV339290 | AAH56648 /// Q91V41 |
| 1428408_a_at | D12Ertd551e | DNA segment, Chr 12, ERATO Doi 551 expressed | 0.85 | BI102044 | AAH59230 /// Q9D5Y7 |
| 1441982_at | Taf7 | TAF7 RNA polymerase II, TATA box binding protein | 0.85 | BB551747 | BAC26429 /// BAC36313 /// Q8BPH4 /// Q8C291 /// Q9R1C0 |
| | | (TBP)-associsted factor | | | |
| 1427464_s_at | Hspa5 | heat shock 70 kD protein 5 (glucose-regulated protein) | 0.85 | AJ002387 | AAH50927 /// BAC36166 /// P20029 /// Q7TMA3 /// Q9DC41 |
| 1437921_x_at | C330029B10Rik | RIKEN cDNA C330029B10 gene | 0.85 | AW744723 | — |
| 1441545_at | 9230115F04Rik | RIKEN cDNA 9230115F04 gene | 0.85 | BM243297 | — |
| 1423725_at | Pls3 | plastin 3 (T-isoform) | 0.85 | BC005459 | Q99K51 |
| 1448899_s_at | Rad51ap1 | RAD51 associated protein 1 | 0.85 | BC003738 | O55219 /// Q8BP36 /// Q8C551 /// Q99L94 /// Q9D0J0 |
| 1435230_at | AI447928 | expressed sequence AI447928 | 0.85 | BB277613 | Q80TP9 /// Q8BR16 /// Q8CBV4 |
| 1425518_at | Rapgef4 | Rap guanine nucleotide exchange factor (GEF) 4 | 0.85 | AK004874 | Q8CCU5 /// Q9CS95 /// Q9EQZ6 |
| 1448760_at | Zfp68 | Zinc finger protein 68 | 0.85 | NM_013844 | O88238 /// Q60910 /// Q8BLK6 /// Q8VEM6 /// Q9Z116 |
| 1455875_x_at | Tm9sf2 | transmembrane 9 superfamily member 2 | 0.85 | BB131843 | BAC33215 /// BAC40645 /// P58021 /// Q8C6H4 /// Q8C7F9 |
| 1434888_a_at | Matr3 | matrin 3 | 0.85 | BM219545 | BAC98009 /// Q7TN66 /// Q8K310 |
| 1435129_at | Ptp4a2 | protein tyrosine phosphatase 4a2 | 0.85 | AW495875 | O70274 |
| 1433542_at | Inpp5f | inositol polyphosphate-5-phosphatase F | 0.85 | BB085335 | AAH67200 /// BAC98059 /// Q8C8G7 /// Q8CBW2 /// Q8CDA1 |
| 1453314_x_at | 2610039C10Rik | RIKEN cDNA 2610039C10 gene | 0.85 | AK012533 | Q9CXR6 /// Q9CZJ6 /// Q9D086 |
| 1435001_at | Ivns1abp | influenza virus NS1A binding protein | 0.85 | BM198417 | Q8C6C4 |
| 1417815_a_at | Tde1 | tumor differentially expressed 1 | 0.85 | NM_012032 | Q8C6L8 /// Q9DCF0 /// Q9QZI9 |
| 1455963_at | 6332401O19Rik | RIKEN cDNA 6332401O19 gene | 0.85 | AV317707 | Q8BN70 |
| 1435174_at | Rsbn1 | rosbin, round spermatid basic protein 1 | 0.85 | AW546080 | Q7TNJ3 /// Q80T69 /// Q8BGC6 /// G8BQ56 /// Q8C2Z3 |
| 1435675_at | Tbc1d12 | TBC1D12: TBC1 domain family, member 12 | 0.85 | BF228251 | Q8K257 |
| 1422437_at | Col5a2 | procollagen, type V, alpha 2 | 0.85 | AV229424 | Q61431 /// Q7TMS0 /// Q80VS8 /// Q8BNA3 |
| 1430992_s_at | 1500009M05Rik | RIKEN cDNA 1500009M05 gene | 0.86 | BE916591 | AAH58279 /// Q9CQB5 /// Q9D0Y0 |
| 1418472_at | Aspa | aspartoacylase (aminoacylase) 2 | 0.86 | BC024934 | Q8BZC2 /// Q8R3P0 |
| 1456856_at | E130120L08Rik | RIKEN cDNA E130120L08 gene | 0.86 | AI854225 | Q8BSS9 |
| 1424353_at | Lrpprc | leucine-rich PPR-motif containing | 0.86 | BC004681 | AAH59862 /// Q8K4V0 /// Q99KF9 /// Q9CRX4 |
| 1419663_at | Ogn | osteoglycin | 0.86 | BB542051 | BAC35462 /// Q62000 |
| 1439409_x_at | Tyrp1 | tyrosinase-related protein 1 | 0.86 | BB006219 | P07147 |
| 1451510_s_at | Thedc1 | thioesterase domain containing 1 | 0.86 | BC025001 | Q8R197 |
| 1435518_at | Rap1b | RAS related protein 1b | 0.86 | BM246972 | AAH33382 /// AAH52480 /// Q99JI6 |
| 1428976_at | Tmpo | thymopoletin | 0.86 | AK017463 | BAB27960 /// Q61029 /// Q61033 /// Q9CPQ7 |
| 1428579_at | Fmnl2 | formin-like 2 | 0.86 | AK017338 | AAH64731 /// Q7TPA8 /// Q80VH6 /// Q8BI52 |
| 1429601_x_at | 1110019K23Rik | RIKEN cDNA 1110019K23 gene | 0.86 | AK003824 | — |
| 1455901_at | Chpt1 | choline phosphotransferase 1 | 0.86 | AI642069 | AAR16089 /// Q8C025 /// Q8K0H2 /// Q91W91 |
| 1420866_at | Zfp161 | zinc finger protein 161 | 0.86 | NM_009547 | BAC29858 /// Q08376 |
| 1450418_a_at | 2310034L04Rik | RIKEN cDNA 2310034L04 gene | 0.87 | NM_026417 | Q8C407 /// Q99KZ9 |
| 1416083_at | Za20d2 | zinc finger, A20 domain containing 2 | 0.87 | AA124553 | BAC36321 /// O88878 /// Q9D3C9 |
| 1426276_at | Ifih1 | interferon induced with helicase C domain 1 | 0.87 | AY075132 | Q8BYC9 /// Q8BZ01 /// Q8K5C7 /// Q8R144 /// Q8R5F7 /// Q8VE79 /// Q99KS4 /// Q9D2Z5 |
| 1440882_at | Lrp8 | low density lipoprotein receptor-related protein 8 | 0.87 | BB750940 | Q924X6 |
| | | apolipoprotein e receptor | | | |
| 1428268_at | Psd2 | pleckstrin and Sec7 domain containing 2 | 0.87 | AK018116 | AAH56437 /// AAH62930 /// AAH65063 /// AAH65055 /// AAH66026 /// AAH66036 /// |
| | | | | | Q8BHR9 /// Q9D3B8 |
| 1456111_at | C130036J11 | hypothetical protein C130036J11 | 0.87 | BB072624 | Q8BKY4 |
| 1434848_at | Gpr27 | G protein-coupled receptor 27 | 0.87 | BB259283 | — |
| 1449175_at | Gpr65 | G-protein coupled receptor 65 | 0.87 | NM_008152 | Q61038 |
| 1433751_at | Slc39a10 | solute carriar family 39 (zinc transporter), member 10 | 0.87 | BM250411 | AAH59214 /// AAH62918 /// Q80TG2 /// Q8BX42 /// Q8C0L2 |
| 1421284_at | Pign | phosphatidylinositol glycan, class N | 0.87 | NM_013784 | Q8VCC3 /// Q9R1S2 /// Q9R1S3 |
| 1452593_a_at | Tceb1 | transcription elongation factor B (SIII), polypeptide 1 | 0.87 | AI019214 | Q63182 |
| 1426407_at | 1600010O03Rik | RIKEN cDNA 1600010O03 gene | 0.87 | BI412951 | — |
| 1455181_at | Rasa2 | RAS p21 protein activator 2 | 0.87 | BM228516 | P58069 |
| 1433441_at | Fbxl5 | F-box and leucine-rich repeat protein 5 | 0.87 | BQ173911 | Q8C2S5 |
| 1423963_at | Wdr26 | WD repeat domain 26 | 0.87 | BC020044 | AAH58601 /// Q8C6G8 |
| 1424782_at | 2610318G18Rik | RIKEN cDNA 2610318G18 gene | 0.88 | BC024458 | Q9CR48 /// Q9D520 /// Q9D835 |
| 1460073_at | — | Mus musculus transcribed sequences | 0.88 | BE980582 | — |
| 1426863_at | Rbmx | RNA binding motif protein, X chromosome | 0.88 | BM123721 | Q8C2U6 /// Q9R0Y0 /// Q9WV02 |
| 1427235_at | Utx | ubiquitously transcribed tetratricopeptide repeat gene, X | 0.88 | BG076105 | O70546 /// Q7TSG4 /// Q8C4Z1 /// Q8R2W5 |
| | | chromosome | | | |
| 1434460_at | Bbs4 | Bardet-Biedl syndrome 4 homolog (human) | 0.88 | BG067572 | Q8C1Z7 |
| 1454736_at | 4921515A04Rik | RIKEN cDNA 4921515A04 gene | 0.88 | BM119297 | Q7TSA5 /// Q8BWN1 /// Q8C0J6 /// Q8C650 |
| 1426707_at | Tubgcp3 | tubulin, gamma complex associated protein 3 | 0.88 | BC025647 | AAH58566 /// P58854 /// Q8BKJ3 |
| 1429033_at | Gcc1 | golgl coiled coil 1 | 0.88 | AV339946 | AAH66807 /// Q8VC84 /// Q9D4H2 |
| 1417191_at | Dnajb9 | DnaJ (Hsp40) homolog, subfamily B, member 9 | 0.88 | NM_013760 | AAH42713 /// Q9QYI6 |
| 1441662_at | Cyp4x1 | cytochrome P450, family 4, subfamily x, polypeptide 1 | 0.88 | BB171122 | Q8BYS0 |
| 1436883_at | Mbtps2 | RIKEmembrane-bound transcription factor protease, | 0.88 | BB264953 | — |
| | | site 2 | | | |
| 1444602_at | — | — | 0.88 | BE136101 | — |
| 1448192_s_at | Prps1 | phosphoribosyl pyrophosphate synthetase 1 | 0.88 | AK011304 | AAH54772 /// BAA84686 /// BAB27530 /// BAC40697 /// Q9D7G0 |
| 1423829_at | 0910001A06Rik | RIKEN cDNA 0910001A06 gene | 0.88 | BC011343 | Q921M7 |
| 1437156_at | Efcbp1 | EF hand calcium binding protein 1 | 0.88 | BB392041 | AAH67055 /// Q80W91 /// Q8BG18 |
| 1441481_at | Mfap3l | microfibrillar-associated protein 3-like | 0.89 | AV262974 | Q80TV6 /// Q9D3X9 |
| 1427971_at | Hrpt2 | hyperparathyroidism 2 homolog (human) | 0.89 | BB622571 | Q8JZM7 |
| 1450928_at | Idb4 | inhibitor of DNA binding 4 | 0.89 | BB121406 | BAC30845 /// P41139 |
| 1420514_at | Tm4sf10 | transmembrane 4 superfamily member 10 | 0.89 | NM_138751 | Q8C0H5 /// Q9JJG6 |
| 1427075_s_at | 5330414D10Rik | RIKEN cDNA 5330414D10 gene | 0.89 | BM117243 | Q8BHD8 |
| 1428950_s_at | Nol8 | nucleolar protein 8 | 0.89 | AK017551 | Q80VB9 /// Q8CDJ7 /// Q9CUR0 |
| 1433930_at | Hpse | heparanase | 0.89 | BG094050 | AAN41636 /// AAQ15188 /// Q8K3K3 |
| 1443638_at | — | Mus musculus transcribed sequences | 0.89 | BM197773 | — |
| 1454642_a_at | Commd3 | COMM domain containing 3 | 0.89 | BB230296 | Q8C9P5 |
| 1457990_at | C030032C09Rik | RIKEN cDNA C030032C09 gene | 0.89 | BB080832 | Q8BIZ1 /// Q8BJ47 /// Q8BJ49 /// Q8BZM2 |
| 1436772_at | Gria4 | glutamate receptor ionotropic AMPA4 (alpha 4) | 0.89 | BB330347 | — |
| 1443924_at | Prkwnk3 | protein kinase, lysine deficient 3 | 0.89 | BB084132 | AAH60731 /// Q80XP9 |
| 1457385_at | Timm8a | translocase of inner mitochondrial membrane 8 | 0.89 | BB796239 | — |
| | | homolog a (yeast) | | | |
| 1459701_x_at | — | — | 0.89 | AI467488 | — |
| 1453070_at | C030033F14Rik | RIKEN cDNA C030033F14 gene | 0.90 | BB305930 | — |
| 1438171_x_at | 0610012D09Rik | RIKEN cDNA 0610012D09 gene | 0.90 | BB056666 | AAH68124 /// Q8BJU4 /// Q8R567 /// Q9CTJ3 /// Q9EPL4 /// Q9JJ88 |
| 1433788_at | — | Mus musculus transcribed sequences | 0.90 | BM942887 | — |
| 1422643_at | Moxd1 | monooxygenase, DBH-like 1 | 0.90 | NM_021509 | AAH57652 /// Q8BUZ7 /// Q8R394 /// Q9CXI3 /// Q9JJA6 |
| 1456812_at | Abcd2 | ATP-binding cassette, sub-family D (ALD), member 2 | 0.90 | AW456685 | Q61285 /// Q8BQ63 /// Q8C486 |
| 1420665_at | Ifgb3bp | integrin beta 3 binding protein (beta3-endonexin) | 0.90 | NM_026348 | Q9CQ82 |
| 1454764_s_at | Slc38a1 | solute carrier femily 38, member 1 | 0.90 | BF165681 | AAH66815 /// Q8BHI3 /// Q8BXE2 /// Q8K2P7 /// Q99PR1 |
| 1437791_s_at | 1700016A15Rik | RIKEN cDNA 1700016A15 gene | 0.90 | AV230748 | Q8BRL0 /// Q8K0U5 /// Q8R2W0 |
| 1441258_at | AF529169 | cDNA sequence AF529169 | 0.90 | BB316516 | Q8BQW5 /// Q8K3V7 |
| 1481772_at | BC016423 | cDNA sequence BC016423 | 0.90 | NM_134063 | BAC87659 /// Q91W76 |
| 1457473_at | Chd1 | chromodomain helicase DNA binding protein 1 | 0.90 | AI851787 | P40201 /// Q8C9F3 /// Q9CRD9 /// Q9D5K6 |
| 1439011_at | 2010109K11Rik | RIKEN cDNA 2010109K11 gene | 0.90 | BB333400 | — |
| 1426725_s_at | Ets1 | E26 avian leukemia oncogene 1,5′ domain | 0.90 | BB151715 | AAR00342 /// AAR87824 /// P27577 /// Q8BVW8 /// Q8K3Q9 /// Q921D8 |
| 1455177_at | Ahi1 | Abelson helper integration site | 0.91 | BQ175532 | AAH65146 /// Q7TNV2 /// Q8K3E4 /// Q8K3E5 /// Q9CVY1 |
| 1434045_at | Cdkn1b | cyclin-dependent kinase inhibitor 1B (P27) | 0.91 | BB354528 | — |
| 1452682_at | 4632404H22Rik | RIKEN cDNA 4632404H22 gene | 0.91 | AK019480 | AAH66167 /// Q8BRG5 /// Q8CBB0 /// Q9D2N2 |
| 1435350_at | Traf6 | Tnf receptor-associated factor 6 | 0.91 | AV377471 | AAH60705 /// P70196 |
| 1428755_at | 3526402H21Rik | RIKEN cDNA 3526402H21 gene | 0.91 | AK014391 | Q9D6D2 |
| 1428749_at | 6430411K14Rik | RIKEN cDNA 6430411K14 gene | 0.91 | AK018275 | — |
| 1419267_at | Nfyb | nuclear transcription factor-Y beta | 0.91 | AV250496 | P22569 /// Q8C590 /// Q9D056 |
| 1438035_at | — | Mus musculus 12 days embryo embryonic body | 0.91 | BB748934 | Q8BSE0 /// Q8CIF1 |
| | | between diaphragm region and neck cDNA, RIKEN full- | | | |
| | | length enriched library, clone: 9430015D03 | | | |
| | | product: hypothetical protein, full insert sequence | | | |
| 1442812_at | Anapc5 | anaphase-promoting complex subunit 5 | 0.91 | BB155332 | — |
| 1448254_at | Ptn | pleiotrophin | 0.91 | BC002064 | AAH61695 /// BAB27557 /// P20935 /// Q9CSX6 |
| 1455880_s_at | Becn1 | beclin 1 (coiled-coil, myosin-like BCL2-interacting | 0.91 | C86082 | O88597 /// Q99J03 |
| | | protein) | | | |
| 1433856_at | AW555814 | expressed sequence AW555814 | 0.91 | AW555814 | BAC97950 /// Q7TPU4 /// Q80V47 /// Q8BWK5 |
| 1460369_at | LOC233987 | similar to zinc finger protein 97 | 0.92 | BC003267 | — |
| 1419925_s_at | 6430411B10Rik | RIKEN cDNA 6430411B10 gene | 0.92 | AV259382 | AAH57139 /// BAC98261 /// Q8BXZ1 /// Q8BZB8 |
| 1428083_at | 2310043N10Rik | RIKEN cDNA 2310043N10 gene | 0.92 | AK018202 | — |
| 1460381_at | LOC232855 | similar to zinc finger protein 113 | 0.92 | BC023179 | Q8R573 |
| 1448484_at | Amd1 | S-adenosylmethionine dacarboxylase 1 | 0.92 | NM_009665 | P31154 |
| 1448689_at | Rras2 | related RAS viral (r-ras) oncogene homolog 2 | 0.92 | NM_025846 | P17082 /// Q8C5D1 /// Q9CTF6 /// Q9D0H6 |
| 1460252_s_at | Zfp105 | zinc finger protein 105 | 0.92 | NM_009544 | O88412 /// Q80WR2 |
| 1422045_a_at | Ptpn12 | protein tyrosine phosphatase, non-receptor type 12 | 0.92 | X63440 | P35831 /// Q80UM4 |
| 1433857_at | Fath | fat tumor suppressor homolog (Drosophila) | 0.92 | AV088463 | AAP82173 /// Q60833 /// Q80VA2 /// Q80XT9 /// Q9QXA3 |
| 1458820_at | — | Mus musculus transcribed sequences | 0.92 | AV300514 | — |
| 1424243_at | Icam1 | intercellular adhesion molecule | 0.92 | AK005797 | Q8CAP7 /// Q9CPR1 |
| 1460403_at | Psip2 | PC4 and SFRS1 interacting protein 2 | 0.92 | BF117241 | Q80WQ7 /// Q99JF7 /// Q99JF8 /// Q99LR4 /// Q9CT03 |
| 1456735_x_at | C130099A20Rik | RIKEN cDNA C130099A20 gene | 0.92 | BB458645 | Q8BHA9 /// Q8BZ12 /// Q8BZD5 |
| 1448954_at | Nrip3 | nuclear receptor interacting protein 3 | 0.92 | NM_020610 | Q9JJR9 |
| 1428663_at | 5133401H06Rik | RIKEN cDNA 5133401H06 gene | 0.92 | AK017223 | — |
| 1435084_at | C730049O14Rik | RIKEN cDNA C730049O14 gene | 0.92 | BB200607 | — |
| 1439441_x_at | Lats2 | large tumor suppressor 2 | 0.93 | BB134767 | Q7TSJ6 /// Q8CDJ4 /// Q8VHE1 /// Q8VHE2 /// Q9JMI3 |
| 1451360_at | 1200009B18Rik | RIKEN cDNA 1200009B18 gene | 0.93 | BC018188 | AAH64749 /// Q9CR89 /// Q9CWM6 /// Q9CYA2 /// Q9D4R1 /// Q9D8Z9 |
| 1455511_at | Sephs1 | selenophosphate synthetase 1 | 0.93 | BB354974 | AAH65165 /// AAH66037 /// Q8BH69 /// Q8BL02 |
| 1423408_a_at | 2500003M10Rik | RIKEN cDNA 2500003M10 gene | 0.93 | BE692070 | Q8C5N4 /// Q99KL5 /// Q9CY57 /// Q9D7T3 /// Q9DB03 /// Q9DC54 /// Q9JJ95 |
| 1438657_x_at | Ptp4a1 | protein tyrosine phosphatase 4a1 | 0.93 | BB043450 | Q63739 |
| 1420174_s_at | Tax1bp1 | Tax1 (human T-cell leukemia virus type I) binding | 0.93 | C85320 | Q91YT6 /// Q9CVF0 /// Q9DC45 |
| | | protein 1 | | | |
| 1451217_a_at | 1500034J20Rik | RIKEN cDNA 1500034J20 gene | 0.93 | BC008259 | Q9CQU8 |
| 1421479_at | Zfp318 | zinc finger protein 318 | 0.93 | NM_021346 | Q8BMX9 /// Q99PP2 /// Q9JJ01 |
| 1425794_at | Pola2 | polymerase (DNA directed), alpha 2 | 0.93 | BC006945 | AAH64795 /// P33611 /// Q8CIL1 /// Q8VDR3 /// Q922M1 /// Q9CTS2 |
| 1426709_a_at | Usp33 | ubiquitin specific protease 33 | 0.93 | BG075953 | Q80TK2 /// Q80VA4 /// Q8K0I3 /// Q8R5K2 /// Q99K22 |
| 1433925_at | — | Mus musculus cDNA clone MGC: 76410 | 0.93 | BM212035 | AAH58645 |
| | | IMAGE: 6405596, complete cds | | | |
| 1438210_at | 9630018L10Rik | RIKEN cDNA 9630018L10 gene | 0.93 | BB126999 | Q80T52 /// Q8BXA3 /// Q8BZC0 |
| 1426271_at | Smc5l1 | SMC5 structural maintenance of chromosomes 5-like 1 | 0.93 | AV257384 | Q80TW7 /// Q8BKX5 /// Q8CG46 /// Q8CHX5 /// Q922K3 |
| | | (yeast) | | | |
| 1423641_s_at | Cnot7 | CCR4-NOT transcription complex, subunit 7 | 0.93 | BC006021 | BAC31969 /// Q60809 |
| 1428178_s_at | Trappc6b | trafficking protein particle complex 6B | 0.93 | BG066452 | Q8CBK8 /// Q9D289 |
| 1429013_at | 5330432J06Rik | RIKEN cDNA 5330432J06 gene | 0.93 | AK021126 | Q8BLE6 /// Q8BMQ4 /// Q8C0A6 /// Q9D2A4 |
| 1433795_at | Tgfbr3 | transforming growth factor, beta receptor III | 0.94 | BM122301 | O88393 |
| 1449079_s_at | Siat10 | sialyltransferase 10 (alpha-2,3-sialyltransferase VI) | 0.94 | NM_018784 | Q80UR7 /// Q8BLV1 /// Q8K0W8 /// Q8VIB3 /// Q9CVW3 /// Q9WVG2 |
| 1460048_at | — | Mus musculus transcribed sequences | 0.94 | BB462453 | — |
| 1453915_a_at | Slc37a3 | solute carrier family 37 (glycerol-3-phosphate | 0.94 | AK012071 | Q8BVX2 /// Q99JR0 |
| | | transporter), member 3 | | | |
| 1430996_at | Etnk1 | ethanolamine kinase 1 | 0.94 | BG867902 | Q8BWV4 /// Q8BXQ0 /// Q8BZY0 /// Q9D4V0 |
| 1455206_at | C130006E23 | hypothetical protein C130006E23 | 0.94 | BQ175276 | — |
| 1449056_at | E330009J07Rik | RIKEN cDNA E330009J07 gene | 0.94 | NM_133929 | Q80TI8 /// Q8C6M2 |
| 1452717_at | Slc25a24 | solute carrier family 25 (mitochondrial carrier, | 0.94 | BM230959 | Q7TPC2 /// Q8BMD8 /// Q8R225 |
| | | phosphate carrier), member 24 | | | |
| 1420618_at | Cpeb4 | cytoplasmic polyadenylation element binding protein 4 | 0.94 | NM_026252 | Q7TN98 /// Q9D5F3 /// Q9D5G2 |
| 1423474_at | Top1 | topoisomerase (DNA) I | 0.94 | BG068053 | Q04750 /// Q8BND6 |
| 1437500_at | — | Mus musculus transcribed sequence with weak | 0.94 | AV306749 | — |
| | | similarity to protein ref: NP_081764.1 (M. musculus) | | | |
| | | RIKEN cDNA 5730493B19 [Mus musculus] | | | |
| 1419821_s_at | Idh1 | isocitrate dehydrogenase 1 (NADP+), soluble | 0.95 | AI788952 | O88844 /// Q8C338 |
| 1448108_at | Tde2 | tumor differentially expressed 2 | 0.95 | AK005203 | Q8C5F9 /// Q9QZI8 |
| 1436957_at | Gabra3 | gamma aminobutyric acid (GABA-A) receptor, subunit | 0.95 | AW557545 | — |
| | | alpha 3 | | | |
| 1417340_at | Txnl2 | thioredoxin-like 2 | 0.95 | NM_023140 | Q9CQM9 |
| 1434461_at | 2610041B18Rik | RIKEN cDNA 2610041B18 gene | 0.95 | AV025957 | AAH57313 /// O88232 /// Q8CGF2 /// Q8CGG0 /// Q9D082 |
| 1418659_at | Clock | circadian locomoter output cycles kaput | 0.95 | BB203106 | BAC97928 /// O08785 /// Q8BRU1 /// Q8C9W6 /// Q8K1L9 |
| 1416488_at | Ccng2 | cyclin G2 | 0.95 | U95826 | O08918 /// Q8C9K5 |
| 1433478_at | Noc4 | neighbor of Cox4 | 0.95 | BQ174254 | AAH09103 /// O70378 |
| 1426476_at | Rasa1 | RAS p21 protein activator 1 | 0.95 | AA124924 | Q91YX7 |
| 1449861_at | Nek4 | NIMA (never in mitosis gene a)-related expressed | 0.95 | BF181187 | AAH57939 /// Q9Z1J2 |
| | | kinase 4 | | | |
| 1431340_a_at | 2310002J21Rik | RIKEN cDNA 2310002J21 gene | 0.95 | AK010048 | — |
| 1448745_s_at | Lor | loricrin | 0.96 | NM_008508 | P18165 |
| 1437263_at | A730089K16Rik | RIKEN cDNA A730089K16 gene | 0.96 | BB138441 | Q8C904 |
| 1429599_a_at | 1110019K23Rik | RIKEN cDNA 1110019K23 gene | 0.96 | AK003824 | — |
| 1420809_a_at | 1500003O03Rik | RIKEN cDNA 1500003O03 gene | 0.96 | NM_019769 | AAH54733 /// AAH64784 /// BAC32532 /// P61022 /// Q8C6H3 |
| 1429534_a_at | Immt | inner membrane protein, mitochondrial | 0.96 | BB222675 | AAH61010 /// Q7TNE2 /// Q8CAQ8 /// Q9D9F6 |
| 1433905_at | Akap7 | A kinase (PRKA) anchor protein 7 | 0.96 | BI730930 | O55074 /// Q7TN79 /// Q8BVR3 |
| 1434776_at | Sema5a | sema domain, seven thrombospondin repeats (type 1 | 0.96 | BQ176610 | AAH65137 /// Q62217 /// Q8BYL6 |
| | | and type 1-like), transmembrane domain (TM) and short | | | |
| | | cytoplasmic domain, (semaphorin) 5A | | | |
| 1427411_s_at | Dleu2 | deleted in lymphocytic leukemia, 2 | 0.96 | BB812902 | — |
| 1439904_at | Fstl5 | ollistatin-like 5 | 0.96 | BB374771 | Q80TG3 /// Q8BFR2 /// Q8C4T3 |
| 1416176_at | Hmgb1 | high mobility group box 1 | 0.96 | BF166000 | AAH64790 /// BAC29902 /// BAC39289 /// P07155 /// Q8BNM0 /// Q8BQ02 /// Q8C7C4 |
| 1440325_at | 2610209L14Rik | RIKEN cDNA 2610209L14 gene | 0.96 | AV332226 | — |
| 1434553_at | C730036B01Rik | RIKEN cDNA C730036B01 gene | 0.96 | BB667728 | Q8CGF5 /// Q9D4Q8 |
| 1436818_a_at | Msi2h | Musashi homolog 2 (Drosophila) | 0.97 | BB479807 | Q920Q6 /// Q920Q7 |
| 1433985_at | Abi2 | abl-interactor 2 | 0.97 | AV263684 | AAH56345 |
| 1452960_at | 1200016D23Rik | RIKEN cDNA 1200016D23 gene | 0.97 | BB274851 | AAH66800 /// Q8BQC9 /// Q8BRJ1 /// Q8C8F4 /// Q9DBQ7 |
| 1428107_at | Sh3bgrl | SH3-binding domain glutamic acid-rich protein like | 0.97 | AK004519 | Q8BHV4 /// Q9JJU8 |
| 1434996_at | Slc25a16 | solute carrier family 25 (mitochondrial carrier, Graves | 0.97 | AV316233 | AAH62168 /// Q8C0K5 |
| | | disease autoantigen), member 16 | | | |
| 1434014_at | Apg4c | APG4 (ATG4) autophagy-related homolog C | 0.97 | BB291836 | AAH58981 |
| | | (S. cerevisiae) | | | |
| 1434687_at | C730026J16 | hypothetical protein C730026J16 | 0.97 | BE456566 | Q8BIL1 /// Q8BW24 /// Q8BW71 |
| 1450858_a_at | Ube2d3 | ubiquitin-conjugating enzyme E2D 3 (UBC4/5 homolog, | 0.97 | AK009276 | P61079 /// Q9D1S1 /// Q9D7F5 |
| | | yeast) | | | |
| 1438933_x_at | — | Mus musculus, Similar to RAS, guanyl releasing protein | 0.97 | BE688720 | O09004 /// Q80WC0 /// Q8BSC8 /// Q9QUG9 |
| | | 2, clone IMAGE: 4481738, mRNA | | | |
| 1441799_at | — | Mus musculus 13 days embryo male testis cDNA, | 0.97 | AI098139 | — |
| | | RIKEN full-length enriched library, clone: 6030422H21 | | | |
| | | product: unknown EST, full insert sequence | | | |
| 1417980_a_at | Insig2 | insulin induced gene 2 | 0.97 | AV257512 | Q8BWP1 /// Q91WG1 |
| 1423032_at | Rpl39 | ribosomal protein L39 | 0.97 | AV107150 | P02404 |
| 1426448_at | Pja1 | praja1, RING-H2 motif containing | 0.97 | BM199789 | O55176 |
| 1422244_at | Pkdrej | polycystic kidney disease (polycystin) and REJ (sperm | 0.97 | NM_011105 | Q8C0Z9 |
| | | receptor for egg jelly, sea urchin homolog)-like | | | |
| 1416267_at | Scoc | short coiled-coil protein | 0.97 | NM_019708 | BAB22159 /// Q8C6K2 /// Q9CWN2 /// Q9CY27 /// Q9WU55 |
| 1449047_at | Hpcl-pending | 2-hydroxyphytanoyl-CoA lyase | 0.97 | NM_019975 | BAC31032 /// BAC34059 /// Q9QXE0 |
| 1427277_at | Six1 | sine oculis-related homeobox homolog (Drosophila) | 0.97 | BB137929 | Q62231 /// Q8BSP4 |
| 1455407_at | Zfp236 | zinc finger protein 236 | 0.97 | BB282741 | Q8BI89 /// Q8BIE3 |
| 1434194_at | Mtap2 | microtubule-associated protein 2 | 0.97 | AV337593 | P20357 /// Q80X35 /// Q80ZL4 |
| 1415864_at | Bpgm | 2,3-bisphosphoglycerate mutase | 0.97 | NM_007563 | BAC31541 /// BAC37133 /// P15327 |
| 1434889_at | A430081P20Rik | RIKEN cDNA A430081P20 gene | 0.97 | BI905111 | Q8BYE3 |
| 1421519_a_at | Zfp120 | zinc finger protein 120 | 0.97 | NM_023266 | Q8BZW4 /// Q9EQK2 /// Q9EQK4 /// Q9JIB8 |
| 1449972_s_at | Zfp97 | zinc finger protein 97 | 0.97 | NM_011765 | — |
| 1456199_x_at | — | Mus musculus, clone IMAGE: 6512643, mRNA | 0.97 | BB106402 | — |
| 1421830_at | Ak4 | adenylate kinase 4 | 0.97 | NM_009647 | Q9WUR9 |
| 1423543_at | Swap70 | SWAP complex protein | 0.97 | AK019882 | AAH65136 /// O88443 |
| 1457124_at | — | Mus musculus transcribed sequences | 0.98 | AV328224 | — |
| 1418070_at | Cdyl | chromodomain protein, Y chromosome-like | 0.98 | AF081260 | AAH62123 /// Q9WTK2 |
| 1426999_at | 1700016A15Rik | RIKEN cDNA 1700016A15 gene | 0.98 | BM198642 | Q8BIY8 /// Q8BJ05 /// Q8R3Q8 /// Q8R3R2 /// Q9DAA8 |
| 1425826_a_at | Sorbs1 | sorbin and SH3 domain containing 1 | 0.98 | AF078667 | Q62417 /// Q80TF8 /// Q8BZI3 /// Q8K3Y2 /// Q921F8 /// Q9Z0Z8 /// Q9Z0Z9 |
| 1430314_at | 4933437F05Rik | RIKEN cDNA 4933437F05 gene | 0.98 | BB217068 | Q9D3S5 |
| 1454626_at | Cltc | clathrin, heavy polypeptide (Hc) | 0.98 | BM211219 | Q80U89 /// Q8K2I5 |
| 1442760_x_at | — | Mus musculus transcribed sequences | 0.98 | BB206454 | — |
| 1423114_at | Ube2d3 | ubiquitin-conjugating enzyme E2D 3 (UBC4/5 homolog, | 0.98 | AK009276 | P61079 /// Q9D1S1 /// Q9D7F5 |
| | | yeast) | | | |
| 1456060_at | Maf | avian musculoaponeurotic fibrosarcoma (v-maf) AS42 | 0.98 | AV284857 | — |
| | | oncogene homolog | | | |
| 1436981_a_at | Ywhaz | tyrosine 3-monooxygenase/tryptophan 5- | 0.98 | BB706206 | — |
| | | monooxygenase activation protein, zeta polypeptide | | | |
| 1421750_a_at | Vbp1 | von Hippel-Lindau binding protein 1 | 0.98 | NM_011692 | Q15765 /// Q9CPZ0 |
| 1417549_at | Zfp68 | Zinc finger protein 68 | 0.99 | NM_013844 | O88238 /// Q60910 /// Q8BLK6 /// Q8VEM6 /// Q9Z116 |
| 1422576_at | Sca10 | spinocerebellar ataxia 10 homolog (human) | 0.99 | NM_016843 | BAC32981 /// BAC37285 /// P28658 /// Q8BWX1 |
| 1434278_at | — | — | 0.99 | BG976607 | — |
| 1445367_at | — | Mus musculus transcribed sequences | 0.99 | C76202 | — |
| 1432195_s_at | Ccnl2 | cyclin L2 | 0.99 | AK008585 | Q60995 /// Q8BLP2 /// Q8CIJ8 /// Q99L73 /// Q9CVZ6 /// Q9D814 /// Q9JJA7 /// Q9QXH5 |
| 1416861_at | Stam | signal transducing adaptor molecule (SH3 domain and | 0.99 | NM_011484 | P70297 |
| | | ITAM motif) 1 | | | |
| 1416998_at | Rrs1 | RRS1 ribosome biogenesis regulator homolog | 0.99 | NM_021511 | Q9CYH6 |
| | | (S. cerevisiae) | | | |
| 1423096_at | Capn7 | calpain 7 | 0.99 | BQ257745 | Q9R1S8 |
| 1456862_at | Six4 | sine oculis-related homeobox 4 homolog (Drosophila) | 0.99 | AI893638 | Q61321 |
| 1427504_s_at | Sfrs2 | splicing factor, arginine/serine-rich 2 (SC-35) | 0.99 | AF250133 | BAC39610 /// BAC40111 /// Q06477 /// Q62093 /// Q8C671 /// Q99MY4 /// Q99MY5 |
| 1428471_at | Sorbs1 | sorbin and SH3 domain containing 1 | 0.99 | BQ176684 | Q62417 /// Q80TF8 /// Q8BZI3 /// Q8K3Y2 /// Q921F8 /// Q9Z0Z8 /// Q9Z0Z9 |
| 1423378_at | Adam23 | a disintegrin and metalloprotease domain 23 | 0.99 | AI838132 | AAS49900 /// AAS49901 /// Q9R1V7 |
| 1418529_at | Osgep | O-sialoglycoprotein endopeptidase | 0.99 | NM_133676 | Q8BWU5 |
| 1454980_at | AU018056 | expressed sequence AU018056 | 0.99 | BB667201 | BAC98282 /// Q7TSQ8 |
| 1428938_at | Gnaq | guanine nucleotide binding protein, alpha q polypeptide | 0.99 | W41915 | AAH57583 /// P21279 /// Q8C6U1 |
| 1418150_at | Mtmr4 | myotubularin related protein 4 | 0.99 | BQ032797 | AAH58091 /// AAH58364 /// Q91XS1 |
| 1424895_at | Gpsm2 | G-protein signalling modulator 2 (AGS3-like, | 0.99 | BC021308 | Q8BLX3 /// Q8VDU0 |
| | | C. elegans) | | | |
| 1451133_s_at | 8430437G11Rik | RIKEN cDNA 8430437G11 gene | 0.99 | BC007160 | Q91VX9 |
| 1450899_at | Nedd1 | neural precursor cell expressed, developmentally down- | 1.00 | BBB29652 | AAH66870 /// P33215 /// Q8BN12 /// Q8BN86 /// Q8BQL9 /// Q9CWK2 |
| | | regulated gene 1 | | | |
| 1419181_at | Zfp326 | zinc finger protein 326 | 1.00 | NM_018759 | O88291 /// Q8BSJ5 /// Q8K1X9 /// Q9CYG9 |
| 1448502_at | Slc16a7 | solute carrier family 16 (monocarboxylic acid | 1.00 | NM_011391 | BAC36415 /// O70451 |
| | | transporters), member 7 | | | |
| 1415894_at | Enpp2 | eclonucleotide pyrophosphatase/phosphodiesterase 2 | 1.00 | BC003264 | AAH58759 /// Q8CAF0 /// Q9R1E6 |
| 1458693_at | — | Mus musculus transcribed sequences | 1.00 | BB461850 | — |
| 1419252_at | Eps15 | epidermal growth factor receptor pathway substrate 15 | 1.00 | BG067649 | P42567 /// Q80ZL3 /// Q8C431 |
| 1433976_at | — | Mus musculus, clone IMAGE: 5355681, mRNA | 1.00 | BI249740 | — |
| 1451047_at | Itm2a | integral membrane protein 2A | 1.00 | BI966443 | Q61500 /// Q8K0H4 /// Q9CRW4 |
| 1448933_at | Pcdhb17 | protocedherin beta 17 | 1.00 | NM_053142 | Q80TB2 /// Q91VD8 /// Q925L4 |
| 1424873_at | Rnf2 | ring finger protein 2 | 1.00 | BC020122 | O35699 /// O35729 /// Q8C1X8 /// Q9CQJ4 |
| 1435890_at | 5730596K20Rik | RIKEN cDNA 5730596K20 gene | 1.00 | BB795103 | — |
| 1425494_s_at | Bmpr1a | bone morphogenetic protein receptor, type 1A | 1.00 | BM939768 | AAQ64630 /// P36895 |
| 1427269_at | 2610019N13Rik | RIKEN cDNA 2610019N13 gene | 1.00 | AW261583 | Q8BV04 /// Q8BYD3 /// Q8C0Z3 /// Q8CFF2 /// Q8CGI3 /// Q9CT17 |
| 1457568_at | C230004L04 | hypothetical protein C230004L04 | 1.00 | BB380198 | — |
| 1449128_at | D11Ertd707e | DNA segment, Chr 11, ERATO Doi 707, expressed | 1.00 | NM_025918 | AAH58520 /// AAH61076 /// Q9CR29 |
| 1424443_at | Tm6sf1 | transmembrane 6 superfamily member 1 | 1.00 | AV378394 | P58749 /// Q8BUN7 |
| 1441145_at | D030065N23Rik | RIKEN cDNA D030065N23 gene | 1.01 | BB448266 | — |
| 1435042_at | 9130004C02Rik | RIKEN cDNA 9130004C02 gene | 1.01 | BG296454 | — |
| 1426558_x_at | 3100002L24Rik | RIKEN cDNA 3100002L24 gene | 1.01 | BB283527 | Q8BHI0 |
| 1442210_at | 1700058C01Rik | RIKEN cDNA 1700058C01 gene | 1.01 | AV273577 | Q8BII1 |
| 1428103_at | Adam10 | a disintegrin and metalloprotease domain 10 | 1.01 | AV327574 | AAH66207 /// O35598 |
| 1420772_a_at | Gilz | glucocorticoid-induced leucine zipper | 1.01 | NM_010286 | Q8K160 /// Q9EQN0 /// Q96EN1 /// Q9EQN2 /// Q9Z2S7 |
| 1428011_a_at | Erbb2ip | Erbb2 interacting protein | 1.01 | BC028256 | Q80TH2 /// Q8BQ14 /// Q8K171 /// Q99JU3 /// Q9JI47 |
| 1452659_at | Dek | DEK oncogene (DNA binding) | 1.02 | AK007546 | Q7TNV0 /// Q80VC5 /// Q8BZV6 /// Q9CVL7 |
| 1434339_at | 2610318I01Rik | RIKEN cDNA 2610318I01 gene | 1.02 | AW548221 | Q8K012 |
| 1428652_at | 0610010F05Rik | RIKEN cDNA 0610010F05 gene | 1.02 | BB469274 | BAC98264 /// Q8BPK6 /// Q9CWA7 |
| 1419750_at | Dnmt2 | DNA methyltransferase 2 | 1.02 | BB010597 | O55055 /// Q8C7F0 /// Q8CE27 |
| 1457741_at | Tex2 | testis expressed gene 2 | 1.02 | AV377040 | — |
| 1448922_at | Dusp19 | dual specificity phosphatase 19 | 1.02 | NM_024438 | Q8K4T5 /// Q99N12 /// Q9CRR3 /// Q9D5P6 |
| 1428693_at | 2610044O15Rik | RIKEN cDNA 2610044O15 gene | 1.02 | AK011776 | Q8BG62 |
| 1442810_x_at | — | Mus musculus transcribed sequences | 1.02 | BB452274 | Q62205 |
| 1435190_at | Chl1 | close homolog of L1 | 1.02 | BB378591 | P70232 /// Q8BS24 /// Q8C6W0 /// Q8C823 |
| 1455029_at | KIf21a | kinesin family member 21A | 1.02 | BB342219 | AAH60698 /// AAH62896 /// BAC98236 /// Q8BWZ9 /// Q8BXF1 /// Q8BXG5 /// Q9QXL2 |
| 1439732_at | — | Mus musculus 16 days neonate cerebellum cDNA, | 1.02 | BB129764 | — |
| | | RIKEN full-length enriched library, clone: 9630041I08 | | | |
| | | product: unknown EST, full insert sequence | | | |
| 1420675_at | Zfp113 | zinc finger protein 113 | 1.03 | NM_019747 | AAH56445 /// Q8C689 |
| 1435181_at | AI461788 | expressed sequence AI461788 | 1.03 | BG073348 | Q8BMU8 /// Q8C107 |
| 1435899_at | 9430079B08Rik | RIKEN cDNA 9430079B08 gene | 1.03 | BE136439 | — |
| 1418649_at | Egln3 | EGL nine homolog 3 (C. elegans) | 1.03 | BB284358 | Q91UZ4 |
| 1448557_at | 1200015N20Rik | RIKEN cDNA 1200015N20 gene | 1.03 | NM_024244 | AAH66835 /// Q8BLV7 /// Q99LJ4 /// Q9DBR2 |
| 1452328_s_at | Pja2 | praja 2, RING-H2 motif containing | 1.03 | BF160731 | Q80U04 /// Q810E3 /// Q91W46 /// Q99KC0 |
| 1440651_at | Dusp16 | dual specificity phosphatase 16 | 1.03 | BM238701 | — |
| 1439906_at | — | Mus musculus adult male spinal cord cDNA, RIKEN | 1.03 | BB184086 | — |
| | | full-length enriched library, clone: A330007G10 | | | |
| | | product: unknown EST, full insert sequence | | | |
| 1450950_at | Cspg6 | chondroitin sulfate proteoglycan 6 | 1.03 | AK005647 | AAH36330 /// AAH57345 /// AAH62935 /// Q9CW03 |
| 1450017_at | Ccng1 | cyclin G1 | 1.04 | BG065754 | AAH05534 /// P51945 |
| 1416015_s_at | Abce1 | ATP-binding cassette, sub-family E (OABP), member 1 | 1.04 | NM_015751 | AAH66794 /// AAH66836 /// P61222 /// Q8C2N8 |
| 1424156_at | Rbl1 | retinoblastoma-like 1 (p107) | 1.04 | U27177 | AAH60124 /// Q64701 /// Q8BTA6 /// Q8CCD4 |
| 1434191_at | A530016O06Rik | RIKEN cDNA A530016O06 gene | 1.04 | AI790538 | Q8BS35 /// Q8C7H5 /// Q8CAA6 |
| 1426088_at | — | — | 1.04 | BC004015 | — |
| 1429690_at | 1300003B13Rik | RIKEN cDNA 1300003B13 gene | 1.04 | AK004870 | Q8K3B4 |
| 1424733_at | P2ry14 | purinergic receptor P2Y, G protein coupled, 14 | 1.04 | AF177211 | BAC30456 /// Q9ESG6 |
| 1422869_at | Mertk | c-mer proto-oncogene tyrosine kinase | 1.04 | NM_008587 | Q60805 /// Q8C584 /// Q8CE52 |
| 1435265_at | — | Mus musculus transcribed sequences | 1.04 | BF466929 | — |
| 1451626_x_at | — | — | 1.05 | U58494 | — |
| 1418308_at | Hus1 | Hus1 homolog (S. pombe) | 1.05 | NM_008316 | AAH61249 /// O70543 /// Q8BQY8 |
| 1459321_at | — | Mus musculus adult male diencephalon cDNA, RIKEN | 1.05 | BB075541 | — |
| | | full-length enriched library, clone: 9330109G15 | | | |
| | | product: unknown EST, full insert sequence | | | |
| 1428522_at | Ttf2 | transcription termination factor, RNA polymerase II | 1.05 | BB283807 | — |
| 1451127_at | AW146242 | expressed sequence AW146242 | 1.05 | BC024822 | Q8C0B7 /// Q8R1C3 |
| 1441980_at | C030007I09Rik | RIKEN cDNA C030007I09 gene | 1.05 | BB355593 | Q8BQT9 |
| 1421546_a_at | Racgap1 | Rac GTPase-activating protein 1 | 1.05 | NM_012025 | Q9WVM1 |
| 1418664_at | Mpdz | Multiple PDZ domain protein | 1.05 | AK019164 | AAH61504 /// AAL37377 /// O08783 /// Q80ZY8 /// Q8C0H8 /// Q8VBV5 /// Q8VBX6 /// |
| | | | | | Q8VBY0 /// Q9Z1K3 |
| 1422702_at | Oazin | omithine decarboxylase antizyme inhibitor | 1.05 | BE626090 | BAC33870 /// BAC40494 /// O35484 /// Q8C2R8 /// Q8K1E5 |
| 1427208_at | Zfp451 | zinc finger protein 451 | 1.05 | BC024435 | AAH62154 /// Q80TA4 /// Q811K1 /// Q8C0P7 /// Q8R0N3 /// Q8R1L1 /// Q8R5E1 /// Q8VCL4 |
| | | | | | /// Q9CUK0 |
| 1416187_s_at | Pnrc2 | proline-rich nuclear receptor coactivator 2 | 1.05 | NM_026383 | Q9CR73 /// Q9CXC6 |
| 1458075_at | — | Mus musculus 10 days neonate cerebellum cDNA, | 1.05 | BB350401 | — |
| | | RIKEN full-length enriched library, clone: B930079L03 | | | |
| | | product: unknown EST, full insert sequence | | | |
| 1439012_a_at | Dck | deoxycytidine kinase | 1.06 | BB030204 | BAB23394 /// BAB27131 /// BAC33307 /// BAC40203 /// P43346 /// Q80US6 |
| 1435590_at | D430047L21Rik | RIKEN cDNA D430047L21 gene | 1.06 | AV325177 | — |
| 1439753_x_at | Six4 | sine oculis-related homeobox 4 homolog (Drosophila) | 1.06 | AI893638 | Q61321 |
| 1415863_at | Eif4g2 | eukaryotic translation initiation factor 4, gamma 2 | 1.06 | NM_013507 | AAH64810 /// Q62448 |
| 1423824_at | 5031439A09Rik | RIKEN cDNA 5031439A09 gene | 1.06 | BC018381 | Q8CD50 /// Q8CDZ6 /// Q8CE42 /// Q9D2B7 |
| 1416700_at | Arhe | ras homolog gene family, member E | 1.06 | BC009002 | BAC28975 /// P61588 |
| 1426405_at | Rnf11 | ring finger protein 11 | 1.06 | BI150320 | Q9QYK7 |
| 1455164_at | Cdgap | Cdc42 GTPase-activating protein | 1.06 | AV308092 | BAC98119 /// Q9WV94 |
| 1429908_at | 6530403A03Rik | RIKEN cDNA 6530403A03 gene | 1.06 | AK004216 | Q8BIU0 /// Q8BJ30 /// Q8BMZ0 /// Q8CFF6 /// Q8VDQ5 /// Q9CSG3 /// Q9CVM5 /// Q9D361 |
| 1435543_at | Lalba | lactalbumin, alpha | 1.06 | BM124893 | P70382 /// Q61315 /// Q8BNP7 /// Q8BRD8 /// Q8C9I9 |
| 1426880_at | 9430077C05Rik | RIKEN cDNA 9430077C05 gene | 1.06 | BM250266 | BAD14929 /// BAD14930 /// Q80VK2 /// Q8BHX8 /// Q8BHY1 /// Q8CHA8 /// Q8R0K6 /// |
| | | | | | Q9CX18 |
| 1443857_at | Hook3 | hook homolog 3 (Drosophila) | 1.06 | BB825115 | Q8BUK6 |
| 1434109_at | Sh3bgrl2 | SH3 domain binding glutamic acid-rich protein like 2 | 1.06 | AV291265 | Q8BG73 /// Q8C073 /// Q8C0Z4 |
| 1422659_at | Camk2d | calcium/calmodulin-dependent protein kinase II, delta | 1.07 | NM_023813 | AAH52894 /// O70459 /// Q8C3F8 /// Q8C4I3 /// Q8C8X9 /// Q8CAC5 /// Q8CCM0 /// Q9CZE2 |
| 1428248_at | Nfx1 | nuclear transcription factor, X-box binding 1 | 1.07 | AK005038 | Q7TPT4 /// Q8C6R0 /// Q8CC59 /// Q9D9E1 /// Q9D8C8 /// Q9JKW7 |
| 1459843_s_at | Smad1 | MAD homolog 1 (Drosophila) | 1.07 | BB257769 | AAH58693 /// BAC35658 /// P70340 /// Q8CB69 |
| 1434413_at | Igf1 | insulin-like growth factor 1 | 1.07 | BG092677 | AAL34535 /// P05017 /// P05018 /// Q8C4U6 /// Q8CAR0 |
| 1455938_x_at | Rad21 | RAD21 homolog (S. pombe) | 1.07 | AV025454 | BAC97860 /// Q61550 /// Q810A8 |
| 1453399_at | Ccnt2 | cyclin T2 | 1.07 | AK013634 | Q77QK0 /// Q8VCM9 /// Q9D6H3 |
| 1436594_at | C630016O21Rik | RIKEN cDNA C630016O21 gene | 1.07 | BB281667 | Q8BIV1 |
| 1423982_at | Fusip1 | FUS interacting protein (serine-arginine rich) 1 | 1.07 | AF060490 | Q8CF51 /// Q9R0U0 |
| 1416114_at | Sparcl1 | SPARC-like 1 (mast9, hevin) | 1.07 | NM_010097 | P70663 |
| 1439026_at | 6330504P12Rik | RIKEN cDNA 6330504P12 gene | 1.07 | BB125842 | — |
| 1428875_at | Golph4 | golgi phosphoprotein 4 | 1.07 | BE981485 | Q8BV17 /// Q8BWP9 /// Q8BXA1 |
| 1457632_s_at | Mrg1 | myeloid ecotropic viral integration site-related gene 1 | 1.07 | BB207647 | AAH17375 /// P97367 |
| 1416688_at | Snap91 | synaptosomal-associated protein 91 | 1.07 | NM_013669 | Q61548 /// Q7TT20 /// Q8BQA2 /// Q8CHE0 /// Q8K0D4 |
| 1428944_at | 5730469D23Rik | RIKEN cDNA 5730469D23 gene | 1.07 | BB417360 | AAH63048 /// Q8C7R4 |
| 1457392_at | AI450757 | expressed sequence AI450757 | 1.07 | BB055966 | AAH63761 /// Q8BZX4 |
| 1434671_at | B230337E12Rik | RIKEN cDNA B230337E12 gene | 1.08 | BM120593 | — |
| 1459991_at | 4732465J09Rik | RIKEN cDNA 4732465J09 gene | 1.08 | BB104162 | Q80Y92 /// Q8C0U0 /// Q8C170 |
| 1426218_at | Glcci1 | glucocorticoid induced transcript 1 | 1.08 | AA152997 | Q80YT1 /// Q8CEA5 /// Q8K3I9 /// Q925C1 /// Q9D6W9 |
| 1452953_at | 1810036I24Rik | RIKEN cDNA 1810036I24 gene | 1.08 | AK017572 | Q9D8T4 |
| 1419062_at | Epb4.1l3 | erythrocyte protein band 4.1-like 3 | 1.08 | NM_013813 | Q8BT38 /// Q9WV92 |
| 1452291_at | Centd1 | centaurin, delta 1 | 1.08 | AV375176 | Q80TX2 /// Q8BY88 /// Q8BYL0 /// Q8BZ05 /// Q8C3T2 /// Q8VEL6 |
| 1448584_at | 1200013F24Rik | RIKEN cDNA 1200013F24 gene | 1.08 | NM_025822 | Q80XR9 /// Q8BR75 /// Q8CF54 /// Q8CFJ0 /// Q9CSR8 /// Q9D0Y1 |
| 1435993_at | — | Mus musculus transcribed sequences | 1.08 | BB027219 | — |
| 1434759_at | Lrrtm3 | leucine rich repeat transmembrane neuronal 3 | 1.08 | BM224801 | Q8BGJ7 /// Q8BZ81 /// Q8BZA0 |
| 1416440_at | Cd164 | CD164 antigen | 1.08 | NM_016898 | Q9CW91 /// Q9R0L9 /// Q9Z317 |
| 1452750_at | — | Mus musculus, clone IMAGE: 3676181, mRNA | 1.08 | BB820846 | — |
| 1427042_at | Mal2 | mal, T-cell differentiation protein 2 | 1.08 | BB127697 | Q8BI08 |
| 1452876_x_at | 2610044O15Rik | RIKEN cDNA 2610044O15 gene | 1.08 | AK011776 | Q8BG62 |
| 1452054_at | 6130401J04Rik | RIKEN cDNA 6130401J04 gene | 1.08 | BB796558 | Q8BVJ8 /// Q8VDW4 /// Q9D5H3 |
| 1444425_at | — | Mus musculus 0 day neonate kidney cDNA, RIKEN full- | 1.08 | BE994902 | — |
| | | length enriched library, clone: D630017L16 | | | |
| | | product: unknown EST, full insert sequence | | | |
| 1426924_at | 2900024N03Rik | RIKEN cDNA 2900024N03 gene | 1.09 | AA709668 | — |
| 1451867_x_at | Arhgap6 | Rho GTPase activating protein 6 | 1.09 | AF177664 | O54834 /// Q8BG83 /// Q8C842 /// Q8C8B2 |
| 1435459_at | Fmo2 | flavin containing monooxygenase 2 | 1.09 | BM936480 | Q8K2I3 /// Q9QZF7 |
| 1424369_at | Psmf1 | proteasome (prosome, macropain) inhibitor subunit 1 | 1.09 | BC012260 | Q8BHL8 /// Q8C0G9 /// Q91X47 |
| 1451033_a_at | Trpc4 | transient receptor potential cation channel, subfamily C, | 1.09 | BB271442 | Q8BNT2 /// Q9QUQ5 |
| | | member 4 | | | |
| 1428883_at | 1110007C24Rik | RIKEN cDNA 1110007C24 gene | 1.09 | AK003528 | AAH56944 /// AAQ64008 /// Q7TQE6 /// Q80SV6 /// Q80YA4 /// Q9CTI0 |
| 1458709_a_at | 2810423G08Rik | RIKEN cDNA 2810423G08 gene | 1.09 | AV274704 | — |
| 1418815_at | Cdh2 | cadherin 2 | 1.09 | BC022107 | AAH22107 /// P15116 /// Q8BSI9 |
| 1456359_at | 4632422M10Rik | RIKEN cDNA 4632422M10 gene | 1.09 | AV233215 | Q8C908 /// Q8CEC6 |
| 1433694_at | Pde3b | phosphodiesterase 3B, cGMP-inhibited | 1.09 | AV270888 | — |
| 1442598_at | Prkrip1 | Prkr interacting protein 1 (IL11 inducible) | 1.09 | AV324577 | Q8BL85 /// Q9CWV6 /// Q9CXA5 /// Q9CY32 |
| 1418357_at | Foxg1 | forkhead box G1 | 1.09 | NM_008241 | Q60987 /// Q80VP3 |
| 1423549_at | Slc1a4 | solute carrier family 1 (glutamate/neutral amino acid | 1.09 | BB277461 | O35874 /// Q8BXT5 /// Q9ESU8 |
| | | transporter), member 4 | | | |
| 1426065_a_at | Ifld2 | induced in fatty liver dystrophy 2 | 1.09 | BC012955 | CAD55728 /// Q8K4K2 |
| 1449686_s_at | Scp2 | sterol carrier protein 2, liver | 1.10 | C76618 | P32020 |
| 1417622_at | Slc12a2 | solute carrier family 12, member 2 | 1.10 | BG069505 | P55012 |
| 1436791_at | Wnt5a | wingless-relatad MMTV integration site 5A | 1.10 | BB067079 | P22725 /// Q8BM17 /// Q8BMF9 /// Q8VCV6 |
| 1433537_at | 4833408C14Rik | RIKEN cDNA 4833408C14 gene | 1.10 | AV112912 | — |
| 1425485_at | Mtmr6 | myotubularin related protein 6 | 1.10 | BC020019 | Q8VE11 |
| 1435637_at | 2310047C21Rik | RIKEN cDNA 2310047C21 gene | 1.10 | AW554709 | Q99KW9 |
| 1451324_s_at | 3830421F13Rik | RIKEN cDNA 3830421F13 gene | 1.10 | BC010204 | Q8BVM8 /// Q8K361 /// Q91Z49 /// Q921B0 /// Q9D6A8 |
| 1443392_at | Trpv1 | transient receptor potential cation channel, subfamily V, | 1.10 | BB346256 | — |
| | | member 1; capsaicin receptor; venilloid receptor | | | |
| | | subtype 1 | | | |
| 1434283_at | Desrt | developmentally and sexually retarded with transient | 1.10 | BB079486 | Q8BM75 |
| | | immune abnormalities | | | |
| 1434860_at | — | Mus musculus transcribed sequences | 1.10 | BQ176197 | — |
| 1447985_s_at | Ankib1 | ankyrin repeat and IBR domain containing 1 | 1.11 | C80642 | BAC98153 |
| 1453152_at | Mamdc2 | MAM domain containing 2 | 1.11 | AK004794 | Q8CG85 |
| 1457164_at | Anktm1 | ANKTM1 | 1.11 | BB309395 | Q8BLA8 |
| 1429371_at | 2810426N06Rik | RIKEN cDNA 2810426N06 gene | 1.11 | AK013166 | AAH27798 /// Q8BUQ3 /// Q9CRL6 /// Q9CZ01 |
| 1434302_at | 9430025M21Rik | RIKEN cDNA 9430025M21 gene | 1.11 | AV307311 | — |
| 1449431_at | Trpc6 | transient receptor potential cation channel, subfamily C, | 1.11 | NM_013838 | AAH67391 /// AAH68310 /// Q61143 |
| | | member 6 | | | |
| 1426840_at | Ythdf3 | YTH domain family 3 | 1.11 | BB183208 | AAH57158 /// AAH67040 /// AAH67042 /// Q7TN20 /// Q8BKB6 /// Q8BVC6 /// Q8BYK6 /// |
| | | | | | Q8R5D2 |
| 1417770_s_at | Psmc6 | proteasome (prosome, macropain) 26S subunit, | 1.11 | AW208944 | AAH57997 /// Q810A6 /// Q8QZS9 /// Q92524 /// Q9CXH9 |
| | | ATPase, 6 | | | |
| 1416152_a_at | Sfrs3 | splicing factor, arginine/serine-rich 3 (SRp20) | 1.11 | NM_013663 | AAH68111 /// BAC37445 /// P23152 /// Q8C3H6 /// Q9D6W4 |
| 1452176_at | Nup153 | nucleoporin 153 | 1.11 | BB292874 | Q80UN3 /// Q80WR0 /// Q8BRF6 /// Q8R2M9 |
| 1433502_s_at | AW550801 | expressed sequence AW550801 | 1.12 | BM213835 | BAC98160 /// Q8K2F5 |
| 1426358_at | 2810468K05Rik | RIKEN cDNA 2810468K05 gene | 1.12 | BB272466 | Q8JZX2 /// Q8VE26 /// Q91VG7 /// Q9D3K9 |
| 1451624_a_at | 1700048E23Rik | RIKEN cDNA 1700048E23 gene | 1.12 | BC025612 | Q9D9M5 |
| 1427167_at | AI448196 | expressed sequence AI448196 | 1.12 | BE865094 | Q8K2R3 /// Q8R103 |
| 1439059_at | BC031748 | cDNA sequence BC031748 | 1.12 | BB709811 | Q8K2D0 |
| 1440226_at | 9430018I06 | hypothetical protein 9430018I06 | 1.12 | BB088782 | C8C9G1 |
| 1437111_at | A230108E06 | hypothetical protein A230108E06 | 1.12 | BB183628 | — |
| 1436202_at | 9430072K23Rik | RIKEN cDNA 9430072K23 gene | 1.12 | AI853644 | — |
| 1451074_at | Rnf13 | ring finger protain 13 | 1.12 | AF037205 | AAH58182 /// O54965 /// Q8CB78 |
| 1439088_at | Pdzk8 | PDZ domain containing 8 | 1.12 | BB102308 | — |
| 1460357_at | Ythdf2 | YTH domain family 2 | 1.13 | BB455932 | Q8BM70 /// Q8BUI8 /// Q8K325 /// Q91YT7 |
| 1453456_at | 2900084O13Rik | RIKEN cDNA 2900084O13 gene | 1.13 | BM117709 | — |
| 1428607_at | Araf | ref-related oncogene | 1.13 | AK010060 | P04627 /// Q8CAD1 |
| 1435167_at | Ranbp6 | RAN binding protein 6 | 1.13 | AW108431 | Q8BIV3 |
| 1455434_a_at | Ktn1 | kinectin 1 | 1.13 | BF162017 | Q61595 /// Q8BG49 /// Q8BHF4 /// Q8BHM8 /// Q8C9Y5 /// Q8CG51 /// Q8CG52 /// Q8CG53 |
| | | | | | /// Q8CG54 /// Q8CG55 /// Q8CG56 /// Q8CG57 /// Q8CG58 /// Q8CG59 /// Q8CG60 /// |
| | | | | | Q8CG61 /// Q8CG62 /// Q8CG63 |
| 1454666_at | 9930027G08Rik | RIKEN cDNA 9930027G08 gene | 1.13 | AV230488 | BAB25371 /// Q60980 /// Q8BV07 |
| 1440354_at | Elovl7 | ELOVL family member 7, elongation of long chain fatty | 1.13 | BB149977 | Q8BX38 /// Q8BYY8 /// Q9D2Y9 |
| | | acids (yeast) | | | |
| 1427683_at | Egr2 | early growth response 2 | 1.13 | X06746 | P08152 |
| 1447522_s_at | 5430432P15Rik | RIKEN cDNA 5430432P15 gene | 1.13 | AI662480 | AAH63101 /// Q8BXH7 |
| 1460044_at | — | Mus musculus 0 day neonate cerebellum cDNA, RIKEN | 1.13 | BB389395 | — |
| | | full-length enriched library, clone: C230062K19 | | | |
| | | product unknown EST, full insert sequence | | | |
| 1417222_a_at | 2310075C12Rik | RIKEN cDNA 2310075C12 gene | 1.13 | NM_133739 | Q8CEX4 /// Q91Z22 |
| 1434717_at | Cul3 | cullin 3 | 1.13 | BM198837 | BAC97984 /// Q9CTE0 /// Q9JLV5 |
| 1433784_at | 9030612M13Rik | RIKEN cDNA 9030612M13 gene | 1.13 | BI076832 | — |
| 1429108_at | Msl2 | male-specific lethal-2 homolog (Drosophila) | 1.13 | BB745314 | Q8CBI7 |
| 1427195_at | — | Mus musculus, clone IMAGE: 3983419, mRNA | 1.14 | W91587 | — |
| 1419087_s_at | Sf3a1 | splicing factor 3a, subunit 1 | 1.14 | BB031765 | Q8C0M7 /// QSC128 /// Q8C175 /// Q8K4Z5 /// Q921T3 |
| 1416018_at | Dr1 | down-ragulator of transcription 1 | 1.14 | NM_026106 | Q91WV0 |
| 1417668_at | Rtn4ip1 | reticulon 4 interacting protein 1 | 1.14 | NM_130892 | QBR1T0 /// Q924D0 |
| 1418736_at | B3galt3 | UDP-Gal: betaGlcNAc beta 1,3-galactosyltransferase, | 1.14 | BC003835 | O54906 /// Q9CTE5 |
| | | polypeptide 3 | | | |
| 1422993_s_at | Thoc4 | THO complex 4 | 1.14 | NM_019484 | O08583 |
| 1427131_s_at | 1810012N18Rik | RIKEN cDNA 1810012N18 gene | 1.14 | AV234245 | — |
| 1425020_at | Ubxd4 | UBX domain containing 4 | 1.15 | AV174556 | Q99KJ0 |
| 1438029_at | 4930535B03Rik | RIKEN cDNA 4930535B03 gene | 1.15 | BB817800 | AAH67054 /// BAC97960 /// Q8BU73 /// Q9D4Z4 |
| 1418997_at | 4930469P12Rik | RIKEN cDNA 4930469P12 gene | 1.15 | BC021522 | Q8VDL7 /// Q91V16 |
| 1454768_at | Kcnf1 | potassium voltage-gated channel, subfamily F, member 1 | 1.15 | AV337635 | Q7TSH7 |
| 1451840_at | Kcnip4 | Kv channel interacting protein 4 | 1.15 | BG261945 | AAH51130 /// Q8CAD0 /// Q8R4I2 /// Q9EQ01 |
| 1416487_a_at | Yap | yes-asociated protein | 1.15 | NM_009534 | AAH39125 /// P46938 /// Q91WL1 |
| 1454795_at | Cobll1 | Cobl-like 1 | 1.15 | AV080881 | AAH67007 /// Q7TQM8 /// Q8BJK8 /// Q8BWB9 /// Q8BWH3 |
| 1429063_s_at | Kif16b | kinesin family member 16B | 1.15 | BG066903 | BAC98211 /// O35056 /// Q8BZZ9 |
| 1436231_at | 2900052N01Rik | RIKEN cDNA 2900052N01 gene | 1.15 | AU067665 | Q8C7N3 /// Q8CAM6 |
| 1424674_at | Slc39a6 | solute carrier family 39 (metal ion transporter), member 6 | 1.16 | BB825002 | Q7TPP9 /// Q7TQE0 /// Q8C145 /// Q8R518 |
| 1453188_at | 6230424C14Rik | RIKEN cDNA 6230424C14 gene | 1.16 | AI553459 | — |
| 1427432_a_at | Sfrs10 | splicing factor, arginine/serine-rich 10 (transformer 2 | 1.16 | BM238387 | AAH61177 /// BAC33819 /// BAC36791 /// BAC37898 /// Q15815 |
| | | homolog, Drosophila) | | | |
| 1442809_at | — | Mus musculus transcribed sequences | 1.16 | BB452274 | Q62205 |
| 1417466_at | Rgs5 | regulator of G-protein signaling 5 | 1.16 | NM_133736 | BAC31773 /// BAC35655 /// O08850 /// Q99JV0 |
| 1454869_at | Wdr40b | WD repeat domain 40B | 1.16 | BB274776 | AAH68319 /// Q8C8E2 /// Q8CA30 /// Q8CAL3 /// Q8CBW4 /// Q8CBX8 |
| 1436432_at | B230343J05Rik | RIKEN cDNA B230343J05 gene | 1.16 | BM941461 | — |
| 1436997_x_at | Sh3bgrl | SH3-binding domain glutamic acid-rich protein like | 1.16 | BB248904 | Q8BHV4 /// Q9JJU8 |
| 1418046_at | Nap1l2 | nucleosome assembly protein 1-like 2 | 1.16 | NM_008671 | P51860 /// Q8K3R9 |
| 1450934_at | Eif4a2 | eukaryotic translation initiation factor 4A2 | 1.16 | BM240314 | BAC36372 /// P10630 /// Q8BTU6 |
| 1419599_s_at | Ms4a6d | membrane-spanning 4-domains, subfamily A, member | 1.16 | NM_026835 | — |
| | | 6D | | | |
| 1447977_x_at | — | — | 1.17 | C77009 | — |
| 1434106_at | Epm2alp1 | EPM2A (laforin) interacting protein 1 | 1.17 | AV340515 | Q80TS4 /// Q8VEH5 |
| 1429940_at | 8430414L16Rik | RIKEN cDNA 8430414L16 gene | 1.17 | BM935271 | Q8JZM7 |
| 1429177_x_at | Sox17 | SRY-box containing gene 17 | 1.17 | AK004781 | AAH60612 /// Q61473 |
| 1437635_at | Dcbld2 | discoidin, CUB and LCCL domain containing 2 | 1.17 | AW146002 | AAH66097 /// Q8BKI4 /// Q91ZH3 /// Q91ZV3 /// Q9D9K5 |
| 1438673_at | Slc4a7 | solute carrier family 4, sodium bicarbonate | 1.17 | AW555750 | Q8BTY2 /// Q8BWZ4 /// Q9JL09 |
| | | cotransporter, member 7 | | | |
| 1459713_s_at | AU040576 | expressed sequence AU040576 | 1.17 | AU040576 | AAH62959 /// Q8BHY3 /// Q8BI26 /// Q99JK1 |
| 1452700_s_at | 1110008P08Rik | RIKEN cDNA 1110008P08 gene | 1.17 | AK003597 | — |
| 1433885_at | — | Mus musculus, Similar to IQ motif containing GTPase | 1.17 | BM240173 | Q7TMU5 /// Q811L1 /// Q8BV47 /// Q8C9I3 |
| | | activating protein 2, clone IMAGE: 3596508, mRNA, | | | |
| | | partial cds | | | |
| 1437403_at | E130306M17Rik | RIKEN cDNA E130306M17 gene | 1.17 | BB308071 | Q8BJC5 |
| 1454084_a_at | Senp8 | SUMO/sentrin specific protease family member 8 | 1.18 | AK018606 | BAC33554 /// Q9D2Z4 |
| 1436325_at | Rora | RAR-related orphan receptor alpha | 1.18 | BB306272 | P51448 /// Q8BRL5 /// Q8C3F5 |
| 1423839_a_at | Btf3 | basic transcription factor 3 | 1.18 | BC008233 | AAH08233 /// AAH64010 /// Q64152 /// Q9D9L3 |
| 1438435_at | Phca | phytoceramidase, alkaline | 1.18 | BB329313 | Q8CIG2 /// Q9D099 |
| 1417974_at | Kpna4 | karyopherin (importin) alpha 4 | 1.18 | BF018653 | O35343 |
| 1452660_s_at | Klhl7 | kelch-like 7 (Drosophila) | 1.18 | AK012326 | Q8BUL5 /// Q8K2Z1 /// Q9CZP4 |
| 1456898_at | — | Mus musculus transcribed sequence with weak | 1.18 | AI426862 | P42669 /// Q00577 /// Q8C6E9 |
| | | similarity to protein pir: A36298 (H. sapiens) A36298 | | | |
| | | proline-rich protein PRB3M (null) - human (fragment) | | | |
| 1426205_at | Ppp1cb | protein phosphatase 1, catalytic subunit, beta isoform | 1.18 | M27073 | AAH46832 /// BAC40636 /// P37140 /// Q8C285 /// Q9DBY2 |
| 1425095_at | BC002059 | cDNA sequence BC002059 | 1.18 | BC002059 | — |
| 1433631_at | Eif5 | eukaryotic translation initiation factor 5 | 1.18 | BQ176989 | AAH56633 /// P59325 /// Q8BVV6 |
| 1416745_x_at | Uap1 | UDP-N-acetylglucosamine pyrophosphorylase 1 | 1.18 | NM_133806 | Q8BG76 /// Q8BXD6 /// Q8VD59 /// Q91YN5 |
| 1433897_at | Smc4l1 | SMC4 structural maintenance of chromosomes 4-like 1 | 1.19 | BQ176744 | — |
| | | (yeast) | | | |
| 1428113_at | 4930403J22Rik | RIKEN cDNA 4930403J22 gene | 1.19 | BB278364 | Q8BG19 /// Q8C4D2 /// Q8CAC3 /// Q8K0I2 /// Q9CS83 /// Q9D5P3 |
| 1436317_at | 9030223K07Rik | RIKEN cDNA 9030223K07 gene | 1.19 | BM115569 | — |
| 1439006_x_at | AW260253 | expressed sequence AW260253 | 1.19 | BB093996 | AAH62956 /// Q8BHS0 /// Q8BHV8 /// Q8BHW5 /// Q8BHZ6 |
| 1433903_at | AU021838 | expressed sequence AU021838 | 1.19 | BM227771 | — |
| 1439807_at | B230382K22Rik | RIKEN cDNA B230382K22 gene | 1.19 | BB816169 | Q8BQU7 |
| 1455604_at | — | Mus musculus transcribed sequences | 1.19 | BB795235 | — |
| 1434659_at | 5830411G16Rik | RIKEN cDNA 5830411G16 gene | 1.20 | BB514213 | Q80U56 |
| 1434468_at | 4930431L18Rik | RIKEN cDNA 4930431L18 gene | 1.20 | BM238914 | Q80TL3 /// Q9CUN2 |
| 1434955_at | 2900024D24Rik | RIKEN cDNA 2900024D24 gene | 1.20 | BB134696 | AAH66008 // Q8C294 /// Q8CBA1 |
| 1453481_at | Zdhhc2 | zinc finger, DHHC domain containing 2 | 1.20 | BB342242 | P59267 |
| 1450938_at | Pnn | pinin | 1.20 | AV135835 | O35691 /// Q8CD89 /// Q9CV89 |
| 1436387_at | C330006P03Rik | RIKEN cDNA C330006P03 gene | 1.20 | BB398124 | — |
| 1456599_at | Nxt2 | nuclear transport factor 2-like export factor 2 | 1.20 | BB745947 | AAH64727 /// AAH68166 /// Q8C070 |
| 1448936_at | Stx12 | syntaxin 12 | 1.20 | BC010669 | Q9ER00 |
| 1428777_at | Spred1 | sprouty protein with EVH-1 domain 1, related sequence | 1.20 | AK017680 | AAH57874 /// Q924S8 |
| 1429771_at | 3110073H01Rik | RIKEN cDNA 3110073H01 gene | 1.21 | AK014252 | — |
| 1452766_at | 2900041A09Rik | RIKEN cDNA 2900041A09 gene | 1.21 | AK013631 | Q7TQD2 |
| 1420859_at | Pkia | protein kinase inhibitor, alpha | 1.21 | AK010212 | AAH48244 /// P27776 |
| 1458704_at | — | Mus musculus transcribed sequences | 1.21 | AI452119 | — |
| 1450642_at | 3110001I20Rik | RIKEN cDNA 3110001I20 gene | 1.21 | NM_133725 | — |
| 1443858_at | 1110039I09Rik | RIKEN cDNA 1110039I09 gene | 1.21 | BI653857 | Q99PP3 /// Q99PP4 /// Q99PP5 /// Q99PP6 |
| 1436719_at | Slc35f1 | solute carrier family 35, member F1 | 1.21 | BB758319 | AAH59075 /// Q8BGK5 /// Q8BKD4 /// Q8BX52 |
| 1429131_at | Ube2v2 | ubiquitin-conjugating enzyme E2 variant 2 | 1.21 | AV010904 | AAH58374 /// Q8BGH6 /// Q8CE99 /// Q8K2V7 /// Q9CYD7 /// Q9D2M8 /// Q9ERI8 |
| 1433761_at | 9430063L05Rik | RIKEN cDNA 9430063L05 gene | 1.21 | AV374669 | Q80U00 /// Q80YT7 /// Q8BKQ2 /// Q8C9H5 /// Q8K240 |
| 1428592_s_at | Usp38 | ubiquitin specific protease 38 | 1.21 | BG064874 | BAC98274 /// Q8BW70 |
| 1450870_at | Rala | v-ral simian leukemia viral oncogene homolog A (ras | 1.22 | BG073338 | AAG23136 /// AAH31741 /// P05810 /// Q9CXY0 |
| | | related) | | | |
| 1419971_s_at | Slc35a5 | solute carrier family 35, member A5 | 1.22 | C86506 | Q921R7 /// Q9DC72 |
| 1416426_at | Rab5a | RAB5A, member RAS oncogene family | 1.22 | NM_025887 | Q8BPE8// Q9CQD1 |
| 1441598_at | Tmeff2 | transmembrane protein with EGF-like and two follistatin- | 1.22 | AV246773 | Q8CDH1 /// Q9JJE3 /// Q9QYM9 |
| | | like domains 2 | | | |
| 1422769_at | Syncrip | synaptotagmin binding, cytoplasmic RNA interacting | 1.22 | BG920261 | Q7TMK9 |
| | | protein | | | |
| 1428915_at | Sirt5 | sirtuin 5 (silent mating type information regulation 2 | 1.22 | AK002609 | Q8K2C6 |
| | | homolog) 5 (S. cerevisiae) | | | |
| 1427185_at | Mef2a | myocyte enhancer factor 2A | 1.22 | AV255689 | AAH61128 |
| 1434039_at | Appbp2 | amyloid beta precursor protein (cytoplasmic tail) binding | 1.22 | BB553604 | Q80U61 /// Q9CUT5 /// Q9DAX9 |
| | | protein 2 | | | |
| 1416744_at | Uap1 | UDP-N-acetylglucosamine pyrophosphorylase 1 | 1.22 | NM_133806 | Q8BG76 /// Q8BXD6 /// Q8VD59 /// Q91YN5 |
| 1455083_at | A330005H02Rik | RIKEN cDNA A330005H02 gene | 1.22 | BG068357 | — |
| 1429167_at | 8430438M01Rik | RIKEN cDNA 8430438M01 gene | 1.23 | BM221159 | — |
| 1438221_at | C130065N10Rik | RIKEN cDNA C130065N10 gene | 1.23 | AI875682 | — |
| 1455009_at | Cpd | carboxypeptidase D | 1.23 | AW550842 | O89001 |
| 1435504_at | — | Mus musculus transcribed sequences | 1.23 | BM217861 | Q8BW09 /// Q8CI96 /// Q921Q4 /// Q9D2S6 |
| 1436816_at | — | Mus musculus cDNA clone IMAGE: 6839226, | 1.23 | BB559624 | Q8CDZ5 /// Q8R0G9 |
| | | partial cds | | | |
| 1417489_at | Npy2r | neuropeptide Y receptor Y2 | 1.23 | NM_008731 | P97295 /// Q8BWV1 |
| 1423535_at | Strn3 | striatin, calmodulin binding protein 3 | 1.23 | BF148627 | Q9ERG2 |
| 1455324_at | — | Mus musculus 15 days embryo head cDNA, RIKEN full- | 1.24 | BQ176176 | — |
| | | length enriched library, clone: D930035P11 | | | |
| | | product: unknown EST, full insert sequence | | | |
| 1415861_at | Tyrp1 | tyrosinase-relaled protein 1 | 1.24 | BB762957 | P07147 |
| 1447757_x_at | Inpp5f | inositol polyphosphate-5-phosphatase F | 1.24 | AV033355 | AAH67200 /// BAC98059 /// Q8C8G7 /// Q8CBW2 /// Q8CDA1 |
| 1416151_at | Sfrs3 | splicing factor, arginine/serine-rich 3 (SRp20) | 1.24 | NM_013663 | AAH68111 /// BAC37445 /// P23152 /// Q8C3H6 /// Q9D8W4 |
| 1428437_at | 2700023B17Rik | RIKEN cDNA 2700023B17 gene | 1.25 | BI662680 | Q8K2F8 /// Q9CTG8 |
| 1443869_at | E430028B21Rik | RIKEN cDNA E430028B21 gene | 1.25 | BM114886 | AAH64450 /// Q8BKH8 /// Q8BTS8 /// Q8BUQ5 /// Q8C3G9 /// Q8CB54 |
| 1435990_at | Adamts2 | a disintegrin-like and metalloprotease (reprolysin type) | 1.25 | BG073461 | Q8C9W3 |
| | | with thrombospondin type 1 motif, 2 | | | |
| 1449176_a_at | Dck | deoxycytidine kinase | 1.25 | NM_007832 | BAB23394 /// BAB27131 /// BAC33307 /// BAC40203 /// P43346 /// Q80US6 |
| 1429335_at | Snapc1 | small nuclear RNA activating complex, polypeptide 1 | 1.25 | AK012317 | Q8K0S9 |
| 1450664_at | Gabpa | GA repeat binding protein, alpha | 1.26 | NM_008065 | Q00422 /// Q7TT22 /// Q91YY8 /// Q9CT91 |
| 1424683_at | 1810015C04Rik | RIKEN cDNA 1810015C04 gene | 1.26 | BC019494 | Q7TMY5 /// Q8VE91 /// Q9CUJ4 /// Q9D8Z5 |
| 1442077_at | 2310076G05Rik | RIKEN cDNA 2310076G05 gene | 1.26 | BB197581 | — |
| 1451077_at | Rpl5 | ribosomal protein L5 | 1.26 | BM114165 | P47962 |
| 1425662_s_at | Trnt1 | tRNA nucleotidyl transferase, CCA-adding, 1 | 1.27 | BM225164 | Q8K1J6 |
| 1435286_at | AW125296 | expressed sequence AW125296 | 1.27 | BB304438 | — |
| 1433585_at | Tnpo1 | transportin 1 | 1.27 | BI696984 | Q8BFY9 |
| 1455602_x_at | 1190030G24 | hypothetical protein 1190030G24 | 1.27 | AV023018 | Q8BNM1 /// Q8C4R5 |
| 1455607_at | Thsd2 | thrombospondin, type I, domain 2 | 1.27 | BG072958 | Q8BVW2 /// Q9CSB2 |
| 1422729_at | Pcdhb10 | protocadherin beta 10 | 1.27 | NM_053135 | Q91VE5 |
| 1416814_at | Tia1 | cytotoxic granule-associated RNA binding protein 1 | 1.28 | BG518542 | BAC40385 /// P52912 /// Q80ZW7 /// Q8BT02 /// Q8CII5 |
| 1417716_at | Got2 | glutamate oxaloacetate transaminase 2, mitochondrial | 1.28 | U82470 | P05202 |
| 1423195_at | Hiat1 | hippocampus abundant gene transcript 1 | 1.28 | BM208682 | P70187 /// Q9DBS0 |
| 1435014_at | Rab39b | RAB39B, member RAS oncogene family | 1.28 | AV162168 | Q8BHC1 |
| 1457707_at | — | Mus musculus transcribed sequences | 1.29 | BB817942 | — |
| 1448176_a_at | Hnrpk | heterogeneous nuclear ribonucleoprotein K | 1.29 | NM_025279 | Q07244 /// Q8BGQ8 |
| 1442226_at | Sema3e | sema domain, immunoglobulin domain (Ig), short basic | 1.29 | AV348197 | AAH57956 /// BAC33823 /// BAC97926 /// P70275 /// Q8CCK6 /// Q9QX23 |
| | | domain, secreted, (semaphorin) 3E | | | |
| 1427915_s_at | Tceb1 | transcription elongation factor B (SIII), polypeptide 1 | 1.29 | AI019214 | Q63182 |
| 1425994_a_at | Asah2 | N-acylsphingosine amidohydrolase 2 | 1.29 | AB037111 | Q8BNP0 /// Q8BQN7 /// Q8R236 /// Q9JHE3 |
| 1440357_at | — | Mus musculus transcribed sequences | 1.29 | BM938290 | — |
| 1418843_at | Slc30a4 | solute carrier family 30 (zinc transporter), member 4 | 1.29 | NM_011774 | O35149 |
| 1438666_at | AI194318 | expressed sequence AI194318 | 1.29 | BB534423 | Q8CCS0 /// Q8CDR7 |
| 1435060_at | — | Mus musculus transcribed sequences | 1.30 | BB667124 | AAH61124 /// Q8BGX9 /// Q9CUK4 /// Q9JKK7 |
| 1434294_at | — | Mus musculus adult male corpora quadrigamina cDNA, | 1.30 | BB183166 | Q8K2D0 |
| | | RIKEN full-length enriched library, clone: B230361M20 | | | |
| | | product: unknown EST, full insert sequence | | | |
| 1449664_s_at | Rnf20 | ring finger protein 20 | 1.30 | AW540162 | Q7TT11 /// Q8BKA8 /// Q8BKN8 /// Q8BUF7/// Q8BVU4 |
| 1429691_at | 5430405N12Rik | RIKEN cDNA 5430405N12 gene | 1.30 | AK017277 | — |
| 1435862_at | Son | Son cell proliferation protein | 1.30 | BG067046 | Q80TM4 /// Q811G3 /// Q8BM30 /// Q8BS91 /// Q8C9T5 /// Q9QX47 |
| 1450394_at | Golph3 | golgi phosphoprotein 3 | 1.30 | AV174110 | Q99KY1 /// Q9CRA5 |
| 1427682_a_at | Egr2 | early growth response 2 | 1.30 | X06746 | P08152 |
| 1438592_at | — | Mus muaculus 12 days embryo spinal cord cDNA, | 1.30 | BB418199 | — |
| | | RIKEN full-length enriched library, clone: C530008K05 | | | |
| | | product: unclassifiable, full insert sequence | | | |
| 1460303_at | Nr3c1 | nuclear receptor subfamily 3, group C, member 1 | 1.30 | NM_008173 | P06537 |
| 1437154_at | 4933426L22Rik | RIKEN cDNA 4933426L22 gene | 1.30 | BB667247 | Q8BJW2 /// Q9D3Z0 |
| 1450387_s_at | Ak4 | adenylate kinase 4 | 1.30 | NM_009647 | Q9WUR9 |
| 1455196_s_at | AA987161 | expressed sequence AA987161 | 1.31 | AA987127 | Q80VN4 |
| 1434585_at | Fbl | fibrillarin | 1.31 | BB667130 | — |
| 1455123_at | St18 | suppression of tumorlgenicity 18 | 1.31 | BB178719 | Q80TY4 /// Q811B4 /// Q8K098 |
| 1448140_at | Ciapin1 | cytokine induced apoptosis inhibitor 1 | 1.31 | NM_134141 | AAS09959 /// Q8VC24 /// Q8WTY4 |
| 1426827_at | A730098D12Rik | RIKEN cDNA A730098D12 gene | 1.31 | AV025877 | Q80V25 /// Q8C4W4 /// Q8R5E6 |
| 1452261_at | Shprh | SNF2 histone linker PHD RING helicase | 1.31 | BC006883 | Q7TPQ3 /// Q7TQ27 /// Q7TQ28 /// Q7TQ29 /// Q8BKE2 /// Q8BUW0 /// Q8BXM1 /// Q922Q3 |
| 1462761_a_at | 8430436O14Rik | RIKEN cDNA 8430436O14 gene | 1.31 | AK018466 | — |
| 1418816_at | 2810405l11Rik | RIKEN cDNA 2810405l11 gene | 1.31 | BG073376 | Q99LU0 /// Q9CXR5 |
| 1428586_at | D7Ertd743e | DNA segment, Chr 7, ERATO Doi 743, expressed | 1.31 | BB823331 | Q8C1X2 /// Q8CBS5 /// Q8CBU5 /// Q8K1A5 |
| 1439295_x_at | 9930105H17Rik | RIKEN cDNA 9930105H17 gene | 1.31 | BB371300 | — |
| 1459882_at | Asf1a | ASF1 anti-silencing functon 1 homolog A (S. cerevisiae) | 1.32 | AV312905 | Q9CQE6 |
| 1420376_a_at | H3f3b | H3 histone, family 3B | 1.32 | NM_008211 | AAH37730 /// BAB22464 /// BAC40130 /// P08351 /// Q8VDJ2 /// Q9D0H3 |
| 1453059_at | 2310046A06Rik | RIKEN cDNA 2310046A06 gene | 1.32 | AK009836 | Q9D6X9 |
| 1418428_at | Kif5b | kinesin family member 5B | 1.33 | BI328541 | Q61768 /// Q8CFE7 /// Q9CUT6 |
| 1456130_at | — | Mus musculus 15 days embryo head cDNA, RIKEN full- | 1.33 | BG068705 | — |
| | | length enriched library, clone: D930002l12 | | | |
| | | product: unclassifiable, full insert sequence | | | |
| 1455142_at | A730004F22Rik | RIKEN cDNA A730004F22 gene | 1.33 | BB244749 | — |
| 1430667_at | Pcdh10 | protocadherin 10 | 1.33 | BB077413 | AAH65695 /// Q80TE2 /// Q8CA99 /// Q8CC37 /// Q925I8 /// Q9CU33 /// Q9Z1B1 |
| 1416653_at | Stxbp3 | syntaxin binding protein 3 | 1.33 | NM_011504 | AAH62901 /// Q60770 /// Q8C7H4 |
| 1422569_at | Yy1 | YY1 transcription factor | 1.34 | BI665246 | Q00899 /// Q8C6B5 |
| 1429062_at | Kif16b | kinesin family member 16B | 1.34 | BG066903 | BAC98211 /// O35056 /// Q8BZZ9 |
| 1434272_at | Cpeb2 | cytoplasmic polyadenylation element binding protein 2 | 1.34 | AV231491 | Q812E0 |
| 1435050_at | — | Mus musculus 10 days neonate cerebellum cDNA, | 1.34 | BB353607 | — |
| | | RIKEN full-length enriched library, clone: B930094H20 | | | |
| | | product: unknown EST, full Insert sequence | | | |
| 1434108_at | Fbxo11 | F-box only protein 11′ | 1.34 | BM250164 | Q7TPD1 |
| 1458841_at | — | Mus musculus transcribed sequences | 1.34 | BB499674 | — |
| 1439779_at | — | Mus musculus 16 days neonate cerebellum cDNA, | 1.34 | BB356939 | — |
| | | RIKEN full-length enriched library, clone: 9630050J22 | | | |
| | | product: unknown EST, full insert sequence | | | |
| 1438223_at | — | — | 1.35 | BG065705 | — |
| 1437995_x_at | — | septin 7 | 1.35 | AV219419 | O55131 /// Q8C2A3 |
| 1455173_at | Gspt1 | G1 to phase transition 1 | 1.35 | AW537663 | Q8BPH0 /// Q8CAS6 /// Q8CCV1 /// Q8K2E1 /// Q8R050 |
| 1445642_at | 4930540I23Rik | RIKEN cDNA 4930540I23 gene | 1.35 | AV156411 | — |
| 1451064_a_at | Psat1 | phosphoserine aminotransferase 1 | 1.35 | BC004627 | BAC33959 /// Q8BTJ1 /// Q99JU9 /// Q99K85 |
| 1415963_at | Hnrph2 | heterogeneous nuclear ribonucleoprotein H2 | 1.35 | NM_019868 | P70333 |
| 1439397_at | — | Mus musculus transcribed sequences | 1.36 | BB164513 | — |
| 1452784_at | Itgav | integrin alpha V | 1.36 | AK003416 | P43406 /// Q80Y67 |
| 1451301_at | Tmod2 | tropomodulin 2 | 1.36 | BB633110 | AAH61124 /// Q8BGX9 /// Q9CUK4 /// Q9JKK7 |
| 1430651_s_at | Zfp191 | zinc finger protein 191 | 1.36 | AI504586 | Q8C2B8 /// Q91VN1 |
| 1416613_at | Cyp1b1 | cytochrome P450, family 1, subfamily b, polypeptide 1 | 1.36 | BI251808 | Q64429 /// Q80V82 /// Q8BRY0 /// Q8C685 /// Q9CUA1 |
| 1454612_at | Rkhd2 | ring finger and KH domain containing 2 | 1.36 | BI656279 | — |
| 1448405_a_at | Cri1 | CREBBP/EP300 inhibitory protein 1 | 1.37 | BC010712 | Q8BP25 /// Q9CQ17 /// Q9CYM0 /// Q9CZL9 /// Q9DCR4 |
| 1427418_a_at | Hif1a | hypoxia Inducible factor 1 alpha subunit | 1.37 | X95580 | Q61221 |
| 1421052_a_at | Sms | spermine synthase | 1.37 | NM_009214 | AAH58688 /// P97355 /// Q8C7P4 /// Q9CT09 |
| 1415688_at | Ube2g1 | ubiquitin-conjugating enzyme E2G 1 (UBC7 homolog, | 1.37 | NM_025985 | Q99462 |
| | | C. elegans) | | | |
| 1440066_at | — | Mus musculus transcribed sequences | 1.38 | BB531653 | — |
| 1428804_at | Mfap3l | microfibrillar-associated protein 3-like | 1.38 | AK017269 | Q80TV6 /// Q9D3X9 |
| 1419754_at | Myo5a | myosin Va | 1.38 | NM_010864 | Q99104 |
| 1448943_at | Nrp | neuropilin | 1.39 | AK011144 | AAH60129 /// P97333 /// Q80X28 |
| 1418501_a_at | Oxr1 | oxidation resistance 1 | 1.39 | AW548944 | Q8C715 /// Q99L06 /// Q99MK1 /// Q99MP4 |
| 1437734_at | Ppp1r12a | protein phoephatese 1, regulatory (Inhibitor) subunit | 1.39 | AV300184 | Q9DBR7 |
| | | 12A | | | |
| 1434075_at | MGC40669 | hypothetical protein MGC40669 | 1.39 | AV374294 | Q8C784 /// Q8K0V1 |
| 1423821_at | 8430437G11Rik | RIKEN cDNA 8430437G11 gene | 1.39 | BC007160 | Q91VX9 |
| 1435120_at | — | Mus musculus transcribed sequences | 1.39 | AV300631 | — |
| 1441928_x_at | EII | elongation factor RNA polymerase II | 1.39 | BB139475 | AAH24894 /// O08856 |
| 1450121_at | — | Mus musculus sodium channel 27 mRNA fragment. | 1.39 | AV336781 | Q62206 |
| 1426252_at | 1190006E07Rik | RIKEN cDNA 1190006E07 gene | 1.39 | AA881383 | Q80UZ4 /// Q8BJF9 /// Q9CT65 |
| 1423297_at | Add3 | adducin 3 (gamma) | 1.40 | BM239642 | Q8BJH2 /// Q8BM29 /// Q8JZT6 /// Q9JLE2 /// Q9QYB5 |
| 1417074_at | Ceacam10 | CEA-related call adhesion molecule 10 | 1.40 | NM_007675 | Q61400 /// Q99LD6 /// Q9D329 |
| 1451179_a_at | QK | quaking | 1.40 | AF090403 | AAH56346 /// O8B972 /// Q61110 /// Q61111 /// Q9CW34 /// Q9QUH4 /// Q9QYS9 /// Q9Z246 |
| 1439249_at | A230035H12Rik | RIKEN cDNA A230035H12 gene | 1.41 | BB822150 | — |
| 1427083_a_at | Map4k5 | mitogen-activated protein kinase kinase kinase kinase 5 | 1.41 | BG067961 | AAH57930 /// Q8BPM2 /// Q8BRE4 |
| 1444615_x_at | Cbfa2t1h | CBFA2T1 identified gene homolog (human) | 1.42 | AV327778 | Q61909 /// Q8C066 |
| 1435251_at | Snx13 | sorting nexin 13 | 1.42 | AV377013 | AAH56394 /// AAH67201 /// Q80TT7 |
| 1456026_at | 8030451K01Rik | RIKEN cDNA 8030451K01 gene | 1.42 | AV303159 | Q8CCH2 /// Q8CDW5 |
| 1456596_at | 6430550H21Rik | RIKEN cDNA 6430550H21 gene | 1.42 | BB093996 | AAH62956 /// Q8BHS0 /// Q8BHV8 /// Q8BHW5 /// Q8BHZ6 |
| 1424752_x_at | Zfp71-rs1 | zinc finger protein 71, related sequence 1 | 1.42 | BC016248 | Q60915 /// Q8BY64 /// Q91W94 |
| 1433740_at | 2610301K12Rik | RIKEN cDNA 2610301K12 gene | 1.43 | BG070008 | Q8BKU8 /// Q8K0G0 /// Q9D001 |
| 1429639_at | 2310032D16Rik | RIKEN cDNA 2310032D16 gene | 1.43 | AK009137 | Q80TD5 /// Q8BKJ7 /// Q8BKW7 /// QBC0L9 /// Q8CFW2 /// Q9D759 |
| 1424717_at | Misc12 | MIS12 homolog (yeast) | 1.43 | BC026790 | Q9CY25 |
| 1437087_at | — | Mus musculus 2 days neonate thymus thymic cells | 1.43 | AV079268 | — |
| | | cDNA, RIKEN full-length enriched library, | | | |
| | | clone: C920025L08 product: hypothetical RNI-like | | | |
| | | structure containing protein, full insert sequence | | | |
| 1434172_at | Cnr1 | cannabinoid receptor 1 (brain) | 1.44 | BQ177934 | — |
| 1437137_at | AW260253 | expressed sequence AW260253 | 1.44 | AV280875 | AAH62956 /// Q8BHS0 /// Q8BHV8 /// Q8BHW5 /// Q8BHZ6 |
| 1429642_at | Anubl1 | AN1, ubiquitin-like, homolog (Xenopus laevis) | 1.44 | AK012639 | Q80ZS6 |
| 1418058_at | Eltd1 | EGF, latrophilin seven transmembrane domain | 1.45 | BC017134 | Q923X1 |
| | | containing 1 | | | |
| 1435132_at | Disp1 | dispatched homolog 1 (Drosophila) | 1.45 | AI505698 | AAH59225 /// Q80ZZ8 /// Q8CGS3 /// Q8CIP6 /// Q8CIQ9 /// Q9CT62 |
| 1434179_at | Mll3 | myeloid/lymphoid or mixed-lineage leukemia 3 | 1.45 | AV297525 | BAC98187 /// Q8BRH4 /// Q8BZX5 |
| 1423042_at | Fin14 | fibroblast growth factor inducible 14 | 1.46 | BF123067 | — |
| 1438606_a_at | Clic4 | chloride intracellular channel 4 (mitochondrial) | 1.46 | BB814844 | BAC33601 /// Q9QYB1 |
| 1449893_a_at | Lrig1 | leucine-rich repeats and immunoglobulin-like domains 1 | 1.46 | NM_008377 | P70193 |
| 1421849_at | Stag2 | stromal antigen 2 | 1.46 | NM_021465 | AAH66041 /// O35638 /// Q8BSB5 |
| 1434677_at | Hps5 | Hermansky-Pudlak syndrome 5 homolog (human) | 1.46 | BG067097 | BAC98075 /// P59438 |
| 1456699_s_at | A730098D12Rik | RIKEN cDNA A730098D12 gene | 1.48 | AA561825 | Q80V25 /// Q8C4W4 /// Q8R5E6 |
| 1433847_at | D330017J20Rik | RIKEN cDNA D330017J20 gene | 1.48 | BB098407 | Q80TI6 /// Q8C7A2 /// Q8C9H6 |
| 1420429_at | Pcdhb3 | protocadherin beta 3 | 1.48 | NM_053128 | Q91XZ7 /// Q925M6 |
| 1431748_a_at | 1700051E09Rik | RIKEN cDNA 1700051E09 gene | 1.48 | AK015806 | AAO42677 /// BAC87665 /// Q9D543 /// Q9D9B1 |
| 1450937_at | Lin7o | lin 7 homolog c (C. elegans) | 1.48 | BQ176612 | O88952 /// Q99KF6 |
| 1455995_at | D10Bwg1379e | DNA segment, Chr 10, Brigham & Women's Genetics | 1.49 | BB125269 | Q80TH0 |
| | | 1379 expressed | | | |
| 1419589_at | C1qr1 | complement component 1, q subcomponent, receptor 1 | 1.49 | BB039247 | BAC37518 /// O89103 /// Q8C5P4 |
| 1417768_at | 1200006O19Rik | RIKEN cDNA 1200006O19 gene | 1.49 | BC019364 | Q8K1N1 /// Q8VEC0 /// Q9CVC9 /// Q9DC20 |
| 1456485_at | Npat | nuclear protein in the AT region | 1.50 | BM207451 | Q8BMA5 /// Q8BWA9 /// Q8BY06 |
| 1428333_at | 6530401D17Rik | RIKEN cDNA 6530401D17 gene | 1.50 | AK013740 | Q8BK31 /// Q9D365 |
| 1457424_at | Eya1 | eyes absent 1 homolog (Drosophila) | 1.50 | BB760085 | AAH60260 /// AAH66860 /// P97767 /// Q8C9D0 |
| 1439968_x_at | — | Mus musculus adult male corpora quadrigemina cDNA, | 1.50 | BE949296 | — |
| | | RIKEN full-length enriched library, clone: B230215D24 | | | |
| | | product: unknown EST, full insert sequence | | | |
| 1426585_s_at | Mapk1 | mitogen activated protein kinase 1 | 1.51 | BM209765 | AAH58258 /// BAC29053 /// BAC33251 /// BAC40044 /// P27703 /// Q922X7 /// Q9D319 |
| 1420895_at | Tgfbr1 | transforming growth factor, beta receptor 1 | 1.51 | BM248342 | Q64729 /// Q9CVP4 |
| 1440177_at | — | hypothetical protein 9630027E11 | 1.51 | BM899529 | — |
| 1451652_a_at | 5033428A16Rik | RIKEN cDNA 5033428A16 gene | 1.51 | BC018498 | — |
| 1439618_at | Pde10a | phosphodiesterase 10A | 1.51 | AI448308 | Q8C8M0 /// Q8CA95 /// Q9WVI1 |
| 1418500_at | Nap1l3 | nucleosome assembly protein 1-like 3 | 1.53 | NM_138742 | O54802 |
| 1430187_at | 6330516O17Rik | RIKEN cDNA 6330516O17 gene | 1.53 | AK018216 | Q9D388 |
| 1455258_at | AW047325 | expressed sequence AW047325 | 1.53 | BQ174236 | — |
| 1420609_at | Axot | axotrophin | 1.53 | NM_020575 | Q9WV66 |
| 1448147_at | Tnfrsf19 | tumor necrosis factor receptor superfamily, member 19 | 1.54 | NM_013869 | Q80T13 /// Q812G3 /// Q8BUM7 /// Q8BWR1 /// Q9JLL3 |
| 1415855_at | Kitl | kit ligand | 1.54 | BB815530 | P20826 /// Q61854 /// Q64384 |
| 1416174_at | Rbbp9 | retinoblastoma binding protein 9 | 1.54 | BC011107 | O88851 /// Q80YU9 |
| 1427898_at | Rnf6 | ring finger protein (C3H2C3 type) 6 | 1.55 | BI738010 | Q8K565 /// Q9DBU5 |
| 1434601_at | Amigo2-pending | amphoterin induced gene and ORF 2 | 1.55 | AV315087 | Q80ZD9 |
| 1440201_at | Mapk10 | mitogen activated protein kinase 10 | 1.56 | BB313689 | Q61831 /// Q80W80 /// Q80W81 /// Q80W82 /// Q8C9D4 |
| 1449322_at | Ptp4a1 | protein tyrosine phosphatase 4a1 | 1.56 | BC003761 | Q63739 |
| 1418488_s_at | Ankrd3 | ankyrin repeat domain 3 | 1.56 | AF302127 | AAH57871 /// Q9CV04 /// Q9ERK0 |
| 1418162_at | Tlr4 | toll-like receptor 4 | 1.56 | AF185285 | Q8K2T5 /// Q9QUK6 |
| 1416967_at | Sox2 | SRY-box containing gene 2 | 1.56 | U31967 | AAH57574 /// BAC75668 /// P48432 /// Q8CCY4 |
| 1437409_s_at | Gpr126 | G protein-coupled receptor 126 | 1.57 | BB812574 | Q811E4 |
| 1435768_at | Arid4b | AT rich interactive domain 4B (Rbp1 like) | 1.57 | AV371758 | Q8BMI8 /// Q8BV50 /// Q8BXV6 /// Q8BYA5 /// Q8BYB0 /// Q8R1E4 |
| 1438079_at | MGC60963 | hypothetical protein MGC60963 | 1.57 | AV290754 | Q80UU4 |
| 1427121_at | Fbxo4 | F-box only protein 4 | 1.58 | BF455337 | Q8CHQ0 /// Q99JG8 /// Q9D4Y5 |
| 1417600_at | Slc15a2 | solute carrier family 15 (H+/peptide transporter), | 1.58 | NM_021301 | Q80XC0 /// Q8VEK9 /// Q9CXC0 /// Q9JM03 |
| | | member 2 | | | |
| 1416444_at | Elovl2 | elongation of very long chain fatty acids (FEN1/Elo2, | 1.58 | NM_019423 | BAC26646 /// BAC32079 /// BAC34236 /// Q9JLJ4 |
| | | SUR4/Elo3, yeast)-like 2 | | | |
| 1427081_at | A630072M18Rik | RIKEN cDNA A630072M18 gene | 1.59 | BB246700 | — |
| 1452974_at | Nol8 | nucleolar protein 8 | 1.59 | AK017551 | Q80VB9 /// Q8CDJ7 /// Q9CUR0 |
| 1440527_at | — | Mus musculus transcribed sequences | 1.59 | BI440542 | — |
| 1451268_at | Tram1l1 | translocation associated membrane protein 1-like 1 | 1.59 | BC027120 | Q8C455 /// Q8C6X6 /// Q8QZR0 |
| 1423592_at | Rock2 | Rho-associated coiled-coil forming kinase 2 | 1.60 | BB761686 | P70336 /// Q8CC95 |
| 1454714_x_at | Phgdh | 3-phosphoglycerate dehydrogenase | 1.61 | AA561726 | Q61753 /// Q8C603 |
| 1444437_at | Usp34 | ubiquitin specific protease 34 | 1.61 | BB086152 | AAH63062 /// BAC97975 /// Q7TMJ6 /// Q8CCH0 |
| 1422032_a_at | Za20d3 | zinc finger, A20 domain containing 3 | 1.61 | NM_022985 | Q9DCH6 |
| 1427670_a_at | Tcf12 | transcription factor 12 | 1.61 | M97636 | Q61286 /// Q8BP24 /// Q8K1X3 |
| 1433571_at | A130038L21Rik | RIKEN cDNA A130038L21 gene | 1.61 | BQ175260 | AAH62131 /// Q80ZH8 /// Q8BHJ6 /// Q8CHM0 |
| 1435514_at | Lztfl1 | leucine zipper transcription factor-like 1 | 1.62 | BB700884 | Q8BRX8 /// Q8CDG8 /// Q8CDS2 /// Q9JHQ5 |
| 1434313_at | 6330407D12Rik | RIKEN cDNA 6330407D12 gene | 1.62 | BB762434 | Q8BIS8 |
| 1436662_at | — | Mus musculus transcribed sequences | 1.62 | BB022723 | — |
| 1417493_at | Bmi1 | B lymphoma Mo-MLV insertion region 1 | 1.63 | M64279 | P25916 |
| 1454783_at | I13ra1 | interleukin 13 receptor, alpha 1 | 1.63 | BI081033 | O09030 /// Q7TT27 /// Q8BNM4 /// Q8C1Z3 /// Q8VDP7 |
| 1456573_x_at | Nnt | nicotinamide nucleotide transhydrogenase | 1.63 | BB205930 | Q61941 /// Q8BGK0 /// Q8C1W8 /// Q8C3H2 /// Q8C9V5 /// Q922E1 /// Q9CTX5 |
| 1433779_at | — | Mus musculus, clone IMAGE: 5068832, mRNA, partial | 1.63 | AV311104 | AAH64446 /// AAR26704 /// AAR26705 /// Q8BQ39 /// Q8C4Z2 /// Q8K2M1 |
| | | cds | | | |
| 1425115_at | C030034J04Rik | RIKEN cDNA C030034J04 gene | 1.63 | BC025874 | Q8R399 |
| 1446899_at | — | Mus musculus transcribed sequence with weak | 1.64 | BB165801 | — |
| | | similarity to protein ref: NP_081764.1 (M. musculus) | | | |
| | | RIKEN cDNA 5730493B19 [Mus musculus] | | | |
| 1420907_at | Cd2ap | CD2-associated protein | 1.64 | BB398671 | Q9JLQ0 |
| 1436948_a_at | 6430550H21Rik | RIKEN cDNA 6430550H21 gene | 1.64 | BB520013 | AAH62956 /// Q8BHS0 /// Q8BHV8 /// Q8BHW5 /// Q8BHZ6 |
| 1448293_at | Ebf1 | early B-receptor 1 | 1.65 | BB125261 | Q07802 /// Q8BSM3 /// Q8C955 /// Q8CBL7 |
| 1437029_at | Tacr3 | tachykinin receptor 3 | 1.65 | AV328460 | AAH66845 /// P47937 |
| 1426895_at | Zfp191 | zinc finger protein 191 | 1.65 | BB579760 | Q8C2B8 /// Q91VN1 |
| 1454862_at | Phldb2 | pleckstrin homology-like domain, family B, member 2 | 1.66 | AV253284 | AAH60683 /// Q80Y16 /// Q8BKV3 /// Q8BZE3 /// Q8K1N2 |
| | | Mus musculus adult male corpora quadrigemina cDNA, | | | |
| | | RIKEN full-length enriched library, clone: B230215D24 | | | |
| 1456943_a_at | — | product: unknown EST, full insert sequence | 1.67 | BE949296 | — |
| 1426371_at | 3732409C05Rik | RIKEN cDNA 3732409C05 gene | 1.67 | BG094874 | Q8BZS2 /// Q922J9 /// Q9CXE8 /// Q9D0Q1 /// Q9DAU2 |
| 1428236_at | Acbd5 | acyl-Coenzyme A binding domain containing 5 | 1.67 | AK005001 | AAH61484 /// Q7TSC2 /// Q8BKU6 /// Q8CI99 /// Q9CW41 |
| 1428664_at | Vip | vasoactive intestinal polypeptide | 1.68 | AK018599 | Q9D2Z7 |
| 1436093_at | — | Mus musculus transcribed sequences | 1.68 | BE981269 | — |
| 1419552_at | Echdc1 | enoyl Coenzyme A hydratase domain containing 1 | 1.68 | NM_025855 | AAH66183 /// Q8C185 /// Q8R5A8 /// Q9CTC5 /// Q9CTK2 /// Q9CTM5 /// Q9D9V3 |
| 1415999_at | Hey1 | hairy/enhancer-of-split related with YRPW motif 1 | 1.68 | NM_010423 | Q9D0R0 /// Q9QUM5 /// Q9WV93 |
| | | dual-specificity tyrosine-(Y)-phosphorylation regulated | | | |
| 1424229_at | Dyrk3 | kinase 3 | 1.69 | BC006704 | Q8BM34 /// Q922Y0 |
| 1452224_at | Zcwcc3 | zinc finger, CW-type with coiled-coil domain 3 | 1.69 | BC026506 | Q8R0R0 |
| 1436590_at | Ppp1r3b | protein phosphatase 1, regulatory (inhibitor) subunit 3B | 1.70 | BG071940 | AAH60261 /// Q8C767 |
| 1419186_a_at | Siat8d | sialyltransferase 8 (alpha-2, 8-sialyltransferase) D | 1.70 | NM_009183 | AAH60112 /// CAA11685 /// Q64692 /// Q8BY70 |
| 1459722_at | 2900036G02Rik | RIKEN cDNA 2900036G02 gene | 1.71 | AI427602 | — |
| 1435588_at | Wdfy1 | WD40 and FYVE domain containing 1 | 1.71 | BQ031098 | Q8R3I5 /// Q9DAD3 |
| 1422449_s_at | Rcn2 | reticulocalbin 2 | 1.71 | NM_011992 | O70341 /// Q8BP39 /// Q8BP92 |
| 1427074_at | 5330414D10Rik | RIKEN cDNA 5330414D10 gene | 1.72 | BM117243 | Q8BHD8 |
| 1434298_at | Zfhx1b | zinc finger homeobox 1b | 1.73 | BQ174116 | AAH60699 /// Q80TX6 /// Q8BSG9 /// Q8CD37 /// Q9R0G7 |
| 1452281_at | Sos2 | Son of sevenless homolog 2 (Drosophila) | 1.74 | Z11664 | Q02384 |
| 1417183_at | Dnaja2 | DnaJ (Hsp40) homolog, subfamily A, member 2 | 1.75 | C77509 | BAC36946 /// BAC38809 /// Q9QYJ0 |
| 1422504_at | Glrb | glycine receptor, beta subunit | 1.75 | NM_010298 | BAC38831 /// P48168 |
| 1428091_at | Klhl7 | kelch-like 7 (Drosophila) | 1.76 | AK012326 | Q8BUL5 /// QBK2Z1 /// Q9CZP4 |
| 1426293_at | 6330581L23Rik | RIKEN cDNA 6330581L23 gene | 1.77 | BG068796 | Q80ZX2 /// Q91VW8 |
| 1434099_at | Casp7 | caspase 7 | 1.78 | BB752393 | Q8BRP2 /// Q8CCT1 |
| 1459750_s_at | Gpr123 | G protein-coupled receptor 123 | 1.79 | AU015577 | — |
| 1436223_at | 4832412O06Rik | RIKEN cDNA 4832412O06 gene | 1.81 | BB504737 | — |
| 1434434_s_at | Tcerg1 | transcription elongation regulator 1 (CA150) | 1.85 | AW557777 | Q8C490 /// Q8CGF7 /// Q8CHT8 /// Q9R0R5 |
| 1416950_at | Tnfalp8 | tumor necrosis factor, alpha-induced protein 8 | 1.85 | NM_134131 | Q8BTH4 /// Q921Z5 |
| 1455851_at | Bmp5 | bone morphogenetic protein 5 | 1.85 | AV032115 | P49003 /// Q8CCE0 |
| 1438752_at | A230058F20Rik | RIKEN cDNA A230058F20 gene | 1.90 | AV327739 | — |
| 1435162_at | Prkg2 | protein kinase, cGMP-dependent, type II | 1.90 | BB823350 | Q61410 /// Q8C4R2 /// Q8CAH8 |
| 1424123_at | BC011209 | cDNA sequence BC011209 | 1.92 | BC011209 | Q8BQK2 /// Q91X85 |
| 1459989_at | — | Mus musculus transcribed sequences | 1.93 | AV271189 | — |
| 1453064_at | 5730466H23Rik | RIKEN cDNA 5730466H23 gene | 1.95 | AK018594 | Q8BZM6 /// Q8K332 /// Q9CS19 /// Q9D2Z9 |
| 1460718_s_at | Mtch1 | mitochondrial carrier homolog 1 (C. elegans) | 1.95 | AF192558 | Q8CEY5 /// Q8R0T0 /// Q8R1T8 /// Q8R2T2 /// Q9QZA5 /// Q9QZP4 |
| 1442542_at | B130023L16Rik | RIKEN cDNA B130023L16 gene | 1.96 | BB363812 | — |
| 1416390_at | Chc1l | chromosome condensation 1-like | 2.00 | NM_134083 | Q8BMG2 /// Q99LJ7 |
| 1416958_at | Nr1d2 | nuclear receptor subfamily 1, group D, member 2 | 2.00 | NM_011584 | Q60674 /// Q8C598 /// Q8C6J1 /// Q8CCE4 /// Q922C3 |
| 1450757_at | Cdh11 | cadherin 11 | 2.02 | NM_009866 | P55288 /// Q8C7Q6 |
| 1418142_at | Kcnj8 | potassium inwardly-rectifying channel, subfamily J, | 2.03 | NM_008428 | P97794 |
| | | member 8 | | | |
| 1428582_at | 2010208K18Rik | RIKEN cDNA 2010208K18 gene | 2.06 | AK008476 | Q9D853 |
| 1423608_at | Itm2a | Integral membrane protein 2A | 2.06 | BI966443 | Q61500 /// Q8K0H4 /// Q9CRW4 |
| 1439795_at | Gpr64 | G protein-coupled receptor, 64 | 2.10 | AV242919 | Q8BLU3 /// Q8CJ12 |
| 1435338_at | 5830411I20 | hypothetical protein 5830411I20 | 2.11 | BM238926 | — |
| 1437967_at | — | Mus musculus transcribed sequences | 2.11 | AV365582 | — |
| 1435098_at | AI317395 | expressed sequence AI317395 | 2.14 | BF658992 | — |
| 1435233_at | Ncoa2 | nuclear receptor coactivator 2 | 2.20 | BM234716 | Q61026 /// Q7TPU7 /// Q8C961 /// Q8CBM5 /// Q8CE59 |
| 1452286_at | 5033405K12Rik | RIKEN cDNA 5033405K12 gene | 2.22 | BG081701 | BAC98175 /// Q8BHV0 /// Q8CI08 /// Q8VCP7 |
| 1433623_at | Zfp367 | zinc finger protein 367 | 2.34 | BE629588 | Q8BH90 /// Q8BI44 /// Q8BI53 /// Q8BI88 |
| 1450655_at | 2310035O07Rik | RIKEN cDNA 2310035O07 gene | 2.38 | AA214868 | BAC36545 /// BAC40525 /// O08586 /// Q8BJP9 /// Q8BSR7 |
| 1447854_s_at | Hist2h2bb | histone 2, H2bb | 2.44 | AV127319 | Q64524 |
| 1448573_a_at | Ceacam10 | CEA-related cell adhesion molecule 10 | 2.50 | NM_007675 | Q61400 /// Q99LD6 /// Q9D329 |

### REFERENCES

Grundy D. Neuroanatomy of visceral nociception: vagal and splanchnic afferent. Gut 2002; 51 Suppl 1:i2-i5.
Zhuo K Ichikawa H, Helke C J. Neurochemistry of the nodose ganglion. Prog Neurobiol 1997; 52:79-107.
Berthoud H R, Blackshaw L A, Brookes S J, Grundy D. Neuroanatomy of extrinsic afferents supplying the gastrointestinal tract. Neurogastroenterol Motil 2004; 16 Suppl 1:28-33.
Gebhart G F. Visceral pain-peripheral sensitisation. Gut 2000; 47 Suppl 4:iv54-iv55.
Ward S M, Bayguinov J, Won K J, Grundy D, Berthoud H R. Distribution of the vanilloid receptor (VR1) in the gastrointestinal tract. J Comp Neurol 2003; 465:121-135.
Dang K, Bielefeldt K, Gebhart G F. Gastric ulcers reduce A-type potassium currents in rat gastric sensory ganglion neurons. Am J Physiol Gastrointest Liver Physiol 2004; 286:G573-G579.
Mayer E A, Naliboff B D, Chang L, Coutinho S V. V. Stress and irritable bowel syndrome. Am J Physiol Gastrointest Liver Physiol 2001; 280:G519-G524.
Tache Y, Martinez V, Million M, Wang L. Stress and the gastrointestinal tract III. Stress-related alterations of gut motor function: role of brain corticotropin-releasing factor receptors. Am J Physiol Gastrointest Liver Physiol 2001; 280:0173-G177.
Mathison R, Davison J S. Capsaicin sensitive nerves in the jejunum of Nippostrongylus brasiliensis-sensitized rats participate in a cardiovascular depressor reflex. Naunyn Schmiedebergs Arch Pharmacol 1993; 348:638-642.
Camilleri M. Pathophysiology in irritable bowel syndrome. Drug News Perspect 2001; 14:268-278.
Stead R H. Nerve remodelling during intestinal inflammation. Ann N Y Acad Sci 1992; 664:443-455.
McLean P G, Picard C, Garcia-Villar R, Ducos dL, More J, Fioramonti J, Bueno L. Role of kinin B1 and B2 receptors and mast cells in post intestinal infection-induced hypersensitivity to distension. Neurogastroenterol Motil 1998; 10:499-508.
Gay J, Fioramonti J, Garcia-Villar R, Bueno L. Enhanced intestinal motor response to cholecystokinin in post-Nippostrongylus brasiliensis-infected rats: modulation by CCK receptors and the vagus nerve. Neurogastroenterol Motil 2001; 13:155-162.
Gay J, More J, Bueno L, Fioramonti J. CCK-induced Fos expression in brain stem is enhanced after intestinal nematode infection in rats. Brain Res 2002; 942:124-127.
Amaratunga D, Cabrera J. Analysis of data from viral DNA microchips. J Am Stat Assoc 2001; 96:1161-1170.
Wouters L, Göhlmann H W, Bijnens L, Kass S U, Molenberghs G, Lewi P J. Graphical exploration of gene expression data:a comparative study of three multivariate methods. Biometrics 2003; 59:1133-1141.
Tusher V G, Tibshirani R, Chu G. Significance analysis of microarrays applied to the ionizing radiation response. Proc Natl Acad Sci U S A 2001; 98:5116-5121.
Storey J D, Tibshirani R. Statistical significance for genomewide studies. Proc Natl Acad Sci USA 2003; 100:9440-9445.
http://faculty.washington.edu/˜jstorey/qvalue/manual.pdf.
   http://faculty.washington.edu/˜jstorey/qvalue/manual.pdf. 2004.
Strober W, Fuss I J, Blumberg R S. The immunology of mucosal models of inflammation. Annu. Rev. Immunol. 2002; 20:495-549
William C. Gause, Joseph F. Urban, Jr and Miguel J. Stadecker The immune response to parasitic helminths: insights from murine models. Trends in Immunology, Vol. 24, No. 5, May 2003
Mogil J S, Wilson S G, Bon K, Lee S E, Chung K, Raber P, Pieper J O, Hain H S, Belknap J K, Hubert L, Elmer G I, Chung J M, Devor M. Heritability of nociception II. ‘Types’ of nociception revealed by genetic correlation analysis. Pain. 1999 March; 80(1-2):83-93.
Elsenbruch S., Wang L., Hollerbach S., Schedlowski M. & Tougas G. Pseudo-affective visceromotor responses and HPA axis activation following colorectal distension in rats with increased cholinergic sensitivity. Neurogastroenterology and Motility 2004; 16:801-809
Yadid G, Nakash R, Deri L Tamar G, Kinor N, Gispan I, Zangen A. Elucidation of the neurobiology of depression: insights from a novel genetic animal model. Prog Neurobiol. 2000 November; 62(4):353-78.
Kamp E H, Jones R C 3rd, Tillman S R, Gebhart G F. Quantitative assessment and characterization of visceral nociception and hyperalgesia in mice. Am J Physiol Gastrointest Liver Physiol. 2003 March; 284(3):G434-444.
Holzer, P. Afferent signalling of gastric acid challenge. J Physiol Pharmacol 2003; 54(4),

1. A method of identifying a compound capable of reducing or preventing prolonged sensory neuron hyper-excitability comprising the steps of:
(a) administering a test compound to an experimental non-human animal having prolonged sensory neuron hyper-excitability;
(b) generating an expression profile of the genes modulated in the Nodose Ganglia (NG) of the animal of step (a);
(c) comparing the expression profile obtained in (b) with the expression profile of a corresponding panel of genes expressed in the NG of an experimental non-human animal having no prolonged sensory neuron hyper-excitability;
wherein a positive correlation of the expression profiles is indicative that the test compound is capable of reducing or preventing prolonged sensory neuron hyper-excitability in NG.

2. The method according to claim 1, wherein the modulated NG genes whose expression is to be compared comprise at least one gene selected from the group consisting of those genes listed in Table 1.

3. The method according to claim 1, wherein the modulated NG genes whose expression is to be compared comprise at least one gene selected from the group consisting the genes listed in Table 2.

4. The method according to claim 1, wherein the modulated genes expressed in the NG are compared at the nucleic acid level.

5. The method according to claim 1 wherein the modulated NG genes whose expression is to be compared comprise at least the vanilloid receptor VR1 (Trpv1), cholecystokinin receptor A (Cckar), serotonin receptor 3A (Htr3a) and somatostatin 2 receptor (Sstr2).

6. The method according to claim 1 wherein the method comprises comparing the expression of a panel of at least 40 genes selected from the group consisting of those genes listed in Table 1.

7. The method according to claim 1 wherein the method comprises comparing the expression of a panel of at least 51 genes comprising those genes listed in Table 2.

8. The method according to claim 1 wherein the expression profile of the NG genes is assessed at the transcript level or at the protein level.

9. The method according to claim 8 wherein the expression profile of the NG genes is assessed at the mRNA level.

10. The method according to claim 1, wherein at least 1 probe which hybridises to the NG modulated gene expression product is affixed to a solid support.

11. The method according to claim 10 wherein the probes are in an arrayed form.

12. A microarray comprising at least 1 nucleic acid probe immobilised on a solid support capable of hydridizing with an expression product of a gene modulating in NG neurons having prolonged sensory neuron hyper-excitability.

13. The microarray according to claim 12 comprising at least 40 nucleic acid probes capable of hybridizing to sequences selected from the group consisting of nucleic acid sequences representing genes from Table 1.

14. The microarray according to claim 12 comprising at least 40 nucleic acid probes capable of hybridizing to sequences selected from the group consisting of nucleic acid sequences representing genes from Table 2.

15. The method according to claim 1, wherein the experimental non-human animal is a rodent.

16. The method according to claim 15, wherein the rodent is a mouse.

17. The method according to claim 15, wherein the rodent is previously infected with a parasitic helminth selected from Table 3.

18. A method of treating a subject with a disease condition related to prolonged sensory neuron hyper-excitability, comprising administering to a subject an effective amount of an agent that modulates the expression or activity of one or more genes products selected from the group encoded by those genes listed in Table 1.

19. (canceled)

20. The method according to claim 18, wherein the agent modulates the expression or activity of one or more gene products selected from the group encoded by those genes listed in Table 2.

21. The method according to claim 18 wherein the agent modulates the expression or activity of one or more receptors selected from the group consisting of the vanilloid receptor VR1 (Trpv1), cholecystokinin receptor A (Cckar), serotonin receptor 3A (Htr3a) and somatostatin 2 receptor (Sstr2).

22. The method according to claim 18, wherein the disease condition associated with prolonged sensory neuron hyper-excitability is a gastrointestinal (GI) tract disorder or stress-related disorder.

23. The method according to claim 22, wherein the disease is a bowel disorder that is ulcerative colitis, Crohn's disease, ileitis, proctitis, celiacdisease, enteropathy associated with arthropathies, microscopic or collagenous colitis, eosinophilic gastroenteritis or pouchitis resulting after proctocolectomy, post ileoanal anastomosis, functional dyspepsia, functional vomiting, oesophagitis, gastric ulcer, duodenal ulcer, irritable bowel syndrome or depression.

24. The method according to claim 22, wherein the disease is irritable bowel syndrome.

25. A pharmaceutical composition for the treatment of a disease of disorder related to prolonged sensory neuron hyper-excitability comprising a compound identified by the method of claim 1 and at least one pharmaceutically acceptable diluent or excipient.

26-29. (canceled)

30. A method of making a pharmaceutical composition for the treatment of a disease or disorder related to prolonged sensory neuron hyper-excitability, comprising combining a compound identified according to the method of claim 1 with a pharmaceutically acceptable diluent or excipient.

31-36. (canceled)

37. A method of validating as pharmaceutical targets any one or more of the genes shown in Table 1 for the treatment of a G.I. tract disorder or stress-related disorder, comprising utilizing antisense nucleotides or gene silencing to block expression of said genes.

38. The method according to claim 37 wherein the gene silencing technique is siRNA.

39. The method according to claim 37 wherein the disorder is ulcerative colitis, Crohn's disease, ileitis, proctitis, celiac disease, enteropathy associated with arthropathies, microscopic or collagenous colitis, eosinophilic gastroenteritis or pouchitis resulting after proctocolectomy, post ileoanal anastomosis, functional dyspepsia, functional vomiting, oesophagitis, gastric ulcer, duodenal ulcer, irritable bowel syndrome or depression.

40-44. (canceled)

45. The method according to claim 17, wherein the rodent is previously infected with Nippostrongylus brasiliensis.

46. A method of making a pharmaceutical composition for the treatment of a disease or disorder related to prolonged sensory neuron hyper-excitability, comprising combining a compound having the modulating activity as defined in claim 20 with a pharmaceutically acceptable diluent or excipient.


SEQUENCE LISTING (15 sequences)

SEQ ID NO: 1 - DNA, 20 bp, Artificial Sequence
  Synthetic Primer
ctgggcaagggtggtaacat

SEQ ID NO: 2 - DNA, 28 bp, Artificial Sequence
  Synthetic Probe
cccaaggaaaactagcatgtgggactca

SEQ ID NO: 3 - DNA, 28 bp, Artificial Sequence
  Synthetic Primer
agttttggcattcaaagctacttattaa

SEQ ID NO: 4 - DNA, 21 bp, Artificial Sequence
  Synthetic Primer
tgtgctcgcttacagcatcac

SEQ ID NO: 5 - DNA, 26 bp, Artificial Sequence
  Synthetic Probe
ctggtcactctctggtccatttggca

SEQ ID NO: 6 - DNA, 20 bp, Artificial Sequence
  Synthetic Primer
ggctgtgcccactcaagaat

SEQ ID NO: 7 - DNA, 19 bp, Artificial Sequence
  Synthetic Primer
gctccaggcccagaacttg

SEQ ID NO: 8 - DNA, 21 bp, Artificial Sequence
  Synthetic Probe
ttgggacgctccttcctagct

SEQ ID NO: 9 - DNA, 22 bp, Artificial Sequence
  Synthetic Primer
ggcagtctctccacctctcagt

SEQ ID NO: 10 - DNA, 18 bp, Artificial Sequence
  Synthetic Primer
tccggagcggaagacatc

SEQ ID NO: 11 - DNA, 20 bp, Artificial Sequence
  Synthetic Probe
accaggtcacacccaggcaa

SEQ ID NO: 12 - DNA, 17 bp, Artificial Sequence
  Synthetic Primer
gccgggcagctgttttc

SEQ ID NO: 13 - DNA, 22 bp, Artificial Sequence
  Synthetic Primer
gcactgcaactgatctctccat

SEQ ID NO: 14 - DNA, 24 bp, Artificial Sequence
  Synthetic Probe
caagcgagagctcaggtttccttc

SEQ ID NO: 15 - DNA, 19 bp, Artificial Sequence
  Synthetic Primer
gctcttgtgtggcctgcat